(19) United States
(12) Patent Application Publication
(10) Pub. No.: US 2021/0269862 A1
(43) Pub. Date: Sep. 2, 2021

(54) METHODS FOR ASSESSING ENDOMETRIAL TRANSFORMATION

(71) Applicants: IGENOMIX, S.L., Valencia (ES); THE BOARD OF TRUSTEES OF THE LELAND STANFORD JUNIOR UNIVERSITY, Stanford, CA (US); CHAN ZUCKERBERG BIOHUB, INC., San Francisco, CA (US)

(72) Inventors: Stephen R. QUAKE, San Francisco, CA (US); Carlos SIMON, Valencia (ES); Wanxin WANG, Stanford, CA (US); Felipe VILELLA, Valencia (ES)

(21) Appl. No.: 17/254,078

(22) PCT Filed: Jun. 18, 2019
(86) PCT No.: PCT/US2019/037814

§ 371 Date: Dec. 18, 2020

Related U.S. Application Data
(60) Provisional application No. 62/686,621, filed on Jun. 18, 2018.

(51) Int. Cl. C12Q 1/6816; C12Q 1/686

(52) U.S. Cl. CPC C12Q 1/6816; C12Q 2600/158; C12Q 1/686

(57) ABSTRACT

The present Application provides in one aspect a method of diagnosing a menstrual cycle event in a subject (e.g., a WOI), comprising detecting in a biological sample a gene signature for one or more endometrial cell types (e.g., unciliated epithelial cells). The present Application in another aspect provides a method comprising determining a gene expression profile in each of a plurality of endometrial cells, wherein said endometrial cells are: (a) in an endometrial sample obtained from a subject, and (b) unciliated epithelial cells. In still another aspect, the present Application provides a method for detecting that a subject is within a window of implantation (WOI), the method comprising: (a) determining a level of expression of a gene signature in a sample of endometrial cells obtained from a subject, (b) comparing the determined level of expression of each gene in the gene signature with a control level; and (c) determining whether the subject is within the WOI, wherein the subject is identified as being within the WOI if the level of the expression of the gene signature is higher than a control level.

### RELATED APPLICATIONS

This application claims priority to U.S. Provisional Patent Application No. 62/686,621, filed Jun. 18, 2018, the contents of which are incorporated herein by reference.

### FIELD OF THE INVENTION

The present Application relates to methods, compositions, and kits for assessing endometrial transformation, including the implantation window.

### BACKGROUND OF THE INVENTION

Despite recent advances in assisted reproductive technologies, implantation rates remain relatively low. Implantation failures are thought to be associated with inadequate endometrium receptivity and/or with defects in the embryo-endometrium dialogue. The endometrium is receptive to blastocyst implantation during a spatially and temporally restricted window, called “the implantation window” or the “window of implantation.” In humans, this period begins 6-10 days after the LH surge and lasts approximately 48 hours. Several parameters have been suggested for assessing endometrium receptivity, including endometrial thickness which is a traditional criterion, endometrial morphological aspect and endometrial and subendometrial blood flow. However, their positive predictive value is still limited.

More recently, transcriptomic approaches have been utilized to identify biomarkers of the human implantation window. Using microarray technology in human biopsy samples, several authors have observed modifications in gene expression profile associated to the transition of the human endometrium from a pre-receptive (early-secretory phase) to a receptive (mid-secretory phase) state (Carson et al., 2002; Riesewijk et al., 2003; Mirkin et al., 2005; Talbi et al., 2006). However, only very few genes were in common between all these studies (Haouzi et al., 2009). Such variability in the results may have several explanations: differences in the day of the endometrial biopsies, different patient profiles, inadequate numbers of endometrial samples studied, and the overall complexity of the endometrium.

The endometrium is unlike any other tissue as it consists of multiple cell types which vary dramatically in state through a monthly cycle as they enter and exit the cell cycle, remodel, and undergo various forms of differentiation with relatively rapid rates. The notable variance in menstrual cycle lengths within and between individuals¹⁰ adds an additional variable to the system. Studies to date including transcriptomic characterizations have been insufficient to understand and characterize hallmark endometrial events, such as the implantation window.

Given these deficiencies in the art, and in view of the broad relevance and importance of human fertility and regenerative and reproductive biology, there has been a long need in the art for a systematic characterization and molecular understanding of endometrial transformation across the natural menstrual cycle that go beyond the traditional histological characterization scheme well established in the art. Such an understanding—including the identification of useful biomarkers associated with hallmark endometrial events, e.g., the implantation window—would make a significant contribution to the art and to the field of medical intervention into human reproductive technologies, e.g., in vitro fertilization and contraception technologies.

### SUMMARY

The present disclosure is based, in part, on the finding that after systemic transcriptomic characterization of the human endometrium across six (6) cell types—including (1) previously uncharacterized ciliated epithelium, (2) unciliated epithelium, (3) stromal cells (e.g., stromal fibroblasts), (4) endothelium cells, (5) macrophages, and (6) lymphocytes—and the different phases of the menstrual cycle (e.g., menstruation, follicular phase, ovulation, and luteal phase), that certain genes (e.g., biomarkers) are indicative and/or provide a gene expression signature for one or more hallmark endometrial events, e.g., a specific phase of endometrial transformation, such as, the implantation window. Accordingly, aspects of the present Application relate to methods and compositions for transcriptomic characterization of human endometrium over the different cell types making up the endometrium as the cells undergo change throughout the complete transformation cycle of the endometrium during a menstrual cycle to identify cell-type-specific gene signatures that may be used to evaluate endometrial samples for the appearance or presence of one or more menstrual cycle events, e.g., implantation window.

In various embodiments, the present invention relates to using the cell-type-specific gene expression signatures (e.g., biomarker panels) to evaluate, assess, or otherwise probe one or more endometrial samples from a subject to detect the appearance or presence of one or more menstrual cycle events, e.g., implantation window. In some embodiments, the endometrial samples can be evaluated in bulk, that is as a complete tissue sample since the gene expression signatures are characteristic of a unique endometrial cell type. In other embodiments, the endometrial sample can be process to separate out one or more specific cell types, e.g., the unciliated epithelial cells, using a means for cell separation (e.g., FACS cell-sorting). The separated endometrial cell subtypes can be separately evaluated using the appropriate gene expression signature for that cell type to detect the appearance or presence of one or more menstrual cycle events, e.g., implantation window, in that tissue or cell sample.

In other aspects, the present Application relates to the identified cell-type-specific gene panel signatures, i.e., sets of biomarkers, which correspond or otherwise mark the appearance, presence, or disappearance of a specific phase of endometrial transformation, such as, for example, the window of implantation. In still other aspects, the present Application describes practical and/or clinical application of the identified gene panel signatures to detect the appearance, presence, or disappearance of a particular transformation state of the endometrium of a subject, i.e., the detection of the window of implantation.

Further, aspects of the present disclosure relate to methods and compositions for detecting the phase of endometrial transformation in a subject by detecting and measuring differentially expressed genes (e.g., biomarkers). In some embodiments, differentially expressed genes (e.g., biomarkers) are detected in a sample from a subject (e.g., a patient). In some aspects, the present disclosure relates to methods to detect the opening of the window of implantation in a subject. In other aspects, the present disclosure relates to methods to detect the opening of decidualization. Some aspects of the present disclosure relate to methods of detecting the early-proliferative, late-proliferative, early-secretory, mid-secretory, and/or late-secretory phase of the menstrual cycle of a subject.

Additional aspects and embodiments of the present invention described herein are as follows.

In one aspect, the Application provides a method of diagnosing a menstrual cycle event in a subject, comprising detecting in a biological sample a gene signature for one or more endometrial cell types. The menstrual cycle event can include the follicular phase, ovulation, or the luteal phase, or a window of implantation (WOI).

In various embodiments, one or more endometrial cell types can be selected from the group consisting of stroma cells, endothelium cells, immune cells, unciliated epithelium cells, and ciliated epithelium cells.

In some embodiments, the one or more endometrial cell types is unciliated cells and the gene signature comprises one or more biomarkers selected from the group consisting of: PLUA, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP. In certain embodiments, CADM1, NPAS3, ATP1A1, and TRAK1 are downregulated and NUPR1 is upregulated relative to WOI.

In other embodiments, the one or more endometrial cell types is stromal cells and the gene signature comprises one or more biomarkers selected from the group consisting of: STC1, NGATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1. In certain embodiments, the NFATC2, BMP2, PMAIP1, ZFYVE21, CILP, SLF2, MATN2, and FGF7 are downregulated and CRYAB is upregulated relative in WOI.

In certain embodiments, the methods may include the step of separating the one or more endometrial cells prior to the detection step. For example, prior to detection of biomarkers in a sample, the stroma cells, endothelium cells, immune cells, unciliated epithelium cells, and ciliated epithelium cells can be separated from one another.

In various embodiments, the cells can separated by fluorescence activated cell sorting (FACS).

In other embodiments, the methods may include the additional step of transferring a fertilized embryo to the uterus of the subject determined to be within the window of implantation.

In still another aspect, the Application provides a method for determining a gene expression profile in each of a plurality of endometrial cells, wherein said endometrial cells are:

(a) in an endometrial sample obtained from a subject, and
(b) unciliated epithelial cells. The unciliated epithelial cells can be separated from ciliated epithelial cells. The gene expression profile of an unciliated epithelial cell can be identified using one or more gene expression markers characteristic of unciliated epithelial cells. The gene expression profile can comprise at least twenty genes selected from the group consisting of the genes shown in FIG. 3B, or Tables 9 or 10.

In certain embodiments, the gene expression markers characteristic of unciliated epithelial cells can comprise PLUA, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP.

In still another aspect, the Application provides method for detecting that a subject is within a window of implantation (WOI), the method comprising: (a) determining a level of expression of at least twenty genes in a sample of endometrial cells obtained from a subject, wherein the twenty genes are selected from the group consisting of the genes shown in FIG. 3B, or Tables 9 or 10; (b) comparing the determined level of expression of each of the at least twenty genes with a control level; and (c) determining whether the subject is within the WOI, wherein the subject is identified as being within the WOI if the level of the expression of at least twenty genes is at least two-fold higher than a control level.

In yet another aspect, the Application provides a method for identifying a subject as being within a window of implantation (WOI), the method comprising: (a) determining a level of expression of at least one gene in an isolated cell population, wherein the at least one gene is selected from the group consisting of PAEP, GPX3, CXCL14, NUPR1, DPP4, MAOA, MT1G, MT1E, MT1X, and MT1F, wherein the isolated cell population has been isolated from a sample of endometrial cells obtained from a subject, wherein the cell population comprises cells having elevated expression of genes associated with epithelial cells and depressed expression of genes associated with cilial function; and (b) comparing the determined level of expression of the at least one gene with a control level; and (c) identifying the subject as being within the WOI, wherein the subject is identified as being within the WOI if the level of the expression of at least one gene is at least two-fold higher than a control level.

In some embodiments, a method of increasing the likelihood of becoming pregnant comprises (a) performing gene expression assay (e.g., to assay the RNA and/or protein level for one or more genes of interest), for example in tissue (e.g., endometrial tissue, or blood) or in one or more cell types of interest to determine whether a subject (e.g., a woman) is within a window of implantation (WOI); and (b) transferring a fertilized embryo to the uterus of the subject determined to be within the window of implantation.

In some embodiments, a method of treating infertility in a subject in need thereof comprises administering an effective amount of an agent that upregulates any one or more of genes associated with a WOI, for example, but not limited to, any one or more of the genes selected from the group consisting of PAEP, GPX3, CXCL14, NUPR1, DPP4, MAOA, MT1G, MT1E, MT1X, and MT1F in one or more of the tissues in the subject in an effective amount to treat the infertility.

In still other aspects, the Application provides a method for detecting a window of implantation (WOI) in a subject, the method comprising: (a) isolating a cell population within a sample of endometrial cells obtained from a subject, wherein the cell population comprises cells having elevated expression of genes associated with epithelial cells and depressed expression of genes associated with cilial function; (b) determining a level of expression of at least one gene in the cell population wherein the at least one gene is selected from the group consisting of PAEP, GPX3, and CXCL14; and (c) determining whether the subject has entered the WOI, wherein the subject is identified as within the WOI if the level of the expression of at least one gene is higher than a predetermined level. In some embodiment, step (a) comprises determining the level of expression of at least two genes from the group consisting of PAEP, GPX3, and CXCL14. In other embodiments, step (a) comprises determining the level of expression of each of the genes from the group consisting of PAEP, GPX3, and CXCL14.

The method in some embodiments may involve determining the level of expression of at least one gene selected from the group consisting of NUPR1, DPP4, MAOA, MT1G, MT1E, MT1X, and MT1F. In other embodiments, the method may involve determining the level of expression of at least two genes selected from the group consisting of NUPR1, DPP4, MAOA, MT1G, MT1E, MT1X, and MT1F. In still other embodiments, the method may involve determining the level of expression of at least three genes selected from the group consisting of NUPR1, DPP4, MAOA, MT1G, MT1E, MT1X, and MT1F. In yet other embodiment, the method may involve determining the level of expression of each gene selected from the group consisting of NUPR1, DPP4, MAOA, MT1G, MT1E, MT1X, and MT1F.

In any of the methods herein, the step of determining the level of expression of a gene comprises determining the amount of a nucleic acid. The level of nucleic acid can be determined using a real-time reverse transcriptase PCR (RT-PCR) assay and/or a nucleic acid microarray. In other embodiments, the nucleic acid can be determined using a hybridization assay and at least one labeled binding agent (e.g., a labeled oligonucleotide binding agent).

In any of the method herein, the step of determining the level of expression of a gene can involved determining an amount of a protein encoded by that gene, such as by using an immunohistochemical assay, an immunoblotting assay, and/or a flow cytometry assay.

In various embodiments, the sample can be selected from the group consisting of a sample of endometrium tissue, endometrial stromal cells, and/or endometrial fluid.

The subject of any of the methods herein may be a human, for example, a woman trying to become pregnant, e.g., an in vitro fertilization candidate/patient.

In yet another aspect, the present Application provides a method of increasing the likelihood of becoming pregnant comprising using the method that includes evaluating the expression level(s) of one or more of the genes described herein (for example in Tables 1-17 or elsewhere in this Application) in a subject to determine whether the subject is approaching, entering, in, or exiting a window of implantation, and implanting a fertilized embryo (e.g., from an in vitro fertilization procedure) if the window of implantation is open. In some embodiments, the gene expression levels are detected in a biological sample obtained from the subject, for example a tissue sample, for example a blood, endometrial tissue, endometrial cells, or endometrial fluid sample. In some embodiments, one or more cell types (e.g., ciliated epithelial cells, unciliated epithelial cells, stromal fibrolasts, and/or other cell types described in this Application, for example, but not limited to, cell types 1-6 described above) are isolated from the biological sample, or the nd sample is enriched for one or more cell types (e.g., ciliated epithelial cells, unciliated epithelial cells, stromal fibrolasts, and/or other cell types described in this Application, for example, but not limited to, cell types 1-6 described above).

In still another aspect, the Application provides a method of treating infertility in a subject in need thereof, comprising administering an effective amount of an agent that upregulates any one or more of the genes selected from the group consisting of PAEP, GPX3, CXCL14, NUPR1, DPP4, MAOA, MT1G, MT1E, MT1X, and MT1F in one or more of the tissues in the subject in an effective amount to treat the infertility. The agent can include a nucleic acid encoding for any one or more of the genes selected from the group consisting of PAEP, GPX3, CXCL14, NUPR1, DPP4, MAOA, MT1G, MT1E, MT1X, and MT1F in an expression system. The administering of the agent can result in the opening of the window of implantation in the subject.

Other aspects of the invention are described in or are obvious from the following disclosure, and are within the ambit of the invention.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1C show the definition of endometrial cell types at transcriptome level. FIG. 1A. Dimension reduction (tSNE) on all cells and top over-dispersed genes revealed six endometrial cell types. (top right inset: tSNE performed on immune cells only) FIG. 1B. Top discriminatory genes (differentially expressed genes expressed in >85% cells in the given type) and canonical markers (starred) for each identified cell type. FIG. 1C. Functional enrichment of uniquely expressed genes in ciliated epithelium. (FC: fold change).

FIGS. 2A-2C show constructing trajectories of endometrial remodeling across the menstrual cycle at single cell resolution. FIG. 2A. Pseudotime assignment of cells across the trajectory of menstrual cycle (trajectories: principal curves, numbers: major phases defined in FIGS. 8A-8D and 9A-9C, start: start of the trajectory). FIG. 2B. Correlation between pseudotime and time (the day of menstrual cycle). FIG. 2C. Correlation of pseudotime between unciliated epithelial and stroma cells from the same woman. (dot: median of all cells from a woman; error bar: median absolute deviation).

FIGS. 3A-3B(C) show temporal transcriptome dynamics across the menstrual cycle. Exemplary phase and sub-phase defining genes, and relation between transcriptomically defined phases and canonical endometrial stages for FIG. 3A unciliated epithelium (epi) and FIG. 3B stroma (str) cells in a human menstrual cycle (C). (Dashed line: continuous transition, WOI: window of implantation).

FIGS. 4A-4E show the identification of subpopulations of unciliated epithelial cells across the trajectory of the menstrual cycle. FIG. 4A. Subpopulations of unciliated epithelial cells independently validated in FIG. 12A. FIGS. 4B-4D. Dynamics of genes FIG. 4B that differentially expressed between the two subpopulations across multiple phases, FIG. 4C that are previously reported to be implicated in endometrial remodeling or embryo implantation, and FIG. 4D that exemplified those that reached maximum differential expression in phase 2. (Dashed lines: boundaries between 4 phases). FIG. 4E. Functional enrichment of genes overexpressed in luminal epithelium during epithelial gland formation. (Indented: terms belonging to the same GO hierarchy but with higher specificity as the term immediately above (highest significance value).

FIGS. 5A-5D show endometrial lymphocytes across the menstrual cycle and their interaction with other cell types during decidualization. FIG. 5A. Phase-associated abundance of endometrial lymphocytes normalized against stromal cells. FIG. 5B. Expression of markers identifying major lymphoid lineages. Cells (columns) were sorted based on % expression of pan-markers for decidualized NK (NK) and NK cell receptors (NKR). FIG. 5C. Median expression of NK functional genes. FIG. 5D. Functional annotation (left) and expression (right) of genes that were overexpressed in decidualized stroma that are implicated in immune responses.

FIG. 6 shows the distribution of a number of cells sampled across the menstrual cycle. (day: the day of menstrual cycle).

FIG. 7 shows the classification and distribution of functional annotations for uniquely expressed genes in ciliated epithelium.

FIGS. 8A-8D show an unbiased definition of phases of endometrial transformation across the menstrual cycle. FIGS. 8A-8B. tSNE using whole transcriptome information and phase assignment using Ward's hierarchical agglomerative clustering method. FIGS. 8C-8D. tSNA cast with time annotation (epi: unciliated epithelium, str: stroma, day: the day of menstrual cycle).

FIGS. 9A-9C show constructing trajectories of endometrial transformation across the menstrual cycle via MI-based approach. FIG. 9A. MI between expression of genes and time (curved line) or permutated time (black). Genes are ranked by MI. FIG. 9B. tSNE using time-associated genes and trajectories of endometrial transformation defined by principal curves. FIG. 9C. Phase assignment using Ward's hierarchical agglomerative clustering method. (epi: unciliated epithelium, str: stroma).

FIGS. 10A-10B show the discontinuity of phase 4 epithelium obtained using different analysis methods. FIG. 10A. First 3 components of multidimensional scaling on unciliated epithelium using whole transcriptome information. FIG. 10B. tSNE on top 50 principal components obtained via principal component analysis on whole transcriptome information. (Numbers 1-4: phase assignment determined in FIG. 9C).

FIGS. 11A-11D show global temporal transcriptome dynamics across the menstrual cycle. FIG. 11A. MI between expression of pseudotime-associated genes (FDR<1E-05) and pseudotime (curved line) or permutated pseudotime (black). FIG. 11B. Dynamics of pseudotime associated genes across the trajectory of menstrual cycle. (epi: unciliated epithelium, str: stroma). FIGS. 11C-11D. Distribution (left) and factional dynamics (right) of cycling cells.

FIG. 12 shows endometrial G1/S and G2/M signatures in endometrial cycling cells. (epi: unciliated epithelium, str: stroma).

FIGS. 13A-13F show deviation of subpopulations of unciliated epithelial cells through the trajectory of the menstrual cycle. FIG. 13A. Dimension reduction (tSNE) on unciliated epithelial cells at the major phases/sub-phases across the menstrual cycle. FIG. 13B. Dynamics of phase-defining and housekeeping genes in subpopulations in unciliated epithelia across the menstrual cycle. FIG. 13C. Dynamics of differentially expressed genes between the two sub-populations during phase 2. FIG. 13D. The relationship of the ambiguous cell population with luminal and glandular cells in early phase 1. Genes shown are differentially expressed genes (−log₁₀(p_adj of a Wilcoxon's rank sum test)>0.05, log₂(FC)>2) between luminal and glandular epithelial cells in early phase 1. Cells (column) are ordered by the ratio of (average expression of genes upregulated in the luminal) and (average of expression of genes upregulated in the glandular) FIG. 13E. Genes over-expressed and under-expressed in the ambiguous cell population over luminal and glandular epithelial cells in early phase 1. Cells (column) are ordered by the ratio of (average expression of genes under-expressed) and (average of expression of genes over-expressed). FIG. 13F. Temporal expression of vimentin (VIM) in unciliated epithelial cells.

FIG. 14 shows the phase-associated abundance of minor endometrial cell types. Abundance was normalized to total number of unciliated epithelial or stromal single cells captured.

FIG. 15 shows fractional dynamics of CD56+ cells in CD3+ and CD3− NK cells.

FIGS. 16A-16B show validation of markers, epithelial lineage, and spatial visualization for endometrial ciliated cells using RNA and antibody co-staining. FIG. 16A. Representative images of human endometrial gland (top panels) and lumen (bottom panels) at day 17 (left panels) and day 25 (right panels) of the menstrual cycle. (Single CDHR3 and C11orf88 RNA molecules appear as dots in in the top insets of both the top and bottom panels. FOXJ1 antibody staining shown in the bottom insets of both the top and bottom panels. Scale bar: 50 μm. Zoomed-in areas contain triple-expressing cells from the white dashed box in the corresponding panel). FIG. 16B. Integrated intensity of FOXJ1 antibody for double RNA positive (++) and negative (−−) cells from all images before (left) and after (right) ovulation. (++: cells expressing ≥4 RNA molecules of both markers. Horizontal line: median. ****: p-value of a Wilcoxon's rank sum test <0.0001).

FIGS. 17A-17E show endometrial lymphocytes across the human menstrual cycle and their interactions with stromal fibroblasts during decidualization. FIG. 17A. Expression of inhibitory and activating NK receptors (NKR). Cells (columns) were sorted based on percent of NKR expressed. FIG. 17B. Dynamics of genes related to lymphocyte functionality (shown are the medians). “CD3+” and “CD3−” cells are classified based on the expression of markers characteristic of T lymphocytes shown in FIG. 23B. FIG. 17C. Functional annotation (left) and expression (right) of genes that were overexpressed in decidualized stromal fibroblasts (phase 4) that are implicated in immune responses. FIGS. 17D-17E. Spatial distribution of CD3 (top panels of FIGS. 17D-17E) and CD56 (bottom panels of FIGS. 17D-17E) positive immune cells (arrow and open arrow) and stromal fibroblast (open arrow) before (FIG. 17D, day 17) and during (FIG. 17E, day 24) decidualization.

FIGS. 18A-18C show constructing single cell resolution trajectories of menstrual cycle using mutual information (MI) based approach. FIG. 18A. Unbiased definition of four major phases of endometrial transformation across the human menstrual cycle via tSNE on all genes detected (Inset: phase assignment using Ward's hierarchical agglomerative clustering). FIG. 18B. MI between expression of genes and time (curved line) or permutated time (black) for unciliated epithelial cells (epi) and stromal fibroblasts (str). (Genes are ranked by MI). FIG. 18C. tSNE using time-associated genes and trajectories of endometrial transformation defined by principal curves. (Inset: Phase assignment using Ward's hierarchical agglomerative clustering) (epi: unciliated epithelia; str: stromal fibroblasts).

FIGS. 19A-19C show discontinuity between phase 3 and 4 unciliated epithelia supported by different analysis methods. Dimension reduction of unciliated epithelial cells (left) and stromal fibroblasts (right) via principal component analysis (linear) (FIG. 19A) and multidimensional scaling (non-linear) (FIG. 19B) using whole transcriptome information. FIG. 19C. tSNE on top 50 principal components obtained via principal component analysis on whole transcriptome information. (Phase 1-4 assignment and color code followed those in FIG. 18C).

FIGS. 20A-20E show transcriptional factors (TF) that are dynamic across the menstrual cycle. FIG. 20A, FIG. 20B. Categorization of all dynamic TFs for unciliated epithelia (epi, FIG. 20A) and stromal fibroblasts (str, FIG. 20B) (genes bracketed by red bar are zoomed in FIG. 20C, FIG. 20D). FIG. 20C, FIG. 20D. TFs that are associated with the entrance/exit of WOI (bottom) or phase-defining (top) in epi (FIG. 20C) and str (FIG. 20D). FIG. 20E. Expression of TFs that are nuclear hormone receptors for estrogen (ESR1), progesterone (PGR), glucocorticoid (NR3C1), and androgen (AR). (For heatmap, TFs were ordered first by pseudotime of the major peak and then pseudotime of the inflection point.)

FIGS. 21A-21D show genes for secretory proteins (secretory genes) that are dynamic across the menstrual cycle. FIG. 21A, FIG. 21B. Categorization of all dynamic secretory genes for unciliated epithelia (epi, FIG. 21A) and stromal fibroblasts (str, FIG. 21B) (genes bracketed by purple bar are zoomed in FIG. 21C, FIG. 21D). FIG. 21C, FIG. 21D. Secretory genes that are associated with the entrance/exit of WOI (bottom) in epi (FIG. 21C) and str (FIG. 21D) (For heatmap, secretory genes were ordered as in FIGS. 20A-20E).

FIG. 22 shows top phase-defining genes for the two proliferative phases.

FIGS. 23A-23C show changes in other endometrial cell types across the menstrual cycle. FIG. 23A. Normalized abundance of other endometrial cell types demonstrated phase-associated dynamics. Normalization was done against total number of unciliated epithelial cells (ciliated epithelium) or stromal fibroblasts (lymphocyte, endothelium, macrophage) captured for each biopsy. FIG. 23B. Expression of markers for major lymphoid lineages. Cells (columns) were sorted based on percent NK receptors expressed (as in FIG. 17A). FIG. 23C. Percent CD56+ cells in all CD3+ and CD3− lymphocytes across major phases of cycle.

FIGS. 24A-24D show data summary. FIG. 24A. Relation between the day of menstrual cycle for a woman and her assignment to one of the four major phases based on single cell transcriptomic analysis. FIG. 24B. Total number of single cells analyzed for each woman. FIG. 24C. Distribution of one of the six cell types identified for each woman. FIG. 24D. Distribution of glandular and luminal epithelial cells for each woman. Gray: cells belonging to the ambiguous cell population as in FIG. 4A. Each dot (FIG. 24A, FIG. 24B) or each bar (FIG. 24C, FIG. 24D) represents a woman. Women were ordered, from left to right, based on the median pseudotimes of her stromal fibroblasts and unciliated epithelia. Phase (x-axis) followed that in FIG. 16A and FIG. 16B.

### DETAILED DESCRIPTION

There has long been a need in the art for a systematic characterization and molecular understanding of endometrial transformation across the natural menstrual cycle that go beyond the traditional histological characterization scheme well established in the art. Such an understanding—including the identification of useful biomarkers associated with hallmark endometrial events, e.g., the implantation window—would make a significant contribution to the art and to the field of medical intervention into human reproductive technologies, e.g., in vitro fertilization and contraception technologies.

In a human menstrual cycle, endometrium undergoes remodeling, shedding, and regeneration, which are processes driven by substantial gene expression changes in the underlying cellular hierarchy. Despite its importance in human fertility and regenerative biology, mechanistic understanding of this unique type of tissue homeostasis has remained rudimentary. Described in the present Application are the transcriptomic transformations of human endometrium at single cell resolution. Further described are dissections of multidimensional cellular heterogeneity of the tissue across the entire natural menstrual cycle. The methods described herein permitted the recognition of six discrete endometrial cell types that were analyzed, including previously uncharacterized ciliated epithelium. Further analysis of gene expression patterns within these newly defined cell types demonstrated characteristic signatures for each cell type and phase during four major phases of endometrial transformation. This resulted in the surprising discovery that the human window of implantation opens up with an abrupt and discontinuous transcriptomic activation in the epithelium, accompanied with widespread decidualized feature in the stroma. Also unexpected was the finding of signatures in luminal and glandular epithelium during epithelial gland reconstruction, suggesting a mechanism for adult gland formation. Described herein are precise and accurate methods for determination of endometrial status, e.g., the implantation window, useful in the treatment and/or management of patients, including but not limited to patients in need of assisted reproduction.

The present disclosure is based, in part, on the finding that certain genes (e.g., biomarkers) are indicative of one or more specific phases of endometrial transformation that occur in the human menstrual cycle. Aspects of the present disclosure relate to methods and compositions for detecting the phase of endometrial transformation in a subject by detecting and measuring differentially expressed genes. In some embodiments, differentially expressed genes are detected in a sample from a subject (e.g., a patient). In some aspects, the present disclosure relates to methods to detect the opening of the window of implantation and/or decidualization in a subject. Some aspects of the present disclosure relate to methods of detecting the early-proliferative, late-proliferative, early-secretory, mid-secretory, and/or late-secretory phase of the menstrual cycle of a subject. The present disclosure is based, in part, on the finding that after systemic transcriptomic characterization of the human endometrium over the entire menstrual cycle, gene expression signatures could be identified that uniquely correspond to one of six identified endometrial cell subtypes (ciliated epithelium, unciliated epithelium, stromal cells, endothelium cells, macrophages, and lymphocytes) and which may be used to identify or detect one or more hallmark endometrial events, e.g., a specific phase of endometrial transformation, such as, the implantation window, in an endometrial sample. In various embodiments, the present invention relates to using the cell-type-specific gene expression signatures (e.g., biomarker panels) to evaluate, assess, or otherwise probe one or more endometrial samples from a subject to detect the appearance or presence of one or more menstrual cycle events, e.g., implantation window. In some embodiments, the endometrial samples can be evaluated in bulk, that is as a complete tissue sample since the gene expression signatures are characteristic of a unique endometrial cell type. In other embodiments, the endometrial sample can be process to separate out one or more specific cell types, e.g., the unciliated epithelial cells, using a means for cell separation (e.g., FACS cell-sorting). The separated endometrial cell subtypes can be separately evaluated using the appropriate gene expression signature for that cell type to detect the appearance or presence of one or more menstrual cycle events, e.g., implantation window, in that tissue or cell sample.

In other aspects, the present Application relates to the identified cell-type-specific gene panel signatures, i.e., sets of biomarkers, which correspond or otherwise mark the appearance, presence, or disappearance of a specific phase of endometrial transformation, such as, for example, the window of implantation. In still other aspects, the present Application describes practical and/or clinical application of the identified gene panel signatures to detect the appearance, presence, or disappearance of a particular transformation state of the endometrium of a subject, i.e., the detection of the window of implantation.

### Definitions

Unless defined otherwise, all technical and scientific terms used herein have the meaning commonly understood by one of ordinary skill in the art to which this invention belongs. The following references provide one of skill in the art to which this invention pertains with a general definition of many of the terms used in this invention: Singleton et al., Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2^{nd} Edition, W.B. Saunders Company. For the purposes of the present invention, the following terms are further defined.

### A/an/the

As used herein and in the claims, the singular forms “a,” “an,” and “the” include the singular and the plural reference unless the context clearly indicates otherwise. Thus, for example, a reference to “an agent” includes a single agent and a plurality of such agents.

### Biomarker and Biomarker Signature

As used herein, a “biomarker,” or “biological marker,” generally refers to a measurable indicator of some biological state or condition. The term is also occasionally used to refer to a substance whose detection indicates the presence of a living organism. Biomarkers are often measured and evaluated to examine normal biological processes, pathogenic processes, or pharmacologic responses to a therapeutic intervention. Combined groups of biomarkers with a uniquely characteristic pattern associated with a condition, disease, or otherwise biological state (e.g., a stage of the menstrual cycle or the window of implantation) may be referred to as a “biomarker signature” or equivalently as a “gene signature” or “gene expression signature” or “gene expression profile.” A gene signature or gene expression signature is a single or combined group of genes in a cell with a uniquely characteristic pattern of gene expression that occurs as a result of a biological process (e.g., a stage of the menstrual cycle) or pathogenic medical condition (e.g., endometriosis). Activating pathways in a regular physiological process (e.g., the transformation pathway along the menstrual cycle) or a physiological response to a stimulus results in a cascade of signal transduction and interactions that elicit altered levels of gene expression, which is classified as the gene signature of that physiological process or response.

The clinical applications of gene signatures breakdown into prognostic, diagnostic, and predictive signatures. The phenotypes that may theoretically be defined by a gene expression signature range from those that predict the survival or prognosis of an individual with a disease, those that are used to differentiate between different subtypes of a disease, to those that predict activation of a particular pathway (e.g., predict the timing of WOI). Ideally, gene signatures can be used to select a group of patients for whom a particular treatment will be effective (e.g., timing of WOI for in vitro fertilization candidates).

Prognostic refers to predicting the likely outcome or course of a disease. Classifying a biological phenotype or medical condition based on a specific gene signature or multiple gene signatures, can serve as a prognostic biomarker for the associated phenotype or condition. This concept termed prognostic gene signature, serves to offer insight into the overall outcome of the condition regardless of therapeutic intervention. Several studies have been conducted with focus on identifying prognostic gene signatures with the hopes of improving the diagnostic methods and therapeutic courses adopted in a clinical settings. It is important to note that prognostic gene signatures are not a target of therapy; they offer additional information to consider when discussing details such as duration or dosage or drug sensitivity etc. In therapeutic intervention. The criteria a gene signature preferably meets to be deemed a prognostic marker include demonstration of its association with the outcomes of the condition, reproducibility and validation of its association in an independent group of patients and lastly, the prognostic value must demonstrate independence from other standard factors in a multivariate analysis.

A diagnostic gene signature serves as a biomarker that distinguishes phenotypically similar medical conditions that have a threshold of severity consisting of mild, moderate or severe phenotypes. Establishing verified methods of diagnosing clinically indolent and significant cases allows practitioners to provide more accurate care and therapeutic options that range from no therapy, preventative care to symptomatic relief. These diagnostic signatures also allow for a more accurate representation of test samples used in research.

A predictive gene signature predicts the effect of treatment in patients or study participants that exhibit a particular disease phenotype. A predictive gene signature unlike a prognostic gene signature can be a target for therapy. The information predictive signatures provide are more rigorous than that of prognostic signatures as they are based on treatment groups with therapeutic intervention on the likely benefit from treatment, completely independent of prognosis. Predictive gene signatures addresses the paramount need for ways to personalize and tailor therapeutic intervention in diseases. These signatures have implications in facilitating personalized medicine through identification of more novel therapeutic targets and identifying the most qualified subjects for optimal benefit of specific treatments.

### Biomarker Status

This Application may reference the “status” or “state” of a biomarker in a sample. In various embodiments, reference to the “abnormal status or state” of a biomarker means the biomarker's status in a particular sample differs from the status generally found in average samples (e.g., healthy samples or average diseased samples). Examples include mutated, elevated, decreased, present, absent, etc. Reference to a biomarker with an “elevated status” means that one or more of the above characteristics (e.g., expression or mRNA level) is higher than normal levels. Generally this means an increase in the characteristic (e.g., expression or mRNA level) as compared to an index value. Conversely reference to a biomarker's “low status” means that one or more of the above characteristics (e.g., gene expression or mRNA level) is lower than normal levels. Generally this means a decrease in the characteristic (e.g., expression) as compared to an index value. In this context, a “negative status” of a biomarker generally means the characteristic is absent or undetectable.

### Comprising

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

### Decidualization

As used herein, “decidualization” is a process that results in significant changes to cells of the endometrium in preparation for, and during, pregnancy. This includes morphological and functional changes to endometrial stromal cells (ESCs), the presence of decidual white blood cells (leukocytes), and vascular changes to maternal arteries. The sum of these changes results in the endometrium changing into a structure called the decidua.

### Epithelial

As used herein, the “epithelium” is one of the four basic types of animal tissue, along with connective tissue, muscle tissue and nervous tissue. Epithelial tissues line the outer surfaces of organs and blood vessels throughout the body, as well as the inner surfaces of cavities in many internal organs, e.g., the uterus.

### Endometrium

As used herein, “endometrium” is the mucous membrane lining the uterus, which thickens during the menstrual cycle in preparation for possible implantation of an embryo.

### Isolated Cell

An “isolated cell” refers to a cell which has been separated from other components and/or cells which naturally accompany the isolated cell in a tissue or mammal.

### Obtaining

The term “obtaining” as in “obtaining the spore associated protein” is intended to include purchasing, synthesizing or otherwise acquiring the spore associated protein (or indicated substance or material).

### Sample

As used herein, a “sample” refers to a composition that comprises biological materials such as (but not limited to) endometrial tissue, endometrial cells, or endometrial fluid from a subject.

### Subject

The term “subject” refers to a subject in need of the analysis described herein. In some embodiments, the subject is a patient (e.g., a female patient). In some embodiments, the subject is a human (e.g., a woman). In some embodiments, the human is trying to become pregnant. The subject in need of the analysis described herein may be a patient suffers from infertility.

### Transcriptome

As used herein, “transcriptome” refers to the collection of all gene transcripts in a given cell and comprises both coding RNA (mRNAs) and non-coding RNAs (e.g., siRNA, miRNA, hnRNA, tRNA, etc.). As used herein, an “mRNA transcriptome” refers to the population of all mRNA molecules present (in the appropriate relative abundances) in a given cell. An mRNA transcriptome comprises the transcripts that encode the proteins necessary to generate and maintain the phenotype of the cell. As used herein, an mRNA transcriptome may or may not further comprise mRNA molecules that encode proteins for general cell existence, e.g., housekeeping genes and the like.

### Window of Implantation

As used herein, the term “window of implantation (“WOI”)” or, equivalently, “implantation window” refers to is defined as that period when the uterus is receptive for implantation of the free-lying blastocyst. This period of receptivity is short and results from the programmed sequence of the action of estrogen and progesterone on the endometrium.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Menstrual Cycle

In various aspect, the present Application relates to transcriptomic assessment of various types of cells making up the endometrium throughout the menstrual cycle. The menstrual cycle is the regular natural change that occurs in the female reproductive system (specifically the uterus and ovaries) that makes pregnancy possible. The cycle is required for the production of oocytes, and for the preparation of the uterus for pregnancy.

The menstrual cycle is complex and is controlled by many different glands and the hormones that these glands produce. The hypothalamus causes the nearby pituitary gland to produce certain chemicals, which prompt the ovaries to produce the sex hormones estrogen and progesterone. The menstrual cycle is a biofeedback system, which means each structure and gland is affected by the activity of the others.

The menstrual cycle is divided into four recognized main phases: menstruation, the follicular phase, ovulation, and the luteal phase. Menstruation is the elimination of the thickened lining of the uterus (endometrium) from the body through the vagina. Menstrual fluid contains blood, cells from the lining of the uterus (endometrial cells) and mucus. The average length of a period is between three days and one week. The follicular phase starts on the first day of menstruation and ends with ovulation. Prompted by the hypothalamus, the pituitary gland releases follicle stimulating hormone (FSH). This hormone stimulates the ovary to produce around five to 20 follicles (tiny nodules or cysts), which bead on the surface. Each follicle houses an immature egg. Usually, only one follicle will mature into an egg, while the others die. This can occur around day 10 of a 28-day cycle. The growth of the follicles stimulates the lining of the uterus to thicken in preparation for possible pregnancy. Ovulation is the release of a mature egg from the surface of the ovary. This generally occurs mid-cycle, around two weeks or so before menstruation starts. During the follicular phase, the developing follicle causes a rise in the level of estrogen. The hypothalamus in the brain recognizes these rising levels and releases a chemical called gonadotrophin-releasing hormone (GnRH). This hormone prompts the pituitary gland to produce raised levels of luteinizing hormone (LH) and FSH. Within two days, ovulation is triggered by the high levels of LH. The egg is funneled into the fallopian tube and towards the uterus by waves of small, hair-like projections. The life span of the typical egg is only around 24 hours. The luteal phase occurs when the egg bursts from its follicle and the ruptured follicle stays on the surface of the ovary. For the next two weeks or so, the follicle transforms into a structure known as the corpus luteum. This structure starts releasing progesterone, along with small amounts of estrogen. This combination of hormones maintains the thickened lining of the uterus, waiting for a fertilized egg to implant during the window of implantation. If a fertilized egg implants in the lining of the uterus, it produces the hormones that are necessary to maintain the corpus luteum. This includes human chorionic gonadotrophin (HCG), the hormone that is detected in a urine test for pregnancy. The corpus luteum keeps producing the raised levels of progesterone that are needed to maintain the thickened lining of the uterus. If pregnancy does not occur, the corpus luteum dies, usually around day 22 in a 28-day cycle. The drop in progesterone levels causes the lining of the uterus to fall away. This is known as menstruation. The cycle then repeats.

This cyclic transformation of the endometrium is executed through dynamic changes in states and interactions of multiple cell types, including luminal and glandular epithelial cells, stromal cells, vascular endothelial cells, and infiltrating immune cells. Although different categorization schemes exist, the transformation has been primarily divided into two major stages by the event of ovulation: the proliferative (pre-ovulatory) and secretory (post-ovulatory) stage.³ During the secretory stage, endometrium enters a narrow window of receptive state that is both structurally and biochemically ideal for embryo to implant,^{4, 5} This, the mid-secretory stage, is known as the window of implantation (WOI). To prepare for this state, the tissue undergoes considerable reconstruction in the proliferative stage, during which one of the most essential elements is the formation of epithelial glands⁶, lined by glandular epithelium.

Despite its importance in human fertility and regenerative biology, mechanistic understanding of endometrium-related tissue homeostasis has remained rudimentary. Described in the present Application are the transcriptomic transformations of human endometrium at single cell resolution. Further described are dissections of multidimensional cellular heterogeneity of the tissue across the entire natural menstrual cycle. The methods described herein permitted the recognition of six discrete endometrial cell types that were analyzed, including previously uncharacterized ciliated epithelium. Further analysis of gene expression patterns within these newly defined cell types demonstrated characteristic signatures for each cell type and phase during four major phases of endometrial transformation. This resulted in the surprising discovery that the human window of implantation opens up with an abrupt and discontinuous transcriptomic activation in the epithelium, accompanied with widespread decidualized feature in the stroma. Also unexpected was the finding of signatures in luminal and glandular epithelium during epithelial gland reconstruction, suggesting a mechanism for adult gland formation. Described herein are precise and accurate methods for determination of endometrial status, e.g., the implantation window, useful in the treatment and/or management of patients, including but not limited to patients in need of assisted reproduction.

As used herein, a “menstrual cycle event” refers to any distinct biological state, phase, or condition that occurs during the course of the menstrual cycle which can be detected by a gene signature or biomarker signature associated with one or more endometrial cell subtypes (e.g., stroma cells, endothelium cells, immune cells, unciliated epithelium cells, and ciliated epithelium cells). An example of a menstrual cycle event is ovulation. Another example of a menstrual cycle event is a window of implantation.

### Transcriptome Analysis/Biomarker Identification

In various aspect, the present Application relates to methods of evaluating the human menstrual cycle with respect to the transcriptome of cells making up the endometrium in order to identifying single biomarkers or combinations of biomarkers (e.g., biomarker panels of biomarker signatures) that characterize, identify, or otherwise are associated with one or more hallmark states of the menstrual cycle, e.g., the window of implantation.

The transcriptome can be assessed on the bulk endometrium tissue at one or time points during that menstrual cycle. In this way, the cells composing the endometrium (e.g., the epithelium, stroma (stratum compactum and stratum spongiosum), glandular epithelium, and the lymphatic and/or blood vessel component therein) can be analyzed in bulk. In another approach, the different cells making up the varied types of endometrial sub-components can be separated first, and the transcriptome can be determined for each isolated cell type.

The transcriptome is the complete set of transcripts in a cell, and their quantity, for a specific developmental stage or physiological condition. Understanding the transcriptome is essential for interpreting the functional elements of the genome and revealing the molecular constituents of cells and tissues, and also for understanding development and disease. The key aims of transcriptomics are: to catalogue all species of transcript, including mRNAs, non-coding RNAs and small RNAs; to determine the transcriptional structure of genes, in terms of their start sites, 5′ and 3′ ends, splicing patterns and other post-transcriptional modifications; and to quantify the changing expression levels of each transcript during development and under different conditions.

Various technologies are well-known in the art for deducing and quantifying the transcriptome, including hybridization- or sequence-based approaches. Hybridization-based approaches typically involve incubating fluorescently labelled cDNA with custom-made microarrays or commercial high-density oligo microarrays. Specialized microarrays have also been designed; for example, arrays with probes spanning exon junctions can be used to detect and quantify distinct spliced isoforms. Genomic tiling microarrays that represent the genome at high density have been constructed and allow the mapping of transcribed regions to a very high resolution, from several base pairs to ˜100 bp. Hybridization-based approaches are high throughput and relatively inexpensive, except for high-resolution tiling arrays that interrogate large genomes. However, these methods have several limitations, which include: reliance upon existing knowledge about genome sequence; high background levels owing to cross-hybridization; and a limited dynamic range of detection owing to both background and saturation of signals. Moreover, comparing expression levels across different experiments is often difficult and can require complicated normalization methods.

In contrast to microarray methods, sequence-based approaches directly determine the cDNA sequence. Initially, Sanger sequencing of cDNA or EST libraries was used, but this approach is relatively low throughput, expensive and generally not quantitative. Tag-based methods were developed to overcome these limitations, including serial analysis of gene expression (SAGE), cap analysis of gene expression (CAGE), and massively parallel signature sequencing (MPSS). These tag-based sequencing approaches are high throughput and can provide precise, ‘digital’ gene expression levels. However, most are based on Sanger sequencing technology, and a significant portion of the short tags cannot be uniquely mapped to the reference genome. Moreover, only a portion of the transcript is analysed and isoforms are generally indistinguishable from each other. These disadvantages limit the use of traditional sequencing technology in annotating the structure of transcriptomes.

Recently, the development of novel high-throughput DNA sequencing methods has provided a new method for both mapping and quantifying transcriptomes. This method, termed RNA-Seq (RNA sequencing), has advantages over existing approaches for determining transcriptomes.

RNA-Seq uses deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq, e.g., the Illumina IG18, Applied Biosystems SOLiD22 and Roche 454 Life Science systems have already been applied for this purpose. The Helicos Biosciences tSMS system is also appropriate and has the added advantage of avoiding amplification of target cDNA. Following sequencing, the resulting reads are either aligned to a reference genome or reference transcripts, or assembled de novo without the genomic sequence to produce a genome-scale transcription map that consists of both the transcriptional structure and/or level of expression for each gene.

Further reference can be made regarding transcriptome analysis and RNA-Seq technologies known in the art: (1) Wang et al., Nat Rev Genet. 2009 January; 10(1): 57-63; (2) Lee et al., Circ Res. 2011 Dec. 9; 109(12):1332-41; (3) Nagalakshimi et al., Curr Protoc Mol Biol. 2010 January; Chapter 4: Unit 4.11.1-13; and (4) Mutz et al., Curr Opin Biotechnol. 2013 February; 24(1):22-30, each of which are incorporated herein by reference.

Transcriptome analysis by next-generation sequencing (RNA-seq) allows investigation of a transcriptome at unsurpassed resolution. One major benefit is that RNA-seq is independent of a priori knowledge on the sequence under investigation, thereby also allowing analysis of poorly characterized Plasmodium species.

The transcriptome can be profiled by high throughput techniques including SAGE, microarray, and sequencing of clones from cDNA libraries. For more than a decade, oligonucleotide microarrays have been the method of choice providing high throughput and affordable costs. However, microarray technology suffers from well-known limitations including insufficient sensitivity for quantifying lower abundant transcripts, narrow dynamic range and biases arising from non-specific hybridizations. Additionally, microarrays are limited to only measuring known/annotated transcripts and often suffer from inaccurate annotations. Sequencing-based methods such as SAGE rely upon cloning and sequencing cDNA fragments. This approach allows quantification of mRNA abundance by counting the number of times cDNA fragments from a corresponding transcript are represented in a given sample, assuming that cDNA fragments sequenced contain sufficient information to identify a transcript. Sequencing-based approaches have a number of significant technical advantages over hybridization-based microarray methods. The output from sequence-based protocols is digital, rather than analog, obviating the need for complex algorithms for data normalization and summarization while allowing for more precise quantification and greater ease of comparison between results obtained from different samples. Consequently the dynamic range is essentially infinite, if one accumulates enough sequence tags. Sequence-based approaches do not require prior knowledge of the transcriptome and are therefore useful for discovery and annotation of novel transcripts as well as for analysis of poorly annotated genomes. However, until recently the application of sequencing technology in transcriptome profiling has been limited by high cost, by the need to amplify DNA through bacterial cloning, and by the traditional Sanger approach of sequencing by chain termination.

The next-generation sequencing (NGS) technology eliminates some of these barriers, enabling massive parallel sequencing at a high but reasonable cost for small studies. The technology essentially reduces the transcriptome to a series of randomly fragmented segments of a few hundred nucleotides in length. These molecules are amplified by a process that retains spatial clustering of the PCR products, and individual clusters are sequenced in parallel by one of several technologies. Current NGS platforms include the Roche 454 Genome Sequencer, Illumina's Genome Analyzer, and Applied Biosystems' SOLiD. These platforms can analyze tens to hundreds of millions of DNA fragments simultaneously, generate giga-bases of sequence information from a single run, and have revolutionized SAGE and cDNA sequencing technology. For example, the 3′ tag Digital Gene Expression (DGE) uses oligo-dT priming for first strand cDNA synthesis, generates libraries that are enriched in the 3′ untranslated regions of polyadenylated mRNAs, and produces base cDNA tags.

### Menstrual Cycle Biomarkers

In various aspects, the present Application relates to menstrual cycle biomarkers, i.e., biomarkers which are associated with the various transformational phases of the menstrual cycle, e.g., menstruation, ovulation, One or more such biomarkers may be present in a specific population of cells (e.g., human endometrial stromal cells (hESCs)) and the level of each biomarker may deviate from the level of the same biomarker in a different population of cells and/or in a different subject (e.g., patient). For example, a biomarker that is indicative of decidualization or the opening of the window of implantation (WOI) may have an elevated level or a reduced level in a sample from a subject relative to the level of the same marker in a control sample.

Exemplary biomarkers indicative of the various phases of endometrial transformation in epithelial cells are shown in Table 1. Exemplary biomarkers indicative of the various phases of endometrial transformation in stromal cells (e.g., stromal fibroblast) are shown in Table 2. In some embodiments, a biomarker is differentially expressed in a sample that has been decidualized compared to a sample that is non-decidualized. In some embodiments, a biomarker is differentially expressed in a sample that has an open WOI compared to a sample that does not have an open WOI.

In various embodiments, assessment of the transcriptome of a cell (e.g., limited to an isolated cell or a single cell type, such as unciliated epithelial cells), or a batch of one or more types of isolated cells or cell types (e.g., unciliated epithelial cells together with stromal cells) can be analyzed by transcriptomic analysis using a method known in the art. As part of the transcriptomic analysis, the gene expression levels may be measured or determined for at least one gene. In other embodiments, the gene expression levels can be measured for between 1 and 10 genes, or between 5 and 20 genes, or between 10 and 40 genes, or between 20 and 80 genes, or between 40 and 160 genes, or between 80 and 320 genes, or between 160 and 640 genes, or more. In still other embodiments, the gene expression levels can be measured for at least 1 gene, at least 10 genes, at least 20 genes, at least 30 genes, at least 40 genes, at least 50 genes, at least 60 genes, at least 70 genes, at least 80 genes, at least 90 genes, at least 100 genes, at least 125 genes, at least 150 genes, at least 175 genes, at least 200 genes, at least 300, 400, 500, 600, 700, 800, 900, or 1000 genes or more, for example of the gene listed in any of Tables 1-17 or other genes described in this Application as indicative of WOI status.

In various embodiments, the following tables provide examples of temporally-changing genes identified as a result of transcriptome analysis of endometrial tissues in bulk and/or isolated endometrial cells (e.g., unciliated epithelial cells or stromal cells) measured along the menstrual cycle.

TABLE I
Epithelial genes identified as changing temporally along the menstrual cycle

| | | | | | | | |
|---|---|---|---|---|---|---|---|
| WNT5A | IFT57 | FAM13B | CNP | OGFOD1 | SSBP1 | FREM2 | IDO1 |
| SFRP4 | CREB5 | KRR1 | CCT4 | HERPUD1 | CSRP1 | NAAA | MGST1 |
| NREP | TSPAN6 | SLC35F2 | DDX1 | POLR2G | PPP4R2 | CKB | CCL20 |
| PTMAP5 | CADM1 | MID1 | ATP1B3 | TLE4 | BCAP29 | MFSD6 | ARSB |
| GBP5 | L3MBTL3 | KMO | NBPF10 | TSTD1 | PER2 | ECHS1 | RASGEF1B |
| IFI6 | ASAP1 | TEK1 | PAPD4 | PTPRJ | TP53I3 | POC1B | CLEC4E |
| AKAP1 | PPP2R3A | TLR2 | PRDM2 | LAMB2 | ATP5F1 | FTH1P10 | KRT23 |
| MMP11 | CD44 | PSMD4 | PPA2 | MEX3D | ITGA6 | RNF183 | SLC15A4 |
| PLXDC2 | ARAP2 | DDOST | REEP5 | ERRC41 | CD99 | ZCCHC6 | TMEM45B |
| ANTXR1 | NINJ1 | LINC00665 | SMG1 | SULT1E1 | MRPS34 | HPRT1 | FAM134B |
| PITHD1 | SOX9 | WBSCR22 | MARCKS | POLR2J3 | NAALADL2 | GSN | GDF15 |
| NECTIN2 | N4BP2 | SBNO1 | ANP32E | POLD2 | PLLP | FAM120B | SIK1 |
| IGFBP3 | NRCAM | MRPS17 | SNRPB | UBE2Q2P2 | PNPO | NEBL | DEPTOR |
| LY6E | HCP5 | PPT1 | CDC123 | PSMA7 | ATP1A1 | ECI2 | COMP |
| SHH | SEMA3E | RBM3 | DFFA | LARS | HK2 | ITGA1 | PPP2R5A |
| BMP2 | TARS | PPIL4 | PGD | RIN2 | CTC-444N24.11 | B3GNT2 | RAB11A |
| FLNA | SIPA1L1 | LINC01138 | GOLIM4 | IGFBP2 | GRHL2 | RAB4A | HN1L |
| COL12A1 | CAB39 | SLFN5 | VCL | COA4 | BAGS | SLC4A7 | FAM65B |
| PTGS2 | KPNA4 | SLC39A6 | HNRNPA1P48 | ITM2C | TMEM256 | CKMT2 | EIF4E3 |
| LINC01588 | RRP15 | PTEN | PLEKHA2 | EIF3M | RFLNB | PYY | CTSA |
| MMP7 | CRISPLD1 | TULP4 | SELENOH | RHOB | RANBP17 | FAM177A1 | PHYHIPL |
| LCN2 | TPGS2 | GAS2 | EIF3D | ID2 | SLF2 | ALDH3B2 | CXCL14 |
| QSOX1 | AGPS | BLOC1S6 | UQCC2 | SRGAP3 | AIFM1 | CD36 | SLC7A2 |
| CSF1 | STARD3NL | NHP2 | SNRPF | DUT | KYAT3 | IDH1 | TSPAN1 |
| GJA1 | F3 | RAPGEF2 | YWHAQ | THSD4 | TFCP2L1 | MPZL2 | ATP6V1A |
| ENC1 | DCP1A | PELI1 | RAN | SERPINA3 | PHB2 | GMPR | RIMKLB |
| RAI14 | SLC25A24 | DCAF16 | SLIRP | TCF20 | KCNK13 | COL1A2 | PIGR |
| LIF | CCT2 | CCNG2 | PRPS2 | ZNF611 | L3MBTL4 | ERLEC1 | TMEM92 |
| TUBA1A | FRK | EYA2 | MDK | C22orf29 | NRA | RHOBTB3 | TC2N |
| CYP1B1 | CXCL3 | UBE2N | TPM3 | PLEKHG1 | PYURF | TPD52L1 | MRPS2 |
| WNT7A | IP6K2 | SMAD9 | AKIRIN1 | CNTLN | FAM213A | CREB3L1 | SEPHS2 |
| LAPTM4B | CORO1C | SPDEF | PLPP2 | AGR3 | IL2ORA | BNIP3L | SLC15A1 |
| HMGA1 | MREG | GUSB | MAP4 | SERPINA5 | LLGL2 | HGD | GRAMD1C |
| ELK3 | PSMD11 | MMADHC | STIP1 | PPP3CA | NPTN | CD81 | ANXA4 |
| USP10 | LTV1 | PDGFC | PDLIM1 | CHD3 | ORAI2 | MAP2K6 | VPS41 |
| BCAT1 | ITGAV | ADAT2 | STMN1 | TCEA3 | DLGAP1 | CPT1A | IRX3 |
| COL18A1 | CCNC | SLC25A26 | ALDH1B1 | SAMHD1 | LIPG | GPT2 | ERNI |
| PROM1 | SMIM15 | STK17A | TPR | HNRNPK | TRAK1 | SQLE | C2CD4B |
| C3 | SREK1 | OTUD6B-AS1 | NCL | GRHPR | ACPP | SNX9 | CXCR4 |
| NRP2 | INO80D | ETNK1 | AHCY | LONRF1 | NAA60 | CYB5A | SCCPDH |
| PIM1 | PLCB1 | SUB1 | DNPH1 | COL9A2 | NOSTRIN | LDLR | DPP4 |
| MFAP2 | SH3RF1 | DYNC1I1 | HACD2 | SEMA3C | DLG5 | SLCO3A1 | G0S2 |
| CYR61 | UCHL3 | GLA | CCT3 | LIMCH1 | TAP1 | AREG | TRAM1 |
| ZDHHC13 | TBL1XR1 | FRMD4B | BROX | DANCR | RNASET2 | TMEM144 | HIST1H2AC |
| LUZP1 | ITIH5 | LCLAT1 | MIA3 | RAB14 | PRKX | PPM1H | TMC5 |
| RBP1 | ACTR3 | USP22 | MRPS25 | ALCAM | JTB | RXFP1 | LAMB3 |
| IL18 | FDPS | CSNK2A2 | ATP5A1 | ERC1 | MCC | TAP2 | C12orf75 |
| PLAU | UTP11 | ZNF516 | DKC1 | HEY2 | RABGAP1L | FKBP5 | SLPI |
| SERPINB9 | RDX | PIP4K2A | NAP1L4 | XYLT2 | SUDS3 | HMGCR | C4BPA |
| AMOTL2 | WDR48 | TSPYL1 | ATP5L | PGRMC1 | NFIB | FAM129B | SNX29 |
| NCEH1 | ZHX2 | GREM2 | TOMM22 | ESR1 | OPRK1 | CALD1 | MAP3K5 |
| CD74 | SLC9A3R1 | MXD1 | SNHG14 | LDHB | ACSL4 | FOXO3 | PAX8 |
| THBS1 | IRF6 | ADNP | PFDN5 | ARID1B | DNAJC15 | DCXR | LEPROT |
| TNF | ARHGAP26 | OLA1 | UBAP2L | SNRPB2 | MUC1 | UBE2D2 | DEFB1 |
| ARHGAP29 | PGM2L1 | KIAA1324 | HSBP1 | FMC1 | ARF5 | CYP26A1 | MITF |
| B4GALT5 | EIF2S1 | SCNN1G | CEP95 | TNKS1BP1 | FARSB | LINC00844 | TNFSF15 |
| EMP1 | GPR22 | C16orf72 | TSPAN14 | PRR15 | C2orf88 | ANXA2P2 | AQP3 |
| TOP2B | TCF7L2 | ZNF252P | MAGI1 | PKM | SLC15A2 | ARHGAP18 | GRN |
| RNF152 | SEMA3A | PAK1IP1 | PDIA3 | SERBP1 | TMEM101 | PLEKHF2 | DHRS3 |
| ADAMTS9 | PRMT1 | CNOT6L | GSTK1 | DLG1 | AFMID | ADAMTS8 | UBBP4 |
| ILF3 | MINOS1 | ANAPC4 | CCND1 | CCT8 | PDXDC1 | IFNGR1 | MUC16 |
| ASPH | MAPK1IP1L | ADCYAP1R1 | NASP | RCN2 | CARMIL1 | BTAF1 | SPP1 |
| XRCC5 | ING3 | ATP5C1 | IFI27 | MYBBP1A | NAMPT | DUOX1 | LINC01320 |
| CFI | RBM22 | RPARP-AS1 | HIST1H4C | TOP1 | ANK3 | ATP6V1G1 | AGR2 |
| MARCKSL1 | MORF4L1P1 | TRIM59 | RSRC1 | TCEAL4 | AK4 | CCNA1 | SRD5A3 |
| TSPAN15 | OCLN | UQCRH | DNMT3A | BARD1 | TPI1P1 | PHB | VEGFA |
| HLA-H | KIF21A | DNAJC19 | CUL5 | TMEM14A | ENAH | TFPI2 | DUSP5 |
| DUSP10 | RC3H1 | UBA3 | DNMT1 | TAF8 | ZCRB1 | TMED4 | ADGRF1 |
| ATIC | GCLC | SAR1A | SNRPD3 | DCBLD2 | USMG5 | LINC00116 | CP |
| MIR4435-2HG | GLIPR1 | EPB41L2 | CCP110 | CHCHD5 | PIKFYVE | SLC39A14 | DCPS |
| IL32 | SIX4 | APOBEC3C | ST13 | NAV2 | SLC7A1 | HLA-DOB | SCGB2A2 |
| BHLHE41 | PHLDA1 | VAMP7 | PRPF40A | PLEKHA3 | MARK1 | EMC4 | NUPR1 |
| IL23A | ARMC8 | PPP1R9A | HNRNPD | UGT2B7 | HDDC2 | WIPI1 | CRYAB |
| RASSF3 | TCERG1 | RPP30 | HSPB1 | GATA2 | SMIM22 | MSMO1 | RASD1 |
| SMAD3 | SERPINA1 | AEN | NPDC1 | GREB1 | LONRF2 | SH3BGRL3 | PAPLN |
| SNHG16 | GPR89A | SMARCA1 | UBE2G1 | ANKRD11 | FAM110C | CAPN6 | PAX8-AS1 |
| RRAS2 | PAPD5 | CASP2 | NAE1 | OXCT1 | MPHOSPH10 | LRRC1 | TXNIP |
| FBXO32 | LSM12 | CYP51A1 | EGFR | RCAN1 | LAMTOR4 | DHRS7 | FAM3C |
| CD47 | MED24 | CTD-3014M21.1 | AP3S1 | TIMM8B | PKHD1L1 | PART1 | ZNF292 |
| IGF2R | USP16 | NME2 | PSMC1 | STXBP6 | ATP6VOB | SMS | TRAK2 |
| B3GNT7 | ZNF644 | OSTC | ALDH16A1 | SCD | SF3B6 | ENPP3 | TNFAIP2 |
| MSN | SLC39A10 | PKP4 | IFITM3 | RREB1 | VDAC1 | TUBB2A | VCAN |
| HMGB1P5 | MDM4 | UTRN | TPM4 | ELF2 | HMGN5 | WHRN | HNMT |
| RBBP8 | RBMXL1 | PAFAH1B2 | CRIP1 | JUN | TM9SF3 | DUOXA1 | MYO9A |
| FHL2 | STEAP1 | OAT | PSAP | BASP1 | ARPC4 | SCGB2A1 | GPR160 |
| MB21D2 | PALLD | NTPCR | ID3 | NEO1 | TM7SF3 | PLIN2 | GPX3 |
| DEFB4A | TMEM33 | INTS6 | MYH10 | SNX5 | ADH5 | RAMP2 | PAEP |
| MED4 | ZNF286A | PLAGL2 | CRIP2 | DBI | SOX17 | ARL4C | STC1 |
| HDAC9 | ATXN1 | TMED10 | DST | PFKL | STRBP | HSD17B2 | TUFT1 |
| RGS10 | TMEM120B | ZMYND8 | MGST2 | GDA | RSRP1 | SORD | NNMT |
| EXT2 | TLE3 | CBWD5 | LSM5 | SH3BGRL | HMGN3 | PAPSS1 | FBLN1 |
| CTSS | SPRY1 | GXYLT2 | RANBP1 | PLOD1 | OFD1 | SLC16A1 | HABP2 |
| DLC1 | DNAJC10 | AEBP2 | EMC10 | PABPN1 | ARL6IP5 | ABCG1 | CYP3A5 |
| E2F3 | S1PR2 | DDAH1 | AC013461.1 | SP100 | NDUFA13 | TLE1 | CLDN10 |
| SVIL | MTPAP | PGR | HSPE1 | SYNJ2BP | CTB-178M22.2 | DENND2C | SYNE2 |
| SEMA3B | NMD3 | CNKSR3 | MYO10 | LGR5 | ATP5I | ATP6V1C2 | HKDC1 |
| ADGRA3 | NPM1P27 | CH17-373J23.1 | PHGDH | MTURN | DLX5 | MT1F | ABCC3 |
| ANKLE2 | SRPK1 | FAM96A | MSH3 | KAT6B | NEK1 | MT1X | SCIN |
| FOSL1 | CLUHP3 | CCDC14 | MGLL | CDK11B | CS | UPK1B | C8orf4 |
| CYTOR | VIM | LRP6 | RCC2 | FBXO21 | B3GNT5 | CDK7 | SLC40A1 |
| CA12 | CPM | POLR2D | PRKDC | SOCS3 | PLA2G4F | SCGB1D4 | NAPSB |
| JARID2 | LIPA | DCUN1D1 | SNRPD1 | FZD6 | NOV | SCGB1D2 | PIK3R1 |
| CXCL1 | SENP5 | METTL7A | CD2AP | PARP14 | APOPT1 | TESMIN | IGFBP7 |
| PABPC4 | MTFMT | EBP | ETV5 | IRF2BPL | ADIPOR2 | MMP26 | SERPING1 |
| MACC1 | AGO3 | R3HDM2 | TP53 | PLA2G4A | NDUFC2 | ST14 | GEM |
| SPECC1 | TUSC3 | CLMN | TBC1D5 | TRIM22 | HSD11B2 | XDH | CYP24A1 |
| RBPJ | ST3GAL5 | HNRNPF | GLG1 | FAM155A | SLAIN1 | AFDN | CXCL2 |
| HNRNPAB | ALDH3A2 | HELB | CHD4 | RNF8 | APOL4 | NHSL1 | CLU |
| NFKB1 | KIZ | POGLUT1 | LYRM2 | WWC2 | HOMER2 | HEY1 | FGL2 |
| LAMC2 | IGFBP4 | BZW2 | PSIP1 | PSAT1 | SORBS2 | LPIN1 | ZBTB20 |
| ANKRD33B | ANO1 | INIP | MCAM | MTPN | RHOU | SYBU | LITAF |
| ARL14 | CDC42EP3 | ZRANB2 | MALT1 | TWSG1 | TOB1 | KCMF1 | TNFSF10 |
| SHISA2 | LINC01480 | SNHG6 | NIPSNAP3A | FAM96B | COX17 | SPHK1 | HES1 |
| MYO6 | TNKS2 | TRAF3IP2 | RIOK1 | VTCN1 | IKZF2 | TIAM1 | ABLIM1 |
| RARRES2 | EMID1 | THYN1 | ANP32B | ARPC1B | NME4 | SDCBP2 | DNAJB1 |
| SMURF2 | ADAM28 | TIMM17A | HTATSF1 | KRTCAP2 | CREG1 | SMIM5 | BICD1 |
| CD83 | TAF9 | RBMX | CTSB | ALPL | CDC42SE2 | MT1E | HSPA1A |
| ATP6V1B2 | YLPM1 | EIF4E | ATP1B1 | UNC5B | OST4 | TMEM154 | HSPH1 |
| TARBP1 | MEST | PHF14 | HMGN2 | TMEM131 | HADHA | MT1G | AXL |
| ITGB6 | ARL3 | EIF4B | PARP1 | NRXN3 | GAPDHP65 | MT2A | LUM |
| PTBP2 | TFDP2 | CEP57 | GPI | MSX2 | FDFT1 | MT1M | MAP1B |
| HSPB8 | EXOSC5 | SRSF2 | CNPY2 | BHLHE40 | COX7A2 | LMO7 | CCND2 |
| RAB11FIP1 | EIF3E | BTF3L4 | FBL | POLG2 | CUTA | MT1H | COL3A1 |
| FAM98B | SLC47A1 | SLC25A6 | FRAS1 | PTGS1 | ABRACL | UTP15 | MMP2 |
| SPIN1 | NSG1 | LRRC75A-AS1 | C21orf33 | PIP5K1B | PSMG3 | SLC18A2 | SERPINE1 |
| DEK | APOOL | GAS7 | PRRG4 | COBL | GOLPH3 | LIG4 | FSTL1 |
| KHDRBS1 | CTSH | PSMA6 | SERINC5 | ANXA3 | EDF1 | SLC30A2 | COL1A1 |
| TRIM33 | TCEAL1 | HMOX2 | C8orf33 | CEBPB | HACD3 | ADGRL2 | AKAP12 |
| CMTM7 | PORCN | PRDX6 | NUDT19 | MTA2 | ALDH18A1 | GAST | TCF4 |
| TNFRSF12A | PSMD12 | GTF2A2 | ARID1A | LPAR3 | GNG11 | FAM84B | TIMP1 |
| SPOCD1 | AGO2 | UBE3A | NDUFS5 | RNF122 | STEAP4 | TCN1 | SYNCRIP |
| TXNRD1 | ZBTB38 | IMPDH2 | ATP5G1 | SLBP | ASRGL1 | RASEF | COL4A1 |
| BCL9 | GAN | EIF1AX | LINC00998 | GPBP1L1 | ELP3 | GCNT3 | NAP1L1 |
| OCIAD2 | DMKN | STON2 | ZNF589 | PPL | GGTA1P | CRISP3 | SPARC |
| ADAM9 | PPP1R2 | PTGFRN | HADH | TMEM184B | ALDH6A1 | RIMKLBP1 | LGALS1 |
| TARDBP | MUM1 | BRD3 | KIAA1143 | PDZD2 | GGCT | ELK4 | IFITM1 |
| RIF1 | PCMTD2 | CBX5 | PARK7 | CAP1 | SH3YL1 | PCDH17 | TMEM98 |
| ZNF608 | NPAS3 | PDCD4 | POMP | SLC26A2 | GABRP | PPFIBP2 | TIMP3 |
| SF3B1 | COLGALT1 | M5I2 | MMAB | ZDHHC9 | PRELID3B | DYNLT3 | DCN |
| UBE2E3 | PAN3 | TXN2 | KRT8 | RNF150 | SEC61G | CDYL2 | THBS2 |
| PSMB4 | DAAM1 | TRIM16 | APRT | RAB27A | CAMK2D | RBL2 | CTSC |
| SF3A3 | AC093673.5 | PLEKHA5 | PCDH7 | HPGD | TALDO1 | SLC34A2 | YTHDC2 |
| PAFAH1B3 | TMEM41B | C6orf48 | SELENOW | HNRNPR | SPATA13 | VNN1 | ID1 |
| MYL6B | BMPR1B | C7orf73 | ARL4A | SLC39A8 | CTAGE5 | SLC3A1 | C11orf96 |
| MRPL44 | BST2 | CHD7 | CCDC170 | AP1S2 | SIAH2 | DDX52 | RGS2 |
| S100A16 | PAM | BEX3 | SH3RF2 | SPATS2 | C19orf53 | BCL2A1 | SAMD4A |
| MTF2 | SFXN2 | HSPD1 | METAP1 | TXNDC16 | AMD1 | TNFAIP6 | PDS5B |
| GPRC5A | COL27A1 | TCTN2 | NDUFA2 | CITED4 | MRFAP1 | TSPAN8 | TIMP2 |
| SUPV3L1 | ERI1 | MECOM | CTTN | NDUFB1 | NPR3 | SLCO4A1 | PTN |
| ATRX | DDHD1 | BOD1L1 | CENPX | THAP4 | MRPL55 | ODC1 | PMEPA1 |
| PIP5K1A | MRPL1 | H2AFZ | FAM84A | SREBF2 | DGUOK | AGPAT5 | HDAC2 |
| TPBG | CWC15 | RAD51C | EEF1E1 | SUFU | OVOL1 | PLA2G16 | NOTCH3 |
| BID | CXADR | SNRPN | SYNGR2 | COX16 | ATPIF1 | LINC01502 | C1S |
| PITPNB | KIAA1456 | CEP290 | FUT8 | FAM174B | TFAP2C | ANKRD55 | NRP1 |
| ITCH | ATP5G3 | TFAM | GDI2 | PREP | ACSL5 | EDNRB | S100A6 |
| STX12 | ZNF121 | EXOSC8 | FH | TMEM261 | APOL2 | SLC22A5 | HSPA1B |
| CSF3 | CDCA7L | FAM111A | CCDC146 | MTHFD2L | CSRP2 | MFSD4A | IFITM2 |
| AP000462.1 | CLNS1A | CHCHD2 | SRRM2 | AK3 | RASSF4 | DUSP6 | HSP90AA2P |
| ZNF827 | NEIL2 | ACTL6A | NDUFA8 | LRIG1 | CNDP2 | FXYD3 | PRSS23 |
| TNFRSF21 | EIF3G | AHSA1 | COX4I1 | CAPNS1 | SEC14L1 | AOX1 | NFATC2 |
| DNTTIP2 | ADAMTS6 | EEF1D | PKP2 | ETFRF1 | MRPL3 | LYPLAL1 | ALDH7A1 |
| HS3ST1 | TM2D3 | STX18 | TRIM2 | ATP6V0E2 | GNG5 | HAL | KLF9 |
| ANKRD28 | DDX6 | PBX1 | EDN3 | RCN1 | ZNF652 | FXYD2 | MEIS1 |
| TNFRSF10B | ARHGAP17 | SLC25A5 | NUCKS1 | PAX2 | WDR1 | CITED2 | CBX1 |
| NELFCD | USP7 | SLC12A2 | KRT19 | ATP5J2 | CTNNA2 | SLC44A1 | MYO1B |
| MPRIP-AS1 | GABPB1-AS1 | HNRNPAO | NBEAL1 | NDUFB6 | THEM4 | ATP2C2 | CRISPLD2 |
| MED17 | OXR1 | EDN1 | AKR1C3 | GMNN | MAGED1 | LINC01207 | COL6A3 |
| CTGF | MLLT3 | NONO | YBX1 | COA3 | DYNLT1 | BACE2 | MAP4K4 |
| NFATC1 | GASS | PAICS | HNRNPM | ZBTB11 | NDUFA1 | ACADSB | TINAGL1 |
| DENND4A | EEF1A1P13 | APEX1 | TNS1 | ANAPC16 | CCDC186 | NABP1 | |
| CMTM6 | DEGS2 | NME1 | CTBP1 | FKBP9 | MBP | MAOA | |
| SDCBP | ARID2 | RBBP7 | WDR77 | PTS | TMEM141 | SLC1A1 | |
| FAM133B | RIDA | REC8 | BTG2 | SLC25A1 | ACTN1 | C2CD4A | |

TABLE 2
Stromal genes identified as changing temporally along the menstrual cycle

| | | | | | | | |
|---|---|---|---|---|---|---|---|
| CXCL8 | ADAM12 | HK2 | ELK3 | POSTN | HELLPAR | NCOA7 | TMEM45A |
| C11orf96 | CKS2 | SDCBP | PSMD7 | CNTN1 | ITGB8 | PLIN2 | APOD |
| PMAIP1 | ZBTB43 | CLEC2B | TNFRSF9 | ZNF704 | TMEM196 | LDHA | SNX10 |
| PER2 | MAP1B | TXNRD1 | AMOTL2 | FREM1 | MME | TIMP3 | TGM2 |
| GEM | TNFAIP2 | CDC14A | LIMS1 | IGFBP7 | LETM1 | MTHFD2 | ALDH1A3 |
| STC1 | GCLC | QKI | LAPTM4B | IL33 | TMEM132B | STOM | CFD |
| TNFRSF12A | CADM1 | FOXP1 | ATP13A3 | PAG1 | REV3L | YBX3 | MGP |
| MAP3K8 | FNDC3B | CD59 | MEST | HIST1H4C | NTRK3 | MEDAG | HAND2 |
| UGCG | CRY1 | TP53BP2 | ITGB1 | TRIB2 | JAZF1 | MIF | HSPB1 |
| ERRFI1 | DNAJB6 | ARID4B | RAB22A | MRC2 | FN1 | TLN1 | PRPS2 |
| INHBA | ADAMTS16 | ATP2B1 | RAN | PPP2R2C | CILP | TWISTNB | BCAT1 |
| CDH2 | CD34 | LTBP1 | SDC2 | MTUS2 | NR2F2-AS1 | NME2 | MYL9 |
| ANXA1 | EZR | SNX9 | SERTAD1 | STMN1 | SEMA5A | DKK1 | TXNIP |
| CYTOR | CREB5 | GSPT1 | CSNK1A1 | RBP7 | PARM1 | DAXX | MAOB |
| TGFBI | CD55 | PLK2 | HSPH1 | OLFM1 | SLC12A2 | RAB31 | TUBB |
| MAP2K3 | SCD | STX3 | EGR3 | PGR | TBL1XR1 | S100A4 | TMEM37 |
| HMGA1 | DDX21 | BACH1 | CPM | RUNX1T1 | INTS6 | DPYSL2 | PLA2G2A |
| B4GALT1 | ZBTB38 | ADNP | MEX3D | BRD8 | PLCL1 | CLIC4 | FOX01 |
| NFATC2 | SLC2A1 | EIF3A | AFF4 | PEBP1 | PLEKHH2 | HLA-C | APCDD1 |
| F13A1 | HSPB8 | ATP6V1G1 | LTBP2 | IGDCC4 | PTN | STAT3 | C1orf21 |
| BZW1 | B4GALT5 | PTRF | IFI6 | SKA2 | EBF1 | FKBP1A | HSPB6 |
| SYNJ2 | MAPK6 | HSP90AA2P | PMEPA1 | BEX3 | ELN | LITAF | LMOD1 |
| MAFF | ITGA6 | ILF3 | PIM2 | N4BP2L2 | POLG2 | S100A11 | EFEMP1 |
| MIR4435-2HG | OTUD4 | LAMC1 | SKIL | ZCCHC11 | ABCA1 | PDIA6 | C1R |
| FOSL1 | PPP2CA | EAF1 | TSKU | CACNA1D | PTGDS | FBLN2 | IGF2 |
| MMP7 | RUNX1 | MXD1 | ZBTB2 | GDF7 | SLC26A7 | HLA-A | PILRA |
| PDGFC | RAP2B | NFE2L2 | AHS Al | ECM1 | WEE1 | CXCL14 | RBP1 |
| PIM3 | H2AFZ | MINOS1 | TFAP2C | ZFYVE21 | ARIH1 | INSR | SDHD |
| ABL2 | PTGS2 | SPRY2 | TMED4 | TRAM1 | AKAP12 | CACNB2 | SLC2A8 |
| FJX1 | PFKFB4 | CDKN1A | TPBG | PIP5K1B | CHD1 | TCEAL4 | C1S |
| ELL2 | ZC3H12A | EIF4E | ZFAND2A | HOXA10 | ELMSAN1 | CRYAB | PAPLN |
| TES | KPNA4 | TNIP1 | MIR29A | ZBTB8A | KLF4 | TAGLN | SPTSSA |
| CD44 | MCL1 | TFPI2 | CYR61 | PKD1L2 | BCL6 | ENPP1 | DSTN |
| SDK2 | ETV5 | KIF1B | ALCAM | FAM213A | SERPINE1 | ALDOA | SLC8A1 |
| CAV1 | CCDC85B | IFNGR2 | ID3 | PDS5B | GPRC5A | TPM2 | LCP1 |
| SGK1 | PSMD11 | NAMPTP1 | HSPE1 | PPIB | THBS1 | SERPINF1 | MCC |
| TWIST1 | SQSTM1 | NAMPT | FKBP9 | DIO2 | EMP1 | SELENOP | ENPEP |
| CXCL1 | CFL1 | UBE2D3 | PPP1R15A | P4HA2 | BHLHE40 | PLCD1 | TGFBR2 |
| NRIP1 | PDE4B | CSF1 | USP22 | TMEM144 | KPNA2 | IRS2 | PSMA4 |
| KLF5 | RTN4 | ISOC1 | CPE | ANO1 | OSER1 | PALMD | NUPR1 |
| LRRFIP1 | ERN1 | LINC01588 | COL27A1 | GLG1 | DNAJB1 | AC005062.2 | MMP2 |
| CD83 | FGFR1 | PSMD6 | PAMR1 | HOXA11 | LDLR | DHRS3 | PIK3R1 |
| NINJ1 | ETS2 | PTP4A1 | PCSK5 | SEC22B | MIR22HG | POLR2L | FBLN5 |
| TNC | LRMP | RAP1B | ISLR | SLF2 | ARC | PDLIM1 | AKAP13 |
| CXCL2 | COQ10B | CDV3 | BGN | TRPS1 | TNFAIP3 | ADAMTS9 | ADCY1 |
| BAZ1A | FBXO33 | XBP1 | MMP11 | ANKRD20A11P | HSPA1A | HLA-B | GPX4 |
| SPSB1 | ATP1B1 | KDM6B | MMP16 | DAAM1 | NFKBIZ | LGALS3 | UBL5 |
| RASSF3 | IER3 | CELF2 | TNFRSF19 | TNRC6B | ANXA2 | LAMB1 | AASS |
| BMP2 | PPPIR15B | PLAU | KLF10 | RASSF2 | CAST | AHCY | PDCD5 |
| RIPK2 | NFKB1 | CXADR | GLIPR1 | GXYLT2 | GFPT2 | MGST1 | SLIRP |
| KRT19 | ALYREF | APIG1 | PGRMC1 | CDK6 | ANXA2P2 | ACTA2 | H19 |
| GADD45A | ANKRD28 | IRF2BP2 | MFAP2 | ZNF532 | TUBAIC | SCARA5 | COLEC11 |
| AMFR | LIF | TOP1 | PRSS23 | HSD11B2 | GPX3 | ATP6V0E1 | GABRA2 |
| GFRA2 | ETS1 | TAXIBP1 | WNT5A | FAM46A | TRIB1 | GPX1 | APLP2 |
| DUSP5 | NR3C1 | EPCAM | GUCY1A2 | F3 | SFMBT2 | SERPING1 | MAF |
| NOCT | SEC24A | PDIA4 | CRABP2 | GARNL3 | LMCD1 | NNMT | MASP1 |
| SLC39A14 | MYADM | GTPBP4 | ANO4 | SPEF2 | FGF7 | PSMA7 | ST3GAL5 |
| KLHL21 | FHL2 | ZSWIM6 | PAM | PPM1H | NR4A1 | SRI | PRLR |
| CTNNAL1 | DUSP14 | PODXL | GJA1 | ARHGAP20 | RDH10 | PSME1 | FBXO32 |
| MAP1LC3B | ANK2 | SDC4 | MFAP4 | SPECC1 | ARID5B | PFN1 | UQCR10 |
| CEBPB | B3GNT2 | TMEM2 | FNDC1 | PDGFRA | PAEP | ABCC9 | HAND2-AS1 |
| ARL4C | KMT2C | RNF152 | ALDH1A1 | FAM198B | CYP4B1 | PPP1R14A | MYL12A |
| LMNA | PARD6B | EIF5 | SFRP1 | RBM6 | ATF3 | CAP1 | RBX1 |
| ADM | TLE3 | PHLDA1 | ETV1 | FABP5 | CORO1C | C3 | GLUL |
| PIM1 | RAB7A | PELI1 | SFRP4 | MATN2 | THBS2 | IGFBP4 | APOC1 |
| WDR43 | REL | MSANTD3 | NREP | RORB | ADAMTS5 | IL15 | |

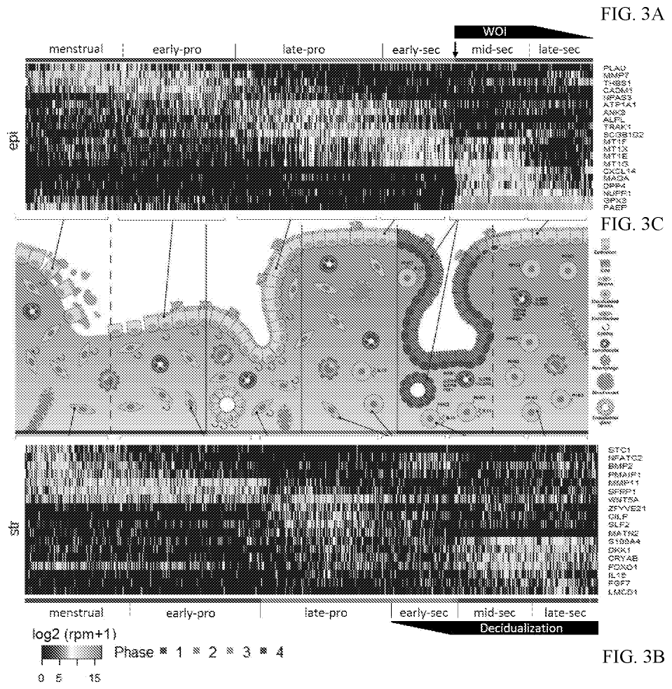

TABLE 3
Short list of Epithelial genes identified as changing
temporally along the menstrual cycle - FIG. 3A

| | | | | |
|---|---|---|---|---|
| PLAU | NPAS3 | TRAK1 | MT1E | DPP4 |
| MMP7 | ATP1A1 | SCGB1D2 | MT1G | NUPR1 |
| THBS1 | ANK3 | MT1F | CXCL14 | GPX3 |
| CADM1 | ALPL | MT1X | MAOA | PAEP |

TABLE 4
Short list of Stromal genes identified as changing
temporally along the menstrual cycle - FIG. 3B

| | | | | |
|---|---|---|---|---|
| STC1 | MMP11 | CILP | DKK1 | FGF7 |
| NFATC2 | SFRP1 | SLF2 | CRYAB | LMCD1 |
| BMP2 | WNT5A | MATN2 | FOXO1 | |
| PMAIP1 | ZFYVE21 | S100A4 | IL15 | |

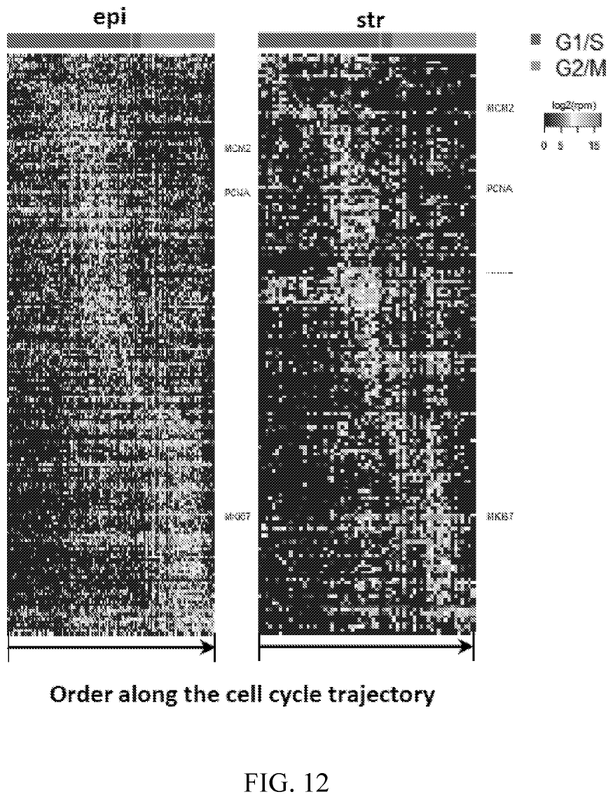

TABLE 5
Epithelial genes identified as expressed in proliferating
cells in proliferative phase endometrium (FIG. 12)

| | | | |
|---|---|---|---|
| MIS18BP1 | CLSPN | MGME1 | ARHGAP11A |
| E2F8 | YEATS4 | TMPO | GTSE1 |
| NUP107 | RFC2 | RFC3 | KIF14 |
| NUDT1 | HELLS | ATAD2 | TACC3 |
| CD320 | MCM7 | GCHFR | FAM64A |
| MRE11 | CKLF | PRIM2 | KIF15 |
| WDHD1 | STIL | KIF23 | CCNA2 |
| ZNF738 | FANCD2 | KNTC1 | PBK |
| CMSS1 | TYMS | BUB1B | MKI67 |
| PKMYT1 | RNASEH2A | HIST1H1E | BUB1 |
| TEX30 | SKA3 | RTKN2 | KNL1 |
| GINS2 | POLE2 | HIST1H1B | PLK4 |
| CHEK1 | KIAA0101 | NUP210 | KIAA1524 |
| ASF1B | TMEM106C | 5PC25 | RACGAP1 |
| FEN1 | LIG1 | CKAP2L | MZT1 |
| MASTL | RFWD3 | DIAPH3 | PRC1 |
| CDK2 | BRIP1 | NUF2 | TPX2 |
| WDR76 | BRI3BP | HIST1H3B | CKS1B |
| CHAF1A | UHRF1 | ANLN | TOP2A |
| CENPH | CDCA5 | CENPK | CEP55 |
| UNG | BRCA2 | KIFC1 | CKAP2 |
| BRCA1 | NCAPG2 | HJURP | KIF20A |
| ORC6 | CDC7 | KIF18A | CDCA2 |
| DTYMK | SLFN13 | ECT2 | CDKN3 |
| RPA3 | VRK1 | NCAPG | NCAPH |
| MCM5 | WHSC1 | TTK | PLK1 |
| CDC6 | ZNF367 | CCNF | DLGAP5 |
| DTL | RAD51 | CDK1 | NCAPD2 |
| TK1 | RAD51AP1 | NUSAP1 | NEK2 |
| CDC45 | MELK | KIF20B | HMMR |
| MCM6 | ATAD5 | SGO1 | NDC1 |
| MCM3 | NRM | CDCA8 | CDC20P1 |
| TMEM97 | MNS1 | CDC25C | SAPCD2 |
| MCM2 | ZWINT | PHF19 | DEPDC1 |
| EIF4EBP1 | CENPM | IQGAP3 | KNSTRN |
| CDCA7 | TTF2 | ASPM | CCNB2 |
| ACOT7 | MAD2L1 | AURKB | ITGB3BP |
| PPIL1 | ESCO2 | KIF11 | CDC20 |
| FAM111B | SMC2 | SPAG5 | PRR11 |
| MCM4 | MYBL2 | KIF18B | KIF4A |
| RFC5 | UBE2T | SPDL1 | TROAP |
| LRRCC1 | LMNB2 | UBE2C | CENPN |
| EXO1 | MIS18A | HMGB2 | CENPF |
| RRM2 | CEP78 | CDCA3 | CSE1L |
| DHFR | C17orf53 | KIF22 | FABP5 |
| MCM10 | CENPU | KIF2C | CENPW |
| MTHFD1 | RRM1 | CCDC34 | BIRC5 |
| TIMELESS | SKA1 | NDC80 | GGH |
| TCOF1 | FANCI | 5GO2 | PTTG1 |
| PCNA | E2F2 | SPC24 | NUP155 |
| ZGRF1 | SASS6 | CENPE | LSM6 |
| DNAJC9 | C19orf48 | CCNB1 | ZWILCH |

TABLE 6
Stromal genes identified as expressed in proliferating cells
in proliferative phase endometrium (FIG. 12)

| | | | |
|---|---|---|---|
| GINS2 | NCAPG | NCAPG2 | CDCA3 |
| MCM4 | MAD2L1 | ST8SIA2 | KIFC1 |
| ATAD5 | CLSPN | TPX2 | ANLN |
| CDT1 | MCM10 | IQGAP3 | NEK2 |
| CENPN | SKA3 | PBK | CDCA2 |
| ZGRF1 | CENPK | TOP2A | CDC20 |
| MCM3 | CDK1 | KIF15 | KIF18B |
| BLM | FANCI | NUSAP1 | MKI67 |
| RBL1 | ESCO2 | KNL1 | CENPF |
| WDR78 | XRCC2 | AURKB | DLGAP5 |
| CHTF18 | TMSB15A | APOBEC3B | CDCA8 |
| RNASEH2A | ORC6 | E2F8 | SMC4 |
| CDC6 | CDK2 | C21orf58 | ARHGAP11A |
| ZIM2-AS1 | CEP152 | KIF2C | KIF11 |
| NT5M | TMPO | SPC25 | TROAP |
| MCM2 | E2F2 | BRIP1 | CCNB1 |
| MCM6 | HMGB3 | RACGAP1 | SGO2 |
| HELLS | NEIL3 | HIST1H1D | KIF14 |
| MMS22L | POC1A | TRIP13 | NUF2 |
| CHAF1A | PSMC3IP | KIF4B | KIF22 |
| DDIAS | RRM2 | CKS1B | AURKA |
| DTL | SPC24 | KIF4A | DLEU2 |
| BRCA2 | UBE2T | MELK | CDKN3 |
| CENPU | WDR76 | ECT2 | CCNB2 |
| ZNF367 | HMGB2 | KIF20B | KIAA1524 |
| SHCBP1 | BARD1 | DNA2 | CENPA |
| RAD51AP1 | KIAA0101 | TTK | CKAP2 |
| TUBG1 | ZWINT | CKAP2L | PRC1 |
| PHF19 | FANCD2 | KIF18A | SGO1 |
| ASF1B | UHRF1 | PRR11 | GTSE1 |
| DTYMK | SMC2 | RAD18 | CEP55 |
| MASTL | NCAPD2 | UBE2C | MZT1 |
| KIF23 | ATAD2 | BRCA1 | TACC3 |
| CENPM | FAM64A | RTKN2 | GINS4 |
| TYMS | DIAPH3 | BUB1 | HMGN2P5 |
| DHFR | SKA1 | HMMR | BIRC5 |
| CDC45 | MYBL2 | SPAG5 | PTTG1 |
| MCM5 | TCF19 | PLK4 | KIF20A |
| MND1 | CDCA5 | CENPE | SAPCD2 |
| PCNA | LMNB2 | DEPDC1 | BUB1B |
| RFC3 | TMEM106C | ASPM | GGH |
| DEPDC1B | HIST1H3B | HJURP | CIT |
| TK1 | HIST1H1A | NDC80 | OIP5 |

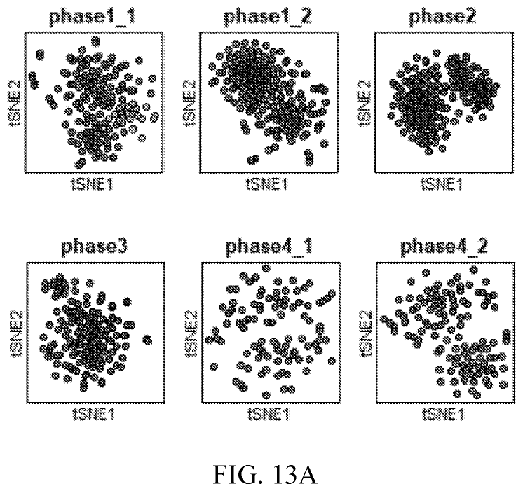

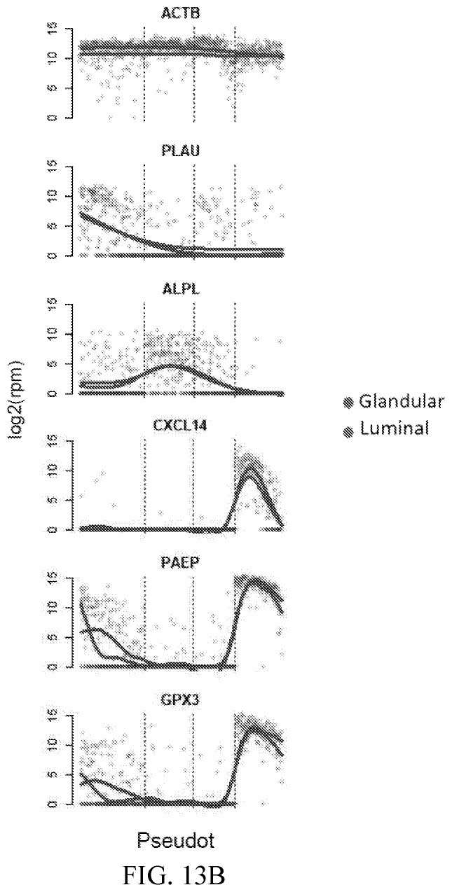

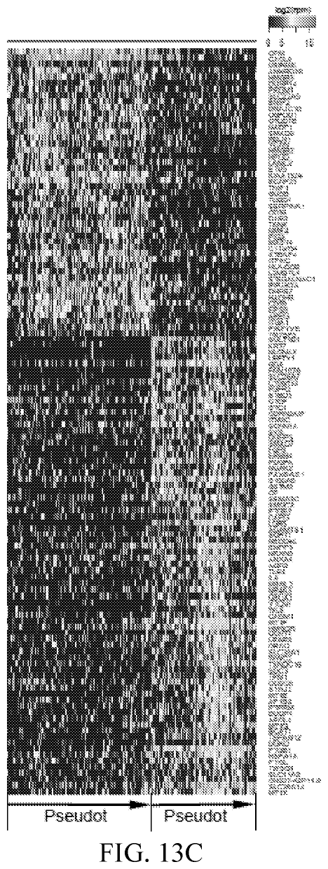

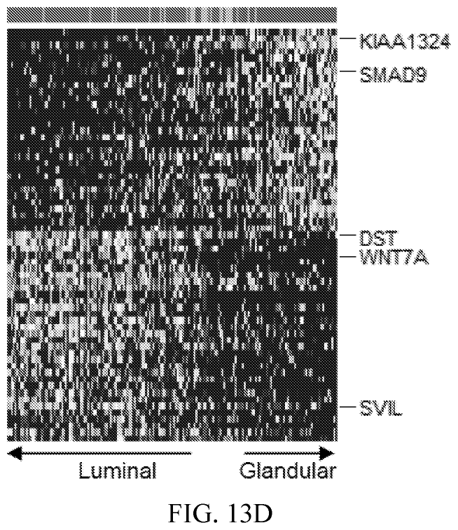

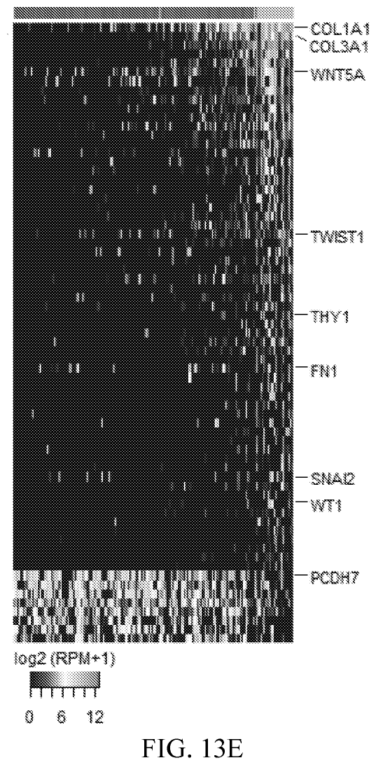

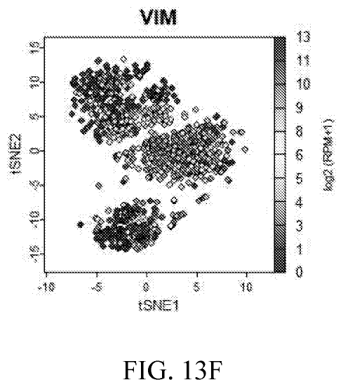

TABLE 7
Genes identified as differentially expressed between luminal and glandular
epithelium during proliferative phase endometrium (Group 1 - Fig 13C -
Upregulated in glandular epithelium)

| | | | | | |
|---|---|---|---|---|---|
| CPM | DNAJC10 | VCAN | TNIP1 | PIGA | PIP4K2A |
| CXCL8 | OGFOD1 | HMGB2 | GUSB | MAST4 | DHRS7 |
| USP6NL | OTUD7B | HPGD | TUBD1 | C11orf54 | HADHB |
| ANKRD28 | NUMA1 | LAMC2 | STXBP2 | GCNT3 | CD59 |
| HMGB3 | CYBA | ETV5 | SERPINA1 | STEAP4 | EPS8 |
| DUSP14 | SEC61A1 | KIAA1324 | CD36 | ITPKC | AREG |
| PRDM1 | NABP1 | EMG1 | DAB2 | HLA-DOB | ITGA1 |
| SLC22A5 | SMAD9 | BCAP29 | TANK | L3MBTL4 | PIKFYVE |
| BNIP2 | FBLN1 | NPDC1 | NME4 | ST6GALNAC1 | TM7SF3 |

TABLE 8
Genes identified as differentially expressed between luminal and glandular
epithelium during proliferative phase endometrium (Group 2 - FIG 13C -
Upregulated in luminal epithelium)

| | | | | | |
|---|---|---|---|---|---|
| SULT1E1 | SCNN1A | SEMA3C | MBNL2 | SDC3 | PTGS1 |
| KRT7 | TMSB4XP4 | SMOC2 | NR4A3 | TPM1 | HSPA1A |
| NLGN4X | IGFBP2 | PTGS2 | QSOX1 | CCDC6 | PYGL |
| LEFTY1 | SVIL | CAPG | VTCN1 | SLC3A1 | TWSG1 |
| GDA | DUSP5 | LGR5 | WLS | SYNJ2 | SLC11A2 |
| FAM107A | SMAD7 | ADAMTS1 | CADM1 | MT1E | CH507-42P11.8 |
| SLC26A7 | FGF9 | SORT1 | MT1F | AP1S2 | RNF122 |
| C19orf33 | ERBB4 | NEDD4L | HMGCR | PTPRM | SLC39A14 |
| FGFR2 | PDGFA | ENPP3 | GCNT4 | DUSP4 | |
| BTBD3 | NUAK2 | NRXN3 | LPAR3 | APOL4 | |
| CTGF | PAX8-AS1 | ANXA4 | ORAI2 | MT1G | |
| STC1 | S100A6 | AGR2 | SLC38A1 | BCAT1 | |
| CDKN2AIP | GSTM3 | TLE4 | WWC2 | TSPAN12 | |
| ITM2C | CP | IL6 | TXNDC16 | DGKD | |

The biomarkers described herein may have a level in a sample obtained from a subject (i.e., patient) that has an open window of implantation (WOI) that deviates (e.g., is increased or decreased) when compared to the level of the same biomarker in a sample obtained from a subject that does not have an open WOI. The biomarkers described herein may have a level in decidualized cells that deviates (i.e., is increased or reduced) from the level of the same marker in non-decidualized cells by at least 20% (e.g., 30%, 50%, 80%, 100%, 2-fold, 5-fold, 10-fold, 20-fold, 50-fold, 100-fold or more). Such a biomarker or set of biomarkers may be used in both diagnostic/prognostic applications and non-clinical applications (e.g., for research purposes).

In some embodiments, epithelial biomarkers are one or more of PLAU, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, PAEP (see FIG. 3A). In some embodiments, stromal biomarkers are one or more of STC1, NFATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1 (see FIG. 3B).

In other embodiments, the unciliated epithelial biomarkers include the following subset or panel of biomarkers that are associated with a window of implantation.

TABLE 9
Unciliated epithelial panel of biomarkers associated
withthe window of implantation

| | UP (+) or DOWN (−) | Biomarker |
|---|---|---|
| Biomarker | regulated | classification |
| PLAU | − | Negative |
| THBS1 | − | Negative |
| CADM1 | − | Negative |
| NPAS3 | − | Negative |
| MMP7 | − | Negative |
| ATP1A1 | − | Negative |
| ANK3 | − | Negative |
| ALPL | − | Negative |
| TRAK1 | − | Negative |
| SCGB1D2 | − | Negative |
| MT1F | + | Type 1 |
| MT1X | + | Type 1 |
| MT1E | + | Type 1 |
| MT1G | + | Type 1 |
| CXCL14 | + | Type 2 |
| MAOA | + | Type 2 |
| DPP4 | + | Type 2 |
| NUPR1 | + | Type 2 |
| GPX3 | + | Type 2 |
| PAEP | + | Type 2 |

In still other embodiments, the stromal biomarkers include the following subset or panel of biomarkers that are associated with a window of implantation.

TABLE 10
Stromal panel of biomarkers associated with the window
of implantation

| | UP (+) or DOWN (−) | Biomarker |
|---|---|---|
| Biomarker | regulated | classification |
| STC1 | − | Negative |
| NFATC2 | − | Negative |
| BMP2 | − | Negative |
| PMAIP1 | − | Negative |
| MMP11 | − | Negative |
| SFRP1 | − | Negative |
| WNT5A | − | Negative |
| ZFYVE21 | − | Negative |
| CILP | − | Negative |
| SLF2 | − | Negative |
| MATN2 | − | Negative |
| S100A4 | + | Type 2 |
| DKK1 | + | Type 2 |
| CRYAB | + | Type 2 |
| FOXO1 | + | Type 2 |
| IL15 | + | Type 2 |
| FGF7 | −/+ | Type 2 |
| LMCD1 | −/+ | Type 2 |

In reference to Tables 9 and 10 with regard to whether the expression of a biomarker (e.g., CADM1) at any point in time during the menstrual cycle (e.g., the point of WOI) considered “up” (+) or “down” (−) regulated depends the relative level of expression of that biomarker at the particular point in time of interest (e.g., point of WOI) relative to the point in the menstrual cycle of peak expression of that biomarker. The peak expression level is determined computationally by a known computation method. Thus, biomarkers such as CADM1 and NPAS3 showed peak expression during the proliferative phase of the menstrual cycle; thus, the expression at the WOI was ascribed a value of “down-regulated.” To the contrary, NUPR1 was ascribed an expression value of “up-regulated” since its expression peaked in the WOI.

The biomarkers of Table 9 and 10 may be further classified into three broad categories:

1. A negative biomarker: its expression falls above a threshold indicates a classification of “out of WOI” (e.g., CADMI, ATP1A1, ALPL, FGF7, or LMCD1). In general, these markers are not expressed in WOI, but are expressed in other major phases of the menstrual cycle. Therefore, considerable expression of these genes would indicate “out of WOI.”

2. A type 1 positive biomarker: its expression falls above a threshold indicates a classification of “likely within early-sec or WOI” (e.g., MT1F, X, E, G). These biomarkers show considerable expression in early-sec or WOI relative to their expression levels in other phases of the menstrual cycle.

3. A type 2 positive biomarker: its expression falls above a threshold indicates a classification of “likely within late-sec or WOI” (e.g., CXCL14, PAEP, FGF7, LMCD1). These biomarkers show considerable expression in late-sec or WOI relative to their expression levels in other phases of the menstrual cycle.

There are many potential ways to build the gene classifiers described herein, as well as other gene classifiers, for predicting one or more phases or events (e.g., WOI) during the menstrual cycle, including determining the thresholds.

In one possible approach, a machine learning based method can be used to build a classifier (e.g., a support vector machine, random forest). The expression profile of the biomarkers would then be used to train a classifier on training sample sets, deriving thresholds for the markers (which would most likely be different for different markers). Then the classifiers would be tested on sample sets. Via cross-validation, the most informative genes and their corresponding thresholds would be able to be determined.

In another approach, a gene set enrichment (GSEA) based method could be used to build a classifier. Given the fact that the genes selected in FIG. 3 are generally binary between stages of interest and other stages, a threshold could be set to indicate when a gene is “expressed” or not, e.g., 5% of the peak expression of the gene (the threshold here may be the same for different markers then). The most informative genes and their particular threshold can be determined using cross-validation.

In certain embodiments, the detection methods may rely on the predictive value of only a single biomarker, such as a biomarker that has a relatively exclusive expression in a certain phase, e.g., in WOI (e.g., IL15). In other embodiments, the detection methods may rely on the predictive value of biomarkers which show up-regulation in WOI relative to late-sec phase (e.g., IL15, CXCL14, MAOA, or DPP4).

In certain other embodiments, the detection methods may rely on a combination of epithelial biomarkers from FIG. 3A from different categories (e.g., a combination of a negative biomarker, a type 1 biomarker, and a type 2 biomarker of Table 9). In still other embodiments, the detection methods may rely on a combination of stromal biomarkers from FIG. 3B from different categories (e.g., a combination of a negative biomarker, a type 1 biomarker, and a type 2 biomarker of Table 10). Combinations of negative, type 1, and type 2 biomarkers from Table 9 (epithelial) and Table 10 (stromal) are also contemplated as giving satisfactory confidence in predictive value of an event, e.g., MOI.

The biomarkers identified in FIG. 3A (PLAU, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP) and FIG. 3B (STC1, NFATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1) are not limited to a particular sequence and can include any variant. Exemplary sequences embraced by the present Application include:

FIG. 3A Exemplary GenBank Accession Nos. and Amino Acid Sequences for
Epithelium Biomarkers

| Gene | Gene name | Function (Uniprot) | Accession | NCBI Reference Sequence |
|---|---|---|---|---|
| PLAU | Plasminogen | Specifically cleaves | NP_001138503.1 | MVFHLRTRYEQANCDCLNGGTCV |
| | Activator, | the zymogen | | SNKYFSNIHWCNCPKKFGGQHCEI |
| | Urokinase | plasminogen to form | | DKSKTCYEGNGHFYRGKASTDTM |
| | | the active enzyme | | GRPCLPWNSATVLQQTYHAHRSD |
| | | plasmin. | | ALQLGLGKHNYCRNPDNRRRPWC |
| | | | | YVQVGLKPLVQECMVHDCADGK |
| | | | | KPSSPPEELKFQCGQKTLRPRFKIIG |
| | | | | GEFTTIENQPWFAAIYRRHRGGSV |
| | | | | TYVCGGSLISPCWVISATHCFI |
| | | | | DYPKKEDYIVYLGRSRLNSNTQGE |
| | | | | MKFEVENLILHKDYSADTLAHHN |
| | | | | DIALLKIRSKEGRCAQPSRTIQT |
| | | | | ICLPSMYNDPQFGTSCEITGFGKEN |
| | | | | STDYLYPEQLKMTVVKLISHRECQ |
| | | | | QPHYYGSEVTTKMLCAADPQW |
| | | | | KTDSCQGDSGGPLVCSLQGRMTLT |
| | | | | GIVSWGRGCALKDKPGVYTRVSH |
| | | | | FLPWIRSHTKEENGLAL |
| | | | | (SEQ ID NO: 1) |
| MMP7 | Matrix | Degrades casein, | NP_002414.1 | MRLTVLCAVCLLPGSLALPLPQEA |
| | Metallopeptidase | gelatins of types I, | | GGMSELQWEQAQDYLKRFYLYDS |
| | 7 | III, IV, and V, and | | ETKNANSLEAKLKEMQKFFGLPI |
| | | fibronectin. | | TGMLNSRVIEIMQKPRCGVPDVAE |
| | | Activates | | YSLFPNSPKWTSKVVTYRIVSYTR |
| | | procollagenase. | | DLPHITVDRLVSKALNMWGKEI |
| | | | | PLHFRKVVWGTADIMIGFARGAH |
| | | | | GDSYPFDGPGNTLAHAFAPGTGLG |
| | | | | GDAHFDEDERWTDGSSLGINFLY |
| | | | | AATHELGHSLGMGHSSDPNAVMY |
| | | | | PTYGNGDPQNFKLSQDDIKGIQKL |
| | | | | YGKRSNSRKK (SEQ ID NO: 2) |
| THBS1 | Thrombospondin | Adhesive | NP_003237.2 | MGLAWGLGVLFLMHVCGTNRIPE |
| | 1 | glycoprotein that | | SGGDNSVFDIFELTGAARKGSGRR |
| | | mediates cell-to-cell | | LVKGPDPSSPAFRIEDANLIPPVPD |
| | | and cell-to-matrix | | DKFQDLVDAVRAEKGFLLLASLR |
| | | interactions. Binds | | QMKKTRGTLLALERKDHSGQVFS |
| | | heparin. May play a | | VVSNGKAGTLDLSLTVQGKQHVV |
| | | role in | | SVEEALLATGQWKSITLFVQEDRA |
| | | dentinogenesis | | QLYIDCEKMENAELDVPIQSVFTR |
| | | and/or maintenance | | DLASIARLRIAKGGVNDNFQGVLQ |
| | | of dentin and dental | | NVRFVFGTTPEDILRNKGCSSSTSV |
| | | pulp (By similarity). | | LLTLDNNVVNGSSPAIRTNYIGHK |
| | | Ligand for CD36 | | TKDLQAICGISCDELSSMVLELRGL |
| | | mediating | | RTIVTTLQDSIRKVTEENKELANEL |
| | | antiangiogenic | | RRPPLCYHNGVQYRNNEEWTVDS |
| | | properties. Plays a | | CTECHCQNSVTICKKVSCPIMPCSN |
| | | role in ER stress | | ATVPDGECCPRCWPSDSADDGWS |
| | | response, via its | | PWSEWTSCSTSCGNGIQQRGRSCD |
| | | interaction with the | | SLNNRCEGSSVQTRTCHIQECDKR |
| | | activating | | FKQDGGWSHWSPWSSCSVTCGDG |
| | | transcription factor 6 | | VITRIRLCNSPSPQMNGKPCEGEAR |
| | | alpha (ATF6) which | | ETKACKKDACPINGGWGPWSPWD |
| | | produces adaptive | | ICSVTCGGGVQKRSRLCNNPTPQF |
| | | ER stress response | | GGKDCVGDVTENQICNKQDCPIDG |
| | | factors (By | | CLSNPCFAGVKCTSYPDGSWKCG |
| | | similarity). | | ACPPGYSGNGIQCTDVDECKEVPD |
| | | | | ACFNHNGEHRCENTDPGYNCLPCP |
| | | | | PRFTGSQPFGQGVEHATANKQVC |
| | | | | KPRNPCTDGTHDCNKNAKCNYLG |
| | | | | HYSDPMYRCECKPGYAGNGIICGE |
| | | | | DTDLDGWPNENLVCVANATYHCK |
| | | | | KDNCPNLPNSGQEDYDKDGIGDA |
| | | | | CDDDDDNDKIPDDRDNCPFHYNP |
| | | | | AQYDYDRDDVGDRCDNCPYNHN |
| | | | | PDQADTDNNGEGDACAADIDGDG |
| | | | | ILNERDNCQYVYNVDQRDTDMDG |
| | | | | VGDQCDNCPLEHNPDQLDSDSDRI |
| | | | | GDTCDNNQDIDEDGHQNNLDNCP |
| | | | | YVPNANQADHDKDGKGDACDHD |
| | | | | DDNDGIPDDKDNCRLVPNPDQKD |
| | | | | SDGDGRGDACKDDFDHDSVPDID |
| | | | | DICPENVDISETDFRRFQMIPLDPK |
| | | | | GTSQNDPNWVVRHQGKELVQTVN |
| | | | | CDPGLAVGYDEFNAVDFSGTFFIN |
| | | | | TERDDDYAGFVFGYQSSSRFYVV |
| | | | | MWKQVTQSYWDTNPTRAQGYSG |
| | | | | LSVKVVNSTTGPGEHLRNALWHT |
| | | | | GNTPGQVRTLWHDPRHIGWKDFT |
| | | | | AYRWRLSHRPKTGFIRVVMYEGK |
| | | | | KIMADSGPIYDKTYAGGRLGLFVF |
| | | | | SQEMVFFSDLKYECRDP |
| | | | | (SEQ ID NO: 3) |
| CADM1 | Cell Adhesion | Mediates | NP_001091987.1 | MASVVLPSGSQCAAAAAAAAPPG |
| | Molecule 1 | homophilic cell-cell | | LRLRLLLLLFSAAALIPTGDGQNLF |
| | | adhesion in a | | TKDVTVIEGEVATISCQVNKSD |
| | | Ca(2+)-independent | | DSVIQLLNPNRQTIYFRDFRPLKDS |
| | | manner. Also | | RFQLLNFSSSELKVSLTNVSISDEG |
| | | mediates | | RYFCQLYTDPPQESYTTITV |
| | | heterophilic cell-cell | | LVPPRNLMIDIQKDTAVEGEEIEVN |
| | | adhesion with | | CTAMASKPATTIRWFKGNTELKG |
| | | CADM3 and | | KSEVEEWSDMYTVTSQLMLKVH |
| | | NECTIN3 in a | | KEDDGVPVICQVEHPAVTGNLQTQ |
| | | Ca(2+)-independent | | RYLEVQYKPQVHIQMTYPLQGLTR |
| | | manner. Acts as a | | EGDALELTCEAIGKPQPVMVTW |
| | | tumor suppressor in | | VRVDDEMPQHAVLSGPNLFINNLN |
| | | non-small-cell lung | | KTDNGTYRCEASNIVGKAHSDYM |
| | | cancer (NSCLC) | | LYVYDSRAGEEGSIRAVDHAVIG |
| | | cells. Interaction | | GVVAVVVFAMLCLLIILGRYFARH |
| | | with CRTAM | | KGTYFTHEAKGADDAADADTAIIN |
| | | promotes natural | | AEGGQNNSEEKKEYFI |
| | | killer (NK) cell | | (SEQ ID NO: 4) |
| | | cytotoxicity and | | |
| | | interferon-gamma | | |
| | | (IFN-gamma) | | |
| | | secretion by CD8+ | | |
| | | cells in vitro as well | | |
| | | as NK cell-mediated | | |
| | | rejection of tumors | | |
| | | expressing CADM3 | | |
| | | in vivo. May | | |
| | | contribute to the less | | |
| | | invasive phenotypes | | |
| | | of lepidic growth | | |
| | | tumor cells. In mast | | |
| | | cells, may mediate | | |
| | | attachment to and | | |
| | | promote | | |
| | | communication with | | |
| | | nerves. CADM1, | | |
| | | together with MITF, | | |
| | | is essential for | | |
| | | development and | | |
| | | survival of mast | | |
| | | cells in vivo. Acts | | |
| | | as a synaptic cell | | |
| | | adhesion molecule | | |
| | | and plays a role in | | |
| | | the formation of | | |
| | | dendritic spines and | | |
| | | in synapse assembly | | |
| | | (By similarity). | | |
| | | May be involved in | | |
| | | neuronal migration, | | |
| | | axon growth, | | |
| | | pathfinding, and | | |
| | | fasciculation on the | | |
| | | axons of | | |
| | | differentiating | | |
| | | neurons. May play | | |
| | | diverse roles in the | | |
| | | spermatogenesis | | |
| | | including in the | | |
| | | adhesion of | | |
| | | spermatocytes and | | |
| | | spermatids to Sertoli | | |
| | | cells and for their | | |
| | | normal | | |
| | | differentiation into | | |
| | | mature spermatozoa. | | |
| NPAS3 | Neuronal PAS | May play a broad | NP_001158221.1 | MAPTKPSFQQDPSRRERITAQHPLP |
| | Domain | role in neurogenesis. | | NQSECRKIYRYDGIYCESTYQNLQ |
| | Protein 3 | May control | | ALRKEKSRDAARSRRGKENFEFYE |
| | | regulatory pathways | | LAKLLPLPAAITSQLDKASIIRLTIS |
| | | relevant to | | YLKMRDFANQGDPPWNLRMEGPP |
| | | schizophrenia and to | | PNTSVKVIGAQRRRSPSALAIEVFE |
| | | psychotic illness (By | | AHLGSHILQSLDGFVFALNQEGKF |
| | | similarity). | | LYISETVSIYLGLSQVELTGSSVFD |
| | | | | YVHPGDHVEMAEQLGMKLPPGRG |
| | | | | LLSQGTAEDGASSASSSSQSETPEP |
| | | | | VESTSPSLLTTDNTLERSFFIRMKST |
| | | | | LTKRGVHIKSSGYKVIHITGRLRLR |
| | | | | VSLSHGRTVPSQIMGLVVVAHALP |
| | | | | PPTINEVRIDCHMFVTRVNMDLNII |
| | | | | YCENRISDYMDLTPVDIVGKRCYH |
| | | | | FIHAEDVEGIRHSHLDLLNKGQCV |
| | | | | TKYYRWMQKNGGYIWIQSSATIAI |
| | | | | NAKNANEKNIIWVNYLLSNPEYKD |
| | | | | TPMDIAQLPHLPEKTSESSETSDSE |
| | | | | SDSKDTSGITEDNENSKSDEKGNQ |
| | | | | SENSEDPEPDRKKSGNACDNDMN |
| | | | | CNDDGHSSSNPDSRDSDDSFEHSD |
| | | | | PENPKAGEDGFGALGAMQIKVER |
| | | | | YVESESDLRLQNCESLTSDSAKDS |
| | | | | DSAGEAGAQASSKHQKRKKRRKR |
| | | | | QKGGSASRRRLSSASSPGGLDAGL |
| | | | | VEPPRLLSSPNSASVLKIKTEISEPIN |
| | | | | FDNDSSIWNYPPNREISRNESPYSM |
| | | | | TKPPSSEHFPSPQGGGGGGGGGGG |
| | | | | LHVAIPDSVLTPPGADGAAARKTQ |
| | | | | FGASATAALAPVASDPLSPPLSASP |
| | | | | RDKHPGNGGGGGGGGGGAGGGG |
| | | | | PSASNSLLYTGDLEALQRLQAGNV |
| | | | | VLPLVHRVTGTLAATSTAAQRVYT |
| | | | | TGTIRYAPAEVTLAMQSNLLPNAH |
| | | | | AVNFVDVNSPGFGLDPKTPMEML |
| | | | | YHHVHRLNMSGPFGGAVSAASLT |
| | | | | QMPAGNVFTTAEGLFSTLPFPVYS |
| | | | | NGIHAAQTLERKED |
| | | | | (SEQ ID NO: 5) |
| ATP1A1 | ATPase | This is the catalytic | NP_000692.2 | MGKGVGRDKYEPAAVSEQGDKK |
| | Na+/K+ | component of the | | GKKGKKDRDMDELKKEVSMDDH |
| | Transporting | active enzyme, | | KLSLDELHRKYGTDLSRGLTSARA |
| | Subunit Alpha | which catalyzes the | | AEILARDGPNALTPPPTTPEWIKFC |
| | 1 | hydrolysis of ATP | | RQLFGGFSMLLWIGAILCFLAYSIQ |
| | | coupled with the | | AATEEEPQNDNLYLGVVLSAVVII |
| | | exchange of sodium | | TGCFSYYQEAKSSKIMESFKNMVP |
| | | and potassium ions | | QQALVIRNGEKMSINAEEVVVGDL |
| | | across the plasma | | VEVKGGDRIPADLRIISANGCKVD |
| | | membrane. This | | NSSLTGESEPQTRSPDFTNENPLET |
| | | action creates the | | RNIAFFSTNCVEGTARGIVVYTGD |
| | | electrochemical | | RTVMGRIATLASGLEGGQTPIAAEI |
| | | gradient of sodium | | EHFIHIITGVAVFLGVSFFILSLILEY |
| | | and potassium ions, | | TWLEAVIFLIGIIVANVPEGLLATV |
| | | providing the energy | | TVCLTLTAKRMARKNCLVKNLEA |
| | | for active transport | | VETLGSTSTICSDKTGTLTQNRMT |
| | | of various nutrients. | | VAHMWFDNQIHEADTTENQSGVS |
| | | | | FDKTSATWLALSRIAGLCNRAVFQ |
| | | | | ANQENLPILKRAVAGDASESALLK |
| | | | | CIELCCGSVKEMRERYAKIVEIPFN |
| | | | | STNKYQLSIHKNPNTSEPQHLLVM |
| | | | | KGAPERILDRCSSILLHGKEQPLDE |
| | | | | ELKDAFQNAYLELGGLGERVLGFC |
| | | | | HLFLPDEQFPEGFQFDTDDVNFPID |
| | | | | NLCFVGLISMIDPPRAAVPDAVGK |
| | | | | CRSAGIKVIMVTGDHPITAKAIAKG |
| | | | | VGIISEGNETVEDIAARLNIPVSQV |
| | | | | NPRDAKACVVHGSDLKDMTSEQL |
| | | | | DDILKYHTEIVFARTSPQQKLIIVEG |
| | | | | CQRQGAIVAVTGDGVNDSPALKK |
| | | | | ADIGVAMGIAGSDVSKQAADMILL |
| | | | | DDNFASIVTGVEEGRLIFDNLKKSI |
| | | | | AYTLTSNIPEITPFLIFIIANIPLPLGT |
| | | | | VTILCIDLGTDMVPAISLAYEQAES |
| | | | | DIMKRQPRNPKTDKLVNERLISMA |
| | | | | YGQIGMIQALGGFFTYFVILAENGF |
| | | | | LPIHLLGLRVDWDDRWINDVEDSY |
| | | | | GQQWTYEQRKIVEFTCHTAFFVSI |
| | | | | VVVQWADLVICKTRRNSVFQQGM |
| | | | | KNKILIFGLFEETALAAFLSYCPGM |
| | | | | GVALRMYPLKPTWWFCAFPYSLLI |
| | | | | FVYDEVRKLIIRRRPGGWVEKETY |
| | | | | Y (SEQ ID NO: 6) |
| ANK3 | Ankyrin 3 | In skeletal muscle, | NP_001140.2 | MALPQSEDAMTGDTDKYLGPQDL |
| | | required for | | KELGDDSLPAEGYMGFSLGARSAS |
| | | costamere | | LRSFSSDRSYTLNRSSYARDSMMIE |
| | | localization of DMD | | ELLVPSKEQHLTFTREFDSDSLRHY |
| | | and betaDAG1 (By | | SWAADTLDNVNLVSSPIHSGFLVS |
| | | similarity). | | FMVDARGGSMRGSRHHGMRIIIPP |
| | | Membrane- | | RKCTAPTRITCRLVKRHKLANPPP |
| | | cytoskeleton linker. | | MVEGEGLASRLVEMGPAGAQFLG |
| | | May participate in | | PVIVEIPHFGSMRGKERELIVLRSE |
| | | the | | NGETWKEHQFDSKNEDLTELLNG |
| | | maintenance/targeting | | MDEELDSPEELGKKRICRIITKDFP |
| | | of ion channels | | QYFAVVSRIKQESNQIGPEGGILSS |
| | | and cell adhesion | | TTVPLVQASFPEGALTKRIRVGLQ |
| | | molecules at the | | AQPVPDEIVKKILGNKATFSPIVTV |
| | | nodes of Ranvier | | EPRRRKFHKPITMTIPVPPPSGEGV |
| | | and axonal initial | | SNGYKGDTTPNLRLLCSITGGTSPA |
| | | segments. | | QWEDITGTTPLTFIKDCVSFTTNVS |
| | | Regulates KCNA1 | | ARFWLADCHQVLETVGLATQLYR |
| | | channel activity in | | ELICVPYMAKFVVFAKMNDPVESS |
| | | function of dietary | | LRCFCMTDDKVDKTLEQQENFEE |
| | | Mg(2+) levels, and | | VARSKDIEVLEGKPIYVDCYGNLA |
| | | thereby contributes | | PLTKGGQQLVFNFYSFKENRLPFSI |
| | | to the regulation of | | KIRDTSQEPCGRLSFLKEPKTTKGL |
| | | renal Mg(2+) | | PQTAVCNLNITLPAHKKIEKTDRR |
| | | reabsorption | | QSFASLALRKRYSYLTEPGMSPQS |
| | | (PubMed: 23903368) | | PCERTDIRMAIVADHLGLSWTELA |
| | | .\|\|Isoform 5: May be | | RELNFSVDEINQIRVENPNSLISQSF |
| | | part of a Golgi- | | MLLKKWVTRDGKNATTDALTSVL |
| | | specific membrane | | TKINRIDIVTLLEGPIFDYGNISGTR |
| | | cytoskeleton in | | SFADENNVFHDPVDGYPSLQVELE |
| | | association with | | TPTGLHYTPPTPFQQDDYFSDISSIE |
| | | beta-spectrin. | | SPLRTPSRLSDGLVPSQGNIEHSAD |
| | | | | GPPVVTAEDASLEDSKLEDSVPLT |
| | | | | EMPEAVDVDESQLENVCLSWQNE |
| | | | | TSSGNLESCAQARRVTGGLLDRLD |
| | | | | DSPDQCRDSITSYLKGEAGKFEAN |
| | | | | GSHTEITPEAKTKSYFPESQNDVGK |
| | | | | QSTKETLKPKIHGSGHVEEPASPLA |
| | | | | AYQKSLEETSKLIIEETKPCVPVSM |
| | | | | KKMSRTSPADGKPRLSLHEEEGSS |
| | | | | GSEQKQGEGFKVKTKKEIRHVEKK |
| | | | | SHS (SEQ ID NO: 7) |
| ALPL | Alkaline | This isozyme may | NP_000469.3 | MISPFLVLAIGTCLTNSLVPEKEKD |
| | Phosphatase, | play a role in | | PKYWRDQAQETLKYALELQKLNT |
| | Liver/Bone/Kidney | skeletal | | NVAKNVIMFLGDGMGVSTVTAA |
| | | mineralization. | | RILKGQLHHNPGEETRLEMDKFPF |
| | | | | VALSKTYNTNAQVPDSAGTATAY |
| | | | | LCGVKANEGTVGVSAATERSRCN |
| | | | | TTQGNEVTSILRWAKDAGKSVGIV |
| | | | | TTTRVNHATPSAAYAHSADRDWY |
| | | | | SDNEMPPEALSQGCKDIAYQLMH |
| | | | | NIRDIDVIMGGGRKYMYPKNKTD |
| | | | | VEYESDEKARGTRLDGLDLVDTW |
| | | | | KSFKPRYKHSHFIWNRTELLTLDP |
| | | | | HNVDYLLGLFEPGDMQYELNRNN |
| | | | | VTDPSLSEMVVVAIQILRKNPKGFF |
| | | | | LLVEGGRIDHGHHEGKAKQALH |
| | | | | EAVEMDRAIGQAGSLTSSEDTLTV |
| | | | | VTADHSHVFTFGGYTPRGNSIFGL |
| | | | | APMLSDTDKKPFTAILYGNGPG |
| | | | | YKVVGGERENVSMVDYAHNNYQ |
| | | | | AQSAVPLRHETHGGEDVAVFSKGP |
| | | | | MAHLLHGVHEQNYVPHVMAYAA |
| | | | | CIGANLGHCAPASSAGSLAAGPLL |
| | | | | LALALYPLSVLF (SEQ ID NO: 8) |
| TRAK1 | Trafficking | Involved in the | NP_001036111.1 | MALVFQFGQPVRAQPLPGLCHGK |
| | Kinesin | regulation of | | LIRTNACDVCNSTDLPEVEIISLLEE |
| | Protein 1 | endosome-to- | | QLPHYKLRADTIYGYDHDDWLHT |
| | | lysosome | | PLISPDANIDLTTEQIEETLKYFLLC |
| | | trafficking, | | AERVGQMTKTYNDIDAVTRLLEE |
| | | including endocytic | | KERDLELAARIGQSLLKKNKTLTE |
| | | trafficking of EGF- | | RNELLEEQVEHIREEVSQLRHELS |
| | | EGFR complexes | | MKDELLQFYTSAAEESEPESVCSTP |
| | | and GABA-A | | LKRNESSSSVQNYFHLDSLQKKLK |
| | | receptors. | | DLEEENVVLRSEASQLKTETITYEE |
| | | | | KEQQLVNDCVKELRDANVQIASIS |
| | | | | EELAKKTEDAARQQEEITHLLSQIV |
| | | | | DLQKKAKACAVENEELVQHLGAA |
| | | | | KDAQRQLTAELRELEDKYAECME |
| | | | | MLHEAQEELKNLRNKTMPNTTSR |
| | | | | RYHSLGLFPMDSLAAEIEGTMRKE |
| | | | | LQLEEAESPDITHQKRVFETVRNIN |
| | | | | QVVKQRSLTPSPMNIPGSNQSSAM |
| | | | | NSLLSSCVSTPRSSFYGSDIGNVVL |
| | | | | DNKTNSIILETEAADLGNDERSKKP |
| | | | | GTPGTPGSHDLETALRRLSLRREN |
| | | | | YLSERRFFEEEQERKLQELAEKGE |
| | | | | LRSGSLTPTESIMSLGTHSRFSEFTG |
| | | | | FSGMSFSSRSYLPEKLQIVKPLEGS |
| | | | | ATLHHWQQLAQPHLGGILDPRPG |
| | | | | VVTKGFRTLDVDLDEVYCLNDFEE |
| | | | | DDTGDHISLPRLATSTPVQHPETSA |
| | | | | HHPGKCMSQTNSTFTFTTCRILHPS |
| | | | | DELTRVTPSLNSAPTPACGSTSHLK |
| | | | | STPVATPCTPRRLSLAESFTNTRES |
| | | | | TTTMSTSLGLVWLLKERGISAAVY |
| | | | | DPQSWDRAGRGSLLHSYTPKMAV |
| | | | | IPSTPPNSPMQTPTSSPPSFEFKCTSP |
| | | | | PYDNFLASKPASSILREVREKNVRS |
| | | | | SESQTDVSVSNLNLVDKVRRFGVA |
| | | | | KVVNSGRAHVPTLTEEQGPLLCGP |
| | | | | PGPAPALVPRGLVPEGLPLRCPTVT |
| | | | | SAIGGLQLNSGIRRNRSFPTMVGSS |
| | | | | MQMKAPVTLTSGILMGAKLSKQT |
| | | | | SLR (SEQ ID NO: 9) |
| SCGB1D2 | Secretoglobin | May bind androgens | NP_006542.1 | MKLSVCLLLVTLALCCYQANAEF |
| | Family 1D | and other steroids, | | CPALVSELLDFFFISEPLFKLSLAKF |
| | Member 2 | may also bind | | DAPPEAVAAKLGVKRCTDQMS |
| | | estramustine, a | | LQKRSLIAEVLVKILKKCSV |
| | | chemotherapeutic | | (SEQ ID NO: 10) |
| | | agent used for | | |
| | | prostate cancer. | | |
| | | May be under | | |
| | | transcriptional | | |
| | | regulation of steroid | | |
| | | hormones. | | |
| MT1F | Metallothionein | Metallothioneins | NP_001288201.1 | MDPNCSCAAGVSCTCAGSCKCKE |
| | 1F | have a high content | | CKCTSCKKSECEAISMVWGCG |
| | | of cysteine residues | | (SEQ ID NO: 11) |
| | | that bind various | | |
| | | heavy metals; these | | |
| | | proteins are | | |
| | | transcriptionally | | |
| | | regulated by both | | |
| | | heavy metals and | | |
| | | glucocorticoids. | | |
| MT1X | Metallothionein | Metallothioneins | NP_005943.1 | MDPNCSCSPVGSCACAGSCKCKEC |
| | 1X | have a high content | | KCTSCKKSCCSCCPVGCAKCAQG |
| | | of cysteine residues | | CICKGTSDKCSCCA |
| | | that bind various | | (SEQ ID NO: 12) |
| | | heavy metals; these | | |
| | | proteins are | | |
| | | transcriptionally | | |
| | | regulated by both | | |
| | | heavy metals and | | |
| | | glucocorticoids. | | |
| | | May be involved in | | |
| | | FAM168A anti- | | |
| | | apoptotic signaling | | |
| | | (PubMed: 23251525) | | |
| MT1E | Metallothionein | Metallothioneins | NP_001350484.1 | MDPNCSCATGGSCTCAGSCKCKE |
| | 1E | have a high content | | CKCTSCKKSECGAISRNLGLWLRL |
| | | of cysteine residues | | GGNSRLALSASFWGTGLSLPSLP |
| | | that bind various | | VSFPLQAFCPKFRWGRTAFFSWDT |
| | | heavy metals; these | | NPNCTPYGFRTELCQTKKSILWVW |
| | | proteins are | | VLSSSQACY (SEQ ID NO: 13) |
| | | transcriptionally | | |
| | | regulated by both | | |
| | | heavy metals and | | |
| | | glucocorticoids. | | |
| MT1G | Metallothionein | Metallothioneins | NP_001288196.1 | MDPNCSCAAAGVSCTCASSCKCK |
| | 1G | have a high content | | ECKCTSCKKSCCSCCPVGCAKCAQ |
| | | of cysteine residues | | GCICKGASEKCSCCA |
| | | that bind various | | (SEQ ID NO: 14) |
| | | heavy metals; these | | |
| | | proteins are | | |
| | | transcriptionally | | |
| | | regulated by both | | |
| | | heavy metals and | | |
| | | glucocorticoids. | | |
| CXCL14 | C—X—C Motif | Potent | NP_004878.2 | MSLLPRRAPPVSMRLLAAALLLLL |
| | Chemokine | chemoattractant for | | LALYTARVDGSKCKCSRKGPKIRY |
| | Ligand 14 | neutrophils, and | | SDVKKLEMKPKYPHCEEKMVII |
| | | weaker for dendritic | | TTKSVSRYRGQEHCLHPKLQSTKR |
| | | cells. Not | | FIKWYNAWNEKRRVYEE |
| | | chemotactic for T- | | (SEQ ID NO: 15) |
| | | cells, B-cells, | | |
| | | monocytes, natural | | |
| | | killer cells or | | |
| | | granulocytes. Does | | |
| | | not inhibit | | |
| | | proliferation of | | |
| | | myeloid progenitors | | |
| | | in colony formation | | |
| | | assays. | | |
| MAOA | Monoamine | Catalyzes the | NP_000231.1 | MENQEKASIAGHMFDVVVIGGGIS |
| | Oxidase A | oxidative | | GLSAAKLLTEYGVSVLVLEARDRV |
| | | deamination of | | GGRTYTIRNEHVDYVDVGGAYVG |
| | | biogenic and | | PTQNRILRLSKELGIETYKVNVSER |
| | | xenobiotic amines | | LVQYVKGKTYPFRGAFPPVWNPIA |
| | | and has important | | YLDYNNLWRTIDNMGKEIPTDAP |
| | | functions in the | | WEAQHADKWDKMTMKELIDKIC |
| | | metabolism of | | WTKTARRFAYLFVNINVTSEPHEV |
| | | neuroactive and | | SALWFLWYVKQCGGTTRIFSVTN |
| | | vasoactive amines in | | GGQERKFVGGSGQVSERIMDLLG |
| | | the central nervous | | DQVKLNHPVTHVDQSSDNIIIETLN |
| | | system and | | HEHYECKYVINAIPPTLTAKIHFRP |
| | | peripheral tissues. | | ELPAERNQLIQRLPMGAVIKCMMY |
| | | MAOA | | YKEAFWKKKDYCGCMIIEDEDAPI |
| | | preferentially | | SITLDDTKPDGSLPAIMGFILARKA |
| | | oxidizes biogenic | | DRLAKLHKEIRKKKICELYAKVLG |
| | | amines such as 5- | | SQEALHPVHYEEKNWCEEQYSGG |
| | | hydroxytryptamine | | CYTAYFPPGIMTQYGRVIRQPVGRI |
| | | (5-HT), | | 1Th AGTETATKWSGYMEGAVEAGE |
| | | norepinephrine and | | RAAREVLNGLGKVTEKDIWVQEP |
| | | epinephrine. | | ESKDVPAVEITHTFWERNLPSVSG |
| | | | | LLKIIGFSTSVTALGFVLYKYKLLP |
| | | | | RS (SEQ ID NO: 16) |
| DPP4 | Dipeptidyl | Cell surface | NP_001926.2 | MKTPWKVLLGLLGAAALVTIITVP |
| | Peptidase 4 | glycoprotein | | VVLLNKGTDDATADSRKTYTLTD |
| | | receptor involved in | | YLKNTYRLKLYSLRWISDHEYLY |
| | | the costimulatory | | KQENNILVFNAEYGNSSVFLENSTF |
| | | signal essential for | | DEFGHSINDYSISPDGQFILLEYNY |
| | | T-cell receptor | | VKQWRHSYTASYDIYDLNKR |
| | | (TCR)-mediated T- | | QLITEERIPNNTQWVTWSPVGHKL |
| | | cell activation. Acts | | AYVWNNDIYVKIEPNLPSYRITWT |
| | | as a positive | | GKEDIIYNGITDWVYEEEVFSA |
| | | regulator of T-cell | | YSALWWSPNGTFLAYAQFNDTEV |
| | | coactivation, by | | PLIEYSFYSDESLQYPKTVRVPYPK |
| | | binding at least | | AGAVNPTVKFFVVNTDSLSSVT |
| | | ADA, CAV1, | | NATSIQITAPASMLIGDHYLCDVT |
| | | IGF2R, and PTPRC. | | WATQERISLQWLRRIQNYSVMDIC |
| | | Its binding to CAV1 | | DYDESSGRWNCLVARQHIEMST |
| | | and CARD11 | | TGWVGRFRPSEPHFTLDGNSFYKII |
| | | induces T-cell | | SNEEGYRHICYFQIDKKDCTFITKG |
| | | proliferation and | | TWEVIGIEALTSDYLYYISN |
| | | NF-kappa-B | | EYKGMPGGRNLYKIQLSDYTKVT |
| | | activation in a T-cell | | CLSCELNPERCQYYSVSFSKEAKY |
| | | receptor/CD3- | | YQLRCSGPGLPLYTLHSSVNDKG |
| | | dependent manner. | | LRVLEDNSALDKMLQNVQMPSKK |
| | | Its interaction with | | LDFIILNETKFWYQMILPPHFDKSK |
| | | ADA also regulates | | KYPLLLDVYAGPCSQKADTVFR |
| | | lymphocyte- | | LNWATYLASTENIIVASFDGRGSG |
| | | epithelial cell | | YQGDKIMHAINRRLGTFEVEDQIE |
| | | adhesion. In | | AARQFSKMGFVDNKRIAIWGWS |
| | | association with | | YGGYVTSMVLGSGSGVFKCGIAV |
| | | FAP is involved in | | APVSRWEYYDSVYTERYMGLPTP |
| | | the pericellular | | EDNLDHYRNSTVMSRAENFKQVE |
| | | proteolysis of the | | YLLIHGTADDNVHFQQSAQISKAL |
| | | extracellular matrix | | VDVGVDFQAMWYTDEDHGIASST |
| | | (ECM), the | | AHQHIYTHMSHFIKQCFSLP |
| | | migration and | | (SEQ ID NO: 17) |
| | | invasion of | | |
| | | endothelial cells into | | |
| | | the ECM. May be | | |
| | | involved in the | | |
| | | promotion of | | |
| | | lymphatic | | |
| | | endothelial cells | | |
| | | adhesion, migration | | |
| | | and tube formation. | | |
| | | When | | |
| | | overexpressed, | | |
| | | enhanced cell | | |
| | | proliferation, a | | |
| | | process inhibited by | | |
| | | GPC3. Acts also as | | |
| | | a serine | | |
| | | exopeptidase with a | | |
| | | dipeptidyl peptidase | | |
| | | activity that | | |
| | | regulates various | | |
| | | physiological | | |
| | | processes by | | |
| | | cleaving peptides in | | |
| | | the circulation, | | |
| | | including many | | |
| | | chemokines, | | |
| | | mitogenic growth | | |
| | | factors, | | |
| | | neuropeptides and | | |
| | | peptide hormones. | | |
| | | Removes N-terminal | | |
| | | dipeptides | | |
| | | sequentially from | | |
| | | polypeptides having | | |
| | | unsubstituted N- | | |
| | | termini provided | | |
| | | that the penultimate | | |
| | | residue is proline. | | |
| NUPR1 | Nuclear | Chromatin-binding | NP_001035948.1 | MATFPPATSAPQQPPGPEDEDSSLD |
| | Protein 1, | protein that converts | | ESDLYSLAHSYLGPLIMPMPTSPLT |
| | Transcriptional | stress signals into a | | PALVTGGGGRKGRTKREAAA |
| | Regulator | program of gene | | NTNRPSPGGHERKLVTKLQNSERK |
| | | expression that | | KRGARR (SEQ ID NO: 18) |
| | | empowers cells with | | |
| | | resistance to the | | |
| | | stress induced by a | | |
| | | change in their | | |
| | | microenvironment. | | |
| | | Interacts with MSL1 | | |
| | | and inhibits its | | |
| | | activity on histone | | |
| | | H4 Lys-16 | | |
| | | acetylation | | |
| | | (H4K16ac). Binds | | |
| | | the RELB promoter | | |
| | | and activates its | | |
| | | transcription, | | |
| | | leading to the | | |
| | | transactivation of | | |
| | | IER3. The | | |
| | | NUPR1/RELB/IER3 | | |
| | | survival pathway | | |
| | | may provide | | |
| | | pancreatic ductal | | |
| | | adenocarcinoma | | |
| | | with remarkable | | |
| | | resistance to cell | | |
| | | stress, such as | | |
| | | starvation or | | |
| | | gemcitabine | | |
| | | treatment. In breast | | |
| | | cancer cells, NUPR1 | | |
| | | overexpression leads | | |
| | | to the activation of | | |
| | | PI3K/AKT signaling | | |
| | | pathway, | | |
| | | CDKN1A/p21 | | |
| | | phosphorylation and | | |
| | | relocalization from | | |
| | | the nucleus to the | | |
| | | cytoplasm, leading | | |
| | | to resistance to | | |
| | | chemotherapeutic | | |
| | | agents, such as | | |
| | | doxorubicin. | | |
| GPX3 | Glutathione | Protects cells and | NP_001316719.1 | MARLLQASCLLSLLLAGFVSQSRG |
| | Peroxidase 3 | enzymes from | | QEKSKAPRQMGNPQMDCHGGISG |
| | | oxidative damage, | | TIYEYGALTIDGEEYIPFKQYAG |
| | | by catalyzing the | | KYVLFVNVASYUGLTGQYIELNAL |
| | | reduction of | | QEELAPFGLVILGFPCNQFGKQEPG |
| | | hydrogen peroxide, | | ENSEILPTLKYVRPGGGFVPN |
| | | lipid peroxides and | | FQLFEKGDVNGEKEQKFYTFLKNS |
| | | organic | | CPPTSELLGTSDRLFWEPMKVHDI |
| | | hydroperoxide, by | | RWNFEKFLVGPDGIPIMRWHHR |
| | | glutathione. | | TTVSNVKMDILSYMRRQAALGVK |
| | | | | RK (SEQ ID NO: 19) |
| PAEP | Progestagen | Glycoprotein that | NP_001018058.1 | MLCLLLTLGVALVCGVPAMDIPQT |
| | Associated | regulates critical | | KQDLELPKAPLRVHITSLLPTPEDN |
| | Endometrial | steps during | | LEIVLHRWENNSCVEKKVLGEKTE |
| | Protein | fertilization and also | | NPKKFKINYTVANEATLLDTDYDN |
| | | has | | FLFLCLQDTTTPIQSMMCQYLARV |
| | | immunomonomodulatory | | LVEDDEIMQGFIRAFRPLPRHLWY |
| | | effects. Four | | LLDLKQMEEPCRF (SEQ ID NO: 20) |
| | | glycoforms, namely | | |
| | | glycodelin-S, -A, -F | | |
| | | and -C have been | | |
| | | identified in | | |
| | | reproductive tissues | | |
| | | that differ in | | |
| | | glycosylation and | | |
| | | biological activity. | | |
| | | Glycodelin-A has | | |
| | | contraceptive and | | |
| | | immunosuppressive | | |
| | | activities | | |
| | | (PubMed: 9918684, | | |
| | | PubMed: 7531163). | | |
| | | Glycodelin-C | | |
| | | stimulates binding | | |
| | | of spermatozoa to | | |
| | | the zona pellucida | | |
| | | (PubMed: 17192260). | | |
| | | Glycodelin-F | | |
| | | inhibits | | |
| | | spermatozoa-zona | | |
| | | pellucida binding | | |
| | | and significantly | | |
| | | suppresses | | |
| | | progesterone- | | |
| | | induced acrosome | | |
| | | reaction of | | |
| | | spermatozoa | | |
| | | (PubMed: 12672671). | | |
| | | Glycodelin-S in | | |
| | | seminal plasma | | |
| | | maintains the | | |
| | | uncapacitated state | | |
| | | of human | | |
| | | spermatozoa | | |
| | | (PubMed: 15883155) | | |

The biomarkers identified in FIG. 3B (STC1, NFATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1) are not limited to a particular sequence and can include any variant. Exemplary sequences embraced by the present Application include:

FIG. 3B Exemplary GenBank Accession Nos. and Amino Acid Sequences
for Stromal Biomarkers

| Gene | Gene name | Function (Uniprot) | Accession No. | NCBI Reference Sequence |
|---|---|---|---|---|
| STC1 | Stanniocalcin 1 | Stimulates renal | NP_003146.1 | MLQNSAVLLVLVISASATHEAEQN |
| | | phosphate | | DSVSPRKSRVAAQNSAEVVRCLNS |
| | | reabsorption, and | | ALQVGCGAFACLENSTCDTDGM |
| | | could therefore | | YDICKSFLYSAAKFDTQGKAFVKE |
| | | prevent | | SLKCIANGVTSKVFLAIRRCSTFQR |
| | | hypercalcemia. | | MIAEVQEECYSKLNVCSIAKR |
| | | | | NPEAITEVVQLPNHFSNRYYNRLV |
| | | | | RSLLECDEDTVSTIRDSLMEKIGPN |
| | | | | MASLFHILQTDHCAQTHPRAD |
| | | | | FNRRRTNEPQKLKVLLRNLRGEED |
| | | | | SPSHIKRTSHESA (SEQ ID NO: 21) |
| NFATC2 | Nuclear Factor | Plays a role in the | NP_001129493.1 | MQREAAFRLGHCHPLRIMGSVDQ |
| | Of Activated T | inducible expression | | EEPNAHKVASPPSGPAYPDDVLDY |
| | Cells 2 | of cytokine genes in | | GLKPYSPLASLSGEPPGRFGEPD |
| | | T-cells, especially in | | RVGPQKFLSAAKPAGASGLSPRIEI |
| | | the induction of the | | TPSHELIQAVGPLRMRDAGLLVEQ |
| | | IL-2, IL-3, IL-4, | | PPLAGVAASPRFTLPVPGFEG |
| | | TNF-alpha or GM- | | YREPLCLSPASSGSSASFISDTFSPY |
| | | CSF. Promotes | | TSPCVSPNNGGPDDLCPQFQNIPAH |
| | | invasive migration | | YSPRTSPIMSPRTSLAEDS |
| | | through the | | CLGRHSPVPRPASRSSSPGAKRRHS |
| | | activation of GPC6 | | CAEALVALPPGASPQRSRSPSPQPS |
| | | expression and | | SHVAPQDHGSPAGYPPVAGS |
| | | WNT5A signaling | | AVIMDALNSLATDSPCGIPPKMWK |
| | | pathway. | | TSPDPSPVSAAPSKAGLPRHIYPAV |
| | | | | EFLGPCEQGERRNSAPESILL |
| | | | | VPPTWPKPLVPAIPICSIPVTASLPP |
| | | | | LEWPLSSQSGSYELRIEVQPKPHHR |
| | | | | AHYETEGSRGAVKAPTGGH |
| | | | | PVVQLHGYMENKPLGLQIFIGTAD |
| | | | | ERILKPHAFYQVHRITGKTVTTTSY |
| | | | | EKIVGNTKVLEIPLEPKNNMR |
| | | | | ATIDCAGILKLRNADIELRKGETDI |
| | | | | GRKNTRVRLVFRVHIPESSGRIVSL |
| | | | | QTASNPIECSQRSAHELPMV |
| | | | | ERQDTDSCLVYGGQQMILTGQNFT |
| | | | | SESKVVFTEKTTDGQQIWEMEATV |
| | | | | DKDKSQPNMLFVEIPEYRNKHI |
| | | | | RTPVKVNFYVINGKRKRSQPQHFT |
| | | | | YHPVPAIKTEPTDEYDPTLICSPTH |
| | | | | GGLGSQPYYPQHPMVAESPSC |
| | | | | LVATMAPCQQFRTGLSSPDARYQ |
| | | | | QQNPAAVLYQRSKSLSPSLLGYQQ |
| | | | | PALMAAPLSLADAHRSVLVHAGS |
| | | | | QGQSSALLHPSPTNQQASPVIHYSP |
| | | | | TNQQLRCGSHQEFQHIMYCENFAP |
| | | | | GTTRPGPPPVSQGQRLSPGSY |
| | | | | PTVIQQQNATSQRAAKNGPPVSDQ |
| | | | | KEVLPAGVTIKQEQNLDQTYLDDE |
| | | | | LIDTHLSWIQNIL (SEQ ID NO: 22) |
| BMP2 | Bone | Induces cartilage | NP_001191.1 | MVAGTRCLLALLLPQVLLGGAAG |
| | Morphogenetic | and bone formation | | LVPELGRRKFAAASSGRPSSQPSDE |
| | Protein 2 | (PubMed: 3201241). | | VLSEFELRLLSMFGLKQRPTPS |
| | | Stimulates the | | RDAVVPPYMLDLYRRHSGQPGSP |
| | | differentiation of | | APDHRLERAASRANTVRSFHHEES |
| | | myoblasts into | | LEELPETSGKTTRRFFFNLSSIP |
| | | osteoblasts via the | | TEEFITSAELQVFREQMQDALGNN |
| | | EIF2AK3-EIF2A- | | SSFHHRINIYEIIKPATANSKFPVTR |
| | | ATF4 pathway. | | LLDTRLVNQNASRWESFDVT |
| | | BMP2 activation of | | PAVMRWTAQGHANHGFVVEVAH |
| | | EIF2AK3 stimulates | | LEEKQGVSKRHVRISRSLHQDEHS |
| | | phosphorylation of | | WSQIRPLLVTFGHDGKGHPLHKRE |
| | | EIF2A which leads | | KRQAKHKQRKRLKSSCKRHPLYV |
| | | to increased | | DFSDVGWNDWIVAPPGYHAFYCH |
| | | expression of ATF4 | | GECPFPLADHLNSTNHAIVQTLVN |
| | | which plays a | | SVNSKIPKACCVPTELSAISMLYLD |
| | | central role in | | ENEKVVLKNYQDMVVEGCGCR |
| | | osteoblast | | (SEQ ID NO: 23) |
| | | differentiation. In | | |
| | | addition stimulates | | |
| | | TMEM119, which | | |
| | | upregulates the | | |
| | | expression of ATF4 | | |
| | | (PubMed: 24362451) | | |
| PMAIP1 | Phorbol-12- | Promotes activation | NP_066950.1 | MPGKKARKNAQPSPARAPAELEV |
| | Myristate-13- | of caspases and | | ECATQLRRFGDKLNFRQKLLNLIS |
| | Acetate- | apoptosis. Promotes | | KLFCSGT (SEQ ID NO: 24) |
| | Induced | mitochondrial | | |
| | Protein 1 | membrane changes | | |
| | | and efflux of | | |
| | | apoptogenic proteins | | |
| | | from the | | |
| | | mitochondria. | | |
| | | Contributes to | | |
| | | p53/TP53- | | |
| | | dependent apoptosis | | |
| | | after radiation | | |
| | | exposure. Promotes | | |
| | | proteasomal | | |
| | | degradation of | | |
| | | MCL1. Competes | | |
| | | with BAK1 for | | |
| | | binding to MCL1 | | |
| | | and can displace | | |
| | | BAK1 from its | | |
| | | binding site on | | |
| | | MCL1 (By | | |
| | | similarity). | | |
| | | Competes with | | |
| | | BIM/BCL2L11 for | | |
| | | binding to MCL1 | | |
| | | and can displace | | |
| | | BIM/BCL2L11 | | |
| | | from its binding site | | |
| | | on MCL1. | | |
| MMP11 | Matrix | May play an | NP_005931.2 | MAPAAWLRSAAARALLPPMLLLL |
| | Metallopeptidase | important role in the | | LQPPPLLARALPPDAHHLHAERRG |
| | 11 | progression of | | PQPWHAALPSSPAPAPATQEAPR |
| | | epithelial | | PASSLRPPRCGVPDPSDGLSARNR |
| | | malignancies. | | QKRFVLSGGRWEKTDLTYRILRFP |
| | | | | WQLVQEQVRQTMAEALKVWSDV |
| | | | | TPLTFTEVHEGRADIMIDFARYWH |
| | | | | GDDLPFDGPGGILAHAFFPKTHRE |
| | | | | GDVHFDYDETWTIGDDQGTDLL |
| | | | | QVAAHEFGHVLGLQHTTAAKALM |
| | | | | SAFYTFRYPLSLSPDDCRGVQHLY |
| | | | | GQPWPTVTSRTPALGPQAGIDTN |
| | | | | EIAPLEPDAPPDACEASFDAVSTIR |
| | | | | GELFFFKAGFVWRLRGGQLQPGYP |
| | | | | ALASRHWQGLPSPVDAAFEDA |
| | | | | QGHIWFFQGAQYWVYDGEKPVLG |
| | | | | PAPLTELGLVRFPVHAALVWGPEK |
| | | | | NKIYFFRGRDYWRFHPSTRRVDS |
| | | | | PVPRRATDWRGVPSEIDAAFQDAD |
| | | | | GYAYFLRGRLYWKFDPVKVKALE |
| | | | | GFPRLVGPDFFGCAEPANTFL |
| | | | | (SEQ ID NO: 25) |
| SFRP1 | Secreted | Soluble frizzled- | NP_003003.3 | MGIGRSEGGRRGAALGVLLALGA |
| | Frizzled | related proteins | | ALLAVGSASEYDYVSFQSDIGPYQ |
| | Related Protein | (sFRPS) function as | | SGRFYTKPPQCVDIPADLRLCHN |
| | 1 | modulators of Wnt | | VGYKKMVLPNLLEHETMAEVKQQ |
| | | signaling through | | ASSWVPLLNKNCHAGTQVFLCSLF |
| | | direct interaction | | APVCLDRPIYPCRWLCEAVRDSC |
| | | with Wnts. They | | EPVMQFFGFYWPEMLKCDKFPEG |
| | | have a role in | | DVCIAMTPPNATEASKPQGTTVCP |
| | | regulating cell | | PCDNELKSEAIIEHLCASEFALR |
| | | growth and | | MKIKEVKKENGDKKIVPKKKKPL |
| | | differentiation in | | KLGPIKKKDLKKLVLYLKNGADCP |
| | | specific cell types. | | CHQLDNLSHHFLIMGRKVKSQYL |
| | | SFRP1 decreases | | LTAIHKWDKKNKEFKNFMKKMK |
| | | intracellular beta- | | NHECPTFQSVFK (SEQ ID NO: 26) |
| | | catenin levels (By | | |
| | | similarity). Has | | |
| | | antiproliferative | | |
| | | effects on vascular | | |
| | | cells, in vitro and in | | |
| | | vivo, and can | | |
| | | induce, in vivo, an | | |
| | | angiogenic | | |
| | | response. In | | |
| | | vascular cell cycle, | | |
| | | delays the G1 phase | | |
| | | and entry into the S | | |
| | | phase (By | | |
| | | similarity). In | | |
| | | kidney | | |
| | | development, | | |
| | | inhibits tubule | | |
| | | formation and bud | | |
| | | growth in | | |
| | | metanephroi (By | | |
| | | similarity). Inhibits | | |
| | | WNT1/WNT4- | | |
| | | mediated TCF- | | |
| | | dependent | | |
| | | transcription. | | |
| WNT5A | Wnt Family | Ligand for members | NP_001243034.1 | MAGSAMSSKFFLVALAIFFSFAQV |
| | Member 5A | of the frizzled | | VIEANSWWSLGMNNPVQMSEVYII |
| | | family of seven | | GAQPLCSQLAGLSQGQKKLCHL |
| | | transmembrane | | YQDHMQYIGEGAKTGIKECQYQF |
| | | receptors. Can | | RHRRWNCSTVDNTSVFGRVMQIG |
| | | activate or inhibit | | SRETAFTYAVSAAGVVNAMSRAC |
| | | canonical Wnt | | REGELSTCGCSRAARPKDLPRDWL |
| | | signaling, depending | | WGGCGDNIDYGYRFAKEFVDARE |
| | | on receptor context. | | RERIHAKGSYESARILMNLHNNEA |
| | | In the presence of | | GRRTVYNLADVACKCHGVSGSCS |
| | | FZD4, activates | | LKTCWLQLADFRKVGDALKEKYD |
| | | beta-catenin | | SAAAMRLNSRGKLVQVNSRFNSPT |
| | | signaling. In the | | TQDLVYIDPSPDYCVRNESTGSLG |
| | | presence of ROR2, | | TQGRLCNKTSEGMDGCELMCCGR |
| | | inhibits the | | GYDQFKTVQTERCHCKFHWCCYV |
| | | canonical Wnt | | KCKKCTEIVDQFVCK |
| | | pathway by | | (SEQ ID NO: 27) |
| | | promoting beta- | | |
| | | catenin degradation | | |
| | | through a GSK3- | | |
| | | independent | | |
| | | pathway which | | |
| | | involves down- | | |
| | | regulation of beta- | | |
| | | catenin-induced | | |
| | | reporter gene | | |
| | | expression. | | |
| | | Suppression of the | | |
| | | canonical pathway | | |
| | | allows | | |
| | | chondrogenesis to | | |
| | | occur and inhibits | | |
| | | tumor formation. | | |
| | | Stimulates cell | | |
| | | migration. | | |
| | | Decreases | | |
| | | proliferation, | | |
| | | migration, | | |
| | | invasiveness and | | |
| | | clonogenicity of | | |
| | | carcinoma cells and | | |
| | | may act as a tumor | | |
| | | suppressor. | | |
| | | Mediates motility of | | |
| | | melanoma cells. | | |
| | | Required during | | |
| | | embryogenesis for | | |
| | | extension of the | | |
| | | primary anterior- | | |
| | | posterior axis and | | |
| | | for outgrowth of | | |
| | | limbs and the genital | | |
| | | tubercle. Inhibits | | |
| | | type II collagen | | |
| | | expression in | | |
| | | chondrocytes. | | |
| ZFYVE21 | Zinc Finger | Plays a role in cell | NP_001185882.1 | MSSEVSARRDAKKLVRSPSGLRM |
| | FYVE-Type | adhesion, and | | VPEHRAFGSPFGLEEPQWVPDKEC |
| | Containing 21 | thereby in cell | | RRCMQCDAKFDFLTRKHHCRRCG |
| | | motility which | | KCFCDRCCSQKVPLRRMCFVDPV |
| | | requires repeated | | RQCAECALVSLKEAEFYDKQLKV |
| | | formation and | | LLSGATFLVTFGNSEKPETMTCRL |
| | | disassembly of focal | | SNNQRYLFLDGDSHYEIEIVHISTV |
| | | adhesions. | | QILTEGFPPGEKDIHAYTSLRGSQP |
| | | Regulates | | ASEGGNARATGMFLQYTVPG |
| | | microtubule-induced | | TEGVTQLKLTVVEDVTVGRRQAV |
| | | PTK2/FAK1 | | AWLVAMHKAAKLLYESRDQ |
| | | dephosphorylation, | | (SEQ ID NO: 28) |
| | | an event important | | |
| | | for focal adhesion | | |
| | | disassembly, as well | | |
| | | as integrin beta- | | |
| | | 1/ITGB1 cell | | |
| | | surface expression. | | |
| CILP | Cartilage | Probably plays a | NP_003604.3 | MVGTKAWVFSFLVLEVTSVLGRQ |
| | Intermediate | role in cartilage | | TMLTQSVRRVQPGKKNPSIFAKPA |
| | Layer Protein | scaffolding. May | | DTLESPGEWTTWFNIDYPGGKGD |
| | | act by antagonizing | | YERLDAIRFYYGDRVCARPLRLEA |
| | | TGF-betal (TGFB1) | | RTTDWTPAGSTGQVVHGSPREGF |
| | | and IGF1 functions. | | WCLNREQRPGQNCSNYTVRFLCP |
| | | Has the ability to | | PGSLRRDTERIWSPWSPWSKCSAA |
| | | suppress IGF1- | | CGQTGVQTRTRICLAEMVSLCSEA |
| | | induced | | SEEGQHCMGQDCTACDLTCPMG |
| | | proliferation and | | QVNADCDACMCQDFMLHGAVSL |
| | | sulfated | | PGGAPASGAAIYLLTKTPKLLTQT |
| | | proteoglycan | | DSDGRFRIPGLCPDGKSILKITKV |
| | | synthesis, and | | KFAPIVLTMPKTSLKAATIKAEFVR |
| | | inhibits ligand- | | AETPYMVMNPETKARRAGQSVSL |
| | | induced IGF1R | | CCKATGKPRPDKYFWYHNDTLL |
| | | autophosphorylation. | | DPSLYKHESKLVLRKLQQHQAGE |
| | | May inhibit | | YFCKAQSDAGAVKSKVAQLIVIAS |
| | | TGFB1-mediated | | DETPCNPVPESYLIRLPHDCFQN |
| | | induction of | | ATNSFYYDVGRCPVKTCAGQQDN |
| | | cartilage matrix | | GIRCRDAVQNCCGISKTEEREIQCS |
| | | genes via its | | GYTLPTKVAKECSCQRCTETRS |
| | | interaction with | | IVRGRVSAADNGEPMRFGHVYMG |
| | | TGFB1. | | NSRVSMTGYKGTFTLHVPQDTERL |
| | | Overexpression may | | VLTFVDRLQKFVNTTKVLPFNKK |
| | | lead to impair | | GSAVFHEIKMLRRKEPITLEAMET |
| | | chondrocyte growth | | NIIPLGEVVGEDPMAELEIPSRSFYR |
| | | and matrix repair | | QNGEPYIGKVKASVTFLDPR |
| | | and indirectly | | NISTATAAQTDLNFINDEGDTFPLR |
| | | promote inorganic | | TYGMFSVDFRDEVTSEPLNAGKV |
| | | pyrophosphate (PPi) | | KVHLDSTQVKMPEHISTVKLWS |
| | | supersaturation in | | LNPDTGLWEEEGDFKFENQRRNK |
| | | aging and | | REDRTFLVGNLEIRERRLFNLDVPE |
| | | osteoarthritis | | SRRCFVKVRAYRSERFLPSEQI |
| | | cartilage. | | QGVVISVINLEPRTGFLSNPRAWG |
| | | | | RFDSVITGPNGACVPAFCDDQSPD |
| | | | | AYSAYVLASLAGEELQAVESSP |
| | | | | KFNPNAIGVPQPYLNKLNYRRTDH |
| | | | | EDPRVKKTAFQISMAKPRPNSAEE |
| | | | | SNGPIYAFENLRACEEAPPSAA |
| | | | | HFRFYQIEGDRYDYNTVPFNEDDP |
| | | | | MSWTEDYLAWWPKPMEFRACYIK |
| | | | | VKIVGPLEVNVRSRNMGGTHRQT |
| | | | | VGKLYGIRDVRSTRDRDQPNVSAA |
| | | | | CLEFKCSGMLYDQDRVDRTLVKVI |
| | | | | PQGSCRRASVNPMLHEYLVNHL |
| | | | | PLAVNNDTSEYTMLAPLDPLGHN |
| | | | | YGIYTVTDQDPRTAKEIALGRCFD |
| | | | | GTSDGSSRIMKSNVGVALTFNCV |
| | | | | ERQVGRQSAFQYLQSTPAQSPAAG |
| | | | | TVQGRVPSRRQQRASRGGQRQGG |
| | | | | VVASLRFPRVAQQPLIN |
| | | | | (SEQ ID NO: 29) |
| SLF2 | SMC5-SMC6 | Plays a role in the | NP_001129595.1 | MTRRCMPARPGFPSSPAPGSSPPRC |
| | Complex | DNA damage | | HLRPGSTAHAAAGKRTESPGDRK |
| | Localization | response (DDR) | | QSIIDFFKPASKQDRHMLDSPQ |
| | Factor 2 | pathway by | | KSNIKYGGSRLSITGTEQFERKLSS |
| | | regulating | | PKESKPKRVPPEKSPIIEAFMKGVK |
| | | postreplication | | EHHEDHGIHESRRPCLSLAS |
| | | repair of UV- | | KYLAKGTNIYVPSSYHLPKEMKSL |
| | | damaged DNA and | | KKKHRSPERRKSLFIHENNEKNDR |
| | | genomic stability | | DRGKTNADSKKQTTVAEADIFN |
| | | maintenance | | NSSRSLSSRSSLSRHHPEESPLGAK |
| | | (PubMed: 25931565). | | FQLSLASYCRERELKRLRKEQMEQ |
| | | The SLF1-SLF2 | | RINSENSFSEASSLSLKSSIE |
| | | complex acts to link | | RKYKPRQEQRKQNDIIPGKNNLSN |
| | | RAD18 with the | | VENGHLSRKRSSSDSWEPTSAGSK |
| | | SMC5-SMC6 | | QNKFPEKRKRNSVDSDLKSTRE |
| | | complex at | | SMIPKARESFLEKRPDGPHQKEKFI |
| | | replication-coupled | | KHIALKTPGDVLRLEDISKEPSDET |
| | | interstrand cross- | | DGSSAGLAPSNSGNSGHHST |
| | | links (ICL) and | | RNSDQIQVAGTKETKMQKPHLPLS |
| | | DNA double-strand | | QEKSAIKKASNLQKNKTASSTTKE |
| | | breaks (DSBs) sites | | KETKLPLLSRVPSAGSSLVPLN |
| | | on chromatin during | | AKNCALPVSKKDKERSSSKECSGH |
| | | DNA repair in | | STESTKHKEHKAKTNKADSNVSSG |
| | | response to stalled | | KISGGPLRSEYGTPTKSPPAAL |
| | | replication forks | | EVVPCIPSPAAPSDKAPSEGESSGN |
| | | (PubMed: 25931565). | | SNAGSSALKRKLRGDFDSDEESLG |
| | | Promotes the | | YNLDSDEEEETLKSLEEIMAL |
| | | recruitment of the | | NFNQTPAATGKPPALSKGLRSQSS |
| | | SMC5-SMC6 | | DYTGHVHPGTYTNTLERLVKEME |
| | | complex to DNA | | DTQRLDELQKQLQEDIRQGRGIK |
| | | lesions | | SPIRIGEEDSTDDEDGLLEEHKEFL |
| | | (PubMed: 25931565) | | KKFSVTIDAIPDHHPGEEIFNFLNSG |
| | | | | KIFNQYTLDLRDSGFIGQS |
| | | | | AVEKLILKSGKTDQIFLTTQGFLTS |
| | | | | AYHYVQCPVPVLKWLFRMMSVH |
| | | | | TDCIVSVQILSTLMEITIRNDTF |
| | | | | SDSPVWPWIPSLSDVAAVFFNMGI |
| | | | | DFRSLFPLENLQPDFNEDYLVSETQ |
| | | | | TTSRGKESEDSSYKPIFSTLP |
| | | | | ETNILNVVKFLGLCTSIHPEGYQDR |
| | | | | EIMLLILMLFKMSLEKQLKQIPLVD |
| | | | | FQSLLINLMKNIRDWNTKVP |
| | | | | ELCLGINELSSHPHNLLWLVQLVP |
| | | | | NWTSRGRQLRQCLSLVIISKLLDEK |
| | | | | HEDVPNASNLQVSVLHRYLVQ |
| | | | | MKPSDLLKKMVLKKKAEQPDGIID |
| | | | | DSLHLELEKQAYYLTYILLHLVGE |
| | | | | VSCSHSFSSGQRKHFVLLCGAL |
| | | | | EKHVKCDIREDARLFYRTKVKDLV |
| | | | | ARIHGKWQEIIQNCRPTQVSFCYTI |
| | | | | SCILNSFAEWHSSYCLK |
| | | | | (SEQ ID NO: 30) |
| MATN2 | Matrilin 2 | Involved in matrix | NP_001304677.1 | MEKMLAGCFLLILGQIVLLPAEAR |
| | | assembly. | | ERSRGRSISRGRHARTHPQTALLES |
| | | | | SCENKRADLVFIIDSSRSVNT |
| | | | | HDYAKVKEFIVDILQFLDIGPDVTR |
| | | | | VGLLQYGSTVKNEFSLKTFKRKSE |
| | | | | VERAVKRMRHLSTGTMTGLAI |
| | | | | QYALNIAFSEAEGARPLRENVPRVI |
| | | | | MIVTDGRPQDSVAEVAAKARDTGI |
| | | | | LIFAIGVGQVDFNTLKSIGSE |
| | | | | PHEDHVFLVANFSQIETLTSVFQKK |
| | | | | LCTAHMCSTLEHNCAHFCINIPGSY |
| | | | | VCRCKQGYILNSDQTTCRIQ |
| | | | | DLCAMEDHNCEQLCVNVPGSFVC |
| | | | | QCYSGYALAEDGKRCVAVDYCAS |
| | | | | ENHGCEHECVNADGSYLCQCHEG |
| | | | | FALNPDKKTCTRINYCALNKPGCE |
| | | | | HECVNMEESYYCRCHRGYTLDPN |
| | | | | GKTCSRVDHCAQQDHGCEQLCLN |
| | | | | TEDSFVCQCSEGFLINEDLKTCSRV |
| | | | | DYCLLSDHGCEYSCVNMDRSFAC |
| | | | | QCPEGHVLRSDGKTCAKLDSCAL |
| | | | | GDHGCEHSCVSSEDSFVCQCFEGY |
| | | | | ILREDGKTCRRKDVCQAIDHGCEH |
| | | | | ICVNSDDSYTCECLEGFRLAED |
| | | | | GKRCRRKDVCKSTHHGCEHICVN |
| | | | | NGNSYICKCSEGFVLAEDGRRCKK |
| | | | | CTEGPIDLVFVIDGSKSLGEENF |
| | | | | EVVKQFVTGIIDSLTISPKAARVGL |
| | | | | LQYSTQVHTEFTLRNFNSAKDMK |
| | | | | KAVAHMKYMGKGSMTGLALKH |
| | | | | MFERSFTQGEGARPLSTRVPRAAI |
| | | | | VFTDGRAQDDVSEWASKAKANGI |
| | | | | TMYAVGVGKAIEEELQEIASEPTN |
| | | | | KHLFYAEDFSTMDEISEKLKKGICE |
| | | | | ALEDSDGRQDSPAGELPKTVQQPT |
| | | | | ESEPVTINIQDLLSCSNFAVQ |
| | | | | HRYLFEEDNLLRSTQKLSHSTKPS |
| | | | | GSPLEEKHDQCKCENLIMFQNLAN |
| | | | | EEVRKLTQRLEEMTQRMEALEN |
| | | | | RLRYR (SEQ ID NO: 31) |
| S100A4 | S100 Calcium | The protein encoded | NP_002952.1 | MACPLEKALDVMVSTFHKYSGKE |
| | Binding | by this gene is a | | GDKFKLNKSELKELLTRELPSFLG |
| | Protein A4 | member of the S100 | | KRTDEAAFQKLMSNLDSNRDNEV |
| | | family of proteins | | DFQEYCVFLSCIAMMCNEFFEGFP |
| | | containing 2 EF- | | DKQPRKK (SEQ ID NO: 32) |
| | | hand calcium- | | |
| | | binding motifs. | | |
| DKK1 | Dickkopf | Antagonizes | NP_036374.1 | MMALGAAGATRVFVAMVAAALG |
| | WNT | canonical Wnt | | GHPLLGVSATLNSVLNSNAIKNLPP |
| | Signaling | signaling by | | PLGGAAGHPGSAVSAAPGILYPG |
| | Pathway | inhibiting LRP5/6 | | GNKYQTIDNYQPYPCAEDEECGTD |
| | Inhibitor 1 | interaction with Wnt | | EYCASPTRGGDAGVQICLACRKRR |
| | | and by forming a | | KRCMRHAMCCPGNYCKNGICVS |
| | | ternary complex | | SDQNHFRGEIEETITESFGNDHSTL |
| | | with the | | DGYSRRTTLSSKMYHTKGQEGSV |
| | | transmembrane | | CLRSSDCASGLCCARHFWSKIC |
| | | protein KREMEN | | KPVLKEGQVCTKHRRKGSHGLEIF |
| | | that promotes | | QRCYCGEGLSCRIQKDHHQASNSS |
| | | internalization of | | RLHTCQRH (SEQ ID NO: 33) |
| | | LRP5/6 | | |
| | | (PubMed: 22000856). | | |
| | | DKKs play an | | |
| | | important role in | | |
| | | vertebrate | | |
| | | development, where | | |
| | | they locally inhibit | | |
| | | Wnt regulated | | |
| | | processes such as | | |
| | | antero-posterior | | |
| | | axial patterning, | | |
| | | limb development, | | |
| | | somitogenesis and | | |
| | | eye formation. In | | |
| | | the adult, Dkks are | | |
| | | implicated in bone | | |
| | | formation and bone | | |
| | | disease, cancer and | | |
| | | Alzheimer disease | | |
| | | (PubMed:17143291). | | |
| | | Inhibits the pro- | | |
| | | apoptotic function | | |
| | | of KREMEN1 in a | | |
| | | Wnt-independent | | |
| | | manner, and has | | |
| | | anti-apoptotic | | |
| | | activity (By | | |
| | | similarity). | | |
| CRYAB | Crystallin | May contribute to | NP_001276736.1 | MDIAIHHPWIRRPFFPFHSPSRLFD |
| | Alpha B | the transparency and | | QFFGEHLLESDLFPTSTSLSPFYLRP |
| | | refractive index of | | PSFLRAPSWFDTGLSEMRL |
| | | the lens. Has | | EKDRFSVNLDVKHFSPEELKVKVL |
| | | chaperone-like | | GDVIEVHGKHEERQDEHGFIS REF |
| | | activity, preventing | | HRKYRIPADVDPLTITSSLSSD |
| | | aggregation of | | GVLTVNGPRKQVSGPERTIPITREE |
| | | various proteins | | KPAVTAAPKK (SEQ ID NO: 34) |
| | | under a wide range | | |
| | | of stress conditions. | | |
| FOXO1 | Forkhead Box | Transcription factor | NP_002006.2 | MAEAPQVVEIDPDFEPLPRPRSCT |
| | O1 | that is the main | | WPLPRPEFSQSNSATSSPAPSGSAA |
| | | target of insulin | | ANPDAAAGLPSASAAAVSADF |
| | | signaling and | | MSNLSLLEESEDFPQAPGSVAAAV |
| | | regulates metabolic | | AAAAAAAATGGLCGDFQGPEAGC |
| | | homeostasis in | | LHPAPPQPPPPGPLSQHPPVPPA |
| | | response to | | AAGPLAGQPRKSSSSRRNAWGNLS |
| | | oxidative stress. | | YADLITKAIESSAEKRLTLSQIYEW |
| | | Binds to the insulin | | MVKSVPYFKDKGDSNSSAGWK |
| | | response element | | NSIRHNLSLHSKFIRVQNEGTGKSS |
| | | (IRE) with | | WWMLNPEGGKSGKSPRRRAASM |
| | | consensus sequence | | DNNSKFAKSRSRAAKKKASLQSG |
| | | 5-TT[G/A]TTTTG- | | QEGAGDSPGSQFSKWPASPGSHSN |
| | | 3 and the related | | DDFDNWSTFRPRTSSNASTISGRLS |
| | | Daf-16 family | | PIMTEQDDLGEGDVHSMVYPP |
| | | binding element | | SAAKMASTLPSLSEISNPENMENLL |
| | | (DBE) with | | DNLNLLSSPTSLTVSTQSSPGTMM |
| | | consensus sequence | | QQTPCYSFAPPNTSLNSPSPN |
| | | 5-TT[G/A]TTTAC- | | YQKYTYGQSSMSPLPQMPIQTLQD |
| | | 3. Activity | | NKSSYGGMSQYNCAPGLLKELLTS |
| | | suppressed by | | DSPPHNDIMTPVDPGVAQPNSR |
| | | insulin. Main | | VLGQNVMMGPNSVMSTYGSQAS |
| | | regulator of redox | | HNKMMNPSSHTHPGHAQQTSAVN |
| | | balance and | | GRPLPHTVSTMPHTSGMNRLTQV |
| | | osteoblast numbers | | KTPVQVPLPHPMQMSALGGYSSVS |
| | | and controls bone | | SCNGYGRMGLLHQEKLPSDLDGM |
| | | mass. Orchestrates | | FIERLDCDMESIIRNDLMDGDTLDF |
| | | the endocrine | | NFDNVLPNQSFPHSVKTTTHSWVS |
| | | function of the | | G (SEQ ID NO: 35) |
| | | skeleton in | | |
| | | regulating glucose | | |
| | | metabolism. Acts | | |
| | | synergistically with | | |
| | | ATF4 to suppress | | |
| | | osteocalcin/BGLAP | | |
| | | activity, increasing | | |
| | | glucose levels and | | |
| | | triggering glucose | | |
| | | intolerance and | | |
| | | insulin insensitivity. | | |
| | | Also suppresses the | | |
| | | transcriptional | | |
| | | activity of RUNX2, | | |
| | | an upstream | | |
| | | activator of | | |
| | | osteocalcin/BGLAP. | | |
| | | In hepatocytes, | | |
| | | promotes | | |
| | | gluconeogenesis by | | |
| | | acting together with | | |
| | | PPARGC1A and | | |
| | | CEBPA to activate | | |
| | | the expression of | | |
| | | genes such as | | |
| | | IGFBP1, G6PC and | | |
| | | PCK1. Important | | |
| | | regulator of cell | | |
| | | death acting | | |
| | | downstream of | | |
| | | CDK1, PKB/AKT1 | | |
| | | and SKT4/MST1. | | |
| | | Promotes neural cell | | |
| | | death. Mediates | | |
| | | insulin action on | | |
| | | adipose tissue. | | |
| | | Regulates the | | |
| | | expression of | | |
| | | adipogenic genes | | |
| | | such as PPARG | | |
| | | during preadipocyte | | |
| | | differentiation and, | | |
| | | adipocyte size and | | |
| | | adipose tissue- | | |
| | | specific gene | | |
| | | expression in | | |
| | | response to | | |
| | | excessive calorie | | |
| | | intake. Regulates | | |
| | | the transcriptional | | |
| | | activity of | | |
| | | GADD45A and | | |
| | | repair of nitric | | |
| | | oxide-damaged | | |
| | | DNA in beta-cells. | | |
| | | Required for the | | |
| | | autophagic cell | | |
| | | death induction in | | |
| | | response to | | |
| | | starvation or | | |
| | | oxidative stress in a | | |
| | | transcription- | | |
| | | independent | | |
| | | manner. Mediates | | |
| | | the function of | | |
| | | MLIP in | | |
| | | cardiomyocytes | | |
| | | hypertrophy and | | |
| | | cardiac remodeling | | |
| | | (By similarity). | | |
| IL15 | Interleukin 15 | Cytokine that | NP_000576.1 | MRISKPHLRSISIQCYLCLLLNSHFL |
| | | stimulates the | | TEAGIHVFILGCFSAGLPKTEANW |
| | | proliferation of T- | | VNVISDLKKIEDLIQSMHID |
| | | lymphocytes. | | ATLYTESDVHPSCKVTAMKCFLLE |
| | | Stimulation by IL- | | LQVISLESGDASIHDTVENLIILANN |
| | | 15 requires | | SLSSNGNVTESGCKECEELE |
| | | interaction of IL-15 | | EKNIKEFLQSFVHIVQMFINTS |
| | | with components of | | (SEQ ID NO: 36) |
| | | IL-2R, including IL- | | |
| | | 2R beta and | | |
| | | probably IL-2R | | |
| | | gamma but not IL- | | |
| | | 2R alpha. | | |
| FGF7 | Fibroblast | Plays an important | NP_002000.1 | MHKWILTWILPTLLYRSCFHIICLV |
| | Growth Factor | role in the regulation | | GTISLACNDMTPEQMATNVNCSSP |
| | 7 | of embryonic | | ERHTRSYDYMEGGDIRVRRLF |
| | | development, cell | | CRTQWYLRIDKRGKVKGTQEMKN |
| | | proliferation and | | NYNIMEIRTVAVGIVAIKGVESEFY |
| | | cell differentiation. | | LAMNKEGKLYAKKECNEDCNFK |
| | | Required for normal | | ELILENHYNTYASAKWTHNGGEM |
| | | branching | | FVALNQKGIPVRGKKTKKEQKTA |
| | | morphogenesis. | | HFLPMAIT (SEQ ID NO: 37) |
| | | Growth factor active | | |
| | | on keratinocytes. | | |
| | | Possible major | | |
| | | paracrine effector of | | |
| | | normal epithelial | | |
| | | cell proliferation. | | |
| LMCD1 | LIM And | Transcriptional | NP_001265162.1 | MDSKYSTLTARVKGGDGIRIYKRN |
| | Cysteine Rich | cofactor that | | RMIMTNPIATGKDPTFDTITYEWA |
| | Domains 1 | restricts GATA6 | | PPGVTQKLGLQYMELIPKEKQP |
| | | function by | | VTGTEGAFYRRRQLMHQLPIYDQ |
| | | inhibiting DNA- | | DPSRCRGLLENELKLMEEFVKQYK |
| | | binding, resulting in | | SEALGVGEVALPGQGGLPKEEGK |
| | | repression of | | QQEKPEGAETTAATTNGSLSDPSK |
| | | GATA6 | | EVEYVCELCKGAAPPDSPVVYSDR |
| | | transcriptional | | AGYNKQWHPTCFVCAKCSEPLV |
| | | activation of | | DLIYFWKDGAPWCGRHYCESLRP |
| | | downstream target | | RCSGCDEIIFAEDYQRVEDLAWHR |
| | | genes. Represses | | KHFVCEGCEQLLSGRAYIVTKGQ |
| | | GATA6-mediated | | LLCPTCSKSKRS (SEQ ID NO: 38) |
| | | trans activation of | | |
| | | lung- and cardiac | | |
| | | tissue-specific | | |
| | | promoters. Inhibits | | |
| | | DNA-binding by | | |
| | | GATA4 and | | |
| | | GATA1 to the | | |
| | | cTNC promoter (By | | |
| | | similarity). Plays a | | |
| | | critical role in the | | |
| | | development of | | |
| | | cardiac hypertrophy | | |
| | | via activation of | | |
| | | calcineurin/nuclear | | |
| | | factor of activated | | |
| | | T-cells signaling | | |
| | | pathway. | | |

### Biomarker Analysis

Any of the biomarkers described herein, either taken alone or in combination (e.g., at least two biomarkers, at least three biomarkers, or more biomarkers), can be used in the assay methods also described herein for analyzing a sample from a subject to determine the one or more specific phases of endometrial transformation that occur in the human menstrual cycle. Results obtained from such assay methods can be used in either clinical applications or non-clinical applications, including, but not limited to, those described herein.

### Obtaining Biological Samples

The methods for identifying biomarkers and subsequently detecting biomarkers may involve with bulk tissues, e.g., bulk endometrial tissues. This is because the inventors have discovered that the biomarkers discussed herein from one subtissue, e.g., those presented in FIG. 3A (Table 3 above, unciliated epithelial markers), are expressed orthogonally with respect to other endometrial tissues, e.g., the biomarkers presented in FIG. 3B (Table 4 above, the stromal markers). That is, the genes generally upregulated or expressed in one endometrial tissue, e.g., unciliated epithelial cells (e.g., FIG. 3A genes), are downregulated or upregulates, and the same genes showed the opposite expression level in a different endometrial tissue type, e.g., stromal cells (e.g., FIG. 3B genes) when evaluated at the same menstrual phase. In other words, the genes are expressed in one cell type but not the other, which means it would be relatively easy to de-convolute their biomarker signatures with respect to different cell types even if a bulk sample of cells is used which comprises both stromal and epithelial cells.

This means that the various endometrial sub-tissues or cell types were found to have unique gene signatures which may be evaluated without first having to separate an endometrial tissue into its component cells.

However, the methods of biomarker detection also contemplate first processing a sample to first separate cell types, thereby conducting the biomarker analysis on only a single type of cell, e.g., unciliated endometrium or stromal cells.

Thus, in various embodiments, the methods disclosed herein may involve the step of processing a sample (e.g., an endometrial sample) by separating out one or more cell types, e.g., separating out unciliated epithelium cells, cilitated epithelium cells, stratum compactum cells (stromal), stratum spongiosum cells (stromal), glandular epithelium cells, luminal epithelium cells, and lymphatic or blood vessel cells from an endometrium sample. Once the cells of the endometrium are separated and collected or pooled, the cells of each individual tissue subtype can be evaluated for biomarker expression based on detection of any of the biomarkers of Tables 1-17.

Methods of Cell Separation are Well-Known in the Art.

Isolation of one or multiple cell types from a heterogeneous population is an integral part of modern biological research and routine clinical diagnosis and treatment. Purification of specific cells is essential for basic cell biology research, cellular enumeration in certain pathologies and cell based regenerative therapies. The main principle of separating any cell type from a population is to utilize one or more properties that are unique to that cell type. The most widely used cell isolation and separation techniques can be broadly classified as based on adherence, morphology (density/size) and antibody binding. The high precision single cell isolation methods are usually based on one or more of these properties while newer techniques incorporating microfluidics make use of some additional cellular characteristics. The recent improvements in cell isolation procedures vis-à-vis purity, yield and viability of cells has resulted in significant advances in the areas of stem cell biology, oncology and regenerative medicine among others.

A cell isolation procedure can either be a positive selection or a negative selection—the former aims at isolating the target cell type from the entire population, usually with specific antibodies while the latter strategy involves the depletion of all cell types of the population resulting in only the target cells remaining. Both types of isolation methods have their own advantages and disadvantages. Due to the use of specific antibodies targeting a particular cell type, positive selection yields a higher purity of the desired population. On the other hand, it is more complex to design an antibody cocktail to deplete all the non-target cells making negative selection less efficient vis-à-vis purity. Furthermore, a cell population isolated through positive selection can be sequentially purified through several cycles of the procedure, a benefit that negative selective cannot provide. However, positively selected cells carry antibodies and other labelling agents that may interfere with downstream culture and assays—if that is a concern, it is preferable to use a negative selection method

To isolate a particular cell type from a heterogeneous population, the unique properties of that cell type can be exploited. Cell isolation techniques are broadly classified into four categories based on the following cellular characteristics:

(1) Surface charge and adhesion—This feature determines the extent of attachment of cells to plastic and other polymer surfaces and can be used to separate adherent cells from suspension/free-floating cells.
(2) Cell size and density—The physical properties of size and density are commonly used for the bulk recovery of cells; either by sedimentation, filtration or density gradient centrifugation.
(3) Cell morphology and physiology—Different cell types can be distinguished on the basis of shape, histological staining, media selective growth, redox potential and other visual and behavioural properties which can then be harnessed to isolate those cells.
(4) Surface markers—Specific binding of surface antigens to either antibodies or aptamers can selectively capture cells of the specific surface phenotype. The captured cells are subsequently detected with the help of measurable probes—usually fluorochromes and magnetic particles—with which the antibodies/aptamers are labelled.

In addition, two or more of the above principles can be combined to further increase the specificity of isolated cells—usually such compound techniques consist of a label free (the first three in the list) method along-with a label incorporating method.

Using these well-known methods and the known properties and characteristics distinguishing the endometrial cell types from one another, the person of ordinary skill in the art can isolate or separate one or more cell types from a bulk endometrial tissue sample without undue experimentation.

In some embodiments, data is obtained for each of a plurality of cells in an endometrial sample. The data is then evaluated and a cell type is assigned to each cell based on one or more characteristic markers (e.g., one or more markers characteristic of a cell type of interest). In some embodiments, the gene expression data is used to determine the cell type, e.g., an unciliated epithelial cell or a stromal cell. For example, one or more of the following non-limiting genes can be used to identify a cell as an unciliated epithelial cell: PLUA, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP. Similarly, one or more of the following non-limiting genes can be used to identify a cell as a stromal cell: STC1, NGATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1.

Alternatively, in some embodiments, gene expression data for a plurality of cells in an endometrial sample can be obtained (e.g., bulk gene expression data) and evaluated to determine patterns of gene expression associated with different cell types within the sample without having to first separate the sample into distinct subcellular populations, i.e., a bulk assessment.

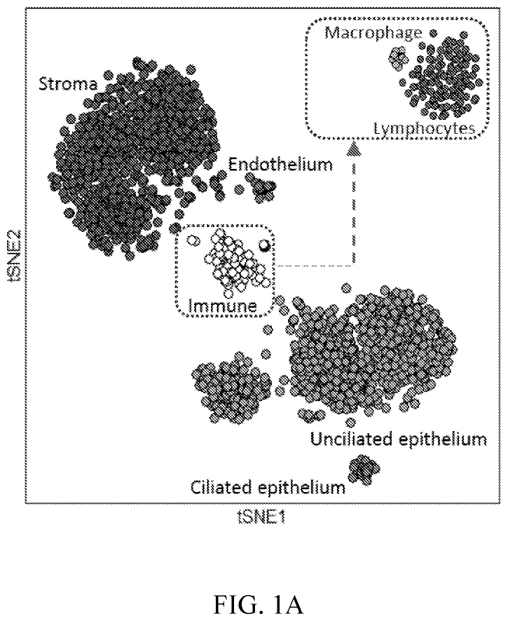

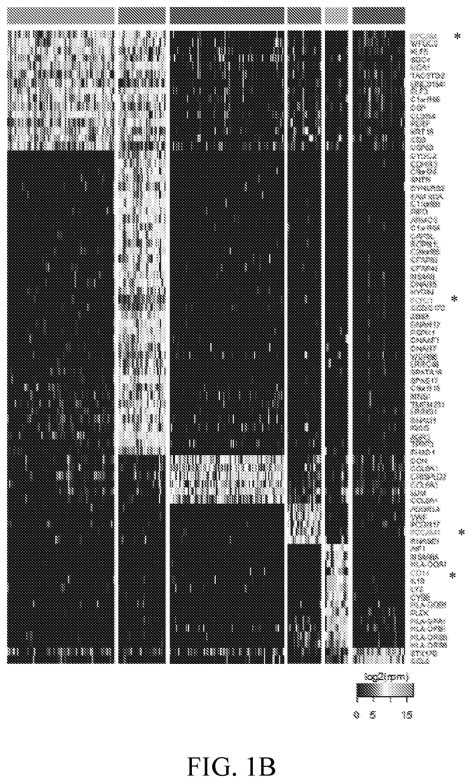

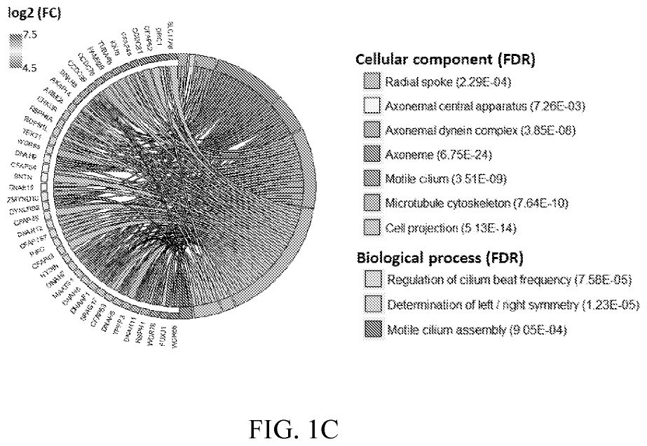

Bulk assessment may involve first using cell-type defining genes in FIG. 1B to estimate relative proportion of major endometrial cells types (e.g., relative proportion of unciliated epithelial cells), and then normalize the expression signatures provided herein, e.g., in Tables 9 and 10, or FIGS. 3A and 3B.

For gene set enrichment analysis (GSEA), one embodiment approach would be a scoring scheme where a (a>0) is added to the total score s if expression (>threshold) of a positive marker is observed, and subtract a from s if expression of a negative marker is seen. Similar to the original GSEA, based on a marker's importance and the category it belongs to, it may be assigned a weight.

### Analysis of Biological Samples

Any sample that may contain a biomarker (e.g., a biological sample such as endometrial tissue, endometrial cells, or endometrial fluid) can be analyzed by the assay methods described herein. A sample may also include a tissue or biological fluid (e.g., blood) which is obtained non-invasively. The methods described herein may include providing a sample obtained from a subject. In some examples, the sample may be from an in vitro assay, for example, an in vitro cell culture (e.g., an in vitro culture of human endometrial unciliated epithelial and/or human endometrial stromal cells (hESCs)). As used herein, a “sample” refers to a composition that comprises biological materials such as (but not limited to) endometrial tissue, endometrial cells, or endometrial fluid from a subject. A sample includes both an initial unprocessed sample taken from a subject as well as subsequently processed, e.g., partially purified or preserved forms. Exemplary samples include endometrial tissue, endometrial stromal cells, placental tissue, blood, plasma, or mucus. Exemplary endometrial tissue includes, but is not limited to, decidua basalis, decidua capsularis, or decidua parietalis. In some embodiments, the sample is a body fluid sample such as an endometrial fluid sample. In some embodiments, multiple (e.g., at least 2, 3, 4, 5, or more) samples may be collected from subject, over time or at particular time intervals, for example to assess the disease progression or evaluate the efficacy of a treatment.

A sample can be obtained from a subject using any means known in the art. In some embodiments, the sample is obtained from the subject by removing the sample (e.g., an endometrial tissue sample) from the subject. In some embodiments, the sample is obtained from the subject by a surgical procedure (e.g., dilation and curettage (D&C)). In some embodiments, the sample is obtained from the subject by a biopsy (e.g., an endometrial biopsy). In some embodiments, the sample is obtained from the subject by aspirating, brushing, scraping, or a combination thereof. In some embodiments, the sample is obtained from a human. In some embodiments, the sample is obtained non-invasively.

Any of the samples described herein can be subject to analysis using the assay methods described herein, which involve measuring the level of one or more biomarkers as described herein. Levels (e.g., the amount) of a biomarker disclosed herein, or changes in levels the biomarker, can be assessed using conventional assays or those described herein.

As used herein, the terms “determining” or “measuring,” or alternatively “detecting,” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, the level of a biomarker is assessed or measured by directly detecting the protein in a sample (e.g., an endometrial tissue sample, endometrial cell sample, or endometrial fluid sample). Alternatively or in addition, the level of a protein can be assessed or measured indirectly in a sample, for example, by detecting the level of activity of the protein (e.g., enzymatic assay).

The level of a protein (e.g., a biomarker protein) may be measured using an immunoassay. Examples of immunoassays include any known assay (without limitation), and may include any of the following: immunoblotting assay (e.g., Western blot), immunohistochemical analysis, flow cytometry assay, immunofluorescence assay (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting a biomarker protein provided herein will be apparent to those of skill in the art.

Such immunoassays may involve the use of an agent (e.g., an antibody) specific to the target biomarker. An agent such as an antibody that “specifically binds” to a target biomarker is a term well understood in the art, and methods to determine such specific binding are also well known in the art. An antibody is said to exhibit “specific binding” if it reacts or associates more frequently, more rapidly, with greater duration and/or with greater affinity with a particular target biomarker than it does with alternative biomarkers. It is also understood by reading this definition that, for example, an antibody that specifically binds to a first target peptide may or may not specifically or preferentially bind to a second target peptide. As such, “specific binding” or “preferential binding” does not necessarily require (although it can include) exclusive binding. Generally, but not necessarily, reference to binding means preferential binding. In some examples, an antibody that “specifically binds” to a target peptide or an epitope thereof may not bind to other peptides or other epitopes in the same antigen. In some embodiments, a sample may be contacted, simultaneously or sequentially, with more than one binding agent that binds different protein biomarkers (e.g., multiplexed analysis).

As used herein, the term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39.)) as well as complete antibodies. An antibody can have the structural features of IgA, IgG, IgE, IgD, IgM (as well as subtypes thereof). Antibodies may be from any source including, but not limited to, primate (human and non-human primate) and primatized (such as humanized) antibodies.

In some embodiments, the antibodies as described herein can be conjugated to a detectable label and the binding of the detection reagent to the peptide of interest can be determined based on the intensity of the signal released from the detectable label. Alternatively, a secondary antibody specific to the detection reagent can be used. One or more antibodies may be coupled to a detectable label. Any suitable label known in the art can be used in the assay methods described herein. In some embodiments, a detectable label comprises a fluorophore. As used herein, the term “fluorophore” (also referred to as “fluorescent label” or “fluorescent dye”) refers to moieties that absorb light energy at a defined excitation wavelength and emit light energy at a different wavelength. In some embodiments, a detection moiety is or comprises an enzyme. In some embodiments, an enzyme is one (e.g., β-galactosidase) that produces a colored product from a colorless substrate.

In some examples, an assay method described herein is applied to measure the level of a cellular biomarker in a sample. Such cells may be collected according to routine practice and the level of cellular biomarkers can be measured via a conventional method.

In other examples, an assay method described herein is applied to measure the level of a circulate biomarker in a sample, which can be any biological sample including, but not limited to, a fluid sample (e.g., a blood sample or plasma sample), a tissue sample, or a cell sample. Any of the assays known in the art including, e.g., immunoassays can be used for measuring the level of such biomarkers.

It will be apparent to those of skill in the art that this disclosure is not limited to immunoassays. Detection assays that are not based on an antibody, such as mass spectrometry, are also useful for the detection and/or quantification of biomarkers as provided herein. Assays that rely on a chromogenic substrate can also be useful for the detection and/or quantification of biomarkers as provided herein.

Alternatively, the level of nucleic acids encoding a biomarker in a sample can be measured via a conventional method. In some embodiments, measuring the expression level of nucleic acid encoding the biomarker comprises measuring mRNA. In some embodiments, the expression level of mRNA encoding a biomarker can be measured using real-time reverse transcriptase (RT) Q-PCR or a nucleic acid microarray. Methods to detect biomarker nucleic acid sequences include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

Any binding agent that specifically binds to a desired biomarker may be used in the methods and kits described herein to measure the level of a biomarker in a sample. In some embodiments, the binding agent is an antibody or an aptamer that specifically binds to a desired protein biomarker. In other embodiments, the binding agent may be one or more oligonucleotides complementary to a coding nucleic acid or a portion thereof. In some embodiments, a sample may be contacted, simultaneously or sequentially, with more than one binding agent that binds different biomarkers (e.g., multiplexed analysis).

To measure the level of a target biomarker, a sample can be in contact with a binding agent under suitable conditions. In general, the term “contact” refers to an exposure of the binding agent with the sample or cells collected therefrom for suitable period sufficient for the formation of complexes between the binding agent and the target biomarker in the sample, if any. In some embodiments, the contacting is performed by capillary action in which a sample is moved across a surface of the support membrane.

In some embodiments, the assays may be performed on low-throughput platforms, including single assay format. For example, a low throughput platform may be used to measure the presence and amount of a protein in a sample (e.g., endometrium tissue, endometrial stromal cells, and/or endometrial fluid) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, the biomarker set in a sample as described herein may be measured using any of the kits and/or detecting devices which are also described herein.

The type of detection assay used for the detection and/or quantification of a biomarker such as those provided herein may depend on the particular situation in which the assay is to be used (e.g., clinical or research applications), on the kind and number of biomarkers to be detected, and/or on the kind and number of patient samples to be run in parallel, to name a few parameters.

In various embodiments, the number of biomarkers that are measured fall between between 1 and 10 genes, or between 5 and 20 genes, or between 10 and 40 genes, or between 20 and 80 genes, or between 40 and 160 genes, or between 80 and 320 genes, or between 160 and 640 genes, or more. In still other embodiments, the gene expression levels can be measured for at least 1 gene, at least 10 genes, at least 20 genes, at least 30 genes, at least 40 genes, at least 50 genes, at least 60 genes, at least 70 genes, at least 80 genes, at least 90 genes, at least 100 genes, at least 125 genes, at least 150 genes, at least 175 genes, at least 200 genes, at least 300, 400, 500, 600, 700, 800, 900, or 1000 genes or more.

The assay methods described herein may be used for both clinical and non-clinical purposes. Some examples are provided herein.

Diagnostic and/or Prognostic Applications

The levels of one or more of the biomarkers in a sample obtained from a subject may be measured by the assay methods described herein and used for various clinical purposes. These clinical purposes may include, but are not limited to: identifying a subject having infertility, detecting or diagnosing the opening and/or closing of the window of implantation (WOI) in a subject trying to become pregnant, transferring an embryo in a subject that has been diagnosed as being within the window of implantation; treating a subject with infertility (e.g., by causing the overexpression or silencing of one or more of the genes disclosed herein using gene therapy), based on the level of one or more biomarkers described herein.

When needed, the level of a biomarker in a sample as determined by an assay methods described herein may be normalized with an internal control in the same sample or with a standard sample (having a predetermined amount of the biomarker) to obtain a normalized value. Either the raw value or the normalized value of the biomarker can then be compared with that in a reference sample or a control sample. A deviated (e.g., increased or reduced) value of the biomarker in a sample obtained from a subject as relative to the value of the same biomarker in the reference or control sample is indicative of whether the WOI is open or closed. Such a sample indicates that the subject from which the sample was obtained may be within the WOI.

In some embodiments, the level of the biomarker in a sample obtained from a subject can be compared to a predetermined threshold value for that biomarker, and a deviated (e.g., elevated or reduced) value of the biomarker may indicate that the window of implantation is open or closed for that subject.

The control sample or reference sample may be a sample obtained from a healthy individual. Alternatively, the control sample or reference sample contains a known amount of the biomarker to be assessed. In some embodiments, the control sample or reference sample is a sample obtained from a control subject.

The control level can be a predetermined level or threshold. Such a predetermined level can represent the level of the protein in a population of subjects that are within the window of implantation (WOI). It can also represent the level of the protein in a population of subjects that are not within the WOI.

The predetermined level can take a variety of forms. For example, it can be single cut-off value, such as a median or mean. In some embodiments, such a predetermined level can be established based upon comparative groups, such as where one defined group is known to be within the window of implantation, and another group is known to not be in the window of implantation. Alternatively, the predetermined level can be a range including, for example, a range representing the levels of the protein in a control population.

The control level as described herein can be determined by any technology known in the field. In some examples, the control level can be obtained by performing a conventional method (e.g., the same assay for obtaining the level of the protein in a test sample as described herein) on a control sample as also described herein. In other examples, levels of the protein can be obtained from members of a control population and the results can be analyzed by any method known in the field (e.g., a computational program) to obtain the control level (a predetermined level) that represents the level of the protein in the control population.

By comparing the level of a biomarker in a sample obtained from a candidate subject to the reference value as described herein, it can be determined whether the candidate subject is within the WOI. For example, if the level of biomarker(s) in a sample from the candidate subject deviates (e.g., is increased or decreased) from the reference value (by e.g., 1%, 5%, 10%, 20%, 30%, 40%, 50%, 60%, 70%, 80%, 90%, 100%, 150%, 200%, 300%, 400%, 500% or more from a reference value), the candidate subject might be identified as being within the WOI.

As used herein, “an absolute value of the ratio” refers to the ratio of the determined level of the biomarker in the sample to the control level of the biomarker. Control levels are described in detail herein. In some embodiments, the absolute value of the ratio is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 300, at least 400, at least 500, or at least 1000. In some embodiments, the absolute value of the ratio is between 2-1000. In some embodiments, the absolute value of the ratio is between 5-1000, between 10-1000, between 15-1000, between 20-1000, between 30-1000, between 40-1000, between 50-1000, between 60-1000, between 70-1000, between 80-1000, between 90-100, between 100-1000, between 200-1000, between 300-1000, between 400-1000, or between 500-1000. In some embodiments, the absolute value of the ratio is between 2-500, between 2-400, between 2-300, between 2-200, between 2-100, between 2-90, between 2-80, between 2-70, between 2-60, between 2-50, between 2-40, between 2-30, between 2-20, between 2-15, between 2-10, or between 2-5.

As used herein, “an elevated level,” “an increased level,” or “a level above a reference value” means that the level of the biomarker is higher than a reference value, such as a predetermined threshold of a level the biomarker in a control sample. An elevated or increased level of a biomarker includes a level of the biomarker that is, for example, 1%, 5%, 10%, 20%, 30%, 40%, 50%, 60%, 70%, 80%, 90%, 100%, 150%, 200%, 300%, 400%, 500% or more above a reference value. In some embodiments, the level of the biomarker in the test sample is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 100, 150, 200, 300, 400, 500, 1000, 10000-fold or more higher than the level of the biomarker in a reference sample.

As used herein, “a reduced level,” “a decreased level,” or “a level below a reference value” means that the level of the biomarker is lower than a reference value, such as a predetermined threshold of a level the biomarker in a control sample. A reduced or decreased level of a biomarker includes a level of the biomarker that is, for example, 1%, 5%, 10%, 20%, 30%, 40%, 50%, 60%, 70%, 80%, 90%, 100%, 150%, 200%, 300%, 400%, 500% or more below a reference value. In some embodiments, the level of the biomarker in the test sample is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 100, 150, 200, 300, 400, 500, 1000, 10000-fold or more less than the level of the biomarker in a reference sample.

In some embodiments, the candidate subject is a human patient trying to become pregnant. If the subject is identified as not responsive to the treatment, a higher dose and/or frequency of dosage of the therapeutic agent (e.g., a gene therapy agent) are administered to the subject identified. In some embodiments, the dosage or frequency of dosage of the therapeutic agent is maintained, lowered, or ceased in a subject identified as responsive to the treatment or not in need of further treatment. Alternatively, an alternative treatment can be administered to a subject who is found to not be responsive to a first or subsequent treatment. In some embodiments, an alternative treatment can be administered to a subject who is found to have a negative reaction to a first or subsequent treatment.

Also within the scope of the present disclosure are methods of evaluating a subject for transfer of one or more fertilized eggs or embryos. To practice this method, the level of one or more biomarkers in a sample collected from a subject trying to become pregnant is measured to determine the phase of menstrual cycle. If the biomarker level or levels indicate that the subject is within the WOI, one or more fertilized eggs or embryos may be transferred to the subject. If the biomarker level or levels indicate that the subject is not within the WOI, or is near or at the end of the WOI, one or more fertilized eggs or embryos may be transferred to the subject during the following menstrual cycle. A fertilized egg or embryo can be transferred to a subject using any means known in the art including, but not limited to, in vitro fertilization (IVF), ultra-sound guided IVF, and surgical embryo transfer (SET).

In some embodiments, the level of expression of a particular gene or biomarker is obtained as the absolute number of copies of mRNA a particular tissue sample or cell (e.g., endometrium tissue or cell sample). In other embodiments, the level of expression of a particular gene or biomarker is obtained by normalizing the amount of an expression product of a particular gene of interest against the amount of expression of a normalizing gene (e.g., one or more housekeeping genes) product. Normalization may be done to generate an index value or simply to help in reducing background noise when determining the expression level of the gene of interest. In one embodiment, for example, in determining the level of expression of a relevant gene in accordance with the present invention, the amount of an expression product of the gene (e.g., mRNA, cDNA, protein) is measured within one or more cells, particularly tumor cells, and normalized against the amount of the expression product(s) of a normalizing gene, or a set of normalizing genes, within the same one or more cells, to obtain the level of expression of the relevant marker gene. For example, when a single gene is used as a normalizing gene, a housekeeping gene whose expression is determined to be independent of endometrial cycling or transformation. A set of such housekeeping genes can also be used in gene expression analysis to provide a combined normalizing gene set. Housekeeping genes are well known in the art, with examples including, but are not limited to, G1/SB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). When a combined normalizing gene set is used in the normalization, the amount of gene expression of such normalizing genes can be averaged, combined together by straight additions or by a defined algorithm. Genes other than housekeeping genes may also be used as normalizing genes.

Those skilled in the art will appreciate how to obtain and use an index value in the methods of the invention. For example, the index value may represent the gene expression levels found in a normal sample obtained from the patient of interest (e.g., a healthy woman during one or more points in the menstrual cycle), in which case an expression level in the sample significantly higher than this index value would indicate, e.g., a poor prognosis or increased likelihood of abnormal menstrual cycle.

Alternatively, the index value may represent the average expression level of for a set of individuals from a diverse population or a subset of the population. For example, one may determine the average expression level of a gene or gene panel in a random sampling of patients at a specific point in the menstrual cycle, e.g., ovulation or the window of implantation. This average expression level may be termed the “threshold index value.”

Alternatively the index value may represent the average expression level of a particular gene marker in a plurality of training patients (e.g., patients within the window of implantation) with similar outcomes whose clinical and follow-up data are available and sufficient to define and categorize the patients by outcome, e.g., recurrence or prognosis. See, e.g., Examples, infra. For example, a “good prognosis index value” can be generated from a plurality of training cancer patients characterized as having “good outcome”, e.g., those who are fertile. A “poor prognosis index value” can be generated from a plurality of training cancer patients defined as having “poor outcome”, e.g., those who are infertile. Thus, a good prognosis index value of a particular gene may represent the average level of expression of the particular gene in patients having a “good outcome,” whereas a poor prognosis index value of a particular gene represents the average level of expression of the particular gene in patients having a “poor outcome.”

### Non-Clinical Applications

Further, levels of any of the biomarkers described herein may be applied for non-clinical uses including, for example, for research purposes. In some embodiments, the methods described herein may be used to study cell behavior and/or cell mechanisms. For example, one or more of the biomarkers described herein may be used to evaluate decidualization, which can be used for various purposes, including studies on decidualization and development of new agents that specifically target decidualization defects.

In some embodiments, the levels of biomarker sets, as described herein, may be relied on in the development of new therapeutics for infertility. For example, the levels of a biomarker may be measured in samples obtained from a subject who has been administered a new therapy (e.g., a clinical trial). In some embodiments, the level of the biomarker set may indicate the efficacy of the new therapeutic prior to, during, or after the administration of the new therapy.

Disclosed herein are methods to recognize a specific cell population within a sample of endometrial cells, and then use the transcriptomic analysis of that specific cell population to detect the opening of the window of implantation. Data disclosed herein demonstrate that the disclosed methods may be used in modified form to both detect and predict other events of interest in the menstrual cycle. Using the same combination of underlying analytical principles—allowing unbiased definition of endometrial cell populations, and then tracking their transcriptomic trajectories using mutual information analyses to enrich the data set for time-associated gene expressions—overcomes the problems posed in detecting the signal in the context of the noise. In this case, the signal comprises short-term changes in the expression status interest of some of the cell types, including transcriptomic shifts from day-to-day in individual patients. On the other hand, the noise is generated by the patient-to-patient variability in the length of menstrual cycles, and variation in the length and onset-timing of reproductively-significant functional changes in the endometrium where the variation between subjects (several days) exceeds or equals the time scale at which it is useful to detect events. Application of the disclosed methods to a reference population have solved this problem by providing both a reference data set against which individual patients can be evaluated, while the same methods provide the means to obtain and evaluate that individual patient's endometrial status without requiring independent knowledge of the length or phase of the patient's menstrual cycle, or more critically, the length and timing of medically useful events within that cycle.

By way of example, the disclosed methods can detect the opening of the WOI, and can also be used to detect the closing of WOI. In some embodiments, the disclosed methods are used to predict the opening or closing of WOI. Both prediction and detection of the opening and closing of the window are useful in the management of patients in need of embryo implantation. In some aspects, the disclosed methods are used to predict or detect the event of ovulation. Such prediction of ovulation is useful in the management of patient fertility and reproduction. In some aspects, the disclosed methods are used to detect the transcriptomic state of unciliated epithelium. These cells were previously unrecognized in the art, and have no distinctive morphological characteristics, but predictably precede ovulation. In some embodiments, the disclosed methods may be used for the detection of transcriptomic differentiation of glandular and luminal epithelial cell types. This also provides an improved method of prediction of ovulation compared to previously established schema.

In some aspects, shifts in the population frequency of endometrial cell populations can also be correlated to events of physiological and medical utility. In some embodiments, using a combination of such data—the recognition of time associated clusters of gene expression within cell sub-populations, differentiation of gene-expression patterns between cell sub-populations, and actual changes in the frequency of sub-populations within the endometrial population as a whole—provides enhanced diagnosis of endometrial status both by using a large number of orthogonal analyses to improve precision and decrease the impact of idiosyncratic expression of small numbers of genes as part of patient-to-patient variation. In some embodiments, enhanced diagnosis of endometrial status is achieved by maximizing the information obtainable from smaller samples, thereby minimizing the invasiveness and increasing safety and acceptability of the sampling procedure required to support the method.

The type of detection assay used for the detection and/or quantification of a biomarker such as those provided herein may depend on the particular situation in which the assay is to be used (e.g., clinical or research applications), on the kind and number of biomarkers to be detected, and/or on the kind and number of patient samples to be run in parallel, to name a few parameters.

### Computer-Based Analyses

In various aspects of the present Application, the results of any analyses can be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various biomarkers of Tables 1-6 can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result (e.g., the window of implantation) can be produced anywhere in the world (e.g., a testing facility) and transmitted to a different location (e.g., a hospital, patient testing laboratory, or a home). As an illustrative example, when an expression level, activity level, or sequencing (or genotyping) assay is conducted outside the United States, the information and data on a test result may be generated, cast in a transmittable form as described above, and then imported into the United States. Accordingly, the present invention also encompasses a method for producing a transmittable form of information on at least one of (a) expression level or (b) activity level for at least one patient sample. The method comprises the steps of (1) determining at least one of (a) or (b) above according to methods of the present invention; and (2) embodying the result of the determining step in a transmittable form. The transmittable form is the product of such a method.

Techniques for analyzing such expression, activity, and/or sequence data (indeed any data obtained according to the invention) will often be implemented using hardware, software or a combination thereof in one or more computer systems or other processing systems capable of effectuating such analysis.

The computer-based analysis function can be implemented in any suitable language and/or browsers. For example, it may be implemented with C language and preferably using object-oriented high-level programming languages such as Visual Basic, SmallTalk, C⁺⁺, and the like. The application can be written to suit environments such as the Microsoft Windows® environment including Windows® 98, Windows® 2000, Windows® NT, and the like, as well as Google®-based systems, e.g., Google Docs®. In addition, the application can also be written for the Apple® computers and MacOS® graphical user interface, SUN®, UNIX or LINUX environments, as well as smart phone computer platforms, e.g., iPhone®-based, Windows®-based, and Android®-based smart phones. In addition, the functional steps can also be implemented using a universal or platform-independent programming language. Examples of such multi-platform programming languages include, but are not limited to, hypertext markup language (HTML), JAVA®, JavaScript®, Flash programming language, common gateway interface/structured query language (CGI/SQL), practical extraction report language (PERL), AppleScript® and other system script languages, programming language/structured query language (PL/SQL), and any internet browser, e.g., Google® Chrome, Microsoft® Windows Explorer, and MacOS Safari. When active content web pages are used, they may include Java® applets or ActiveX® controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out gene status analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Thus one aspect of the present invention provides a system for determining the state of menstruation, e.g., detecting the occurrence of the implantation window (WOI). Generally speaking, the system comprises (1) computer means for receiving, storing, and/or retrieving a patient's gene status data (e.g., expression level or activity level of measured biomarkers) and optionally clinical parameter data (e.g., traditional histological menstrual cycle data); (2) computer means for querying this patient data; (3) computer means for determining the state of menstruation, e.g., the WOI, on this patient data; and (4) computer means for outputting/displaying this conclusion. In some embodiments, this means for outputting the conclusion may comprise a computer means for informing a health care professional of the conclusion.

One example of such a system includes a computer system that may include at least one input module for entering patient data into the computer system. The computer system may include at least one output module for indicating the state of the patient's menstrual cycle and/or indicating suggested treatments determined by the computer system. The computer system may include at least one memory module in communication with the at least one input module and the at least one output module.

The at least one memory module may include, e.g., a removable storage drive, which can be in various forms, including but not limited to, a magnetic tape drive, a floppy disk drive, a VCD drive, a DVD drive, an optical disk drive, etc. The removable storage drive may be compatible with a removable storage unit such that it can read from and/or write to the removable storage unit. The removable storage unit may include a computer usable storage medium having stored therein computer-readable program codes or instructions and/or computer readable data. For example, the removable storage unit may store patient data. Example of removable storage units are well known in the art, including, but not limited to, floppy disks, magnetic tapes, optical disks, and the like. The at least one memory module may also include a hard disk drive, which can be used to store computer readable program codes or instructions, and/or computer readable data.

In addition, the at least one memory module may further include an interface and a removable storage unit that is compatible with the interface such that software, computer readable codes or instructions can be transferred from the removable storage unit into computer system. Examples of the interface and the removable storage unit pairs include, e.g., removable memory chips and sockets associated therewith, program cartridges and cartridge interface, and the like.

The computer system may include at least one processor module. It should be understood that the at least one processor module may consist of any number of devices. The at least one processor module may include a data processing device, such as a microprocessor or microcontroller or a central processing unit. The at least one processor module may include another logic device such as a DMA (Direct Memory Access) processor, an integrated communication processor device, a custom VLSI (Very Large Scale Integration) device or an ASIC (Application Specific Integrated Circuit) device. In addition, the at least one processor module may include any other type of analog or digital circuitry that is designed to perform the processing functions described herein. The at least one memory module [606] may be configured for storing patient data entered via the at least one input module [630] and processed via the at least one processor module [602]. Patient data relevant to the present invention may include expression level, activity level, copy number and/or sequence information for PTEN and/or a CCG. Patient data relevant to the present invention may also include clinical parameters relevant to the patient's disease. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module may include a computer-implemented method stored therein. The at least one processor module may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient being tested for menstrual cycle state. For example, the computer-implemented method may be configured to inform a physician (e.g., an in vitro fertilization specialist) that a particular patient's menstrual cycle is at a window of implantation. Alternatively or additionally, the computer-implemented method may be configured to actually suggest a particular course of treatment based on the answers to/results for various queries.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and others. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2.sup.nd ed., 2001); see also, U.S. Pat. No. 6,420,108, which are incorporated herein by reference.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170, which are incorporated herein by reference. Additionally, the present invention may have embodiments that include methods for providing genetic information over networks such as the Internet as shown in U.S. Ser. No. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354), which are incorporated herein by reference.

The assay methods described herein may be used for both clinical and non-clinical purposes. Some examples are provided herein.

### Kits and Detecting Devices for Measuring Biomarkers

The present disclosure also provides kits and devices for use in measuring the level of a biomarker set as described herein. Such a kit or device can comprise one or more binding agents that specifically bind to a gene product of target biomarkers, such as the biomarkers listed in any of Tables 1-17. For example, such a kit or detecting device may comprise at least one binding agent that is specific to one or more protein biomarkers selected from Tables 1-17. In some instances, the kit or detecting device comprises binding agents specific to two or more members of the protein biomarker set described herein.

Levels of specific expression products of genes (e.g., NUPR1, CADM1, NPAS3, ATP1A1, and/or TRAK1; CRYAB, NFATC2, BMP2, PMAIP1, ZFYVE21, CILP, SLF2, MATN2, and/or FGF7) can be assessed by any appropriate method. In some embodiments, the levels of specific expression products are analyzed using one or more assays comprising any solid support (e.g., one or more chips). For example, a solid support (e.g., a chip) may be used to analyze at least one (e.g., 1, 2, 3, 4, 5, 6, 7, 8, 9, 10, or more) biological sample(s) of or from a subject.

Sections of the solid support (e.g., the chip) may be modified with one binding partner or more than one binding partner. The solid support may be linked in any manner to the binding partner(s). As a non-limiting example, the binding partner(s) may be physisorbed or otherwise bound (e.g., bound directly) onto the surface of the solid support or covalently linked through appropriate coupling chemistry in any manner including, but not limited to: linkage through a epoxide on the surface, creation of an amido link (i.e., through NHS EDC chemistry) using a amine or carboxylic acid group present on the surface, linkage between a thiol and a thiol reactive group (i.e., a maleimide group), formation of a Schiff base between aldehyde and amines, reaction to an anhydride present on the surface, and/or through a photo-activatable linker.

The binding partner may be any binding partner useful for the instant compositions or methods. For example, the binding partner may be a protein (with naturally occurring amino acids or artificial amino acids), one or more nucleic acids made of naturally occurring bases or artificial bases (including, for example, DNA or RNA), sugars, carbohydrates, one or more small molecules (including, but not limited to one or more of: a vitamin, hormone, cofactor, heme group, chelate, fatty acid, or other known small molecule, and/or a phage).

The binding partners may be applied to the surface of the substrate by deposition of a droplet at a pre-defined location in any manner and using any device including, but not limiting to: the use of a pipette, a liquid dispenser, plotter, nano-spotter, nano-plotter, arrayer, spraying mechanism or other suitable fluid handling device.

In some embodiments, antibodies or antigen-binding fragments are provided that are suited for use in the instant methods and compositions. Immunoassays utilizing such antibody or antigen-binding fragments useful for the instant compositions and methods may be competitive or non-competitive immunoassays in either a direct or an indirect format. Non-limiting examples of such immunoassays are Enzyme Linked Immunoassays (ELISA), radioimmunoassays (RIA), sandwich assays (immunometric assays), flow cytometry-based assays, western blot assays, immunoprecipitation assays, immunohistochemistry assays, immuno-microscopy assays, lateral flow immuno-chromatographic assays, and proteomics arrays. For example, the binding partners may be antibodies (or antibody-binding fragments thereof) with specificity towards a protein of interest including one or more of unciliated epithelial biomarkers NUPR1, CADM1, NPAS3, ATP1A1, and/or TRAK1; or one or more of stromal biomarkers CRYAB, NFATC2, BMP2, PMAIP1, ZFYVE21, CILP, SLF2, MATN2, and/or FGF7.

In some embodiments, oligonucleotide binding partners are used to assess the levels of specific expression products of genes. The oligonucleotide binding partners may be of any type known or used. As a set of non-limiting examples, in certain embodiments the oligonucleotide probes may be RNA oligonucleotides, DNA oligonucleotides, a mixture of RNA oligonucleotides and DNA nucleotides, and/or oligonucleotides that may be mixtures of RNA and DNA. The oligonucleotide binding partners may be naturally occurring or synthetic. The oligonucleotide binding partners may be of any length. As a set of non-limiting examples, the length of the oligonucleotide binding partners may range from about 5 to about 50 nucleotides, from about 10 to about 40 nucleotides, or from about 15 to about 40 nucleotides. The array may comprise any number of oligonucleotide binding partners specific for each target gene. For example, the array may comprise less than 10 (e.g., 9, 8, 7, 6, 5, 4, 3, 2, or 1) oligonucleotide probes specific for each target gene. As another example, the array may comprise more than 10, more than 50, more than 100, or more than 1000 oligonucleotide binding partners specific for each target gene.

The array may further comprise control binding partners such as, for example mismatch control oligonucleotide binding partners or control antibodies or antigen binding fragments thereof. Where mismatch control oligonucleotide binding partners are present, the quantifying step may comprise calculating the difference in hybridization signal intensity between each of the oligonucleotide binding partners and its corresponding mismatch control binding partner. Where control antibodies or antigen binding fragments thereof are present, the quantifying step may comprise calculating the difference in hybridization signal intensity between antibodies or antigen binding fragments for the genes under examination (e.g., NUPR1, CADM1, NPAS3, ATP1A1, and/or TRAK1; CRYAB, NFATC2, BMP2, PMAIP1, ZFYVE21, CILP, SLF2, MATN2, and/or FGF7) and a control or “housekeeping” antibody or antigen binding fragment thereof. The quantifying may further comprise calculating the average difference in hybridization signal intensity between each of the oligonucleotide probes and its corresponding mismatch control probe for each gene.

The array (e.g., chip) may contain any number of analysis regions. As a set of non-limiting examples, the array may contain one or more than one (e.g., 2, 3, 4, 5, 6, 7, 8, 9, 10, 20, 25, 30, 35, 40, or more) analysis regions. Each analysis region may comprise any number of binding partners immobilized to a substrate portion therein. As a non-limiting set of examples, each analysis region may comprise between one and 1,000 binding partners, one and 500 binding partners, one and 250 binding partners, one and 100 binding partners, two and 1,000 binding partners, two and 500 binding partners, two and 250 binding partners, two and 100 binding partners, three and 1,000 binding partners, three and 500 binding partners, three and 250 binding partners, or three and 100 binding partners immobilized to a substrate portion therein.

Binding partners including, but not limited to, antibodies or antigen-binding fragments that bind to the specific antigens of interest can be immobilized, e.g., by binding to a solid support (e.g., a chip, carrier, membrane, columns, proteomics array, etc.). In one set of embodiments, a material used to form the solid support has an optical transmission of greater than 90% between 400 and 800 nm wavelengths of light (e.g., light in the visible range). Optical transmission may be measured through a material having a thickness of, for example, about 2 mm (or in other embodiments, about 1 mm or about 0.1 mm). In some instances, the optical transmission is greater than or equal to 80%, greater than or equal to 85%, greater than or equal to 88%, greater than or equal to 92%, greater than or equal to 94%, or greater than or equal to 96% between 400 and 800 nm wavelengths of light. In some embodiments, the material used to form the solid support has an optical transmission of less than or equal to 99.9%, less than or equal to 96%, less than or equal to 94%, less than or equal to 92%, less than or equal to 90%, less than or equal to 85%, less than or equal to 80%, less than or equal to 50%, less than or equal to 30%, or less than or equal to 10% between 400 and 800 nm wavelengths of light. Combinations of the above-referenced ranges are also possible.

The array may be fabricated on a surface of virtually any shape (e.g., the array may be planar) or even a multiplicity of surfaces. Non-limiting examples of solid support materials useful for the compositions and methods described herein may include glass, plastics, elastomeric materials, membranes, or other suitable materials for performing immunoassays. The solid support may be formed from one material, or it may be formed from two or more materials.

Specific solid support materials may include, but are not limited to: any type of glass (e.g., fused silica, borosilicate glass, Pyrex®, or Duran®). In one embodiment, the solid support is a glass chip. The solid support may also comprise a non-glass substrate (e.g., a plastic substrate) coated with a glass film dioxide produced by a process such as sputtering, oxidation of silicon, or through reaction of silane reagents. The glass surface may be further modified with functionalized silane reagents including, for example: amine-terminated silanes (aminopropyltriethoxy silane) and epoxide-terminated silanes (glycidoxypropyltrimethoxysilane).

Additional specific solid support materials may include, but are not limited to: thermoplastic polymers and may comprise one or more of: polystyrene, polycarbonate, polymethylmethacrylate, cyclic olefin copolymers, polyethylene, polypropylene, polyvinyl chloride, polyvinylidene difluoride, any fluoropolymers (e.g., polytetrafluoroethylene, also known as Teflon®), polylactic acid, poly(methyl methacrylate) (also known as PMMA or acrylic; e.g., Lucite®, Perspex®, and Plexiglas®), and acrylonitrile butadiene styrene.

Additional specific solid support materials may include, but are not limited to: one or more elastomeric materials including polysiloxanes (silicones such as polydimethylsiloxane) and rubbers (polyisoprene, polybutadiene, chloroprene, styrene-butadiene, nitrile rubber, polyether block amides, ethylene-vinyl acetate, epichlorohydrin rubber, isobutene-isoprene, nitrile, neoprene, ethylene-propylene, and hypalon).

Additional specific solid support materials may include, but are not limited to: one or more membrane substrates such as dextran, amyloses, nylon, Polyvinylidene fluoride (PVDF), fiberglass, and natural or modified celluloses (e.g., cellulose, nitrocellulose, CNBr-activated cellulose, and cellulose modified with polyacrylamides, agaroses, and/or magnetite). The nature of the support can be either fixed or suspended in a solution (e.g., beads).

In some embodiments, the material and dimensions (e.g., thickness) of a solid support (e.g., a chip) is substantially impermeable to water vapor. In some embodiments, a cover may also be present. In some embodiments, the cover is substantially impermeable to water vapor. For instance, a solid support (e.g., a chip) may include a cover comprising a material known to provide a high vapor barrier, such as metal foil, certain polymers, certain ceramics and combinations thereof. Examples of materials having low water vapor permeability are provided below. In other cases, the material is chosen based at least in part on the shape and/or configuration of the chip. For instance, certain materials can be used to form planar devices whereas other materials are more suitable for forming devices that are curved or irregularly shaped.

A material used to form all or portions of a section or component of any composition described herein may have, for example, a water vapor permeability of less than about 5.0 g·mm/m²·d, less than about 4.0 g·mm/m²·d, less than about 3.0 g·mm/m²·d, less than about 2.0 g·mm/m²·d, less than about 1.0 g·mm/m²·d, less than about 0.5 g·mm/m²·d, less than about 0.3 g·mm/m²·d, less than about 0.1 g·mm/m²·d, or less than about 0.05 g·mm/m²·d. In some cases, the water vapor permeability may be, for example, between about 0.01 g·mm/m²·d and about 2.0 g·mm/m²·d, between about 0.01 g·mm/m²·d and about 1.0 g·mm/m²·d, between about 0.01 g·mm/m²·d and about 0.4 g·mm/m²·d, between about 0.01 g·mm/m²·d and about 0.04 g·mm/m²·d, or between about 0.01 g·mm/m²·d and about 0.1 g·mm/m²·d. The water vapor permeability may be measured at, for example, 40° C. at 90% relative humidity (RH). Combinations of materials with any of the aforementioned water vapor permeabilities may be used in the instant compositions or methods.

In some embodiments, the material and dimensions of a solid support (e.g., a chip) and/or cover may vary. For example, the chip may be configured to provide one or more regions (e.g., liquid containment regions). In certain embodiments, the chip may be configured to provide two or more regions (e.g., liquid containment regions). In certain embodiments, two or more of the regions are fluidically separated from other regions. In one embodiment, all of the regions are fluidically separated from other regions. In some embodiments, all of the regions are fluidically connected. The chip may comprise any number of liquid containment regions. As a non-limiting example, the chip may comprise one, two, three, four, five, six, seven, eight, nine, or ten liquid containment regions, each of which may be fluidically separated from one another. In other embodiments, the chip may comprise one, two, three, four, five, six, seven, eight, nine, or ten liquid containment regions that are fluidically connected to one another.

A solid support (e.g., a chip) described herein may have any suitable volume for carrying out an analysis such as a chemical and/or biological reaction or other process. The entire volume of the solid support may include, for example, any reagent storage areas, analysis regions, liquid containment regions, waste areas, as well as one or more identifiers. In some embodiments, small amounts of reagents and samples are used and the entire volume of the a liquid containment region is, for example, less than or equal to 10 mL, less than or equal to 5 mL, less than or equal to 1 mL, less than or equal to 500 μL, less than or equal to 250 μL, less than or equal to 100 μL, less than or equal to 50 μL, less than or equal to 25 μL, less than or equal to 10 μL, less than or equal to 5 μL, or less than or equal to 1 μL. In some embodiments, small amounts of reagents and samples are used and the entire volume of the a liquid containment region is, for example, at least 10 mL, at least 5 mL, at least 1 mL, at least 500 μL, at least 250 μL, at least 100 μL, at least 50 μL, at least 25 μL, at least 10 μL, at least 5 μL, or at least 1 μL. Combinations of the above-referenced values are also possible.

The length and/or width of the solid support (e.g., chip) may be, for example, less than or equal to 300 mm, less than or equal to 200 mm, less than or equal to 150 mm, less than or equal to 100 mm, less than or equal to 95 mm, less than or equal to 90 mm, less than or equal to 85 mm, less than or equal to 80 mm, less than or equal to 75 mm, less than or equal to 70 mm, less than or equal to 65 mm, less than or equal to 60 mm, less than or equal to 55 mm, less than or equal to 50 mm, less than or equal to 45 mm, less than or equal to 40 mm, less than or equal to 35 mm, less than or equal to 30 mm, less than or equal to 25 mm, or less than or equal to 20 mm. In some embodiments, the length and/or width of the chip may be, for example, at least 300 mm, at least 200 mm, at least 150 mm, at least 100 mm, at least 95 mm, at least 90 mm, at least 85 mm, at least 80 mm, at least 75 mm, at least 70 mm, at least 65 mm, at least 60 mm, at least 55 mm, at least 50 mm, at least 45 mm, at least 40 mm, at least 35 mm, at least 30 mm, at least 25 mm, or at least 20 mm. Combinations of the above-referenced values are also possible. In some embodiments, the thickness of the solid support (e.g., chip) may be, for example, less than or equal to 5 mm, less than or equal to 3 mm, less than or equal to 2 mm, less than or equal to 1 mm, less than or equal to 0.9 mm, less than or equal to 0.8 mm, less than or equal to 0.7 mm, less than or equal to 0.5 mm, less than or equal to 0.4 mm, less than or equal to 0.3 mm, less than or equal to 0.2 mm, or less than or equal to 0.1 mm. In some embodiments, the thickness of the solid support (e.g., chip) may be, for example, at least 5 mm, at least 3 mm, at least 2 mm, at least 1 mm, at least 0.9 mm, at least 0.8 mm, at least 0.7 mm, at least 0.5 mm, at least 0.4 mm, at least 0.3 mm, at least 0.2 mm, or at least 0.1 mm. Combinations of the above-referenced values are also possible. One or more solid supports (e.g., chips) may be analyzed at the same time by any suitable device. An adapter may be used with the one or more solid supports (e.g., chips) in order to insert and securely hold them in the analyzer.

In some embodiments, the solid support (e.g., chip) includes one or more identifiers. Any method or type of identification may be used. For example, an identifier may be, but is not limited to, any type of label such as a bar code or an RFID tag. The identifier may include the name, patient number, social security number, or any other method of identification for a subject. The identifier may also be a randomized identifier of any type useful in a clinical setting.

It should be understood that the solid supports (e.g., chips) and their respective components described herein are exemplary and that other configurations and/or types of solid supports (e.g., chips) and components can be used with the systems and methods described herein.

The binding of a one or more binding partners (e.g., to detect the binding of a protein or other substance of interest including, but not limited to, antigen-bound antibody complexes) may be quantified by any method known in the art. The quantification may, for example, be performed by detection or interrogation of an active molecule bound to an antibody. In a multiplexed format, where more than one assay is being performed on a continuous area, the signals associated with each assay must be differentiable from the other assays. Any suitable strategy known in the art may be used including, but not limited to: (1) using a label with substantially non-overlapping spectral and/or electrochemical properties: (2) using a signal amplification chemistry that remains attached or deposited in close proximity to the tracer itself.

In some embodiments, labeled binding partners (e.g., antibodies or antigen binding fragments) may be used as tracers to detect binding (e.g., using antigen bound antibody complexes). Examples of the types of labels which may be useful for the instant methods and compositions include enzymes, radioisotopes, colloidal metals, fluorescent compounds, magnetic, chemiluminescent compounds, electrochemiluminescent groups, metal nanoparticles, and bioluminescent compounds. Radiolabeled binding partners (e.g., antibodies) may be prepared using any known method and may involve coupling a radioactive isotope such as ¹⁵³Eu, ³H, ³²P, ³⁵S, ⁵⁹Fe, or ¹²⁵I, which can then be detected by gamma counter, scintillation counter or by autoradiography. Binding partners (e.g., antibodies or antigen binding fragments) may alternatively be labeled with enzymes such as yeast alcohol dehydrogenase, horseradish peroxidase, alkaline phosphatase, and the like, then developed and detected spectrophotometrically or visually. The label may be used to react a chromogen into a detectable chromophore (including, for example, if the chromogen is a precipitating dye).

Suitable fluorescent labels may include, but are not limited to: fluorescein, fluorescein isothiocyanate, fluorescamine, rhodamine, Alexa Fluor® dyes (such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, or Alexa Fluor® 790), cyanine dyes including, but not limited to: Cy2, Cy3, Cy3.5, Cy5, Cy5.5, Cy7, and Cy7.5, and the like. The labels may also be time-resolved fluorescent (TRF) atoms (e.g., Eu or Sr with appropriate ligands to enhance TRF yield). More than one fluorophore capable of producing a fluorescence resonance energy transfer (FRET) may also be used. Suitable chemiluminescent labels may include, but are not limited to: acridinium esters, luminol, imidazole, oxalate ester, luciferin, and any other similar labels.

Suitable electrochemiluminescent groups for use may include, as a non-limiting example: Ruthenium and similar groups. A metal nanoparticle may also be used as a label. The metal nanoparticle may be used to catalyze a metal enhancement reaction (such as gold colloid for silver enhancement).

Any of the labels described herein or known in the field may be linked to the tracer using covalent or non-covalent means. The label may be presented on or inside an object like a bead (including, for example, a plain bead, hollow bead, or bead with a ferromagnetic core), and the bead is then attached to the binding partner (e.g., an antibody or antigen-binding fragment thereof). The label may also be a nanoparticle including, but not limited to, an up-converting phosphorescent system, nanodot, quantum dot, nanorod, and/or nanowire. The label linked to the antibody may also be a nucleic acid, which might then be amplified (e.g., using PCR) before quantification by one or more of optical, electrical or electrochemical means.

In some embodiments, the binding partner is immobilized on the solid support prior to formation of binding complexes. In other embodiments, immobilization of the antibody and antigen-binding fragment is performed after formation of binding complexes.

In one embodiment, immunoassay methods disclosed herein comprise immobilizing binding partners (e.g., antibodies or antigen-binding fragments) to a solid support (e.g., a chip); applying a sample (e.g., an endometrial fluid sample) to the solid support under conditions that permit binding of the expression product of a biomarker (e.g., a protein) to one or more binding partners (e.g., one or more antibodies or antigen-binding fragments), if present in the sample; removing the excess sample from the solid support; detecting the bound complex (using, e.g., detectably labeled antibodies or antigen-binding fragments) under conditions that permit binding (e.g., of an expression product to the antigen-bound immobilized antibodies or antigen-binding fragments); washing the solid support and assaying for the label.

Reagents can be stored in or on a chip for various amounts of time. For example, a reagent may be stored for longer than 1 hour, longer than 6 hours, longer than 12 hours, longer than 1 day, longer than 1 week, longer than 1 month, longer than 3 months, longer than 6 months, longer than 1 year, or longer than 2 years. Optionally, the chip may be treated in a suitable manner in order to prolong storage. For instance, chips having stored reagents contained therein may be vacuum sealed, stored in a dark environment, and/or stored at low temperatures (e.g., below 4° C. or 0° C.). The length of storage depends on one or more factors such as the particular reagents used, the form of the stored reagents (e.g., wet or dry), the dimensions and materials used to form the substrate and cover layer(s), the method of adhering the substrate and cover layer(s), and how the chip is treated or stored as a whole. Storing of a reagent (e.g., a liquid or dry reagent) on a solid support material may involve covering and/or sealing the chip prior to use or during packaging.

Any solid state assay device described herein may be included in a kit. The kit may include any packaging useful for such devices. The kit may include instructions for use in any format or language. The kit may also direct the user to obtain further instructions from one or more locations (physical or electronic). The included instructions can comprise a description of how to use the components contained in the kit for measuring the level of a biomarker set (e.g., protein biomarker or nucleic acid biomarker) in a biological sample collected from a subject, such as a human patient. The instructions relating to the use of the kit generally include information as to the amount of each component and suitable conditions for performing the assay methods described herein.

The components in the kits may be in unit doses, bulk packages (e.g., multi-dose packages), or sub-unit doses. The kit can also comprise one or more buffers as described herein but not limited to a coating buffer, a blocking buffer, a wash buffer, and/or a stopping buffer.

The kits of this present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an PCR machine, a nucleic acid array, or a flow cytometry system.

Kits may optionally provide additional components such as interpretive information, such as a control and/or standard or reference sample. Normally, the kit comprises a container and a label or package insert(s) on or associated with the container. In some embodiments, the present disclosure provides articles of manufacture comprising contents of the kits described above.

### EXAMPLES

### Materials and Methods

### Subject Details

All procedures involving human endometrium were conducted in accordance with the Institutional Review Board (IRB) guidelines for Stanford University under the IRB code IRB-35448 and IVI/University of Valencia under the IRB code 1603-IGX-016-CS, including informed consent for tissue collection from all subjects. Collection of endometrial biopsies was approved by the IRB code 1603-IGX-016-CS. There were no medical reasons to obtain the endometrial biopsies. Healthy ovum donors were recruited in the context of the research project approved by the IRB. Informed written consent was obtained from each woman before an endometrial biopsy was performed in their natural menstrual cycle (no hormone stimulation). De-identified human endometrium was obtained from women aged 18-34, with regular menstrual cycle (3-4 days every 28-30 days), BMI ranging 19-29 kg/m² (inclusive), and negative serological tests for HIV, HBV, HCV, RPR and normal karyotype. Women with the following conditions were excluded from tissue collection: with recent contraception (IUD in past 3 months; hormonal contraceptives in past 2 months), uterine pathology (endometriosis, leiomyoma, or adenomyosis; bacterial, fungal, or viral infection), and polycystic ovary syndrome.

### Endometrium Tissue Dissociation and Population Enrichment

A two-stage dissociation protocol was used to dissociate endometrium tissue and separate it into stromal fibroblast and epithelium enriched single cell suspensions. Prior to the dissociation, the tissue was rinsed with DMEM (Sigma) on a petri dish to remove blood and mucus. Excess DMEM was removed after the rinsing. The tissue was then minced into pieces as small as possible, and dissociated in collagenase A1 (Sigma) overnight at 4° C. in a 50 mL falcon tube at horizontal position. This primary enzymatic step dissociates stromal fibroblasts into single cells while leaving epithelium glands and lumen mostly undigested. The resulting tissue suspension was then briefly homogenized and left un-agitated for 10 mins in a 50 mL Falcon tube at vertical position, during which epithelial glands and lumen sedimented as a pellet and stromal fibroblasts stayed suspended in the supernatant. The supernatant was therefore collected as the stromal fibroblast-enriched suspension. The pellet was washed twice in 50 mL DMEM to further remove residual stromal fibroblasts. The washed pellet was then dissociated in 400 μL TrypLE Select (Life technology) for 20 mins at 37° C., during which homogenization was performed via intermittent pipetting. DNaseI (100 μL) was then added to the solution to digest extracellular genomic DNA. The digestion was quenched with 1.5 mL DMEM after 5 min incubation. The resulting cell suspension was then pipetted, filtered through a 50 μm cell strainer, and centrifuged at 1000 rpm for 5 min. The pellet was re-suspended as the epithelium-enriched suspension.

Single Cell Capture, Imaging, and cDNA Generation

For cell suspension of both portions, live cells were enriched via MACS dead cell removal kit (Miltenyi Biotec) following the manufacture's protocol. The resulting cell suspension was diluted in DMEM into a final concentration of 300-400 cells/μL before being loaded onto a medium C1 chip for mRNA Seq (Fluidigm). Live dead cell stain (Life Technology) was added directly into the cell suspension. Single cell capture, mRNA reverse-transcription, and cDNA amplification were performed on the Fluidigm C1 system using default scripts for mRNA Seq. All capture site images were recorded using an in-house built microscopic system at 20× magnification through phase, GFP, and Y3 channels. 1 μL pre-diluted ERCC (Ambion) was added into the lysis mix, resulting in a final dilution factor of 1:80,000 in the mix.

### Single Cell RNAseq Library Generation

Single-cell cDNA concentration and size distribution were analyzed on a capillary electrophoresis-based automated fragment analyzer (Advanced Analytical). Fragmented and barcoded cDNA libraries were prepared only for cells imaged as singlet or empty at the capture site and with >0.06 ng/uL cDNA generated. Library preparation was performed using Nextera XT DNA Sample Preparation kit (Illumina) on a Mosquito HTS liquid handler (TTP Labtech) following Fluidigm's single cell library preparation protocol with a 4× scale-down of all reagents. Dual-indexed single-cell libraries were pooled and sequenced in pair-end reads on Nextseq (Illumina) to a depth of 1-2×10⁶ reads per cell. Bcl2fastq v2.17.1.14 was used to separate out the data for each single cell by using unique barcode combinations from the Nextera XT preparation and to generate *.fastq files.

### Single Cell RNAseq Data Analysis

Raw reads in the *.fastq files were trimmed to 75 bp using fastqx, aligned to Ensembl human reference genome GRCh38.87 (dna.primary_assembly) using STAR (Dobin et al., 2013) with default parameters, duplicate-removed using picard MarkDuplicates with default parameters. Aligned reads were converted to counts using HTSeq (Anders et al., 2015) and Ensembl GTF for GRCh38.87 under the setting -m intersection-strict \-s no. Downstream data analysis was performed in R and Java. For each cell, counts were normalized to log transformed reads per million (log 2(rpm+1)) by the equation

$${log}_{2} ( r p m ) = {log}_{2} ( 1 + \frac{c {t}_{ij} * 1 e 06}{Σ c {t}_{i}} )$$

where i is for cell i and j for gene j.

### Quality Filtering of Single Cells

For quality filtering, fraction of reads mapped to ERCC (f_{ERCC}) was used as the quality metric and empirical cumulative distribution of f_{ERCC} in empty capture sites recorded on the C1 chip was calculated and used as the null model (ecdfₙᵤₗₗ). Single cells retained for downstream analysis were those with (ecdfₙᵤₗₗ(f_{ERCC}))<0.05. 2149 cells were retained for downstream analysis.

### Differential Expression Analysis

To obtain differentially expressed genes for a cell type or state, for each gene, Wilcoxon's rank sum test (Mann and Whitney, 1947) was performed and 2) fold change (FC, dummy variable=1E-02) was calculated between cells within a cell type/state and the cells from other cell types/states. P-values obtained from the Wilcoxon's rank sum test were adjusted for multiple comparisons by Benjamini-Hochberg's procedure (Benjamini and Hochberg, 1995) to obtain p.adj. To evaluate the “sensitivity” and “specificity” of a gene in identifying a cell type/state, the percent of cells was also calculated within the cell type/state of interest that are expressing the gene (pctin) and the percent of cells from other cell types/states expressing the gene (pctout), as well as the ratio between the pctin and pctout.

### Gene Ontology Functional Enrichment

Functional enrichment analysis was performed using Gene Ontology Enrichment Analysis (geneontology.org) and each enriched ontology hierarchy (FDR<0.05) was reported with two terms in the hierarchy: the term with the highest significant value and 2) the term with the highest specificity.

### Enrichment of “Time-Associated” Genes Via Mutual Information (MI) Based Approach

The “time-associatedness” of a gene was calculated as the MI between the expression of a gene and time (or pseudotime) using the Java implementation of ARACNe-AP (Lachmann et al., 2016). For each gene, MIi=MI((e1i, e2i, . . . , eni), (t1, t2, . . . , tn)), where i is for gene i, eni is for expression of gene i in cell n, and tn is the time (or pseudotime) annotation of cell n. The statistical significance of the MIi was evaluated using the null model where the time (or pseudotime) annotation was permutated for 1000 times with respect to cells, based on which an empirical cumulative distribution function (ecdfnull,i) between the expression of gene i and the permutated time (or pseudotime) was constructed using R function ecdf. The p-value for MIi was calculated as (1-ecdfnull,i(MIi)). The p-values were then adjusted for multiple comparisons by Benjamini-Hochberg's procedure (Benjamini and Hochberg, 1995) to obtain FDR for each gene.

### Cell Heterogeneity Analysis

Over-dispersion of genes was calculated as

$$\frac{{CV}_{i}^{2}}{{CV}_{e}^{2}} ,$$

where CVᵢ² is the squared variation of coefficient of gene i across cells of interest and CVₑ² is the expected squared variation of coefficient given mean, fitted using non-ERCC counts. All pairwise distances between cells were calculated as (1-Pearson's correlation). Dimensional reduction was performed using R implementation of tSNE (Rtsne).
Smoothing of “Time-Associated” Genes and Assignment into Characteristic Phases

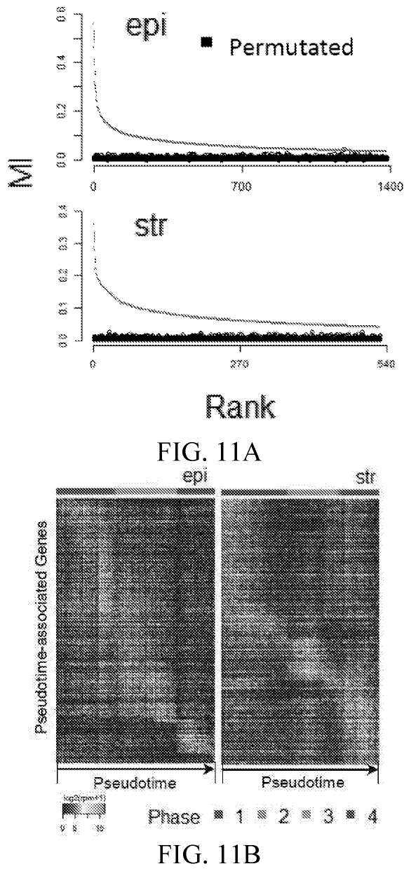

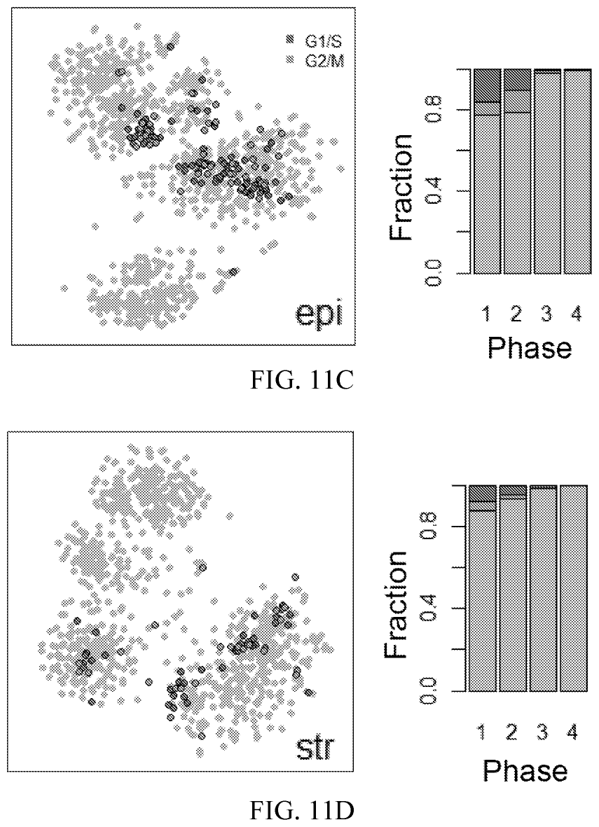

To estimate the pseudotime at which a gene reached maximum expression (pseudotimeₘₐₓ), smoothing of gene expression was performed with respect to pseudotime using the R function smooth spline( ) (spar=1) and the pseudotime(s) at which a smoothed curve reached local maximum was estimated using the R function peaks( ) and inflection point estimated using custom R script. Characteristic signatures for phase 1-4 were identified by assigning each pseudotime-associated gene that was identified (FIG. 11A-11B) to the phase where its peak expression occurred (i.e., pseudotimeₘₐₓ).

### Characterization of Global Transcriptional Factor and Secretory Gene Dynamics

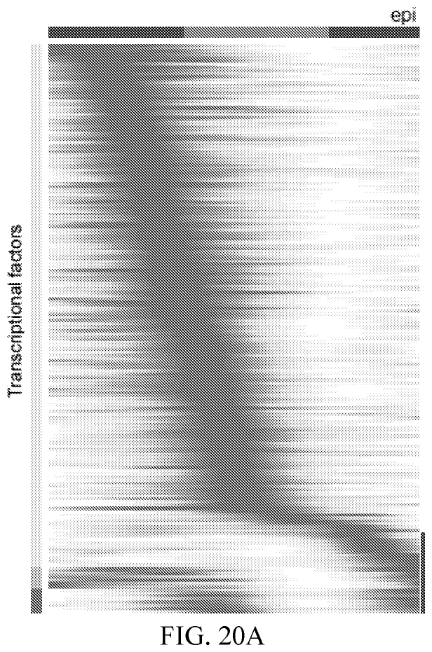

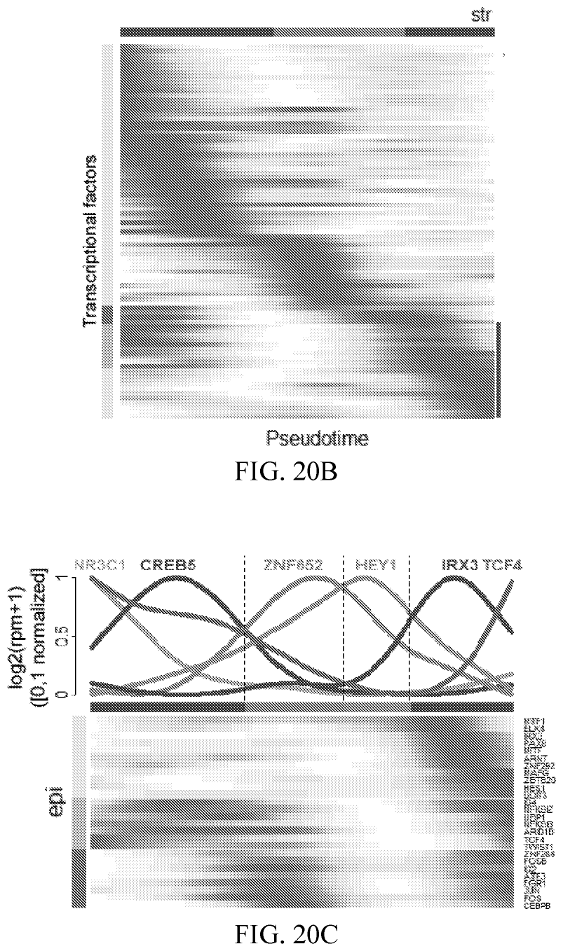

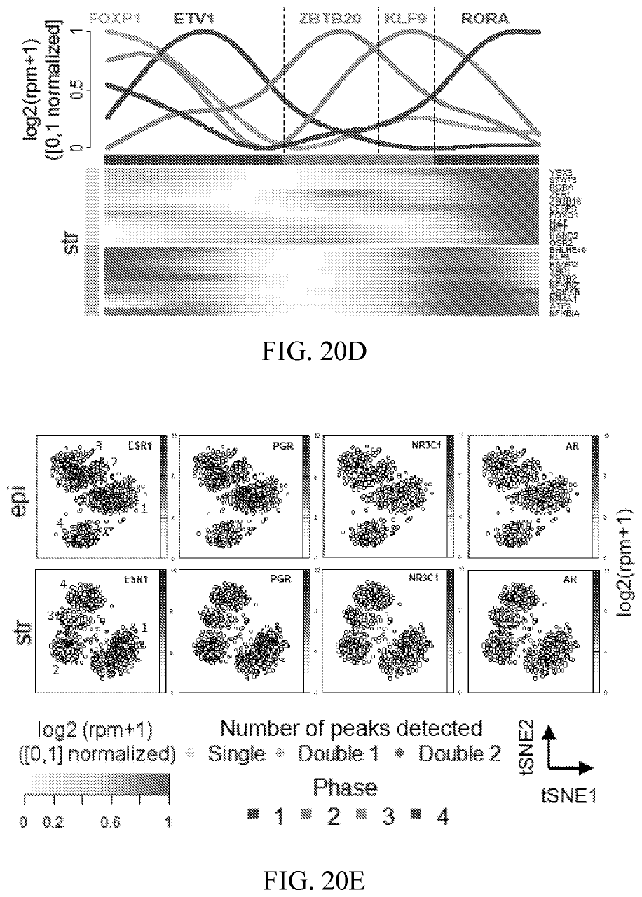

A dynamic transcriptional factor (FIG. 20A-20E) was defined as a “time-associated” gene (FIG. 11B) annotated as a transcriptional regulator by the Human Protein Atlas (Uhlen et al., 2015). Dynamic TFs were first categorized into major groups using hierarchical clustering on smoothed and [0,1] normalized curves. In each group, TFs were ordered by the pseudotime where a peak or a major peak (for curves with two peaks) occurred, and ties were broken by the pseudotime where an inflection point occurred.

### Cell Cycle Analysis

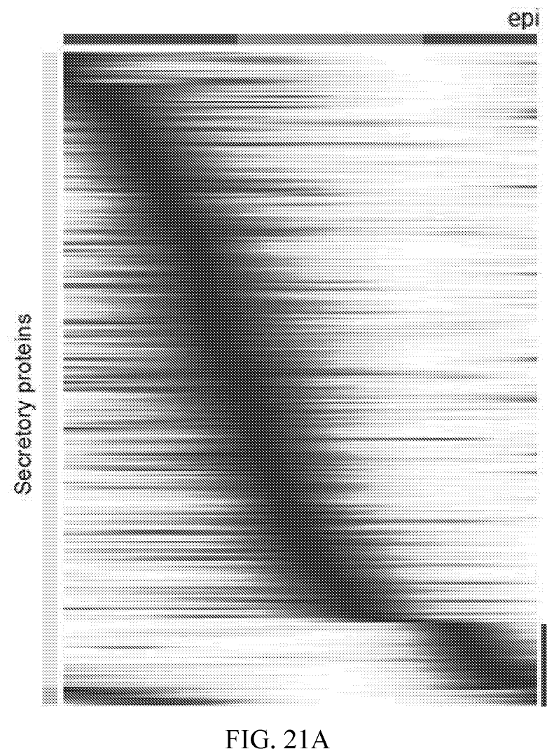

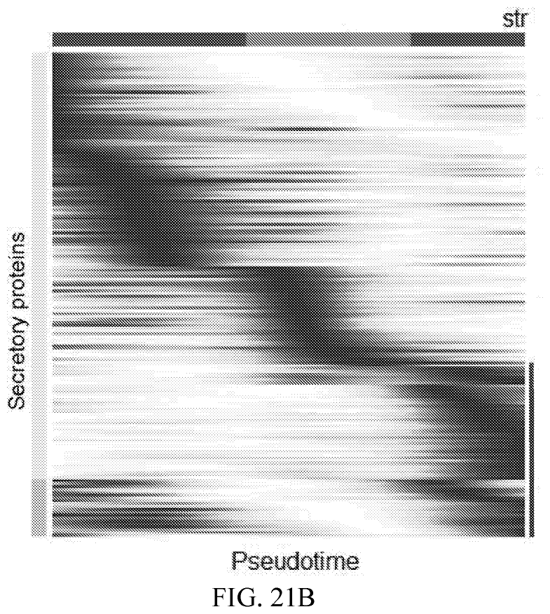

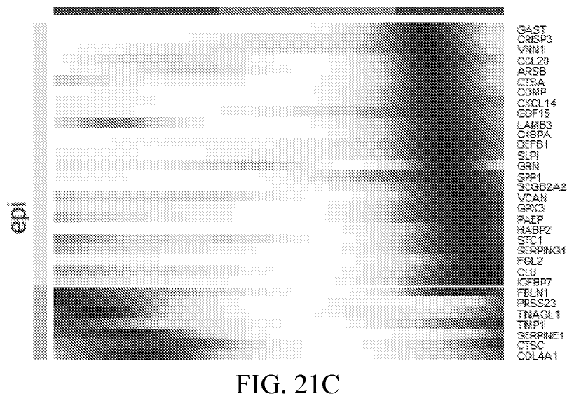

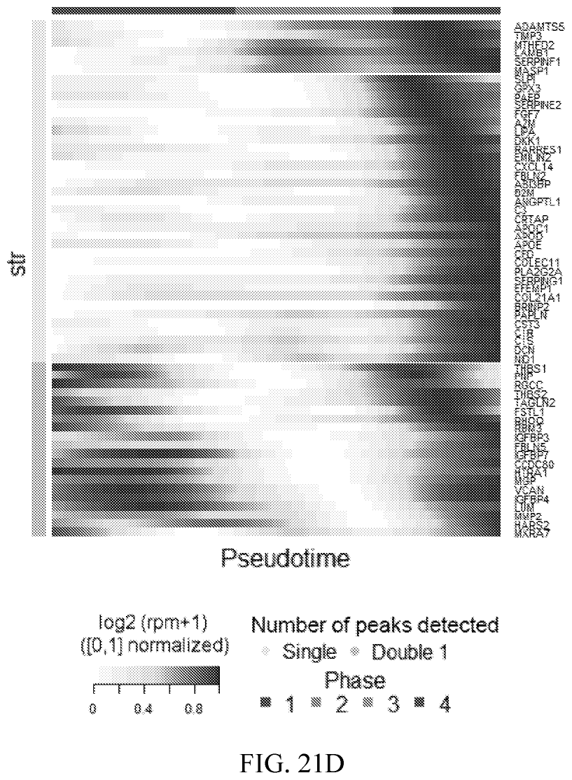

A two-step approach was taken in identifying cycling cells and defining endometrium-specific cell cycle signatures. A published gene set encompassing 43 G1/S and 55 G2/M genes (Tirosh et al., 2016), was used, representing the intersection of four previous gene sets (Kowalczyk et al., 2015; Macosko et al., 2015; Whitfield, 2002), and calculated a G1/S and a G2/M score for all single cells in unciliated epithelial and stromal fibroblasts, respectively, following the scoring scheme in (Tirosh et al., 2016). Briefly, cells with at least 2× average expression of either G1/S or G2/M genes than the average of all cells in the respective cell type was assigned as putative cycling cells. Wilcoxon's rank sum test (Mann and Whitney, 1947) was performed between the putative cycling cells and the rest of cells in the cell type to enrich for cell-cycle associated transcriptome signatures that were specific to endometrium (FIG. 7, and FIG. 21A). To assign cells into G1/S or G2/M stages, dimension reduction was performed on putative cycling cells using the identified signature, which revealed two major populations enriched in known G1/S or G2/M signatures. Genes were assigned as either G1/S or G2/M associated by estimating the population at which peak expression of the gene occurred. The G1/S and G2/M scores were then recalculated for each cell using the signature customized for endometrium and finalized the assignment of G1/S and G2/M cells with at least 2× average G1/S or G2/M expression with respect to all cells in that cell type.

### Identification of Putative Ligand-Receptor Interactions Between Unciliated Epithelial Cells and Stromal Fibroblasts

For each identified phase and subphase, the expression of a known ligand or receptor was evaluated as the percent of unciliated epithelial cells or stromal fibroblasts expressing the genes to obtain p_{(epi, j)} and p_{(str, j)}, where j is for phase j. A ligand or receptor is only considered expressed by a cell type in a phase if p is greater than 25%. The interaction between a ligand-receptor pair is established if when a ligand is expressed in one cell type and its known receptor is expressed in the other. The ligand-receptor pairing information was based on the database provided by (Ramilowski et al., 2015). Ligand-receptor pairs were sorted, from top to bottom, left to right, by the level of interaction, quantified as the total number of interactions normalized by the total number of possible interactions between the two cell types within a phase. This information can be used to identify one or more ligand-receptor pairs that can be used to determine the menstrual status of a subject, for example to determine whether the subject is within the WOI.

### Tissue Preparation for In Situ Hybridizations

Endometrial tissues were fixed for 24-48 h in 4% paraformaldehyde (PFA) at room temperature, trimmed, embedded in paraffin, and sectioned into 3 μm in thickness onto APES-coated slides.

### Immunofluorescence

Tissue sections were baked at 60° C. for 1 h, deparaffined with Histoclear and rehydrated with ethanol series. Antigen retrieval was performed by boiling tissue sections in 10 mM sodium citrate buffer (pH 6.0) for 20 min, followed by immediate cool down in cold water for 10 min. Tissue permeabilization was done with 0.25% Triton X 100 in PBS for 5 min, followed by wash in 0.05% Triton X100 in PBS for 5 min twice. Non-specific binding was blocked with 5% BSA-0.05% Triton X100-4% goat serum in PBS for 1 h at room temperature. Tissue sections were then incubated with primary antibodies over night at 4° C. and secondary antibodies for 1 h at room temperature. Primary antibodies used and dilution ratios are Vimentin (2 μg/mL, ab8978, Abcam), Prolactin (1:10, PA5-26006, Thermo Fischer Scientific), CD3 (1:100, ab5690, Abcam), CD56 (1:50, ab133345, Abcam). Secondary antibodies used and dilution ratios are: Goat anti-mouse IgG (H+L) Superclonal™ Alexa Fluor 488 (1:200, A27034, Thermo Fischer Scientific) and Goat anti-rabbit IgG (H+L) Superclonal™ Alexa Fluor 555 (1:200, A27039, Thermo Fisher Scientific). All sections were counterstained with 4′, 6′-diamidino-2-phenylindole (DAPI) (Thermo Fisher Scientific) and mounted with Aquatex® (Merck-Millipore). Images were captured with a confocal microscope (FV1000, Olympus) at 20× and 60× magnification with oil immersion and analyzed using Imaris (Bitplane).

### RNAscope for Ciliated Cells

Combined RNA and antibody in situ hybridizations were performed according to the manufacturer's technical note “RNAscope Multiplex Fluorescent v2 Assay combined with Immunofluorescence” for FFPE samples (Advanced Cell Diagnostics). 15 min and 30 min incubation were used for target retrieval and Protease Plus treatment, respectively. RNA probes (Advanced Cell Diagnostics) with the following channel assignment (C), fluorophore, and dilution in TSA buffer were used: CDHR3 (C1, cyanine 3, 1:1500), C11orf88 (C2, cyanine 5, 1:750); C20orf85 (C1, cyanine 3, 1:1500), FAM183A (C2, cyanine 5, 1:1500). Tissue sections were blocked with SuperBlock (PBS) blocking buffer (Fisher Scientific) for 30 min at room temperature, incubated in anti-human FOXJ1 (1:500, eBioscience) over night at 4° C. and goat anti-mouse IgG secondary antibody (1:500, Life Technologies) for 2 h at room temperature. All sections were mounted with Prolong Diamond Antifade Mountant (Thermo Fisher Scientific). Imaging was carried out on an Axio-plan epifluorescence microscope equipped with an Axiocam 506 mono camera (Zeiss) using a 20×/0.8 Plan-Apochromat objective (Zeiss). For each sample, 8-10 fields of view were captured with 10-15 z-stacks.

### Analysis of RNAscope Images

Z-stacks were projected (maximum intensity projection, MIP) using ImageJ. The resulting MIP images were analyzed using CellProfiler 3.0.0 as follows: 1) Correct background by subtracting the lower quartile of the intensity measured from the whole image. 2) Detect cell nuclei using the DAPI channel and cell boundaries using Voronoi distance (25 pixels) from the nuclei. 3) Enhance RNA signals using a tophat filter (5 pixels) and detect signals by intensity threshold (0.004 and 0.002 for Cy3 and Cy5, respectively). 4) Measure antibody intensity for each detected cell. All images were analyzed in the same way, with no image excluded.

### Example 1—Human Endometrium Consists of Six Cell Types Across the Menstrual Cycle

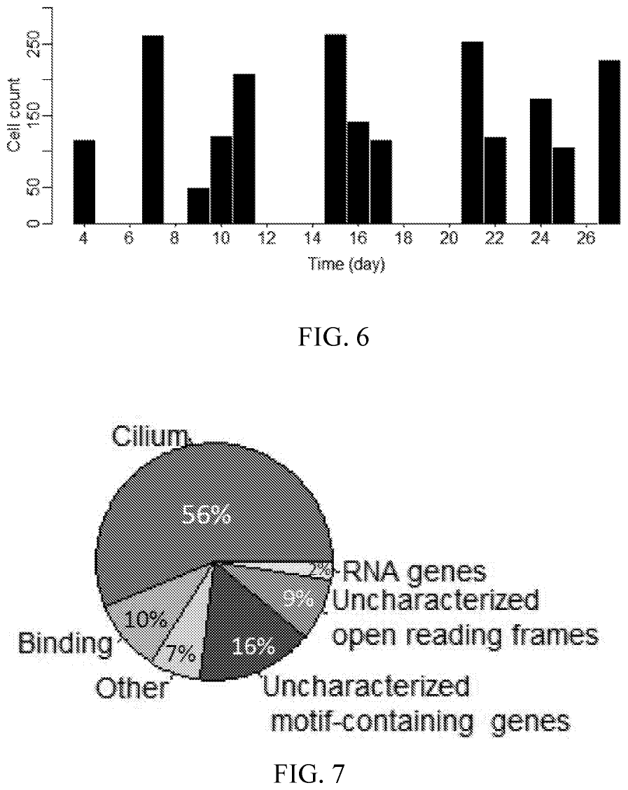

To characterize endometrial transformation across the natural human menstrual cycle, endometrial biopsies from 19 healthy and fertile females were collected, 4-27 days after the onset of her latest menstrual bleeding (FIG. 6). All females were on regular menstrual cycles, with no influence from exogenous hormone or obstetrical pathology. Single cells were captured and cDNA was generated using Fluidigm C1 medium chips. Fraction of reads mapped to ERCC was used as the metric for quality filtering (Method).

Dimensional reduction via t-distributed stochastic neighbor embedding tSNE) (Maaten and Hinton, 2008) on the top over-dispersed genes (Method) revealed clear segregation of cells into distinct groups (FIG. 1A). Cell types were defined as segregations that are not time-associated, i.e., groups encompassing cells sampled across the menstrual cycle. Six cell types were thus identified; canonical markers and highly differentially expressed genes enabled straightforward identification of four of these: stromal fibroblast, endothelium, macrophage, and lymphocyte (FIG. 1B). The two remaining cell types both express epithelium-associated markers; one of these cell types was characterized by an extensive list of uniquely expressed genes. Functional analysis (Ashburner et al., 2000; Mi et al., 2017; The Gene Ontology Consortium, 2017) revealed that 56% of genes in this list were annotated with a cilium-associated cellular component or biological process (FIG. 1C, FIG. 7), thereby identifying this cell type as “ciliated epithelium”, specifically with motile cilia (Mitchison and Valente, 2017; Zhou and Roy, 2015). The other epithelial cell type was defined as “unciliated epithelium.”

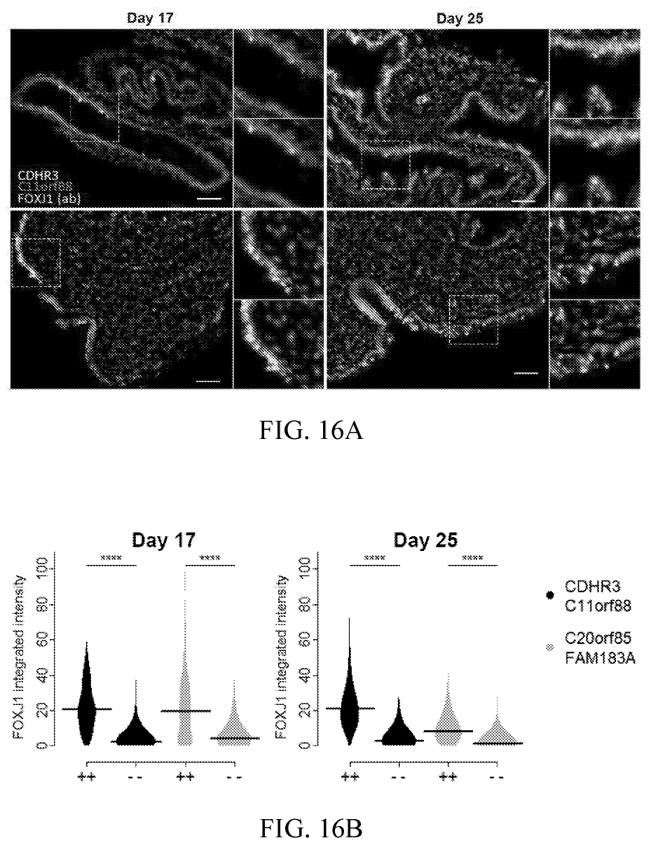

Using RNA and antibody co-staining (Method), previously unannotated discriminatory markers and epithelial lineage identity were validated, and the spatial distribution of ciliated epithelium was visualized in situ. Four genes were selected for RNA staining: they were identified as highly discriminatory for the cell type (FIG. 1B) but either have no previous functional annotation (C11orf88, C20orf85, FAM183A) or are annotated with non-cilia-associated functionality (CDHR3). Consistent co-expression of all four genes was found with FOXJ1 (canonical master regulator for motile cilia with epithelial lineage identity) antibody staining in both glandular and luminal epithelia at day 17 (FIG. 16A, left panels) and day 25 (FIG. 16A, right panels) of the menstrual cycle. The results validated these ciliated cells as an epithelial subpopulation of both luminal and glandular epithelia in healthy human endometrium across the menstrual cycle. This data also demonstrates the consistent discriminatory power of the new markers that were identified (FIG. 16B) across the cycle. Lastly, the co-expression of these unannotated markers in ciliated cells helps confirm a likely cilia-associated functionality for them and for other unannotated markers that were identified, which constituted 44% of all markers identified for this cell type (FIG. 7, Table 11). Accordingly, one or more of the genes in Table 11 may be used as biomarkers for identifying cells with cilia-associated functionality. In some embodiments, an assay is performed to monitor the expression level of one or more of the biomarkers disclosed in Table 11 to identify cells with cilia-associated functionality. In some embodiments, one or more of the biomarkers disclosed in Table 11 can be subject to analysis using any of the assay methods described herein, including, but not limited to, measuring the level of one or more biomarkers as described in Table 11. In some embodiments, the level of a biomarker in Table 11 is assessed or measured by directly detecting the protein in a sample, or measured indirectly in a sample, for example, by detecting the level of activity of the protein. In some embodiments, the level of nucleic acids encoding a biomarker in Table 11 is assessed or measured. In some embodiments, measuring the expression level of nucleic acid encoding the biomarker comprises measuring mRNA. In some embodiments, the number of biomarkers from Table 11 that are measured is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers. In some embodiments, co-expression of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers in Table 11 in a cell is indicative of cilia-associated functionality.

TABLE 11

| | | | | |
|---|---|---|---|---|
| C11orf88 | CCDC17 | LRRC46 | CDHR4 | MDH1B |
| C1orf194 | CCDC173 | MORN5 | EFCAB1 | MS4A8 |
| C1orf87 | DTHD1 | VWA3A | EFCAB10 | MUC13 |
| C20orf85 | DYDC2 | ZBBX | SPATA17 | PPIL6 |
| C5orf49 | FAM183A | AC013264.2 | ADGB | DLEC1 |
| C6orf118 | FAM216B | PCAT19 | CASC1 | CFAP69 |
| C9orf135 | FAM81B | CAPSL | ARMC3 | |
| ANKRD66 | FHAD1 | CDHR3 | MAP3K19 | |

### Example 2—Human Endometrial Transformation Consists of Four Major Phases Across the Menstrual Cycle

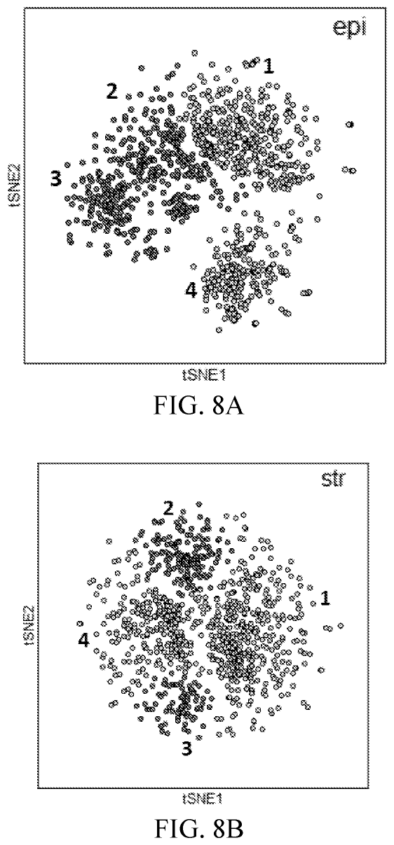

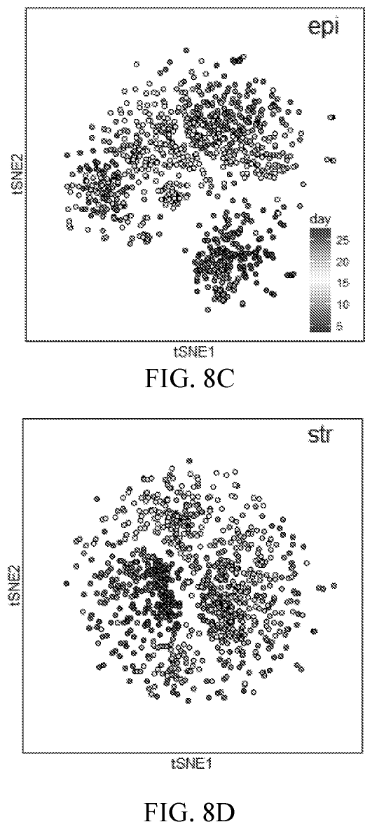

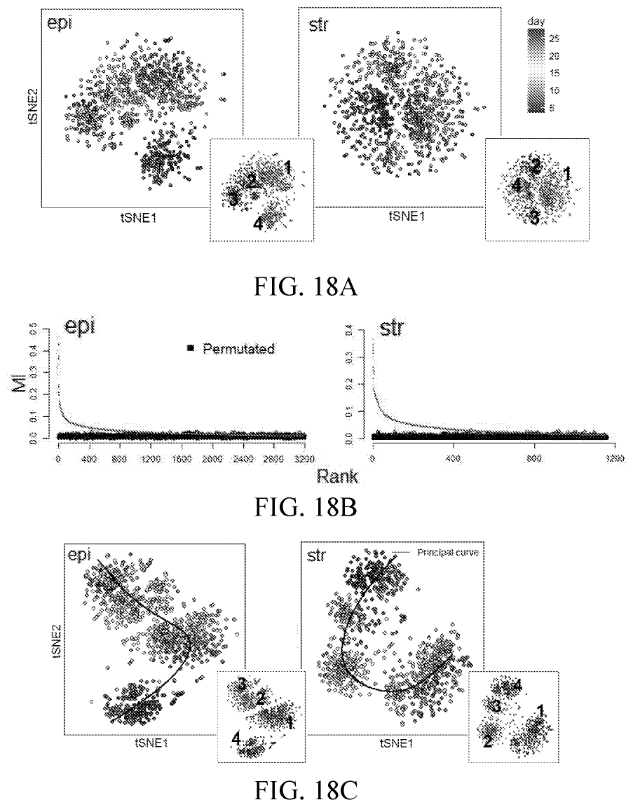

Samples were taken throughout the menstrual cycle and annotated by the day of menstrual cycle (the number of days after the onset of last menstrual bleeding). While the time variable serves as an informative proxy for assigning endometrial states, it is susceptible to bias due to variances in menstrual cycle lengths between and within women (Guo et al., 2006), and limited in resolution due to variance of cells within an individual. To study transcriptomes of endometrial transformation in an unbiased manner, within-cell type dimension reduction (tSNE) was performed using whole transcriptome data from unciliated epithelium and stromal fibroblast, respectively. The results revealed four major phases for both cell types, which are referred to as phases 1-4 (FIGS. 8A, 8B, and 18A insets). The four phases were clearly time-associated, confirming the overall validity of the time annotation (FIGS. 8C, 8D, and 18A). Examples where the orders between two women in their phase assignments and time annotation were reversed and cases where cells with the same time annotation were assigned into different phases, demonstrated the bias and limited resolution if time were to be used directly for characterizations (FIG. 8 and FIG. 18A).

### Example 3—Constructing Single Cell Resolution Trajectories of Menstrual Cycle Using Mutual Information Based Approach

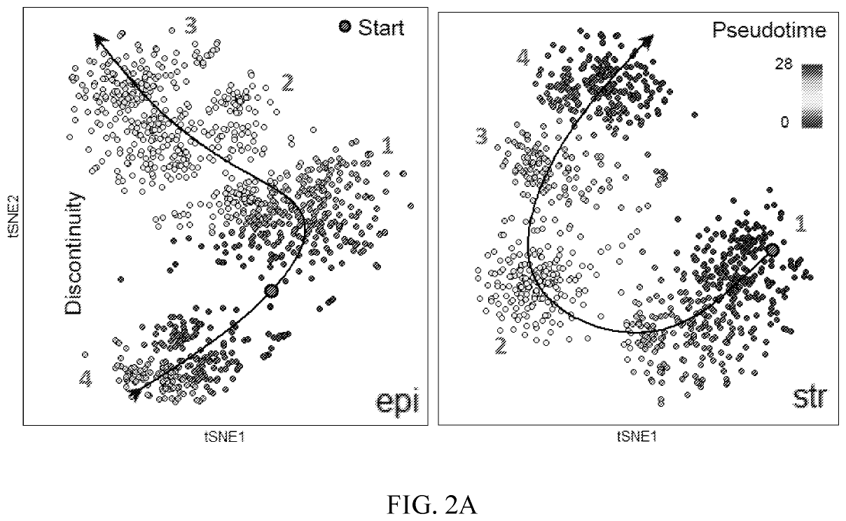

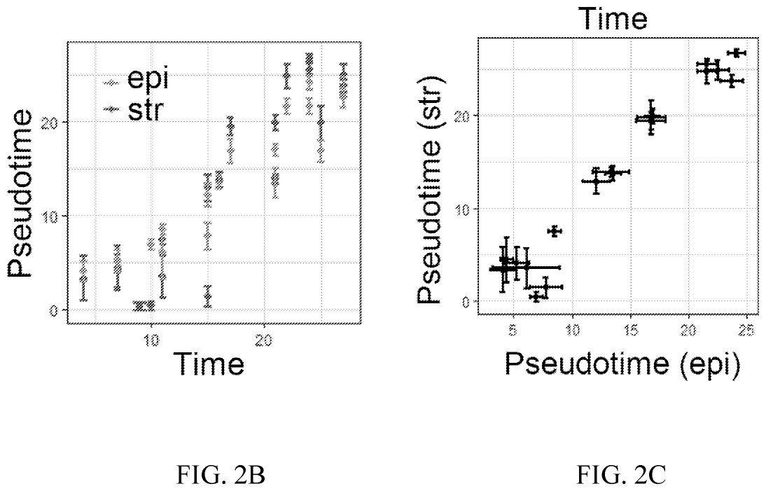

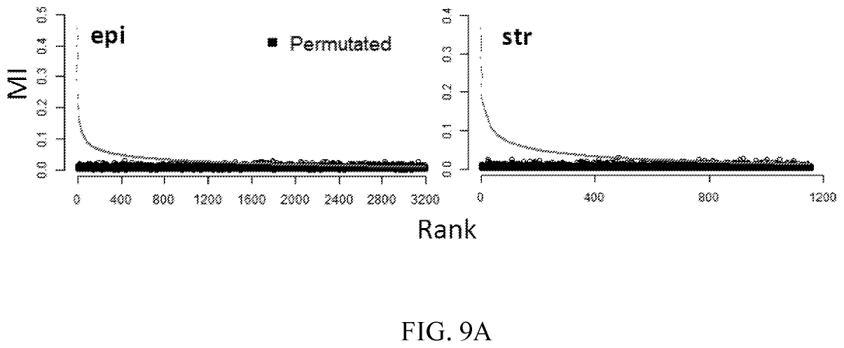

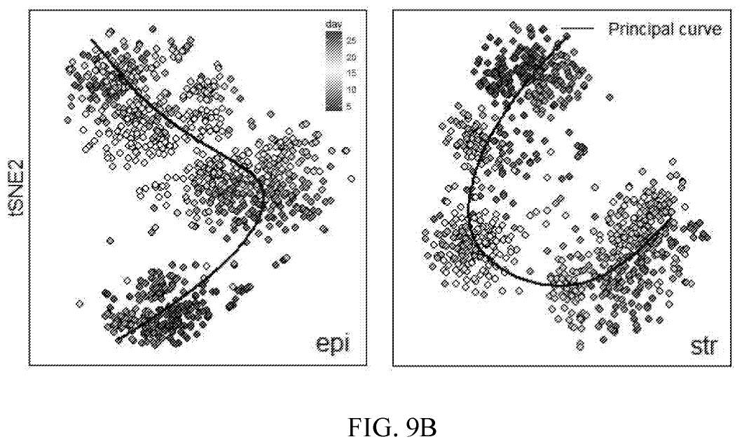

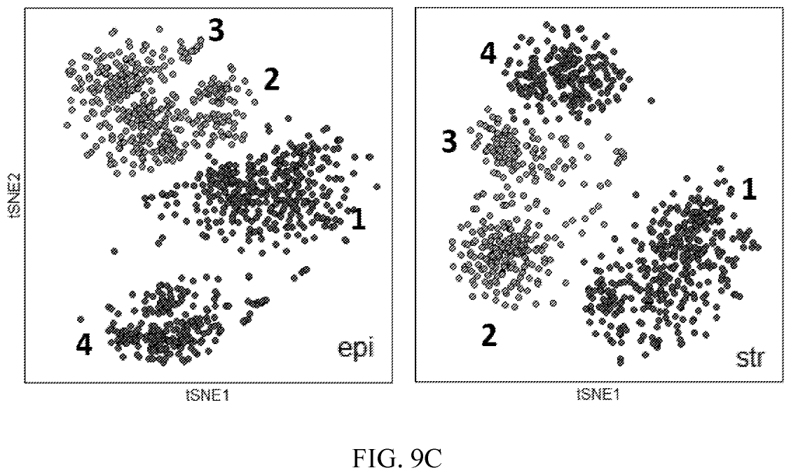

Endometrial transformation over the menstrual cycle is at least in part a continuous process. A model that not only retains phase-wise characteristics but also allows delineation of continuous features between and within phases will enable higher precision characterizations. To build such a model, a mutual information (MI) (Tkačik and Walczak, 2011) based approach was used, such that the information provided by the time annotation was exploited, its limitation noted in the previous section minimized, and potential continuity between and within phases accounted for. Briefly, enrich for genes that were changing across the menstrual cycle based on the MI between gene expression and time annotation regardless of underlying model of dynamics (Method). In total 3,198 and 1,156 “time-associated” genes for unciliated epithelium and stromal fibroblast were obtained, respectively (FDR<0.05) (FIGS. 9A and 18B). For both cell types, dimensional reduction (tSNE) using time-associated genes revealed the same four major phases that were obtained using unsupervised approach (FIGS. 9B, 9C, and 18C insets), demonstrating that the MI-based approach reduced the bias of the time annotation to the same extent as unsupervised approach. Meanwhile, the MI-based approach enabled identification of a clear trajectory that connected the phases and was time-associated within phases. The trajectories were defined using the principal curve (Hastie and Stuetzle, 1989) (FIG. 2A), and assigned each cell an order along the trajectory based on its projection on the curve (Ji and Ji, 2016; Kim et al., 2016; Marco et al., 2014; Petropoulos et al., 2016), which are referred to as pseudotime (FIG. 2A). High correlations between time and pseudotime for both unciliated epithelium and stromal fibroblast were observed (FIG. 2B). The high correlation between pseudotimes of the two cell types from the same woman (FIG. 2C) further supported the validity of the trajectories.

### Example 4—the WOI Opens with an Abrupt and Discontinuous Transcriptomic Activation in Unciliated Epithelium

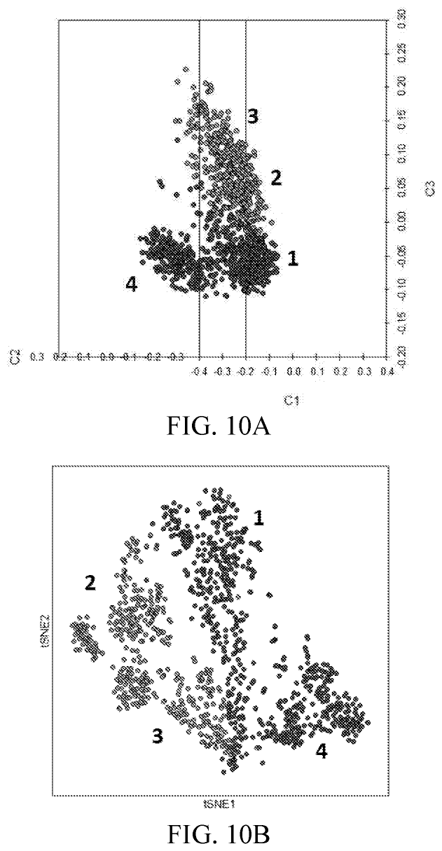

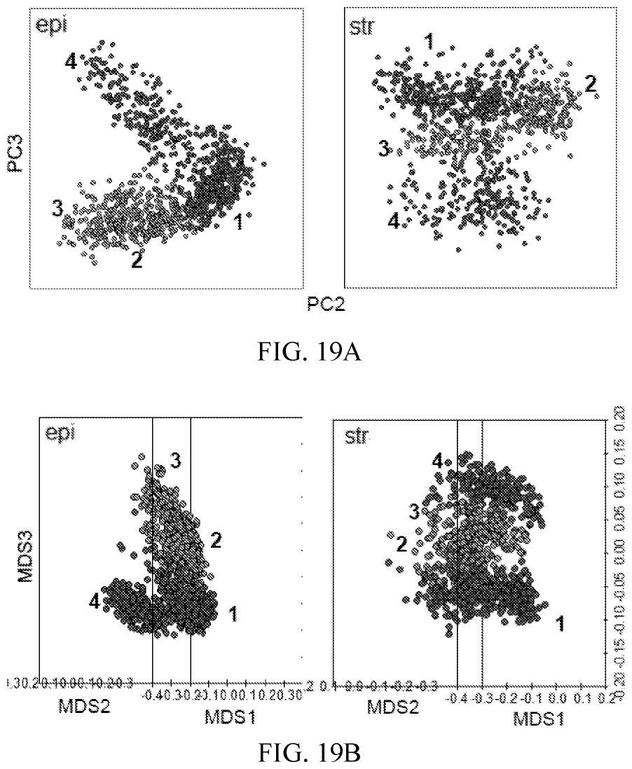

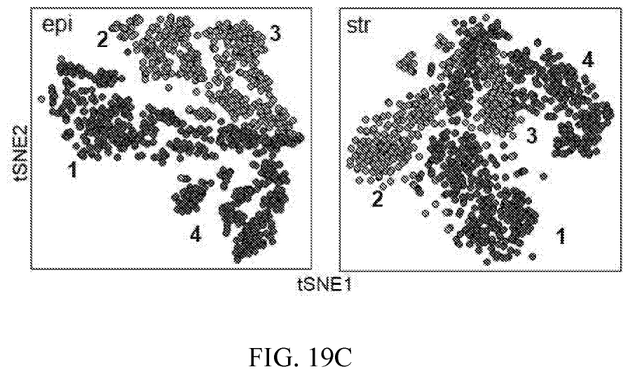

Interestingly, notable discontinuity in the trajectory of unciliated epithelia between phase 4 and the preceding phases was observed (FIG. 2A, left). This discontinuity was consistently observed regardless of the method used for dimension reduction (FIGS. 10A, 19A, and 19B) or feature enrichment (FIGS. 10B, and 19C). It was also unlikely to be an artifact of sampling density given that the involved biopsies were taken with a maximum interval of one day (FIG. 6) and that a similar discontinuity was not observed in the stromal fibroblast counterpart (FIG. 2A, right). To understand the nature of this discontinuity, the genes and their dynamics that contributed to it were explored. Briefly, genes that were dynamically changing along the single-cell trajectories of endometrial transformation were identified by calculating the MI between gene expression and pseudotime, obtaining 1,382 and 527 genes for unciliated epithelial cells and stromal fibroblasts, respectively (FDR<1E-05, FIG. 11A). Ordering these genes based on the pseudotime at which their global maximum was estimated to occur (pseudotimeₘₐₓ, Method) revealed the global features of transcriptomic dynamics across the menstrual cycle (FIG. 11B). In unciliated epithelium, the dynamics demonstrated an overall continuous feature across phase 1-3, until an abrupt and uniform activation of a gene module marked the entrance into phase 4 (FIG. 3A, FIG. 11B). Genes in this module included PAEP, GPX3, and CXCL14 (FIG. 3A), which were relatively consistently reported by bulk transcriptomic profilings as overexpressed in the WOI despite notable discrepancies among bulk profiling results (Díaz-Gimeno et al., 2011; Talbi et al., 2006; reviewed by Ruiz-Alonso et al., 2012). Thus, entrance into phase 4 can be identified with the opening of the WOI. Analysis revealed that this transition into the receptive phase of the tissue occurs with an abrupt and discontinuous transcriptomic activation that is uniform among all cells and activated genes in the unciliated epithelium.

### Example 5—the WOI is Characterized by Widespread Decidualized Features in Stromal Fibroblasts

Unlike their epithelial counterparts, transcriptomic dynamics in stromal fibroblasts demonstrated more stage-wise characteristics, where genes were up-regulated in a modular form, revealing boundaries between phases (FIG. 3B, FIG. 11B left). In phase 4 stromal fibroblasts, the up-regulated gene module included DKK1, S100A4, and CRYAB, among a few others that were recapitulated by consensus among bulk analysis and further confirm the identity of WOI (Diaz-Gimeno et al., 2011; Talbi et al., 2006; reviewed by Ruiz-Alonso et al., 2012), although the transition was not as abrupt as in their epithelial counterparts (FIG. 3A). In the same module, the decidualization initiating transcriptional factor FOXO1 (Park et al., 2016) and decidualized stromal marker IL15 (Okada et al., 2014) were noticed. Importantly, while their upregulation in phase 4 was obvious, their expression was already noticeable in phase 3 in a lower percentage of cells and with lower expression level. Decidualization is the transformation of stromal fibroblasts, where they change from elongated fibroblast-like cells into enlarged round cells with specific cytoskeleton modifications, playing essential roles for embryo invasion and for pregnancy development (for review see Ramathal et al., 2010). Data suggested that this process initiated before the opening of WOI in a small percentage of stromal fibroblasts, and that at the receptive state of tissue, decidualized features are widespread in stromal fibroblasts.

### Example 6—the WOI Closes with Continuous Transcriptomic Transitions

While the WOI opened up with an abrupt transcriptomic transition in unciliated epithelial cells, it closed with a more continuous transition dynamics (FIG. 3A, FIG. 11B, left). Genes expressed in phase 4 unciliated epithelium were featured by three major groups with distinct dynamic characteristics. Group 1 genes (e.g., PAEP, GPX3) had sustained expression throughout the entire phase 4, and their expression remained noticeable until phase 1 of a new cycle. Group 2 genes (e.g., CXCL14, MAOA, DPP4 and the metallothioneins (MT1G, MT1E, MT1F, MT1X)), on the other hand, gradually decreased to zero towards the later part of phase 4, whereas group 3 genes (e.g., THBS1, MMP7) were upregulated at a later part of the phase and their expression is sustained in phase 1 of a new cycle. These characteristics indicate a continuous and gradual transition from mid-secretory to late-secretory phase (Talbi et al., 2006; reviewed by Ruiz-Alonso et al., 2012), and hence the closure of the WOI.

The parallel transition in stromal fibroblasts was also characterized with three similar groups of genes (FIG. 3B, FIG. 11B, right) and continuous dynamics. Specifically a transition towards the later part of phase 4 was observed: gradual down-regulation of decidualization-associated genes (e.g., FOXO1 and IL15) and up-regulation of a separate module of genes (e.g., LMCD1, FGF7). These transitions reveal the final phase of decidualization at the transcriptomic level, which, differing from that during pregnancy, ultimately leads to the shedding of the endometrium in a natural menstrual cycle.

### Example 7—WOI Associated Transcriptional Regulators are Featured with Characteristic Regulatory Roles at the Opening and Closure of WOI

Cell type identity and cell state are primarily driven by small groups of transcriptional regulators. Therefore, it was sought to identify WOI-associated transcriptional factors (TF) to understand what drives the opening and closure of WOI. All TFs that are dynamic across the menstrual cycle (Method) and found for both unciliated epithelia and stromal fibroblasts were first characterized; these TFs can be primarily assigned to two main categories (FIG. 20A, FIG. 20B, Tables 12 and 13), i.e., with 1 or 2 peak(s) of expression detected within one menstrual cycle. Similar to what was observed at whole transcriptome level, the global TF dynamics of the two cell types are notably distinct at the opening of WOI, where in unciliated epithelia a single major discontinuity occurred (FIG. 20A), whereas in stromal fibroblasts no comparable discontinuity was observed (FIG. 20B). These, at the level of transcriptional regulators, validated the WOI-associated transcriptomic dynamics described in previous sections. Accordingly, one or more of the transcriptional factors (TF) in Tables 12 and 13 may be used as biomarkers for identifying the opening and/or closing of the WOI. In some embodiments, an assay is performed to monitor the expression level of one or more of the TF disclosed in Tables 12 and 13 to identify whether the WOI is opening, open, closing or closed. In some embodiments, the expression of one or more TF shown in Table 12 in unciliated epithelial cells is indicative that the WOI is opening and/or open. In some embodiments, the expression of one or more TF shown in Table 13 in stromal fibroblasts is indicative that the WOI is opening and/or open. In some embodiments, one or more of the TFs disclosed in Tables 12 and 13 can be subject to analysis using any of the assay methods described herein, including, but not limited to, measuring the level of one or more TFs as described herein. In some embodiments, the level of a TF in Tables 12 and 13 is assessed or measured by directly detecting the protein in a sample, or measured indirectly in a sample, for example, by detecting the level of activity of the protein. In some embodiments, the level of nucleic acids encoding a TF in Tables 12 and 13 is assessed or measured. In some embodiments, measuring the expression level of nucleic acid encoding the TF comprises measuring mRNA. In some embodiments, the number of TFs from Tables 12 and 13 that are measured is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers. In some embodiments, the number of TFs that are measured are at least 1 gene, at least 10 genes, at least 20 genes, at least 30 genes, at least 40 genes, at least 50 genes, at least 60 genes, at least 70 genes, at least 80 genes, at least 90 genes, at least 100 genes, at least 125 genes, at least 150 genes, at least 175 genes, or at least 180 genes. In some embodiments, the number of genes measured is between 1 and 5 genes, or between 1 and 10 genes, or between 5 and 20 genes, or between 10 and 40 genes, or between 20 and 80 genes, or between 40 and 160 genes, or between 80 and 180 genes.

TABLE 12

| | | | | | | |
|---|---|---|---|---|---|---|
| NFATC2 | NFAT5 | ARID2 | ZNF451 | BBX | CASZ1 | IRX3 |
| TCF7 | SOX9 | TOX4 | ZNF516 | ZBTB11 | NR4A2 | PAX8 |
| ZNF618 | ZHX2 | SMARCA1 | PLAGL2 | YBX1 | TFCP2L1 | MITF |
| RELB | TCF7L2 | ZNF138 | ZNF148 | ZC3H4 | THAP4 | ARNT |
| ADNP2 | IRF6 | LEF1 | ZFP91 | ETV3 | HOXB7 | ZNF292 |
| DNMT1 | ZNF33A | CDC5L | ARID4B | STAT3 | HMBOX1 | MAFG |
| FOSL1 | ZNF644 | SMARCE1 | SPDEF | ESR1 | PAX2 | ZBTB20 |
| RBPJ | ZNF286A | REL | KLF10 | KAT6B | HOXB3 | HES1 |
| DNAJC2 | SOX4 | ADNP | MECOM | GATA2 | SOX17 | DDIT3 |
| RLF | ZBTB38 | SFPQ | ZFHX3 | ZNF611 | DLX6 | ID4 |
| NONO | VEZF1 | ZNF131 | TFDP1 | HEY2 | CXXC1 | NFKBIZ |
| MYC | SIX4 | TFAM | TCF12 | SOX13 | ZNF816 | UBP1 |
| POU2F3 | SSRP1 | ZBED6 | ID3 | XBP1 | NFIB | NFKBIB |
| ARID3B | CNOT4 | MXD1 | TCF3 | FOSL2 | TFAP2C | ARID1B |
| ZNF827 | HMGXB3 | CREB3L4 | ETV5 | BHLHE40 | ZNF331 | TCF4 |
| JARID2 | MIER1 | AEBP2 | ARID1A | KLF9 | DEAF1 | TWIST1 |
| NFATC1 | CREB1 | HIVEP1 | YBX3 | MSX1 | ZNF652 | ZNF284 |
| NFKB1 | MYNN | ZNF506 | ZNF320 | MSX2 | OVOL1 | FOSB |
| ZNF800 | HMGXB4 | SMAD9 | TRPS1 | BCL6 | FOXO3 | ID2 |
| CREB5 | PBX1 | GATAD1 | KLF6 | DLX5 | CREB3L1 | ATF3 |
| FOXN2 | ZNF587 | SMARCC1 | CEBPD | ELMSAN1 | IRF2 | EGR1 |
| ETV6 | TFDP2 | ATF6 | ELF2 | STAT2 | HEY1 | JUN |
| ZNF160 | NPAS3 | PGR | ZNF28 | SREBF2 | KLF3 | FOS |
| PBRM1 | ZNF3 | ARID4A | NFIC | GZF1 | PRDM1 | CEBPB |
| ZNF267 | ATRX | ZNF83 | IRF1 | GRHL2 | MTF1 | |
| KLF7 | ZNF121 | POGZ | LRRFIP1 | NFIA | ELK4 | |

TABLE 13

| | | | | | |
|---|---|---|---|---|---|
| ZBTB1 | ATRX | GATA2 | FOXL2 | KLF7 | ATF3 |
| SOX17 | ZNF462 | MTA2 | CREBZF | EBF1 | NFKBIA |
| BACH1 | NR3C1 | PGR | TCF4 | ADNP | YBX3 |
| PRDM1 | PHTF2 | AR | ZNF22 | KLF9 | STAT3 |
| TWIST1 | SOX4 | KLF10 | ZNF445 | ELMSAN1 | RORA |
| NFATC2 | SP100 | TEAD1 | HOXA10 | KLF4 | ZEB1 |
| ELF1 | ZBTB38 | NR1D1 | HOXA11 | BHLHE40 | ZBTB16 |
| CREB5 | JUND | TSC22D1 | HOXA3 | KLF6 | CEBPD |
| LRRFIP1 | MXD1 | BNC2 | ZNF292 | HIVEP2 | FOXO1 |
| HMGA1 | EGR3 | KAT6B | ZNF160 | XBP1 | MAF |
| FOXP1 | TFAP2C | ESR1 | RORB | ZBTB2 | MITF |
| ETS1 | ETV5 | PRRX1 | ZNF83 | NFKBIZ | HAND2 |
| MAFF | MIER1 | ETV1 | ZBTB20 | ARID5B | OSR2 |
| ELK3 | ID3 | ATF6 | ZNF516 | NR4A1 | |

Next, WOI-associated TFs were defined as those with a peak expression detected after the opening of WOI (FIG. 20C, FIG. 20D), i.e., the boundary between phase 3 and 4. These TFs were further divided into 1) those that peaked during, and 2) those peaked at the end of phase 4, with the hypothesis that the former are more like related to the opening of the WOI and the latter the closure. Interestingly, it was found that these two groups of TFs are enriched with notably different functional roles. For unciliated epithelia, group 1) TFs are dominated by regulators of early developmental process, especially in differentiation (IRX3, PAX8, MITF, ZBTB20); whereas group 2) TFs include those associated with ER stress (DDIT3) and immediate early genes (FOS, FOSB, JUN). For stromal fibroblasts, group 1) TFs are primarily consisted of regulators of chondrocyte differentiation via cAMP pathways (BHLHE40, ATF3), hence are likely drivers for decidualization, and HIVEP2-binder to the enhancer of MHC class I genes (discussed more in later sections on immune cells); group 2) TFs include those with roles in ER stress (YBX3, ZBTB16) as well as in regulation of inflammatory (XEBPD) and apoptosis (STAT3). Of note, the concurrent upregulation of MTF1, activates the promoter of metallothionein I (FIG. 20A), with metallothionein I genes (MT1F, 1X, 1E, 1G, FIG. 3A) in unciliated epithelia, revealing these heavy metal binding proteins as a key regulatory module associated with WOI.

In summary, the analysis enabled the identification of key drivers for the opening and closure of the human WOI as well as transitions between other major cycle phases (FIG. 20C, FIG. 20D, top panels). The dynamics of nuclear receptors for major classes of steroid hormones (FIG. 20E), are also highlighted as a special group of TFs mediating the communication between endometrium and other female reproductive organs. Similar analyses were also performed on genes encoding secretory proteins (FIGS. 21A-21D, Tables 14 and 15) to identify those associated with the WOI (FIG. 21C, FIG. 21D). In some embodiments, one or more transcriptional factors can be monitored. Table 14 shows examples of secretory proteins for which expression levels change throughout the menstrual cycle for unciliated epithelia. Table 15 shows examples of secretory proteins for which expression levels change throughout the menstrual cycle for stromal fibroblasts. Accordingly, in some embodiments, levels of one or more of secretory proteins of Table 14 (e.g., 1-5, 5-10, 10-25, 25-50, 50-100, 100-150, or more or all) can be evaluated and/or monitored in unciliated epithelia to determine the status of the menstrual cycle of a subject (e.g., to determine whether the window of implantation is open, opening, closed, closing, etc. for a subject). Further, in some embodiments, levels of one or more of secretory proteins of Table 15 (e.g., 1-5, 5-10, 10-25, 25-50, 50-100, 100-150, or more or all) can be evaluated and/or monitored in stromal fibroblasts to determine the status of the menstrual cycle of a subject (e.g., to determine whether the window of implantation is open, opening, closed, closing, etc. for a subject). In some embodiments, the level of one or more of secretory proteins (e.g., for unciliated epithelia, and/or stromal fibroblasts) associated with a particular status of the menstrual cycles can be determined by comparing the levels of one or more of these genes to reference levels associated with a known menstrual cycle status (e.g., a known status with respect to the window of implantation) in one or more reference subjects.

TABLE 14

| | | | | | | |
|---|---|---|---|---|---|---|
| WNT5A | DEFB4A | COL27A1 | NHLRC3 | IGFBP2 | C6orf15 | GAST |
| COL12A1 | CLCF1 | CTSH | MALSU1 | WNT5B | LIPG | CRISP3 |
| IPO9 | RARRES2 | PIGL | LGALS3BP | MANF | WFDC2 | VNN1 |
| PSAP | SEMA3B | EMID1 | C7orf73 | RCN2 | PAX2 | CCL20 |
| SERPINH1 | CTSS | COLGALT1 | METTL17 | HIBADH | RNASET2 | ARSB |
| CERCAM | LAMC2 | PPT1 | FAM96A | AGR3 | EPS15 | CTSA |
| SFRP4 | RASA2 | MRPL32 | LRPAP1 | NUCB1 | AK4 | COMP |
| CHSY1 | GLS | DMKN | HEXA | MRPL24 | LCN12 | CXCL14 |
| FKBP10 | EHMT2 | MATR3 | MEIS1 | MBNL1 | CUTA | GDF15 |
| NPC2 | NCBP2-AS2 | C12orf10 | PON2 | SERPINA3 | COASY | LAMB3 |
| PLTP | EDN2 | TRH | CNOT9 | MTX2 | CREG1 | C4BPA |
| LTBP4 | CTGF | STARD7 | PCYOX1 | DDX17 | DEAF1 | DEFB1 |
| SELENON | UXS1 | DHX30 | NME1 | GREM2 | TFPI2 | SLPI |
| PTGS2 | MRPL52 | EHBP1 | PDIA3 | SEMA3C | NOV | GRN |
| MFAP2 | CSF3 | CEP89 | KMT5A | GOT2 | LNX1 | SPP1 |
| COL18A1 | PDE7A | NDUFA10 | PDIA4 | ID1 | B4GALT4 | SCGB2A2 |
| C3 | HEXB | POGLUT1 | NOG | PLA2G12A | NAAA | VCAN |
| TWSG1 | TAGLN2 | PDGFC | KDM6A | OXCT1 | COL1A2 | GPX3 |
| LTBP1 | TEPP | APOOL | TCF12 | PCF11 | GSN | PAEP |
| MDK | EDN1 | CCNB1IP1 | FKBP9 | PLOD1 | PYY | HABP2 |
| BCAR1 | HS3ST1 | IHH | KDELC2 | RCN1 | HADHB | STC1 |
| RTF1 | PRG4 | METTL9 | STOML2 | PFN1 | FAM177A1 | SERPING1 |
| CYR61 | COG3 | MRPL22 | CLPX | THOC3 | ADAMTS8 | FGL2 |
| FSTL1 | CXCL3 | RBM3 | NBPF26 | HS6ST1 | PHB | CLU |
| PLAU | ITIH5 | ZNF207 | RSF1 | CCDC134 | ERLEC1 | IGFBP7 |
| COL4A2 | AGPS | CEP57 | MRPS28 | SNTB2 | VPS37B | FBLN1 |
| B4GALT5 | SEMA3A | GUSB | NDUFS8 | ACTL10 | PEBP4 | PRSS23 |
| PFKP | GGH | MRPL21 | WNK1 | LEFTY1 | BCKDHB | TINAGL1 |
| FGFBP1 | GPD2 | TFAM | CNPY2 | SERPINA5 | C10orf10 | TIMP1 |
| TSKU | SERPINA1 | CALU | GXYLT2 | PTGS1 | SCGB1D2 | SERPINE1 |
| CFI | C1GALT1 | FXR1 | EDN3 | CRELD2 | DHRS7 | CTSC |
| FJX1 | HCCS | FUCA2 | PDZD8 | SUDS3 | PRCP | COL4A1 |
| B3GNT7 | KDM1A | CHID1 | COL9A2 | NDP | SCGB1D4 | |
| IL32 | NUP214 | HSPA5 | METRNL | NUDT9 | SCGB2A1 | |
| CXCL1 | MIER1 | NUP155 | NUDT19 | CD24 | MMP26 | |
| RASSF3 | LIPA | ERP29 | XYLT2 | SRP14 | CABLES1 | |
| LAMA3 | GALNT12 | AGA | LAMB2 | SMARCA2 | MT1G | |

TABLE 15

| | | | | | |
|---|---|---|---|---|---|
| HGF | MEST | MFAP2 | CNOT9 | LAMB1 | COL21A1 |
| CLEC2B | CD24 | BMP1 | TWSG1 | SERPINF1 | BRINP2 |
| RASSF3 | COL7A1 | SFRP1 | SPARCL1 | MASP1 | PAPLN |
| CXCL1 | DKK3 | WNT5A | SULF2 | SLPI | CST3 |
| BMP2 | LOXL1 | WNT2 | CSAD | GPX3 | C1R |
| LAMC2 | PDGFC | SCG5 | P4HA2 | PAEP | C1S |
| CXCL8 | LTBP1 | ISLR | PLOD1 | SERPINE2 | DCN |
| CXCL2 | HSPA5 | FNDC1 | EMILIN3 | FGF7 | NID1 |
| ADM | PDIA4 | CPQ | LRPAP1 | A2M | THBS1 |
| INHBA | WFDC2 | COL1A2 | GXYLT2 | LIPA | PNP |
| STC1 | MIER1 | COL5A1 | SPON1 | DKK1 | RGCC |
| IGFBP6 | CPE | FREM1 | VWA5B2 | RARRES1 | THBS2 |
| FJX1 | TSKU | MFAP4 | MATN2 | EMILIN2 | TAGLN2 |
| COL12A1 | FKBP9 | POSTN | IGF1 | CXCL14 | FSTL1 |
| RSPO3 | TIMP2 | NPC2 | CILP | FBLN2 | RHOQ |
| TNC | COL27A1 | PRSS12 | FN1 | ABI3BP | RBM3 |
| LAMC1 | COL1A1 | CNTN1 | CTSH | B2M | IGFBP3 |
| PLAU | MMP11 | CNTN4 | COL18A1 | ANGPTL1 | FBLN5 |
| LACTB | VEGFA | VWC2 | IGFBP5 | C3 | IGFBP7 |
| IL32 | OLFML2B | COL3A1 | PTN | CRTAP | CCDC80 |
| LOX | NBL1 | EDN3 | ELN | APOC1 | HTRA1 |
| CALR | PAMR1 | PRKD1 | SLIT3 | APOD | MGP |
| HSP90B1 | BGN | ZFYVE21 | SCGB1D2 | APOE | VCAN |
| TGFBI | SFRP4 | COL14A1 | PTGDS | CFD | IGFBP4 |
| LTBP2 | LAMA4 | ECM1 | CCL4L2 | COLEC11 | LUM |
| SCUBE3 | FKBP10 | GDF7 | ADAMTS5 | PLA2G2A | MMP2 |
| SEC31A | PRSS23 | OLFM1 | TIMP3 | SERPING1 | HARS2 |
| ADAMTS16 | MDK | DDX17 | MTHFD2 | EFEMP1 | MXRA7 |

### Example 8—the Relationship Between Endometrial Phases Identified at the Transcriptome Level is Consistent with Canonically Defined Endometrial Phases

Since its formalization in 1950 (Noyes et al., 1950), a histological definition of endometrial phases, i.e., the proliferative, early-, mid-, and late-secretory phases, has been used as the gold standard in determining endometrial state. It also usually serves as the ground truth in bulk-based profiling studies in categorizing endometrial phases. Given that there were clear differences between the phase definition as used herein and the canonical definition, the relationship between the two were investigated.

Cell mitosis is one of the most distinct features of the pre-ovulatory (proliferative) endometrium, hence the naming of proliferative phase. Thus, to identify the boundary between proliferative and secretory phases, cell cycle activities across the menstrual cycle were explored. Specifically, endometrial cell cycle associated genes were defined (FIGS. 11C, 11D, and 12, Method) and assigned cells into G1/S, G2/M, or non-cycling states. For both unciliated epithelial cells and stromal fibroblasts, cell cycling was observed in only a small fraction of cells across the menstrual cycle (FIGS. 11C, and 11D, left, and FIG. 12). This fraction demonstrated phase-associated dynamics, where it was most elevated in phase 1, slightly decreased in phase 2, and almost completely ceased in later phases (FIGS. 11C, and 11D, right, and FIG. 12) indicating that the transition from phase 2 to 3 is between pre-ovulatory to post-ovulatory phases.

To further validate this assignment, characteristic signatures for phase 1-4 were defined and major hierarchies of biological processes that were enriched by the signatures were identified. While phase 1 was characterized with processes such as tissue regeneration, e.g., Wnt signaling pathways (unciliated epithelium: epi), tissue morphogenesis (epi), wound healing (stromal fibroblasts: str), and angiogenesis (str) and phase 2 by cell proliferation (epi), phase 3 was dominated by negative regulation of growth (epi) and response to ions (epi) and phase 4 by secretion (epi) and implantation (epi). The transition from a positive to a negative regulation in growth from phase 2 to 3 further confirmed a pre-ovulatory to post-ovulatory transition (Talbi et al., 2006).

Lastly, previous bulk tissue analyses were used to help differentiate the pre-ovulatory and post-ovulatory phases. It was reasoned that although bulk data is confounded by the varying proportion of the major cell types, i.e., stromal fibroblasts and unciliated epithelial cells, bulk and single cell data taken together should have high level of consensus on genes that 1) are in synchrony between the two cell types or 2) have negligible expression in one cell type but significant phase-specific dynamics in another. Therefore, genes were identified with these characteristics using the single cell data (FIGS. 3A-3B). As expected, among these genes that were identified are those that have been consistently reported by bulk studies to be characteristic of canonical endometrial phases, confirming the validity of using them to identify the WOI. Particularly, the upregulation of the metallothioneins (MT1F, X, E, G) from phase 2 and 3 was characteristic of proliferative to early-secretory transition based on bulk reports (Ruiz-Alonso et al., 2012; Talbi et al., 2006). Therefore, considering all of the evidence above, phases 1 and 2 can be identified as pre-ovulatory (proliferative) phases, and phases 3 and 4 as post-ovulatory (secretory) phases. With the anchor provided by the WOI, phase 3 can thus be identified as the early secretory phase.

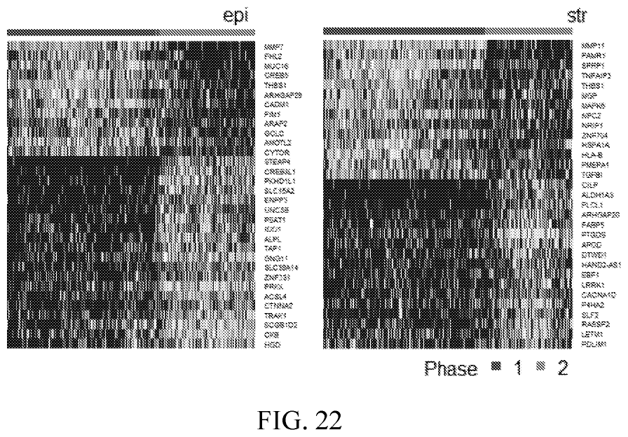

In phase 1, sub-phases were observed in both unciliated epithelial cells and stromal fibroblasts that are primarily characterized with genes that are gradually decreasing or increasing towards later part of the phases (FIGS. 3A, 3B, and 11B). In the unciliated epithelium, the gradually decreasing genes included phase 4 genes (e.g., PAEP, GPX3), as well as PLAU, which activates the degradation of blood plasma proteins. The down-regulation of these genes suggested the end of menstruation, and hence the transition from menstrual to proliferative phase in the canonical definition. Phase 2 can therefore be identified as a second proliferate phase at the transcriptome level. At histological level, transformation in the proliferative endometrium was reported to be featured with morphological changes so gradual that they do not permit the recognition of distinct sub-phases (Noyes et al., 1950). However, it has been discovered that at the transcriptomic level, proliferative endometrium can be divided into two subphases in both unciliated epithelial cells and stromal fibroblasts that can be quantitatively identified by transcriptomic signatures (FIG. 22).

Examples of genes that have expression peaks in different phases (phase 1, 2, 3, or 4) in ciliated epithelia and stromal fibroblasts are provided in Tables 16 and 17, respectively. Accordingly, one or more of these genes can be evaluated (e.g., using RNA and/or protein expression levels) in one or more of these cell types to determine whether a subject is in menstrual phase 1, 2, 3, or 4, for example to determine whether the subject is approaching, entering, in, or exiting a WOI. For example, the expression level of one or more genes (e.g., 1-10, 10-25, 25-50, 50-100, 100-250, 250-500, 500-1,000 or more or all of the genes) characteristic of one or more phases (for example, one or more genes for each phase) can be assayed and compared to a reference level (e.g., for each gene) associated with one of the phases (e.g., for phase 1, phase 2, phase 3, phase 4, or 2, 3, or all thereof) to determine whether a subject has a gene expression level that is indicative of being in phase 1, phase 2, phase 3, phase 4, of for example approaching, entering, in, or exiting a WOI.

Lastly, interactions between unciliated epithelial cells and stromal fibroblasts were explored by identifying ligand-receptor pairs that were expressed by the two cell types across the major phases/subphases of the cycle (Method). One major feature be noted within the identified ligand-receptor pairs: they are dominated by a diverse repertoire of extracellular matrix (ECM) proteins paired with integrin receptors, suggesting that ECM-integrin interaction is a major route of communication between the two cell types. Key interactions were identified at the WOI such as between LIF and IL6ST, with LIF being a key gene implicated in endometrial receptivity (Evans et al., 2009, 2016; White et al., 2007).

TABLE 16
genes ordered by peak pseudotime normalized with ascending order for unciliated
epithelia (phase 1-4, with phase 1 genes shown in italics, phase 2 genes shown in underline,
phase 3 genes shown in italics-underline, and phase 4 genes shown in bold).

| | | | | | | |
|---|---|---|---|---|---|---|
| WNT5A | DCP1A | GREM2 | NPDC1 | CDK11B | ABRACL | SLCO4A1 |
| SFRP4 | SLC25A24 | MXD1 | UBE2G1 | FBXO21 | PSMG3 | ODC1 |
| NREP | CCT2 | ADNP | NAE1 | SOCS3 | GOLPH3 | AGPAT5 |
| PTMAP5 | FRK | OLA1 | EGFR | FZD6 | EDF1 | PLA2G16 |
| GBP5 | CXCL3 | KIAAI324 | AP3S1 | PARP14 | HACD3 | LINC01502 |
| IFI6 | IP6K2 | SCNN1G | PSMC1 | IRF2BPL | ALDH18A1 | ANKRD55 |
| AKAP1 | CORO1C | C16orf72 | ALDH16A1 | PLA2G4A | GNG11 | EDNRB |
| MMP11 | MREG | ZNF252P | IFITM3 | TRIM22 | STEAP4 | SLC22A5 |
| PLXDC2 | PSMD11 | PAKIIP1 | TPM4 | FAM155A | ASRGL1 | MFSD4A |
| ANTXR1 | LTV1 | CNOT6L | CRIP1 | RNF8 | ELP3 | DUSP6 |
| PITHD1 | ITGAV | ANAPC4 | PSAP | WWC2 | GGTA1P | FXYD3 |
| NECTIN2 | CCNC | ADCYAP1R1 | ID3 | PSAT1 | ALDH6A1 | AOX1 |
| IGFBP3 | SMIM15 | ATP5C1 | MYH10 | MTPN | GGCT | LYPLAL1 |
| LY6E | SREK1 | RPARP-AS1 | CRIP2 | TWSG1 | SH3YL1 | HAL |
| SHH | INO80D | TRIM59 | DST | FAM96B | GABRP | FXYD2 |
| BMP2 | PLCB1 | UQCRH | MGST2 | VTCN1 | PRELID3B | CITED2 |
| FLNA | SH3RF1 | DNAJC19 | LSM5 | ARPC1B | SEC61G | SLC44A1 |
| COL12A1 | UCHL3 | UBA3 | RANBP1 | KRTCAP2 | CAMK2D | ATP2C2 |
| PTGS2 | TBL1XR1 | SAR1A | EMC10 | ALPL | TALDO1 | LINC01207 |
| LINC01588 | ITIH5 | EPB41L2 | AC013461.1 | UNC5B | SPATA13 | BACE2 |
| MMP7 | ACTR3 | APOBEC3C | HSPE1 | TMEM131 | CTAGE5 | ACADSB |
| LCN2 | FDPS | VAMP7 | MYO10 | NRXN3 | SIAH2 | NABP1 |
| QSOX1 | UTP11 | PPP1R9A | PHGDH | MSX2 | C19orf53 | MAOA |
| CSF1 | RDX | RPP30 | MSH3 | BHLHE40 | AMD1 | SLC1A1 |
| GJA1 | WDR48 | AEN | MGLL | POLG2 | MRFAP1 | C2CD4A |
| ENC1 | ZHX2 | SMARCA1 | RCC2 | PTGS1 | NPR3 | IDO1 |
| RAI14 | SLC9A3R1 | CASP2 | PRKDC | PIP5K1B | MRPL55 | MGST1 |
| LIF | IRF6 | CYP51A1 | SNRPD1 | COBL | DGUOK | CCL20 |
| TUBA1A | ARHGAP26 | CTD-3014M21.1 | CD2AP | ANXA3 | OVOL1 | ARSB |
| CYP1B1 | PGM2L1 | NME2 | ETV5 | CEBPB | ATPIF1 | RASGEF1B |
| WNT7A | EIF2S1 | OSTC | TP53 | MTA2 | TFAP2C | CLEC4E |
| LAPTM4B | GPR22 | PKP4 | TBC1D5 | LPAR3 | ACSL5 | KRT23 |
| HMGA1 | TCF7L2 | UTRN | GLG1 | RNF122 | APOL2 | SLC15A4 |
| ELK3 | SEMA3A | PAFAH1B2 | CHD4 | SLBP | CSRP2 | TMEM45B |
| USP10 | PRMT1 | OAT | LYRM2 | GPBP1L1 | RASSF4 | FAM134B |
| BCAT1 | MINOS1 | NTPCR | PSIP1 | PPL | CNDP2 | GDF15 |
| COL18A1 | MAPK1IP1L | INTS6 | MCAM | TMEM184B | SEC14L1 | SIK1 |
| PROM1 | ING3 | PLAGL2 | MALT1 | PDZD2 | MRPL3 | DEPTOR |
| C3 | RBM22 | TMED10 | NIPSNAP3A | CAP1 | GNG5 | COMP |
| NRP2 | MORF4L1P1 | ZMYND8 | RIOK1 | SLC26A2 | ZNF652 | PPP2R5A |
| PIM1 | OCLN | CBWD5 | ANP32B | ZDHHC9 | WDR1 | RAB11A |
| MFAP2 | KIF21A | GXYLT2 | HTATSF1 | RNF150 | CTNNA2 | HN1L |
| CYR61 | RC3H1 | AEBP2 | CTSB | RAB27A | THEM4 | FAM65B |
| ZDHHC13 | GCLC | DDAH1 | ATP1B1 | HPGD | MAGED1 | EIF4E3 |
| LUZP1 | GLIPR1 | PGR | HMGN2 | HNRNPR | DYNLT1 | CTSA |
| RBP1 | SIX4 | CNKSR3 | PARP1 | SLC39A8 | NDUFA1 | PHYHIPL |
| IL18 | PHLDA1 | CH17-373123.1 | GPI | AP1S2 | CCDC186 | CXCL14 |
| PLAU | ARMC8 | FAM96A | CNPY2 | SPATS2 | MBP | SLC7A2 |
| SERPINB9 | TCERG1 | CCDC14 | SEPT7 | TXNDC16 | TMEM141 | TSPAN1 |
| AMOTL2 | SERPINA1 | LRP6 | FBL | CITED4 | ACTN1 | ATP6V1A |
| NCEH1 | GPR89A | POLR2D | FRAS1 | NDUFB1 | FREM2 | RIMKLB |
| CD74 | PAPD5 | DCUN1D1 | C21orf33 | THAP4 | NAAA | PIGR |
| THBS1 | LSM12 | METTL7A | PRRG4 | SREBF2 | CKB | TMEM92 |
| TNF | MED24 | EBP | SERINC5 | SUFU | MFSD6 | TC2N |
| ARHGAP29 | USP16 | R3HDM2 | C8orf33 | COX16 | ECHS1 | MRPS2 |
| B4GALT5 | ZNF644 | CLMN | NUDT19 | FAM174B | POC1B | SEPHS2 |
| EMP1 | SLC39A10 | HNRNPF | ARID1A | PREP | FTH1P10 | SLC15A1 |
| TOP2B | MDM4 | HELB | NDUFS5 | TMEM261 | RNF183 | GRAMD1C |
| RNF152 | RBMXL1 | POGLUT1 | ATP5G1 | MTHFD2L | ZCCHC6 | ANXA4 |
| ADAMTS9 | STEAP1 | BZW2 | LINC00998 | AK3 | HPRT1 | VPS41 |
| ILF3 | PALLD | INIP | ZNF589 | LRIG1 | GSN | IRX3 |
| ASPH | TMEM33 | ZRANB2 | HADH | CAPNS1 | FAM120B | ERN1 |
| XRCC5 | ZNF286A | SNHG6 | KIAA1143 | ETFRF1 | NEBL | C2CD4B |
| CFI | ATXN1 | TRAF3IP2 | PARK7 | ATP6V0E2 | ECI2 | CXCR4 |
| MARCKSL1 | TMEM120B | THYN1 | POMP | RCN1 | ITGA1 | SCCPDH |
| TSPAN15 | TLE3 | TIMM17A | MMAB | PAX2 | B3GNT2 | DPP4 |
| HLA-H | SPRY1 | RBMX | KRT8 | ATP5J2 | RAB4A | G0S2 |
| DUSP10 | DNAJC10 | EIF4E | APRT | NDUFB6 | SLC4A7 | TRAM1 |
| ATIC | S1PR2 | PHF14 | PCDH7 | GMNN | CKMT2 | HIST1H2AC |
| MIR4435-2HG | MTPAP | EIF4B | SELENOW | COA3 | PYY | TMC5 |
| IL32 | NMD3 | CEP57 | ARL4A | ZBTB11 | FAM177A1 | LAMB3 |
| BHLHE41 | NPM1P27 | SRSF2 | CCDC170 | ANAPC16 | ALDH3B2 | C12orf75 |
| IL23A | SRPK1 | BTF3L4 | SH3RF2 | FKBP9 | CD36 | SLPI |
| RASSF3 | CLUHP3 | SLC25A6 | METAP1 | PTS | IDH1 | C4BPA |
| SMAD3 | VIM | LRRC75A-AS1 | NDUFA2 | SLC25A1 | MPZL2 | SNX29 |
| SNHG16 | CPM | GAS7 | CTTN | SSBP1 | GMPR | MAP3K5 |
| RRAS2 | LIPA | PSMA6 | CENPX | CSRP1 | COL1A2 | PAX8 |
| FBXO32 | SENP5 | HMOX2 | FAM84A | PPP4R2 | ERLEC1 | LEPROT |
| CD47 | MTFMT | PRDX6 | EEF1E1 | BCAP29 | RHOBTB3 | DEFB1 |
| IGF2R | AGO3 | MARCH6 | SYNGR2 | PER2 | TPD52L1 | MITF |
| B3GNT7 | TUSC3 | GTF2A2 | FUT8 | TP53I3 | CREB3L1 | TNFSF15 |
| MSN | ST3GAL5 | UBE3A | GDI2 | ATP5F1 | BNIP3L | AQP3 |
| HMGB1P5 | ALDH3A2 | IMPDH2 | FH | ITGA6 | HGD | GRN |
| RBBP8 | KIZ | EIF1AX | CCDC146 | CD99 | CD81 | DHRS3 |
| FHL2 | IGFBP4 | STON2 | SRRM2 | MRPS34 | MAP2K6 | UBBP4 |
| MB21D2 | ANO1 | PTGFRN | NDUFA8 | NAALADL2 | CPT1A | MUC16 |
| DEFB4A | CDC42EP3 | BRD3 | COX4I1 | PLLP | GPT2 | SPP1 |
| MED4 | LINC01480 | CBX5 | PKP2 | PNPO | SQLE | LINC01320 |
| HDAC9 | TNKS2 | PDCD4 | TRIM2 | ATP1A1 | SNX9 | AGR2 |
| RGS10 | EMID1 | MSI2 | EDN3 | HK2 | CYB5A | SRD5A3 |
| EXT2 | ADAM28 | TXN2 | NUCKS1 | CTC-444N24.11 | LDLR | VEGFA |
| CTSS | TAF9 | TRIM16 | KRT19 | GRHL2 | SLCO3A1 | DUSP5 |
| DLC1 | YLPM1 | PLEKHA5 | NBEAL1 | BAG5 | AREG | ADGRF1 |
| E2F3 | MEST | C6orf48 | AKR1C3 | TMEM256 | TMEM144 | CP |
| SVIL | ARL3 | C7orf73 | YBX1 | RFLNB | PPM1H | DCPS |
| SEMA3B | TFDP2 | CHD7 | HNRNPM | RANBP17 | RXFP1 | SCGB2A2 |
| ADGRA3 | EXOSC5 | BEX3 | TNS1 | SLF2 | TAP2 | NUPR1 |
| ANKLE2 | EIF3E | HSPD1 | CTBP1 | AIFM1 | FKBP5 | CRYAB |
| FOSL1 | SLC47A1 | TCTN2 | WDR77 | KYAT3 | HMGCR | RASD1 |
| CYTOR | NSG1 | MECOM | BTG2 | TFCP2L1 | FAM129B | PAPLN |
| CA12 | APOOL | BOD1L1 | OGFOD1 | PHB2 | CALD1 | PAX8-AS1 |
| JARID2 | CTSH | H2AFZ | HERPUD1 | KCNK13 | FOXO3 | TXNIP |
| CXCL1 | TCEAL1 | RAD51C | POLR2G | L3MBTL4 | DCXR | FAM3C |
| PABPC4 | PORCN | SNRPN | TLE4 | NFIA | UBE2D2 | ZNF292 |
| MACC1 | PSMD12 | CEP290 | TSTD1 | PYURF | CYP26A1 | TRAK2 |
| SPECC1 | AGO2 | TFAM | PTPRJ | FAM213A | LINC00844 | TNFAIP2 |
| RBPJ | ZBTB38 | EXOSC8 | LAMB2 | IL20RA | ANXA2P2 | VCAN |
| HNRNPAB | GAN | FAM111A | MEX3D | LLGL2 | ARHGAP18 | HNMT |
| NFKB1 | DMKN | CHCHD2 | LRRC41 | NPTN | PLEKHF2 | MYO9A |
| LAMC2 | PPP1R2 | ACTL6A | SULT1E1 | ORAI2 | ADAMTS8 | GPR160 |
| ANKRD33B | MUM1 | AHSA1 | POLR2J3 | DLGAP1 | IFNGR1 | GPX3 |
| ARL14 | PCMTD2 | EEF1D | POLD2 | LIPG | BTAF1 | PAEP |
| SHISA2 | NPAS3 | STX18 | UBE2Q2P2 | TRAK1 | DUOX1 | STC1 |
| MYO6 | COLGALT1 | PBX1 | PSMA7 | ACPP | ATP6V1G1 | TUFT1 |
| RARRES2 | PAN3 | SLC25A5 | LARS | NAA60 | CCNA1 | NNMT |
| SMURF2 | DAAM1 | SLC12A2 | RIN2 | NOSTRIN | PHB | FBLN1 |
| CD83 | AC093673.5 | HNRNPAO | IGFB P2 | DLG5 | TFPI2 | HABP2 |
| ATP6V1B2 | TMEM41B | EDN1 | COA4 | TAP1 | TMED4 | CYP3A5 |
| TARBP1 | BMPR1B | NONO | ITM2C | RNASET2 | LINC00116 | CLDN10 |
| ITGB6 | BST2 | PAICS | EIF3M | PRKX | SLC39A14 | SYNE2 |
| PTBP2 | PAM | APEX1 | RHOB | JTB | HLA-DOB | HKDC1 |
| HSPB8 | SFXN2 | NME1 | ID2 | MCC | EMC4 | ABCC3 |
| RAB11FIP1 | COL27A1 | RBBP7 | SRGAP3 | RABGAP1L | WIPI1 | SCIN |
| FAM98B | ERI1 | REC8 | DUT | SUDS3 | MSMO1 | C8orf4 |
| SPIN1 | DDHD1 | CNP | THSD4 | NFIB | SH3BGRL3 | SLC40A1 |
| DEK | MRPL1 | CCT4 | SERPINA3 | OPRK1 | CAPN6 | NAPSB |
| KHDRBS1 | CWC15 | DDX1 | TCF20 | ACSL4 | LRRC1 | PIK3R1 |
| TRIM33 | CXADR | ATP1B3 | ZNF611 | DNAJC15 | DHRS7 | IGEBP7 |
| CMTM7 | KIAA1456 | NBPF10 | C22orf29 | MUC1 | PART1 | SERPING1 |
| TNFRSF12A | ATP5G3 | PAPD4 | PLEKHG1 | ARF5 | SMS | GEM |
| SPOCD1 | ZNF121 | PRDM2 | CNTLN | FARSB | ENPP3 | CYP24A1 |
| TXNRD1 | CDCA7L | PPA2 | AGR3 | C2orf88 | TUBB2A | CXCL2 |
| BCL9 | CLNS1A | REEP5 | SERPINA5 | SLC15A2 | WHRN | CLU |
| OCIAD2 | NEIL2 | SMG1 | PPP3CA | TMEM101 | DUOXA1 | FGL2 |
| ADAM9 | EIF3G | MARCKS | CHD3 | AFMID | SCGB2A1 | ZBTB20 |
| TARDBP | ADAMTS6 | ANP32E | TCEA3 | PDXDC1 | PLIN2 | LITAF |
| RIF1 | TM2D3 | SNRPB | SAMHD1 | CARMIL1 | RAMP2 | TNFSF10 |
| ZNF608 | DDX6 | CDC123 | HNRNPK | NAMPT | ARL4C | HES1 |
| SF3B1 | ARHGAP17 | DFFA | GRHPR | ANK3 | HSD17B2 | ABLIM1 |
| UBE2E3 | USP7 | PGD | LONRF1 | AK4 | SORD | DNAJB1 |
| PSMB4 | GABPB1-AS1 | GOLIM4 | COL9A2 | TPI1P1 | PAPSS1 | BICD1 |
| SF3A3 | OXR1 | VCL | SEMA3C | ENAH | SLC16A1 | HSPA1A |
| PAFAH1B3 | MLLT3 | HNRNPA1P48 | LIMCH1 | ZCRB1 | ABCG1 | HSPH1 |
| MYL6B | GAS5 | PLEKHA2 | DANCR | USMG5 | TLE1 | AXL |
| MRPL44 | EEF1A1P13 | SELENOH | RAB14 | PIKFYVE | DENND2C | LUM |
| S100A16 | DEGS2 | EIF3D | ALCAM | SLC7A1 | ATP6V1C2 | MAP1B |
| MTF2 | ARID2 | UQCC2 | ERC1 | MARK1 | MT1F | CCND2 |
| GPRC5A | RIDA | SNRPF | HEY2 | HDDC2 | MT1X | COL3A1 |
| SUPV3L1 | FAM13B | YWHAQ | XYLT2 | SMIM22 | UPK1B | MMP2 |
| ATRX | KRR1 | RAN | PGRMC1 | LONRF2 | CDK7 | SERPINE1 |
| PIP5K1A | SLC35F2 | SLIRP | ESR1 | FAM110C | SCGB1D4 | FSTL1 |
| TPBG | MID1 | PRPS2 | LDHB | MPHOSPH10 | SCGB1D2 | COL1A1 |
| BID | KMO | MDK | ARID1B | LAMTOR4 | TESMIN | AKAP12 |
| PITPNB | TLK1 | TPM3 | SNRPB2 | PKHD1L1 | MMP26 | TCF4 |
| ITCH | TLR2 | AKIRIN1 | FMC1 | ATP6V0B | ST14 | TIMP1 |
| STX12 | PSMD4 | PLPP2 | TNKS1BP1 | SF3B6 | XDH | SYNCRIP |
| CSF3 | DDOST | MAP4 | PRR15 | VDAC1 | AFDN | COL4A1 |
| AP000462.1 | LINC00665 | STIP1 | PKM | HMGN5 | NHSL1 | NAP1L1 |
| ZNF827 | WBSCR22 | PDLIM1 | SERBP1 | TM9SF3 | HEY1 | SPARC |
| TNFRSF21 | SBNO1 | STMN1 | DLG1 | ARPC4 | LPIN1 | LGALS1 |
| DNTTIP2 | MRPS17 | ALDH1B1 | CCT8 | TM7SF3 | SYBU | IFITM1 |
| HS3ST1 | PPT1 | TPR | RCN2 | ADH5 | KCMF1 | TMEM98 |
| ANKRD28 | RBM3 | NCL | MYBBP1A | SOX17 | SPHK1 | TIMP3 |
| TNFRSF10B | PPIL4 | AHCY | TOP1 | STRBP | TIAM1 | DCN |
| NELFCD | LINC01138 | DNPH1 | TCEAL4 | RSRP1 | SDCBP2 | THBS2 |
| MPRIP-AS1 | SLFN5 | HACD2 | BARD1 | HMGN3 | SMIM5 | CTSC |
| MED17 | SLC39A6 | CCT3 | TMEM14A | OFD1 | MT1E | YTHDC2 |
| CTGF | PTEN | BROX | TAF8 | ARL6IP5 | TMEM154 | ID1 |
| NFATC1 | TULP4 | MIA3 | DCBLD2 | NDUFA13 | MT1G | C11orf96 |
| DENND4A | GAS2 | MRPS25 | CHCHD5 | CTB-178M22.2 | MT2A | RGS2 |
| CMTM6 | BLOC1S6 | ATP5A1 | NAV2 | ATP5I | MT1M | SAMD4A |
| SDCBP | NHP2 | DKC1 | PLEKHA3 | DLX5 | LMO7 | PDS5B |
| FAM133B | RAPGEF2 | NAP1L4 | UGT2B7 | NEK1 | MT1H | TIMP2 |
| IFT57 | PELI1 | ATP5L | GATA2 | CS | UTP15 | PTN |
| CREB5 | DCAF16 | TOMM22 | GREB1 | B3GNT5 | SLC18A2 | PMEPA1 |
| TSPAN6 | CCNG2 | SNHG14 | ANKRD11 | PLA2G4F | LIG4 | HDAC2 |
| CADM1 | EYA2 | PFDN5 | OXCT1 | NOV | SLC30A2 | NOTCH3 |
| L3MBTL3 | UBE2N | UBAP2L | RCAN1 | APOPT1 | ADGRL2 | C1S |
| ASAP1 | SMAD9 | HSBP1 | TIMM8B | ADIPOR2 | GAST | NRP1 |
| PPP2R3A | SPDEF | CEP95 | STXBP6 | NDUFC2 | FAM84B | S100A6 |
| CD44 | GUSB | TSPAN14 | SCD | HSD11B2 | TCN1 | HSPA1B |
| ARAP2 | MMADHC | MAGI1 | RREB1 | SLAIN1 | RASEF | IFITM2 |
| NINJ1 | PDGFC | PDIA3 | ELF2 | APOL4 | GCNT3 | HSP90AA2P |
| S0X9 | ADAT2 | GSTK1 | JUN | HOMER2 | CRISP3 | PR5523 |
| N4BP2 | SLC25A26 | CCND1 | BASP1 | SORBS2 | RIMKLBP1 | NFATC2 |
| NRCAM | STK17A | NASP | NEO1 | RHOU | ELK4 | ALDH7A1 |
| HCP5 | OTUD6B-AS1 | IFI27 | SNX5 | TOB1 | PCDH17 | KLF9 |
| SEMA3E | ETNK1 | HIST1H4C | DBI | COX17 | PPFIBP2 | MEIS1 |
| TARS | SUB1 | RSRC1 | PFKL | IKZF2 | DYNLT3 | CBX1 |
| SIPA1L1 | DYNC1I1 | DNMT3A | GDA | NME4 | CDYL2 | MYO1B |
| CAB39 | GLA | CUL5 | SH3BGRL | CREG1 | RBL2 | CRISPLD2 |
| KPNA4 | FRMD4B | DNMT1 | PLOD1 | CDC42SE2 | SLC34A2 | COL6A3 |
| RRP15 | LCLAT1 | SNRPD3 | PABPN1 | OST4 | VNN1 | MAP4K4 |
| CRISPLD1 | U5P22 | CCP110 | SP100 | HADHA | SLC3A1 | TINAGL1 |
| TPGS2 | CSNK2A2 | ST13 | SYNJ2BP | GAPDHP65 | DDX52 | |
| AGPS | ZNF516 | PRPF40A | LGR5 | FDFT1 | BCL2A1 | |
| STARD3NL | PIP4K2A | HNRNPD | MTURN | COX7A2 | TNFAIP6 | |
| F3 | TSPYL1 | HSPB1 | KAT6B | CUTA | TSPAN8 | |

TABLE 17
genes ordered by peak pseudotime normalized with ascending order for stromal
fibroblasts (phase 1-4, with phase 1 genes shown in italics, phase 2 genes shown
in underline, phase 3 genes shown in italics-underline, and phase 4 genes shown
in bold).

| | | | | | |
|---|---|---|---|---|---|
| CXCL8 | ITGA6 | CDV3 | POSTN | POLG2 | ADAMTS9 |
| C11orf96 | OTUD4 | XBP1 | CNTN1 | ABCA1 | HLA-B |
| PMAIP1 | PPP2CA | KDM6B | ZNF704 | PTGDS | LGALS3 |
| PER2 | RUNX1 | CELF2 | FREM1 | SLC26A7 | LAMB1 |
| GEM | RAP2B | PLAU | IGFBP7 | WEE1 | AHCY |
| STC1 | H2AFZ | CXADR | IL33 | ARIH1 | MGST1 |
| TNFRSF12A | PTGS2 | AP1G1 | PAG1 | AKAP12 | ACTA2 |
| MAP3K8 | PFKFB4 | IRF2BP2 | HIST1H4C | CHD1 | SCARA5 |
| UGCG | ZC3H12A | TOP1 | TRIB2 | ELMSAN1 | ATP6V0E1 |
| ERRFI1 | KPNA4 | TAX1BP1 | MRC2 | KLF4 | GPX1 |
| INHBA | MCL1 | EPCAM | PPP2R2C | BCL6 | SERPING1 |
| CDH2 | ETV5 | PDIA4 | MTUS2 | SERPINE1 | NNMT |
| ANXA1 | CCDC85B | GTPBP4 | STMN1 | GPRC5A | PSMA7 |
| CYTOR | PSMD11 | ZSWIM6 | RBP7 | THBS1 | SRI |
| TGFBI | SQSTM1 | PODXL | OLFM1 | EMP1 | PSME1 |
| MAP2K3 | CFL1 | SDC4 | PGR | BHLHE40 | PFN1 |
| HMGA1 | PDE4B | TMEM2 | RUNX1T1 | KPNA2 | ABCC9 |
| B4GALT1 | RTN4 | RNF152 | BRD8 | OSER1 | PPP1R14A |
| NFATC2 | ERN1 | EIF5 | PEBP1 | DNAJB1 | CAP1 |
| F13A1 | FGFR1 | PHLDA1 | IGDCC4 | LDLR | C3 |
| BZW1 | ETS2 | PELI1 | SKA2 | MIR22HG | IGFBP4 |
| SYNJ2 | LRMP | MSANTD3 | BEX3 | ARC | IL15 |
| MAFF | COQ10B | ELK3 | N4BP2L2 | TNFAIP3 | TMEM45A |
| MIR4435- | FBXO33 | PSMD7 | ZCCHC11 | HSPA1A | APOD |
| 2HG | | | | | |
| FOSL1 | ATP1B1 | TNFRSF9 | CACNA1D | NFKBIZ | SNX10 |
| MMP7 | IER3 | AMOTL2 | GDF7 | ANXA2 | TGM2 |
| PDGFC | PPP1R15B | LIMS1 | ECM1 | CAST | ALDH1A3 |
| PIM3 | NFKB1 | LAPTM4B | ZFYVE21 | GFPT2 | CFD |
| ABL2 | ALYREF | ATP13A3 | TRAM1 | ANXA2P2 | MGP |
| FJX1 | ANKRD28 | MEST | PIP5K1B | TUBA1C | HAND2 |
| ELL2 | LIF | ITGB1 | HOXA10 | GPX3 | HSPB1 |
| TES | ETS1 | RAB22A | ZBTB8A | TRIB1 | PRPS2 |
| CD44 | NR3C1 | RAN | PKD1L2 | SFMBT2 | BCAT1 |
| SDK2 | SEC24A | SDC2 | FAM213A | LMCD1 | MYL9 |
| CAV1 | MYADM | SERTAD1 | PDS5B | FGF7 | TXNIP |
| SGK1 | FHL2 | CSNK1A1 | PPIB | NR4A1 | MAOB |
| TWIST1 | DUSP14 | HSPH1 | DIO2 | RDH10 | TUBB |
| CXCL1 | ANK2 | EGR3 | P4HA2 | ARID5B | TMEM37 |
| NRIP1 | B3GNT2 | CPM | TMEM144 | PAEP | PLA2G2A |
| KLF5 | KMT2C | MEX3D | ANO1 | CYP4B1 | FOXO1 |
| LRRFIP1 | PARD6B | AFF4 | GLG1 | ATF3 | APCDD1 |
| CD83 | TLE3 | LTBP2 | HOXA11 | CORO1C | C1orf21 |
| NINJ1 | RAB7A | IFI6 | SEC22B | THBS2 | HSPB6 |
| TNC | REL | PMEPA1 | SLF2 | ADAMTS5 | LMOD1 |
| CXCL2 | HK2 | PIM2 | TRPS1 | NCOA7 | EFEMP1 |
| BAZ1A | SDCBP | SKIL | ANKRD20A11P | PLIN2 | C1R |
| SPSB1 | CLEC2B | TSKU | DAAM1 | LDHA | IGF2 |
| RASSF3 | TXNRD1 | ZBTB2 | TNRC6B | TIMP3 | PILRA |
| BMP2 | CDC14A | AHSA1 | RASSF2 | MTHFD2 | RBP1 |
| RIPK2 | QKI | TFAP2C | GXYLT2 | STOM | SDHD |
| KRT19 | FOXP1 | TMED4 | CDK6 | YBX3 | SLC2A8 |
| GADD45A | CD59 | TPBG | ZNF532 | MEDAG | C1S |
| AMFR | TP53BP2 | ZFAND2A | HSD11B2 | MIF | PAPLN |
| GFRA2 | ARID4B | MIR29A | FAM46A | TLN1 | SPTSSA |
| DUSP5 | ATP2B1 | CYR61 | F3 | TWISTNB | DSTN |
| NOCT | LTBP1 | ALCAM | GARNL3 | NME2 | SLC8A1 |
| SLC39A14 | SNX9 | ID3 | SPEF2 | DKK1 | LCP1 |
| KLHL21 | GSPT1 | HSPE1 | PPM1H | DAXX | MCC |
| CTNNAL1 | PLK2 | FKBP9 | ARHGAP20 | RAB31 | ENPEP |
| MAP1LC3B | STX3 | PPP1R15A | SPECC1 | S100A4 | TGFBR2 |
| CEBPB | BACH1 | USP22 | PDGFRA | DPYSL2 | PSMA4 |
| ARL4C | ADNP | CPE | FAM198B | CLIC4 | NUPR1 |
| LMNA | EIF3A | COL27A1 | RBM6 | HLA-C | MMP2 |
| ADM | ATP6V1G1 | PAMR1 | FABP5 | STAT3 | PIK3R1 |
| PIM1 | PTRF | PCSK5 | MATN2 | FKBP1A | FBLN5 |
| WDR43 | HSP90AA2P | ISLR | RORB | LITAF | AKAP13 |
| ADAM12 | ILF3 | BGN | HELLPAR | S100A11 | ADCY1 |
| CKS2 | LAMC1 | MMP11 | ITGB8 | PDIA6 | GPX4 |
| ZBTB43 | EAF1 | MMP16 | TMEM196 | FBLN2 | UBL5 |
| MAP1B | MXD1 | TNFRSF19 | MME | HLA-A | AASS |
| TNFAIP2 | NFE2L2 | KLF10 | LETM1 | CXCL14 | PDCD5 |
| GCLC | MINOS1 | GLIPR1 | TMEM132B | INSR | SLIRP |
| CADM1 | SPRY2 | PGRMC1 | REV3L | CACNB2 | H19 |
| FNDC3B | CDKN1A | MFAP2 | NTRK3 | TCEAL4 | COLEC11 |
| CRY1 | EIF4E | PRSS23 | JAZF1 | CRYAB | GABRA2 |
| DNAJB6 | TNIP1 | WNT5A | FN1 | TAGLN | APLP2 |
| ADAMTS16 | TFPI2 | GUCY1A2 | CILP | ENPP1 | MAF |
| CD34 | KIF1B | CRABP2 | NR2F2-AS1 | ALDOA | MASP1 |
| EZR | IFNGR2 | ANO4 | SEMA5A | TPM2 | ST3GAL5 |
| CREB5 | NAMPTP1 | PAM | PARM1 | SERPINF1 | PRLR |
| CD55 | NAMPT | GJA1 | SLC12A2 | SELENOP | FBXO32 |
| SCD | UBE2D3 | MFAP4 | TBL1XR1 | PLCD1 | UQCR10 |
| DDX21 | CSF1 | FNDC1 | INTS6 | IRS2 | HAND2-AS1 |
| ZBTB38 | ISOC1 | ALDH1A1 | PLCL1 | PALMD | MYL12A |
| SLC2A1 | LINC01588 | SFRP1 | PLEKHH2 | AC005062.2 | RBX1 |
| HSPB8 | PSMD6 | ETV1 | PTN | DHRS3 | GLUL |
| B4GALT5 | PTP4A1 | SFRP4 | EBF1 | POLR2L | APOC1 |
| MAPK6 | RAP1B | NREP | ELN | PDLIM1 | |

### Example 9—Transcriptome Signatures in Deviating Glandular and Luminal Epithelium Supports a Mechanism for Adult Epithelial Gland Formation

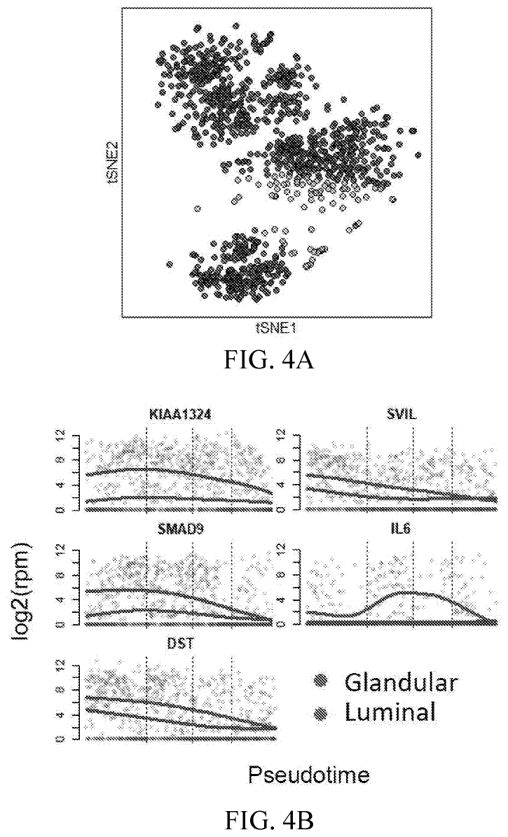

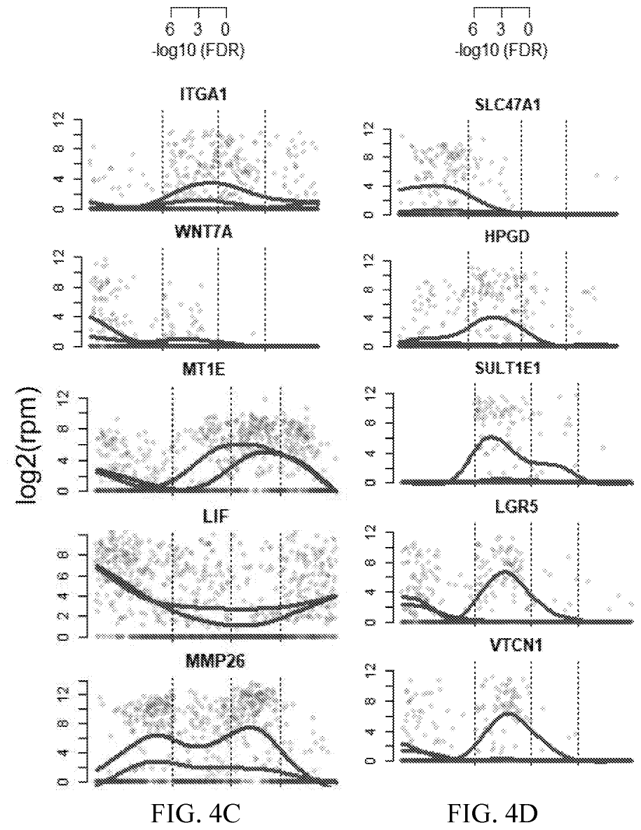

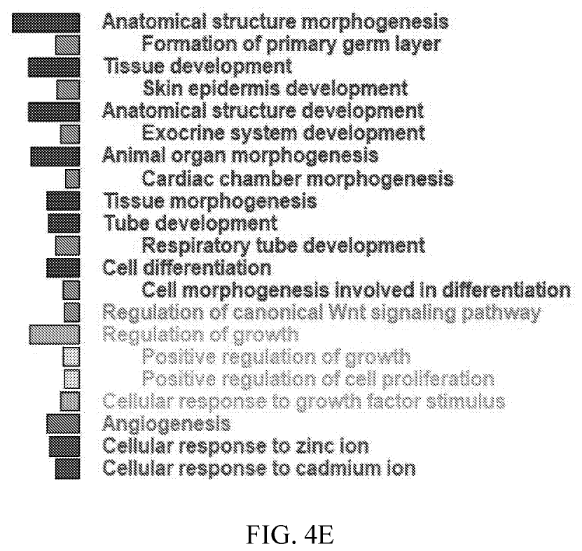

In unciliated epithelial cells, further segregation of cells was noticed (FIG. 4A) in the direction perpendicular to the overall trajectory of the menstrual cycle. Independently performed dimension reduction (tSNE) on cells from each of the major phases (FIG. 13A), excluding genes associated with cell cycles (FIG. 12), confirmed the observed segregations when tSNA was done on all unciliated epithelial cells (FIGS. 4A and 13A).

To identify the nature of the segregation, differential expression analysis was performed and genes were found that consistently differentiated the subpopulations across multiple phases (FIG. 4B). Immunohistochemistry staining of these genes was examined in the Human Protein Atlas (Uhlen et al., 2015) and it was found that genes upregulated in one population stained intensely in epithelial glands, whereas genes upregulated in the other demonstrated no to low staining. Moreover, among these genes, a few that were associated with luminal and glandular epithelium were found. ITGA1, which was reported to be consistently upregulated in glandular epithelium than in luminal epithelium (Lessey et al., 1996), started to differentially express between the two populations at phase 2 and the differential expression persisted for the rest of cycle. WNT7A, reported to be exclusively expressed in luminal epithelium of both humans (Tulac et al., 2003) and mice (Yin and Ma, 2005), is overexpressed in the other population in all proliferative phases (FIG. 4C). SVIL, differentially expressed in the same population in all but phase 4, encodes supervillin, which was associated with microvilli structure responsible for plasma membrane transformation on luminal epithelium (Khurana and George, 2008). Taking the above evidence together, the deviating subpopulations can be identified as the glandular and luminal epithelium.

Genes that were previously reported to be critical for endometrial remodeling and embryo implantation were noticed within the differentially expressed genes (FIG. 4C). They were characterized with unique dynamic features. For example, the metallothioneins (MT1E, MT1G, MT2A, MT1F) were upregulated in the luminal and glandular cells with a consistent lag in one phase. LIF, which was implicated in endometrial receptivity (Evans et al., 2009, 2016; White et al., 2007), was down-regulated in glandular epithelium throughout phase 2, 3, and early phase 4. MMP26, a metalloproteinase reported to be up-regulated in proliferative endometrium (Ruiz-Alonso et al., 2012), was differentially expressed in glandular epithelium until phase 4. Of note, no such differential expression in phase-defining genes presented in the earlier sections or housekeeping genes was observed. (FIG. 13B).

Compared to the consistent distinction between the ciliated and unciliated epithelium, the deviation between luminal and glandular epithelium at transcriptome level was subtler and more dynamic: it became noticeable at late phase 1 and was most pronounced in phase 2 (FIG. 4A and FIG. 13A). This observation is further supported the dynamics of differentially expressing genes such as HPGD, SULT1E1, LGR5, VTCN1, and ITGA1 (FIG. 4C), among many others (FIG. 13C), in that the maximum deviation of their expression in luminal and glandular cells was reached in phase 2 (the latest phase before ovulation).

Functional enrichment analysis of genes overexpressed in the luminal epithelium in proliferative phase revealed extensive enrichments in morphogenesis and tubulogenesis which lead to development of anatomic structures as well as morphogenesis at cell level that lead to differentiation (FIG. 4D). The Wnt signaling pathway, associated with gland formation during the development of the human fetal uterus, was also enriched in this gene group, along with growth, ion transport, and angiogenesis. On the other hand, the most pronounced feature of the glandular subpopulation in the same phase was a consistently higher fraction of cycling cells compared to their luminal counterparts (FIG. 12C, and FIG. 22, left). The co-occurrence of the ceasing cell cycle activity and maximized deviation between the two subpopulations in phase 2 also suggests that the important role proliferation plays in the process.

In addition, a third cell group was identified in the first three biopsies on the pseudotime trajectory (ordered by the median of pseudotime of all cells from a woman) (FIGS. 4A, 13A, and 24). This cell group is transcriptomically in between luminal and glandular epithelial cells (FIG. 13D), expressing markers from both, suggesting either an intermediate state undergoing transition between two populations or a bipotential progenitor state giving rise to both populations. To explore whether this data supports one state over the other, genes were examined that are overexpressed in this cell group over both luminal and glandular epithelial cells (FIG. 13E), where genes were found that are of mesenchymal origin, including CD90 (THY1) and fibrillar collagens (COL1A1, COL3A1) as well as transcriptional factors that are associated with transitions between mesenchymal and epithelial states, including TWIST1, slug (SNAI2) (reviewed by Zeisberg and Neilson, 2009), and WT1 (reviewed by Miller-Hodges and Hohenstein, 2011). The downregulation of these genes from the ambiguous cell group to unciliated epithelial cells later in the pseudotime trajectory suggested that it is a bipotential mesenchymal progenitor population that develops into luminal and epithelial cells through mesenchymal to epithelial transition (MET). In fact, the transition between epithelial and mesenchymal states was observed in cells both at the earliest and the latest timepoints on the pseudotime trajectory (FIG. 4A), indicating that the transition peaked both immediately before and after menstruation. This characteristic dynamic is further evidenced by the temporal expression of vimentin (VIM), a canonical mesenchymal marker, in unciliated epithelial cells (FIG. 13F), where its expression is sustained in phase 1 and 2 (menstrual and proliferative phases), repressed in phase 3 and early phase 4 (early- and mid-secretory phases) and rises again in late phase 4 (late-secretory phase). Surprisingly, several previously proposed markers for endometrial cells with clonogenic and mesenchymal characteristics (reviewed by Evans et al., 2016) including MCAM (CD146) and PDGFRB (Schwab and Gargett, 2007) as well as SUSD2 (Miyazaki et al., 2012) were not significantly upregulated in the ambiguous cell group.

Adult human endometrial gland formation in menstrual cycles have been proposed to originate from the clonogenic epithelial, or mesenchymal progenitors, or both, in the unshed layer of the uterus (basalis) (Nguyen et al., 2017; W. C. et al., 1997). The present data indicates that endometrial re-epithelization is through MET from mesenchymal progenitors, a process that has been demonstrated in transgenic mouse models (Cousins et al., 2014; Huang et al., 2012; Patterson et al., 2013) but had yet to be observed in human. The present data also shows that following re-epithelization, endometrial gland reconstruction in adult human endometrium is driven by tubulogenesis in luminal epithelium, which involves the formation of either linear or branched tube structures from a simple epithelial sheet (Hogan and Kolodziej, 2002; Iruela-Arispe and Beitel, 2013)—a mechanism that also contributes to gland formation during the development of human fetal uterus (for review, see Cunha et al., 2017; Robboy et al., 2017). This process is also characterized by proliferation activities that are locally concentrated at glandular epithelium.

### Example 10—Relative Abundance of Other Endometrial Cell Types Demonstrated Phase-Associated Dynamics

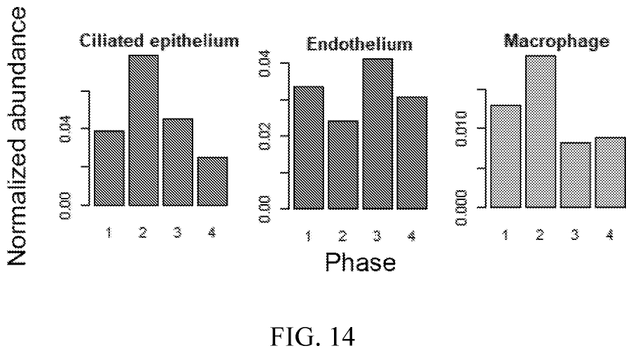

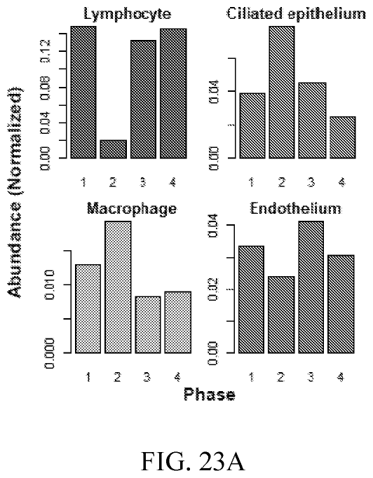

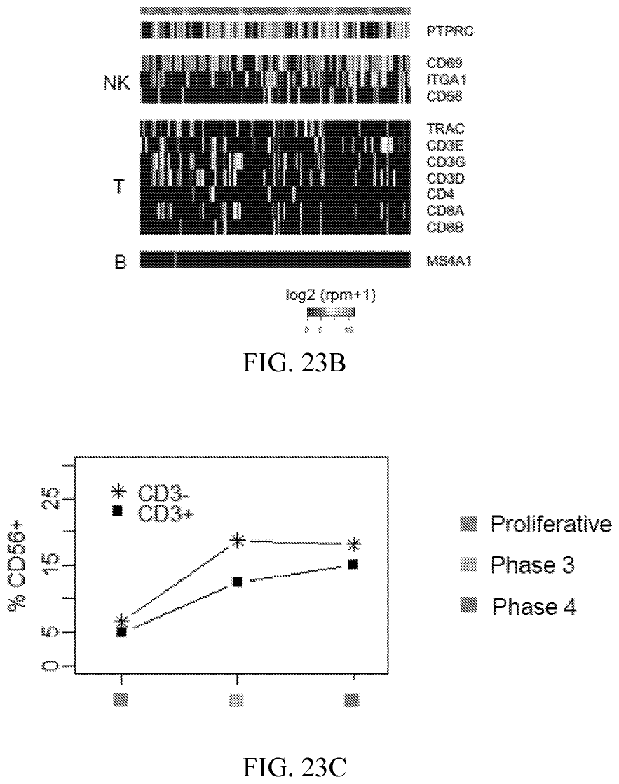

Using the phase definition of unciliated epithelial cells and stromal fibroblast, other endometrial cell types from the same woman were assigned into their respective phases, and quantified for their abundance across the cycle (FIG. 14, and FIG. 23A). An overall increase in ciliated epithelial cells across proliferative phases and a subsequent decrease in secretory phases was observed as well as a notable rise in lymphocyte abundance from late-proliferative to secretory phases. The change in macrophages was contrary to previous histological reports (Bonatz et al., 1991; Kamat and Isaacson, 1987). Factors such as sampling size for a low abundance cell type and sampling bias in choice of spatial locations in microscopic observations may have caused the discrepancy and should be taken into account for future studies.

### Example 11—Decidualization in Natural Menstrual Cycle was Characterized with Direct Interplay Between Lymphocytes and Stroma Cells

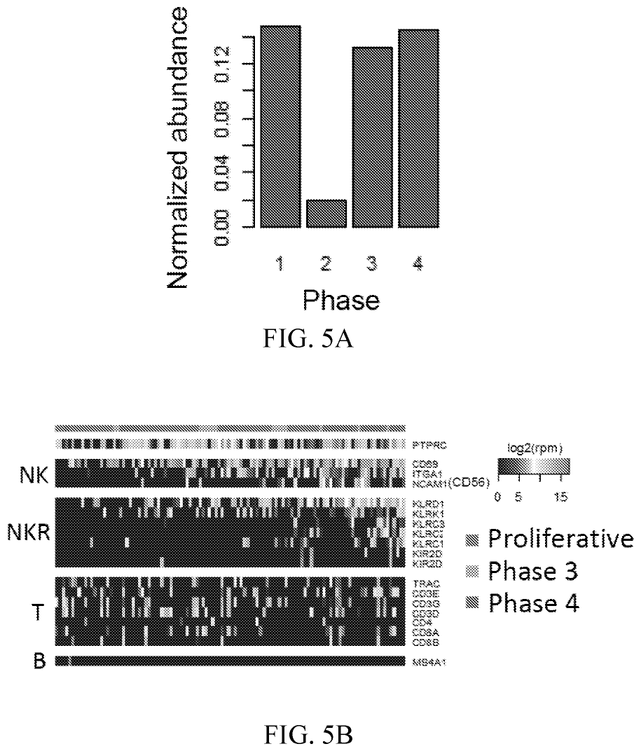

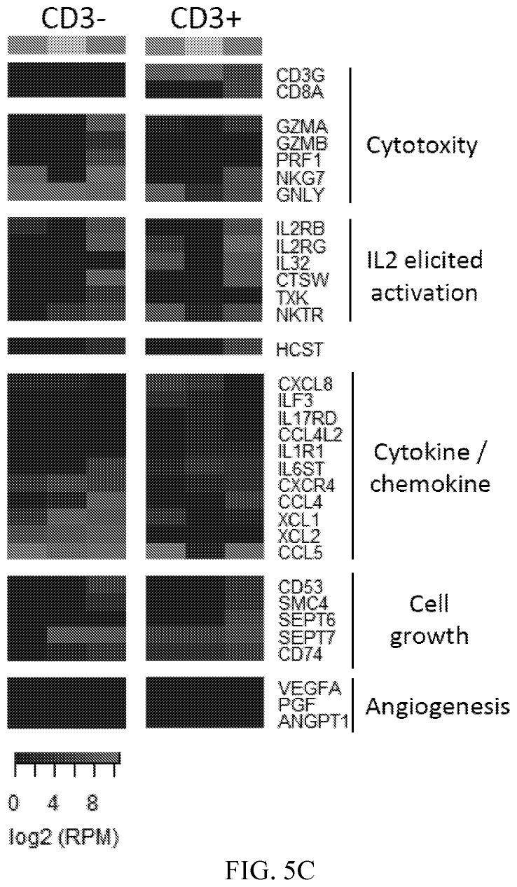

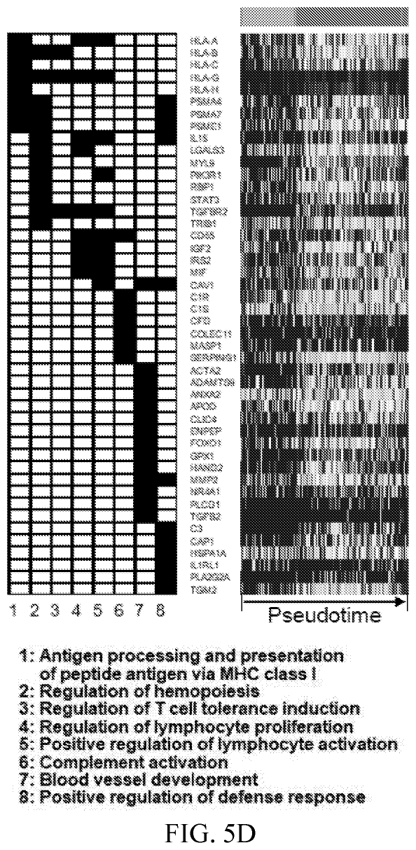

Infiltrating lymphocytes were reported to play essential roles in decidualization during pregnancy, where they were primarily involved in decidual angiogenesis and regulating trophoblastic invasion³⁰ (Hanna et al., 2006). Their functions in decidualization during the natural human menstrual cycle, however, remain to be defined. The dramatic increase in lymphocyte abundance in the early secretory phase in the data strongly suggests their involvement in decidualization (FIG. 5A and FIG. 23A). Their transcriptomic dynamics across the menstrual cycle were characterized to explore their roles and their interactions with other endometrial cell types during decidualization.

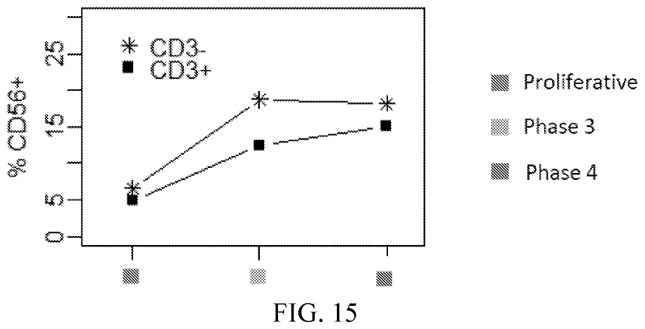

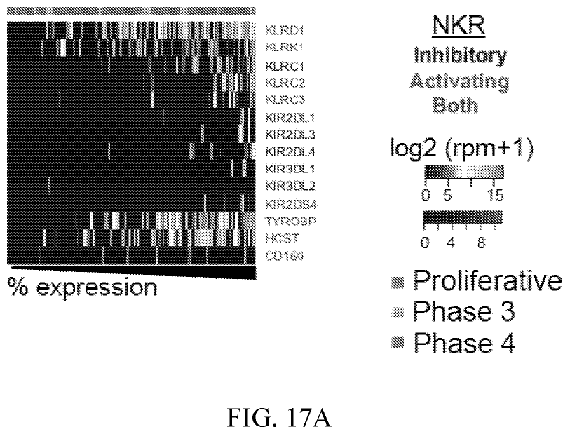

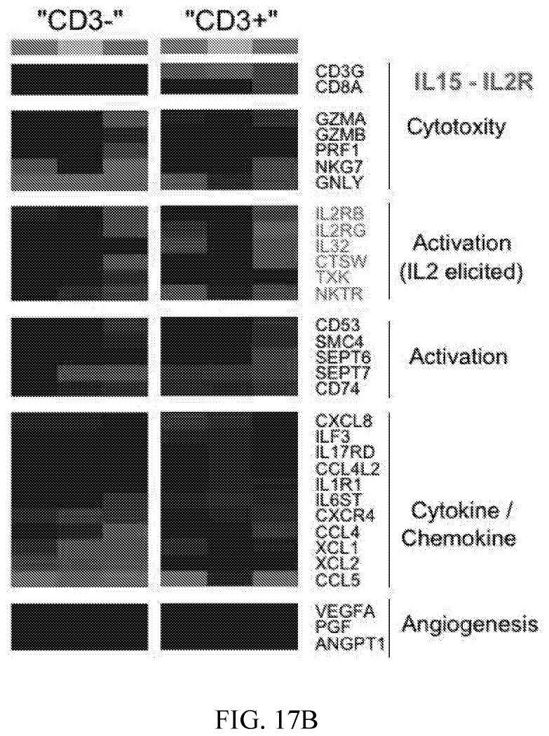

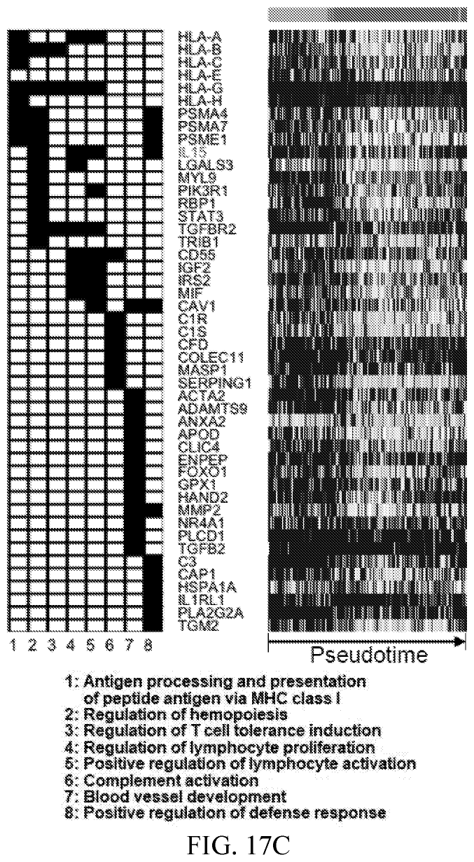

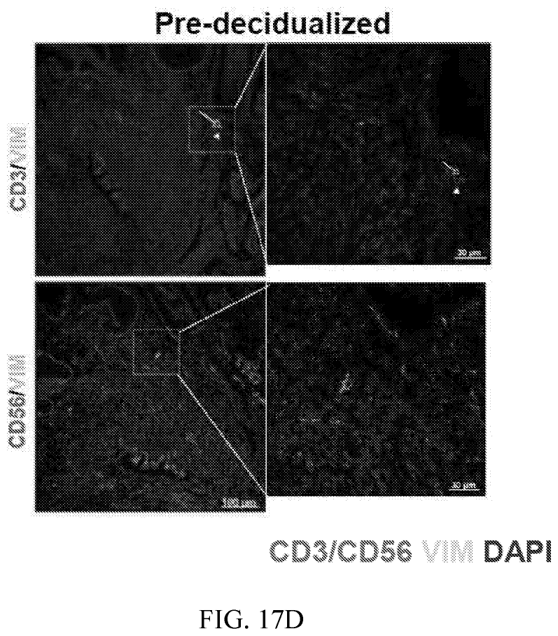

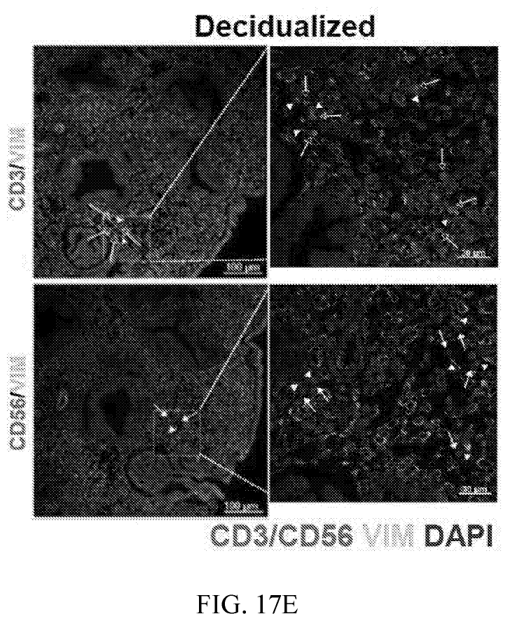

Compared to their counterparts in non-decidualized endometrium (i.e., secretory (phase 3) and proliferative phases), lymphocytes in decidualized endometrium (phase 4) in natural menstrual cycle have increased expression of markers that characteristic of uterine NK cells during pregnancy (CD69, ITGA1, NCAM1/CD56) (FIG. 5B and FIG. 23B). More interestingly, they express a more diverse repertoire of both activating and inhibitory NK receptors (NKR) responsible for recognizing major histocompatibility complex (MHC) class I molecules (FIG. 5B and FIG. 17A). Lineage-wise, lymphocytes expressing both NK and T cell markers and those expressing only NK markers were observed (FIG. 5B and FIG. 23B), and were therefore classified as “CD3+” and “CD3−” cells. Particularly, for both “CD3+” and “CD3−” cells, a noticeable rise in the fraction of cells expressing CD56, the canonical NK marker during pregnancy, occurs as early as the tissue transitioned from proliferative to secretory phase (FIG. 15 and FIG. 23C), suggesting that decidualization was initiated before the opening the WOI.

Next, genes were identified that are dynamically changing in the immune cells across the menstrual cycle, and those that are associated with NK functionality were characterized (FIG. 5C and FIG. 17B). In “CD3−” cells, a significant rise in cytotoxic granule genes was observed in decidualized endometrium (phase 4), with the exception of GNLY. In “CD3+” cells, this rise in cytotoxic potential was manifested by an increase in CD8, while the elevation in cytotoxic granule genes was only moderate. For both “CD3+” and “CD3−” cells, the increase in IL2 receptors expression was noticeable in phase 4. Equally notable were genes involved in IL2 elicited cell activation. As for the cytokine/chemokine repertoire, “CD3−” decidualized cells expressed high level of chemokines. Their “CD3+” counterparts, although expressing a more diverse cytokine repertoire, demonstrated much lower chemokine expression. Lastly, both “CD3+” and “CD3−” cells in decidualized endometrium have negligible expression in angiogenesis associated genes (FIG. 5C and FIG. 17C), contrary to their counterparts during pregnancy.

Intriguingly, decidualized stromal fibroblasts upregulated immune-related genes that reciprocated those upregulated in phase 4 immune cells. With the diversification of NKR observed in immune cells in the decidualized endometrium, an overall elevation in MHC class I genes in decidualized stromal fibroblasts was observed (FIG. 5D and FIG. 17C), including HLA-A and HLA-B, which are recognized by activating NKR, as well as HLA-G, recognized by inhibitory NKR. Worth noting was concurrent upregulation of HIVEP2 (FIG. 20D), a TF responsible for MHC class I gene upregulation. With the IL2-elicited activation observed in immune cells in the decidualized endometrium, also noticed was not only the elevation of IL15, which plays similar roles as IL2, as well as IL15-involved pathways that regulate lymphocyte activation and proliferation. Lastly, an angiogenesis associated pathway was elevated in decidualized stromal fibroblasts, complementing the lack of this functionality observed in NK cells in the same phase.

Using immunofluorescence, the spatial proximity between the identified immune subsets and stromal fibroblasts before (FIG. 17D top and bottom panels) and during (FIG. 17E top and bottom panels) decidualization was compared. A notable increase in the number of both CD3+(top panels of FIG. 17D and FIG. 17E) and CD56+(bottom panels of FIG. 17D and FIG. 17E) subsets were observed that are in close proximity with stromal fibroblasts during decidualization compared to pre-decidualization, which further validates the direct interplay between the immune and stromal subsets during decidualization.

The human menstrual cycle is not shared with many other species. Similar cycles have only been consistently observed in human, apes, and old world monkeys,^{1, 2} and not in any of the model organisms which undergo sexual reproduction such as mouse, zebrafish, or fly. This cyclic transformation is executed through dynamic changes in states and interactions of multiple cell types, including luminal and glandular epithelial cells, stromal cells, vascular endothelial cells, and infiltrating immune cells. Although different categorization schemes exist, the transformation has been primarily divided into two major stages by the event of ovulation: the proliferative (pre-ovulatory) and secretory (post-ovulatory) stage.³ During the secretory stage, endometrium enters a narrow window of receptive state that is both structurally and biochemically ideal for embryo to implant,^{4, 5} This, the mid-secretory stage, is known as the window of implantation (WOI). To prepare for this state, the tissue undergoes considerable reconstruction in the proliferative stage, during which one of the most essential elements is the formation of epithelial glands⁶, lined by glandular epithelium.

Given the broad relevance in human fertility and regenerative biology, a systematic characterization of endometrial transformation across the natural menstrual cycle has been long pursued. Histological characterizations established the morphological definition of menstrual, proliferative, early-, mid-, and late-secretory stages.³ Bulk level transcriptomic profiling advanced the characterization to a molecular and quantitative level,^{7, 8} and demonstrated the feasibility of translating the definition into clinical diagnosis of WOI.⁹ However, it has been a challenge to derive unbiased or mechanism-linked characterization from bulk-based readouts due to the uniquely heterogeneous and dynamic nature of endometrium.

The complexity of endometrium is unlike any other tissue: it consists of multiple cell types which vary dramatically in state through a monthly cycle as they enter and exit the cell cycle, remodel, and undergo various forms of differentiation with relatively rapid rates. The notable variance in menstrual cycle lengths within and between individuals¹⁰ adds an additional variable to the system. Thus, improved transcriptomic characterization of endometrial transformation, at the current stage of understanding, required that cell types and states be defined with minimum bias. High precision characterization and mechanistic understanding of hallmark events, such as WOI, required study of both the static and dynamic aspects of the tissue. Single cell RNAseq provided an ideal platform for these purposes. A systematic transcriptomic delineation of human endometrium across the natural menstrual cycle at single cell resolution was performed, and the results are disclosed herein.

In the present work, both static and dynamic characteristics of the human endometrium across the menstrual cycle with single cell resolution were studied. At the transcriptomic level, an unbiased approach was used to identify 6 major endometrial cell types, including a ciliated epithelial cell type, and 4-four major phases of endometrial transformation. For the unciliated epithelial cells and stromal fibroblasts, high-resolution trajectories were used to track their remodeling through the menstrual cycle with minimum bias. Based on these fundamental units and structures, the receptive state of the tissue was identified and characterized with high precision, and the dynamic cellular and molecular transformations that lead to the receptive state were studied.

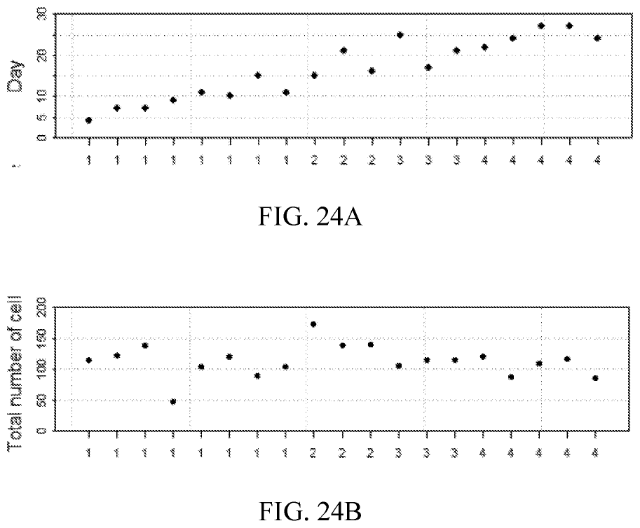

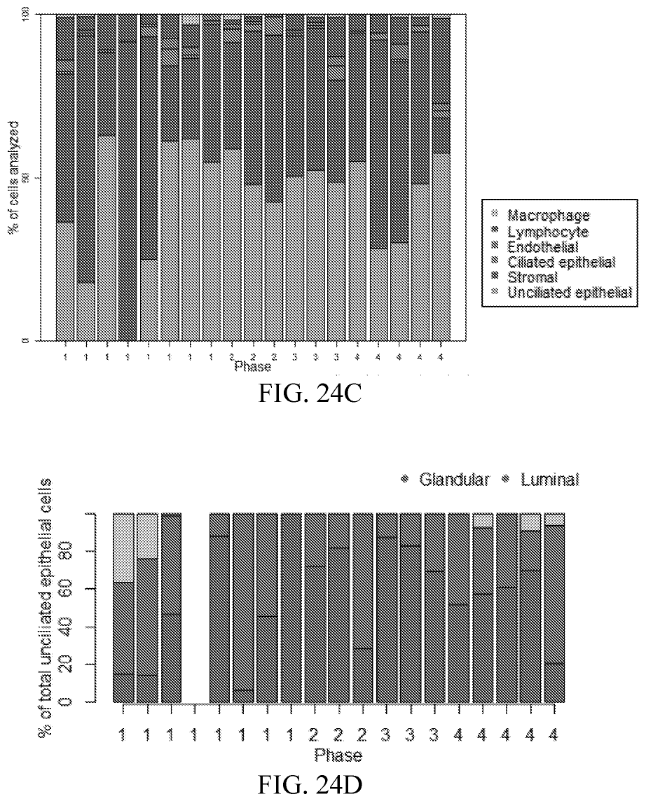

The use of single cell RNAseq to characterize human endometrium is at an early stage. Using endometrial biopsies, a previous study was only limited to the most abundant stromal fibroblasts (late-secretory phase, Krjutskov et al., 2016). Coincident with the present work, the feasibility of generating data from other endometrial cell types was also demonstrated by a group using full-thickness uterus (secretory phase, Wu et al., 2018), but cell types were only analyzed at a single time point on a single patient who underwent hysterectomy due to leiomyoma—a gynecological pathology known to cause menstrual abnormalities. Another coincident study modeled decidualization using in vitro cultures of human endometrial stromal fibroblasts and compared the result to the transition of stromal fibroblasts from mid- to late-secretory phase biopsies (Lucas et al., 2018). In the present study, biopsies were sampled from 19 healthy women across the entire menstrual cycle. Each of the reported biological phenotypes was supported by multiple biological replicates (i.e., women, FIG. 24), such that none of the biological results reported in the study were due to “individual-specific” results, undersampling, or confounded by pathological conditions.

An important result of the present work is the molecular characterization of the ciliated epithelium as a transcriptomically distinct endometrial cell type; these cells are consistently present but dynamically changing in abundance across the menstrual cycle (FIG. 14 and FIG. 23A). Although the existence of ciliated cells in the human endometrium has been speculated upon based on microscope studies since the 1890's (Benda, 1894), researchers have been hesitant to include them as an endometrial cell type due to two persisting controversies: 1) whether they exist solely due to pathological conditions (Novak and Rutledge, 1948) and 2) whether they persist across the entire menstrual cycle. The controversies have not been satisfyingly resolved by studies in the 1970's or recently, due to the confounding gynecological conditions of the examined tissue (Ferenczy et al., 1972; Masterton et al., 1975; Wu et al., 2018) and undersampling (Bartosch et al., 2011). In addition, no standardizable features or signatures were available to identify or isolate this cell type from endometrium. In addition to providing strong evidence that this cell type exists in healthy endometrium throughout the menstrual cycle, this study provides a comprehensive transcriptomic signature along with functional annotations which can serve as molecular anchors for future studies.

In general, ciliary motility facilitates the material transport (e.g., fluid or particles). The notable increase of ciliated epithelia in the second proliferative phase (FIG. 23A) suggests that they may play a role in sperm transport towards fallopian tubes through the uterine cavity. Moreover, their epithelial lineage identity and their consistent presence in glandular epithelia, as shown by the present in situ results, suggest they may function as a mucociliary transport apparatus, similar to those in the respiratory tract, to transport the secretions and provide a proper biochemical milieu. Further elucidation of this role may facilitate more accurate diagnosis of infertility. In addition, highlighted are the notably high fraction of genes (˜25%) in the derived signature with no functional annotations (FIG. 7). Co-expression of these genes (FIG. 1C and FIG. 16) with known cilium-associated genes and their exclusive activation in ciliated epithelium provides evidence for their cilium-associated functionality, e.g., in signal sensing and transduction (Bisgrove and Yost, 2006, PNAS Mao et al.), whose dysfunction can lead to both organ-specific diseases and multi-system syndromes^{31, 32} (Bisgrove and Yost, 2006; Fliegauf et al., 2007). Thus, functional studies that link the roles of these un-annotated genes with cilia functionality will also facilitate understanding of this organelle. While it remains biologically intriguing that many genes comprising the transcriptomic signal lack an assigned function, they are demonstrably associated with the switching of endometrial state, and thus remain useful in a multigene transcriptomic analysis in improving the accuracy and precision with which the signal can be characterized in a subject.

The opening of WOI was identified, and a method diagnosing the unique transcriptomic dynamics accompanying both the entrance and the closure of the WOI. It was previously postulated that a continuous dynamic would better describe the entrance of WOI, since human embryo implantation doesn't seem to be controlled by a single hormonal factor as in mice^{33, 34} (Hoversland et al., 1982; Paria et al., 1993), while discontinuous characteristics were also speculated based on morphological observation of plasma membrane transformation³⁵ (Murphy, 2004). The present data suggest that the WOI opens with an abrupt and discontinuous transcriptomic transition in unciliated epithelium, accompanied by a more continuous transition in stromal fibroblasts. The abruptness of the transition also suggests that it should be possible to diagnose the opening of the WOI with high precision in clinical practices of in vitro fertilization and embryo transfer.

It is intriguing that the mid- and late-secretory phases fall into the same major phase at the transcriptomic level, especially since the physiological differences between mid- (high progesterone level, embryo implantation) and late-secretory phase (progesterone withdraw, preparing for tissue desquamation) seem to be as large as that between early- to mid-secretory phase, if not larger. In fact, the characteristic transition at the closure of the WOI is largely contributed to by the same group of genes that contributed to the abrupt opening of the WOI, except that while at the opening their upregulation is rapid and uniform across all cells, at the closure the downregulation was executed less uniformly and across a longer period of time. From a dynamic perspective, the difference suggests that the transition between mid-to-late secretory phases, although in magnitude may be similar to that between early-to-mid secretory phases, is slower in rate, perhaps reflective of the rate of progesterone withdrawal. From a molecular perspective, the less uniform downregulation of genes suggests that the closure of the WOI may be mediated through paracrine factors and cell-cell communications.

The abrupt opening of the WOI also allowed elucidation of the relationship between the WOI and decidualization. As noted earlier, decidualization is the transformation of stromal fibroblasts that is necessary for pregnancy in both human and mouse, and supports the development of an implanted embryo. However, contrary to the mouse, where decidualization is triggered by implanting embryo(s)³⁶ (Cha et al., 2012) and thus occurs exclusively during pregnancy, in humans, decidualization occurs spontaneously during natural human menstrual cycles independent of the presence of an embryo²¹ (Evans et al., 2016). Thus, the relative timing between the WOI and the initiation of decidualization in human is unclear. While histological observation suggests that decidualization starts around mid-secretory phase, the present data indicates that decidualization is initiated before the opening of the WOI, and that at the opening of the WOI decidualized features are widespread in stromal fibroblasts at the transcriptomic level. This lag of morphological signals relative to transcriptomic signals could result from the delay of phenotypic manifestation after transcription either due to inherent delay between transcription and translation or through post-transcriptional modifications.

The transcriptomic signature in luminal and glandular epithelium during epithelial gland formation was identified. The original definition of luminal and glandular epithelia was established based on the distinct morphology and physical location between the two. Their distinction at the transcriptome level had not previously been established. Markers were found that differentiate the two across multiple phases of the menstrual cycle. Moreover, signatures were discovered that are differentially up-regulated in glandular and luminal epithelium during the formation of epithelial glands. Epithelial glands create proper biochemical milieu for embryo implantation and subsequent development of pregnancy. In humans, the mechanism for their reconstruction during proliferative phases, however, is unclear. Previous studies through clonogenic assays reported that the cyclic regeneration of both glandular and luminal epithelium was executed by progenitors with sternness characteristics in the unshed layer of the uterus (basalis) (Huang et al., 2012; Nguyen et al., 2017; W. C. et al., 1997). The present analysis suggests a mechanism that involves MET for re-epithelization followed by tubulogenesis in the luminal epithelium as well as proliferation activities that were locally, concentrated at glandular epithelium for reformation of epithelial glands. The data however cannot rule out the possibility that cells that re-epitheliate the endometrium are the progeny of previously reported candidates with stemness characteristics.

Lastly, evidence was provided for the direct interplay between stroma and lymphocytes during decidualization in menstrual cycle. Analysis suggested that, during decidualization in cycling endometrium, stromal fibroblasts are directly responsible for the activation of lymphocytes through IL2-elicited pathways. The diversification of activating and inhibitory NKR in immune cells and the overall up-regulation of MHC class I molecules in stromal fibroblasts is particularly interesting. During pregnancy, cytotoxic NK cells were tolerant towards the semi-allogeneic fetus³⁷ (Schmitt et al., 2007). This paradoxical phenomenon was hypothesized to be mediated by 1) the upregulation of non-classical MHC class I molecule (HLA-G)³⁸ (Apps et al., 2007), the ligand to NK inhibitory receptor, and 2) the downregulation of classical MHC class I molecules (HLA-A, HLA-B)^{39, 40} (Moffett-King, 2002; Sivori et al., 2000) that engage with NK activating receptors. Results demonstrate that similar suppression in NK cells with high cytotoxic potential occurs during natural menstrual cycle, however exerted by decidualized stromal fibroblasts.

In summary, the human endometrium was systematically characterized across the menstrual cycle from both a static and a dynamic perspective. The high resolution of the data and the analytical framework allowed previously unresolved questions that are centered on the tissue's receptivity to embryo implantation to be answered. These findings and the molecular signatures that were discovered provide conceptual foundations and practical molecular anchors for reproductive and clinical applications.

### REFERENCES

The following references are cited within the present Application. Each is incorporate herein by reference in their entireties.
1. R. D. Martin, The evolution of human reproduction: A primatological perspective. Yearb. Phys. Anthropol. 50 (2007), pp. 59-84.
2. D. Emera, R. Romero, G. Wagner, The evolution of menstruation: A new model for genetic assimilation: Explaining molecular origins of maternal responses to fetal invasiveness. BioEssays. 34, 26-35 (2012).
3. R. W. Noyes, A. T. Hertig, J. Rock, Dating the Endometrial Biopsy. Fertil. Steril. 1, 3-25 (1950).
4. H. B. Croxatto et al., Studies on the duration of egg transport by the human oviduct. II. Ovum location at various intervals following luteinizing hormone peak. Am. J. Obstet. Gynecol. 132, 629-634 (1978).
5. A. J. Wilcox, D. D. Baird, C. R. Weinberg, Time of Implantation of the Conceptus and Loss of Pregnancy. N. Engl. J. Med. 340, 1796-1799 (1999).
6. J. Filant, T. E. Spencer, Uterine glands: Biological roles in conceptus implantation, uterine receptivity and decidualization. Int. J. Dev. Biol. 58 (2014), pp. 107-116.
7. A. Riesewijk et al., Gene expression profiling of human endometrial receptivity on days LH+2 versus LH+7 by microarray technology. Mol. Hum. Reprod. 9, 253-64 (2003).
8. M. Ruiz-Alonso, D. Blesa, C. Simon, The genomics of the human endometrium. Biochim. Biophys. Acta—Mol. Basis Dis. 1822, 1931-1942 (2012).
9. P. Díaz-Gimeno et al., A genomic diagnostic tool for human endometrial receptivity based on the transcriptomic signature. Fertil. Steril. 95, 50-60 (2011).
10. Y. Guo, A. K. Manatunga, S. Chen, M. Marcus, Modeling menstrual cycle length using a mixture distribution. Biostatistics. 7, 100-114 (2006).
11. L. Van Der Maaten, G. Hinton, Visualizing Data using t-SNE. J. Mach. Learn. Res. 1. 620, 267-84 (2008).
12. F. Zhou, S. Roy, SnapShot: Motile Cilia. Cell. 162 (2015), p. 224-224.e1.
13. H. M. Mitchison, E. M. Valente, Motile and non-motile cilia in human pathology: from function to phenotypes. J. Pathol. 241 (2017), pp. 294-309.
14. T. Hastie, W. Stuetzle, Principal curves. J. Am. Stat. Assoc. 84, 502-516 (1989).
15. P. Díaz-Gimeno, M. Ruíz-Alonso, D. Blesa, C. Simón, Transcriptomics of the human endometrium. Int. J. Dev. Biol. 58, 127-137 (2014).
16. Y. Park, M. C. Nnamani, J. Maziarz, G. P. Wagner, Cis-regulatory evolution of forkhead box O1 (FOXO1), a terminal selector gene for decidual stromal cell identity. Mol. Biol. Evol. 33, 3161-3169 (2016).
17. H. Okada et al., Regulation of decidualization and angiogenesis in the human endometrium: Mini review. J. Obstet. Gynaecol. Res. 40 (2014), pp. 1180-1187.
18. C. Y. Ramathal, I. C. Bagchi, R. N. Taylor, M. K. Bagchi, Endometrial decidualization: Of mice and men. Semin. Reprod. Med. 28 (2010), pp. 17-26.
19. M. Uhlen et al., Tissue-based map of the human proteome. Science (80-.). 347, 1260419-1260419 (2015).
20. S. Khurana, S. P. George, Regulation of cell structure and function by actin-binding proteins: Villin's perspective. FEBS Lett. 582 (2008), pp. 2128-2139.
21. J. Evans et al., Fertile ground: Human endometrial programming and lessons in health and disease. Nat. Rev. Endocrinol. 12 (2016), pp. 654-667.
22. C. a White et al., Blocking LIF action in the uterus by using a PEGylated antagonist prevents implantation: a nonhormonal contraceptive strategy. Proc. Natl. Acad. Sci. U.S.A. 104, 19357-62 (2007).
23. J. Evans et al., Prokineticin 1 mediates fetal-maternal dialogue regulating endometrial leukemia inhibitory factor. FASEB J. 23, 2165-75 (2009).
24. M. Ashburner et al., Gene ontology: Tool for the unification of biology. Nat. Genet. 25 (2000), pp. 25-29.
25. The Gene Ontology Consortium, Expansion of the Gene Ontology knowledgebase and resources. Nucleic Acids Res. 45, D331-D338 (2017).
26. H. Mi et al., PANTHER version 11: Expanded annotation data from Gene Ontology and Reactome pathways, and data analysis tool enhancements. Nucleic Acids Res. 45, D183-D189 (2017).
27. O. W. C., A. C. I., S. R., Zonal changes in proliferation in the rhesus endometrium during the late secretory phase and menses. Proc. Soc. Exp. Biol. Med. 214 (1997), pp. 132-138.
28. C. C. Huang, G. D. Orvis, Y. Wang, R. R. Behringer, Stromal-to-epithelial transition during postpartum endometrial regeneration. PLoS One. 7 (2012), doi:10.1371/journal.pone.0044285.
29. P. S. Cooke, T. E. Spencer, F. F. Bartol, K. Hayashi, Uterine glands: Development, function and experimental model systems. Mol. Hum. Reprod. 19 (2013), pp. 547-558.
30. J. Hanna et al., Decidual NK cells regulate key developmental processes at the human fetal-maternal interface. Nat. Med. 12, 1065-1074 (2006).
31. B. W. Bisgrove, H. J. Yost, The roles of cilia in developmental disorders and disease. Development. 133, 4131-4143 (2006).
32. M. Fliegauf, T. Benzing, H. Omran, When cilia go bad: Cilia defects and ciliopathies. Nat. Rev. Mol. Cell Biol. 8 (2007), pp. 880-893.
33. R. C. Hoversland, S. K. Dey, D. C. Johnson, Catechol estradiol induced implantation in the mouse. Life Sci. 30, 1801-1804 (1982).
34. B. C. Paria, Y. M. Huet-Hudson, S. K. Dey, Blastocyst's state of activity determines the “window” of implantation in the receptive mouse uterus. Proc. Natl. Acad. Sci. U.S.A. 90, 10159-62 (1993).
35. C. R. Murphy, Uterine receptivity and the plasma membrane transformation. Cell Res. 14 (2004), pp. 259-267.
36. J. Cha, X. Sun, S. K. Dey, Mechanisms of implantation: Strategies for successful pregnancy. Nat. Med. 18 (2012), pp. 1754-1767.
37. C. Schmitt, B. Ghazi, A. Bensussan, in Reproductive BioMedicine Online (2008), vol. 16, pp. 192-201.
38. R. Apps, L. Gardner, A. M. Sharkey, N. Holmes, A. Moffett, A homodimeric complex of HLA-G on normal trophoblast cells modulates antigen-presenting cells via LILRB1. Eur. J. Immunol. 37, 1924-1937 (2007).
39. A. Moffett-King, Natural killer cells and pregnancy. Nat. Rev. Immunol. 2 (2002), pp. 656-663.
40. S. Sivori et al., Triggering receptors involved in natural killer cell-mediated cytotoxicity against choriocarcinoma cell lines. Hum. Immunol. 61, 1055-1058 (2000).
41. A. Dobin et al., STAR: Ultrafast universal RNA-seq aligner. Bioinformatics. 29, 15-21 (2013).
42. S. Anders, P. T. Pyl, W. Huber, HTSeq-A Python framework to work with high-throughput sequencing data. Bioinformatics. 31, 166-169 (2015).
43. Y. Benjamini, Y. Hochberg, Controlling the false discovery rate: a practical and powerful approach to multiple testing. J. R. Stat. Soc. B. 57 (1995), pp. 289-300.
44. D. Yekutieli, Y. Benjamini, Resampling-based false discovery rate controlling multiple test procedures for correlated test statistics. J. Stat. Plan. Inference. 82, 171-196 (1999).
45. A. Lachmann, F. M. Giorgi, G. Lopez, A. Califano, ARACNe-AP: Gene network reverse engineering through adaptive partitioning inference of mutual information. Bioinformatics. 32, 2233-2235 (2016).
46. I. Tirosh et al., Single-cell RNA-seq supports a developmental hierarchy in human oligodendroglioma. Nature. 539, 309-313 (2016).
47. E. Z. Macosko et al., Highly parallel genome-wide expression profiling of individual cells using nanoliter droplets. Cell. 161, 1202-1214 (2015).
48. M. S. Kowalczyk et al., Single-cell RNA-seq reveals changes in cell cycle and differentiation programs upon aging of hematopoietic stem cells. Genome Res. 25, 1860-1872 (2015).
49. M. L. Whitfield, Identification of Genes Periodically Expressed in the Human Cell Cycle and Their Expression in Tumors. Mol. Biol. Cell. 13, 1977-2000 (2002).
50. H. B. Mann, D. R. Whitney, On a Test of Whether one of Two Random Variables is Stochastically Larger than the Other. Ann. Math. Stat. 18, 50-60 (1947).

What is claimed is:

1. A method of diagnosing a menstrual cycle event in a subject, the method comprising detecting in a biological sample a gene signature for one or more endometrial cell types.

2. The method of claim 1, wherein the menstrual cycle event is follicular phase, ovulation, or the luteal phase of a menstrual cycle.

3. The method of claim 1, wherein the menstrual cycle event is a window of implantation (WOI).

4. The method of claim 1, wherein the one or more endometrial cell types is selected from the group consisting of stromal cells (for example stromal fibroblasts), endothelium cells, immune cells, unciliated epithelium cells, and ciliated epithelium cells.

5. The method of claim 1, wherein the one or more endometrial cell types is unciliated epithelium cells.

6. The method of claim 1, wherein the one or more endometrial cell types is unciliated cells and the gene signature comprises one or more biomarkers selected from the group consisting of: PLUA, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP.

7. The method of claim 6, wherein CADM1, NPAS3, ATP1A1, and TRAK1 are downregulated and NUPR1 is upregulated relative to an index.

8. The method of claim 1, wherein the one or more endometrial cell types is a stromal cell (for example a stromal fibroblast) and the gene signature comprises one or more biomarkers selected from the group consisting of: STC1, NGATC2, BMP2, PMAIP1, MMP11, SFRP1, WNT5A, ZFYVE21, CILP, SLF2, MATN2, S100A4, DKK1, CRYAB, FOXO1, IL15, FGF7, and LMCD1.

9. The method of claim 8, wherein NFATC2, BMP2, PMAIP1, ZFYVE21, CILP, SLF2, MATN2, and FGF7 are downregulated and CRYAB is upregulated relative to an index.

10. The method of claim 1, wherein prior to the detection step, the one or more endometrial cells are separated from one another.

11. The method of claim 4, wherein prior to the detection step, the stromal cells, endothelium cells, immune cells, unciliated epithelium cells, and ciliated epithelium cells are separated from one another.

12. The method of claim 10 or 11, wherein the cells are separated by fluorescence activated cell sorting (FACS).

13. The method of claim 5, wherein the unciliated epithelium cells are first separated by fluorescence activated cell sorting (FACS).

14. The method of claim 3, further comprising the step of transferring a fertilized embryo to the uterus of the subject determined to be within the window of implantation.

15. A method comprising determining a gene expression profile in each of a plurality of endometrial cells, wherein said endometrial cells are:
(a) in an endometrial sample obtained from a subject, and
(b) unciliated epithelial cells.

16. The method of claim 15, wherein the unciliated epithelial cells are separated from ciliated epithelial cells.

17. The method of claim 15, wherein the gene expression profile of an unciliated epithelial cell is identified using one or more gene expression markers characteristic of unciliated epithelial cells.

18. The method of claim 15, wherein the gene expression profile comprises at least twenty genes selected from the group consisting of the genes shown in FIG. 3B, or in any one of Tables 1-17.

19. The method of claim 15, wherein the gene expression markers characteristic of unciliated epithelial cells comprise PLUA, MMP7, THBS1, CADM1, NPAS3, ATP1A1, ANK3, ALPL, TRAK1, SCGB1D2, MT1F, MT1X, MT1E, MT1G, CXCL14, MAOA, DPP4, NUPR1, GPX3, and PAEP.

20. A method for detecting that a subject is within a window of implantation (WOI), the method comprising:
(a) determining a level of expression of at least twenty genes in a sample of endometrial cells obtained from a subject, wherein the twenty genes are selected from the group consisting of the genes shown in FIG. 3B, or Tables 9 or 10;
(b) comparing the determined level of expression of each of the at least twenty genes with a control level; and
(c) determining whether the subject is within the WOI, wherein the subject is identified as being within the WOI if the level of the expression of at least twenty genes is at least two-fold higher than a control level.

21. A method for identifying a subject as being within a window of implantation (WOI), the method comprising:
(a) determining a level of expression of at least one gene in an isolated cell population, wherein the at least one gene is selected from the group consisting of PAEP, GPX3, CXCL14, NUPR1, DPP4, MAOA, MT1G, MT1E, MT1X, and MT1F, wherein the isolated cell population has been isolated from a sample of endometrial cells obtained from a subject, wherein the cell population comprises cells having elevated expression of genes associated with epithelial cells and depressed expression of genes associated with cilial function; and
(b) comparing the determined level of expression of the at least one gene with a control level; and
(c) identifying the subject as being within the WOI, wherein the subject is identified as being within the WOI if the level of the expression of at least one gene is at least two-fold higher than a control level.

22. A method of increasing the likelihood of becoming pregnant comprising:
(a) performing the method of any of the above claims to determine whether the subject is within a window of implantation (WOI); and
(b) transferring a fertilized embryo to the uterus of the subject determined to be within the window of implantation.

23. A method of treating infertility in a subject in need thereof, comprising administering an effective amount of an agent that upregulates any one or more of the genes selected from the group consisting of PAEP, GPX3, CXCL14, NUPR1, DPP4, MAOA, MT1G, MT1E, MT1X, and MT1F in one or more of the tissues in the subject in an effective amount to treat the infertility.

24. A method for detecting a window of implantation (WOI) in a subject, the method comprising:
(a) isolating a cell population within a sample of endometrial cells obtained from a subject, wherein the cell population comprises cells having elevated expression of genes associated with epithelial cells and depressed expression of genes associated with cilial function;
(b) determining a level of expression of at least one gene in the cell population wherein the at least one gene is selected from the group consisting of PAEP, GPX3, and CXCL14; and
(c) determining whether the subject has entered the WOI, wherein the subject is identified as within the WOI if the level of the expression of at least one gene is higher than a predetermined level.

25. The method of claim 24, wherein step (a) comprises determining the level of expression of at least two genes from the group consisting of PAEP, GPX3, and CXCL14.

26. The method of claim 24, wherein step (a) comprises determining the level of expression of each of the genes from the group consisting of PAEP, GPX3, and CXCL14.

27. The method of any of claims 24-26 further comprising determining the level of expression of at least one gene selected from the group consisting of NUPR1, DPP4, MAOA, MT1G, MT1E, MT1X, and MT1F.

28. The method of any of claims 24-26, comprising determining the level of expression of at least two genes selected from the group consisting of NUPR1, DPP4, MAOA, MT1G, MT1E, MT1X, and MT1F.

29. The method of any of claims 24-26, comprising determining the level of expression of at least three genes selected from the group consisting of NUPR1, DPP4, MAOA, MT1G, MT1E, MT1X, and MT1F.

30. The method of any of claims 24-26, comprising determining the level of expression of each gene selected from the group consisting of NUPR1, DPP4, MAOA, MT1G, MT1E, MT1X, and MT1F.

31. The method of any of the preceding claims, wherein the determining the level of expression of a gene comprises determining the amount of a nucleic acid.

32. The method of claim 31, wherein the amount of the nucleic acid is determined using a real-time reverse transcriptase PCR (RT-PCR) assay and/or a nucleic acid microarray.

33. The method of claim 31, wherein the amount of nucleic acid is determined using a hybridization assay and at least one labeled binding agent.

34. The method of claim 33, wherein the at least one labeled binding agent is at least one labeled oligonucleotide binding agent.

35. The method of any of claims 24-34, wherein determining the level of expression of a gene comprises determining an amount of a protein encoded by that gene.

36. The method of claim 35, wherein the amount of the protein is determined using an immunohistochemical assay, an immunoblotting assay, and/or a flow cytometry assay.

37. The method of any of the preceding claims, wherein the sample is selected from the group consisting of a sample of endometrium tissue, endometrial stromal cells, and/or endometrial fluid.

38. The method of any of the preceding claims, wherein the subject is a human.

39. The method of claim 38, wherein the human is trying to become pregnant.

40. The method of claim 38 further comprising transferring an embryo into the uterus of the subject.

41. The method of claim 40, wherein the embryo is implanted in the uterus of the subject.

42. A method of increasing the likelihood of becoming pregnant comprising using the method of any of claims A1-A17 to detect a window of implantation (WOI) in a subject, and implanting a fertilized embryo if the window of implantation is open.

43. A method of treating infertility in a subject in need thereof, comprising administering an effective amount of an agent that upregulates any one or more of the genes selected from the group consisting of PAEP, GPX3, CXCL14, NUPR1, DPP4, MAOA, MT1G, MT1E, MT1X, and MT1F in one or more of the tissues in the subject in an effective amount to treat the infertility.

44. The method of claim 43, wherein the agent comprises a nucleic acid encoding for any one or more of the genes selected from the group consisting of PAEP, GPX3, CXCL14, NUPR1, DPP4, MAOA, MT1G, MT1E, MT1X, and MT1F in an expression system.

45. The method of claim 43 or 44, wherein the administering of the agent results in the opening of the window of implantation in the subject.

46. The method of claim 43, wherein the method further comprises implanting a fertilized embryo in the subject.

47. The method of 44, wherein the implanting a fertilized embryo results in a higher rate of conception and/or a live birth.


SEQUENCE LISTING (38 sequences)

SEQ ID NO: 1 - protein, 414 aa, Artificial Sequence
  Synthetic Polypeptide
MVFHLRTRYEQANCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 2 - protein, 267 aa, Artificial Sequence
  Synthetic Polypeptide
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 3 - protein, 1170 aa, Artificial Sequence
  Synthetic Polypeptide
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 4 - protein, 414 aa, Artificial Sequence
  Synthetic Polypeptide
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDSRAGEEGSIRAVDHAVIGGVVAVVVFAMLCLLIILGRYFARHKGTYFTHEAKGADDAADADTAIINAEGGQNNSEEKKEYFI

SEQ ID NO: 5 - protein, 933 aa, Artificial Sequence
  Synthetic Polypeptide
MAPTKPSFQQDPSRRERITAQHPLPNQSECRKIYRYDGIYCESTYQNLQALRKEKSRDAARSRRGKENFEFYELAKLLPLPAAITSQLDKASIIRLTISYLKMRDFANQGDPPWNLRMEGPPPNTSVKVIGAQRRRSPSALAIEVFEAHLGSHILQSLDGFVFALNQEGKFLYISETVSIYLGLSQVELTGSSVFDYVHPGDHVEMAEQLGMKLPPGRGLLSQGTAEDGASSASSSSQSETPEPVESTSPSLLTTDNTLERSFFIRMKSTLTKRGVHIKSSGYKVIHITGRLRLRVSLSHGRTVPSQIMGLVVVAHALPPPTINEVRIDCHMFVTRVNMDLNIIYCENRISDYMDLTPVDIVGKRCYHFIHAEDVEGIRHSHLDLLNKGQCVTKYYRWMQKNGGYIWIQSSATIAINAKNANEKNIIWVNYLLSNPEYKDTPMDIAQLPHLPEKTSESSETSDSESDSKDTSGITEDNENSKSDEKGNQSENSEDPEPDRKKSGNACDNDMNCNDDGHSSSNPDSRDSDDSFEHSDFENPKAGEDGFGALGAMQIKVERYVESESDLRLQNCESLTSDSAKDSDSAGEAGAQASSKHQKRKKRRKRQKGGSASRRRLSSASSPGGLDAGLVEPPRLLSSPNSASVLKIKTEISEPINFDNDSSIWNYPPNREISRNESPYSMTKPPSSEHFPSPQGGGGGGGGGGGLHVAIPDSVLTPPGADGAAARKTQFGASATAALAPVASDPLSPPLSASPRDKHPGNGGGGGGGGGGAGGGGPSASNSLLYTGDLEALQRLQAGNVVLPLVHRVTGTLAATSTAAQRVYTTGTIRYAPAEVTLAMQSNLLPNAHAVNFVDVNSPGFGLDPKTPMEMLYHHVHRLNMSGPFGGAVSAASLTQMPAGNVFTTAEGLFSTLPFPVYSNGIHAAQTLERKED

SEQ ID NO: 6 - protein, 1023 aa, Artificial Sequence
  Synthetic Polypeptide
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 7 - protein, 1001 aa, Artificial Sequence
  Synthetic Polypeptide
MALPQSEDAMTGDTDKYLGPQDLKELGDDSLPAEGYMGFSLGARSASLRSFSSDRSYTLNRSSYARDSMMIEELLVPSKEQHLTFTREFDSDSLRHYSWAADTLDNVNLVSSPIHSGFLVSFMVDARGGSMRGSRHHGMRIIIPPRKCTAPTRITCRLVKRHKLANPPPMVEGEGLASRLVEMGPAGAQFLGPVIVEIPHFGSMRGKERELIVLRSENGETWKEHQFDSKNEDLTELLNGMDEELDSPEELGKKRICRIITKDFPQYFAVVSRIKQESNQIGPEGGILSSTTVPLVQASFPEGALTKRIRVGLQAQPVPDEIVKKILGNKATFSPIVTVEPRRRKFHKPITMTIPVPPPSGEGVSNGYKGDTTPNLRLLCSITGGTSPAQWEDITGTTPLTFIKDCVSFTTNVSARFWLADCHQVLETVGLATQLYRELICVPYMAKFVVFAKMNDPVESSLRCFCMTDDKVDKTLEQQENFEEVARSKDIEVLEGKPIYVDCYGNLAPLTKGGQQLVFNFYSFKENRLPFSIKIRDTSQEPCGRLSFLKEPKTTKGLPQTAVCNLNITLPAHKKIEKTDRRQSFASLALRKRYSYLTEPGMSPQSPCERTDIRMAIVADHLGLSWTELARELNFSVDEINQIRVENPNSLISQSFMLLKKWVTRDGKNATTDALTSVLTKINRIDIVTLLEGPIFDYGNISGTRSFADENNVFHDPVDGYPSLQVELETPTGLHYTPPTPFQQDDYFSDISSIESPLRTPSRLSDGLVPSQGNIEHSADGPPVVTAEDASLEDSKLEDSVPLTEMPEAVDVDESQLENVCLSWQNETSSGNLESCAQARRVTGGLLDRLDDSPDQCRDSITSYLKGEAGKFEANGSHTEITPEAKTKSYFPESQNDVGKQSTKETLKPKIHGSGHVEEPASPLAAYQKSLEETSKLIIEETKPCVPVSMKKMSRTSPADGKPRLSLHEEEGSSGSEQKQGEGFKVKTKKEIRHVEKKSHS

SEQ ID NO: 8 - protein, 524 aa, Artificial Sequence
  Synthetic Polypeptide
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 9 - protein, 953 aa, Artificial Sequence
  Synthetic Polypeptide
MALVFQFGQPVRAQPLPGLCHGKLIRTNACDVCNSTDLPEVEIISLLEEQLPHYKLRADTIYGYDHDDWLHTPLISPDANIDLTTEQIEETLKYFLLCAERVGQMTKTYNDIDAVTRLLEEKERDLELAARIGQSLLKKNKTLTERNELLEEQVEHIREEVSQLRHELSMKDELLQFYTSAAEESEPESVCSTPLKRNESSSSVQNYFHLDSLQKKLKDLEEENVVLRSEASQLKTETITYEEKEQQLVNDCVKELRDANVQIASISEELAKKTEDAARQQEEITHLLSQIVDLQKKAKACAVENEELVQHLGAAKDAQRQLTAELRELEDKYAECMEMLHEAQEELKNLRNKTMPNTTSRRYHSLGLFPMDSLAAEIEGTMRKELQLEEAESPDITHQKRVFETVRNINQVVKQRSLTPSPMNIPGSNQSSAMNSLLSSCVSTPRSSFYGSDIGNVVLDNKTNSIILETEAADLGNDERSKKPGTPGTPGSHDLETALRRLSLRRENYLSERRFFEEEQERKLQELAEKGELRSGSLTPTESIMSLGTHSRFSEFTGFSGMSFSSRSYLPEKLQIVKPLEGSATLHHWQQLAQPHLGGILDPRPGVVTKGFRTLDVDLDEVYCLNDFEEDDTGDHISLPRLATSTPVQHPETSAHHPGKCMSQTNSTFTFTTCRILHPSDELTRVTPSLNSAPTPACGSTSHLKSTPVATPCTPRRLSLAESFTNTRESTTTMSTSLGLVWLLKERGISAAVYDPQSWDRAGRGSLLHSYTPKMAVIPSTPPNSPMQTPTSSPPSFEFKCTSPPYDNFLASKPASSILREVREKNVRSSESQTDVSVSNLNLVDKVRRFGVAKVVNSGRAHVPTLTEEQGPLLCGPPGPAPALVPRGLVPEGLPLRCPTVTSAIGGLQLNSGIRRNRSFPTMVGSSMQMKAPVTLTSGILMGAKLSKQTSLR

SEQ ID NO: 10 - protein, 90 aa, Artificial Sequence
  Synthetic Polypeptide
MKLSVCLLLVTLALCCYQANAEFCPALVSELLDFFFISEPLFKLSLAKFDAPPEAVAAKLGVKRCTDQMSLQKRSLIAEVLVKILKKCSV

SEQ ID NO: 11 - protein, 44 aa, Artificial Sequence
  Synthetic Polypeptide
MDPNCSCAAGVSCTCAGSCKCKECKCTSCKKSECEAISMVWGCG

SEQ ID NO: 12 - protein, 61 aa, Artificial Sequence
  Synthetic Polypeptide
MDPNCSCSPVGSCACAGSCKCKECKCTSCKKSCCSCCPVGCAKCAQGCICKGTSDKCSCCA

SEQ ID NO: 13 - protein, 127 aa, Artificial Sequence
  Synthetic Polypeptide
MDPNCSCATGGSCTCAGSCKCKECKCTSCKKSECGAISRNLGLWLRLGGNSRLALSASFWGTGLSLPSLPVSFPLQAFCPKFRWGRTAFFSWDTNPNCTPYGFRTELCQTKKSILWVWVLSSSQACY

SEQ ID NO: 14 - protein, 62 aa, Artificial Sequence
  Synthetic Polypeptide
MDPNCSCAAAGVSCTCASSCKCKECKCTSCKKSCCSCCPVGCAKCAQGCICKGASEKCSCCA

SEQ ID NO: 15 - protein, 111 aa, Artificial Sequence
  Synthetic Polypeptide
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 16 - protein, 527 aa, Artificial Sequence
  Synthetic Polypeptide
MENQEKASIAGHMFDVVVIGGGISGLSAAKLLTEYGVSVLVLEARDRVGGRTYTIRNEHVDYVDVGGAYVGPTQNRILRLSKELGIETYKVNVSERLVQYVKGKTYPFRGAFPPVWNPIAYLDYNNLWRTIDNMGKEIPTDAPWEAQHADKWDKMTMKELIDKICWTKTARRFAYLFVNINVTSEPHEVSALWFLWYVKQCGGTTRIFSVTNGGQERKFVGGSGQVSERIMDLLGDQVKLNHPVTHVDQSSDNIIIETLNHEHYECKYVINAIPPTLTAKIHFRPELPAERNQLIQRLPMGAVIKCMMYYKEAFWKKKDYCGCMIIEDEDAPISITLDDTKPDGSLPAIMGFILARKADRLAKLHKEIRKKKICELYAKVLGSQEALHPVHYEEKNWCEEQYSGGCYTAYFPPGIMTQYGRVIRQPVGRIFFAGTETATKWSGYMEGAVEAGERAAREVLNGLGKVTEKDIWVQEPESKDVPAVEITHTFWERNLPSVSGLLKIIGFSTSVTALGFVLYKYKLLPRS

SEQ ID NO: 17 - protein, 766 aa, Artificial Sequence
  Synthetic Polypeptide
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 18 - protein, 100 aa, Artificial Sequence
  Synthetic Polypeptide
MATFPPATSAPQQPPGPEDEDSSLDESDLYSLAHSYLGPLIMPMPTSPLTPALVTGGGGRKGRTKREAAANTNRPSPGGHERKLVTKLQNSERKKRGARR

SEQ ID NO: 19 - protein, 235 aa, Artificial Sequence
  Synthetic Polypeptide
  misc_feature (82)..(82): Xaa is selenocysteine
MARLLQASCLLSLLLAGFVSQSRGQEKSKAPRQMGNPQMDCHGGISGTIYEYGALTIDGEEYIPFKQYAGKYVLFVNVASYXGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 20 - protein, 158 aa, Artificial Sequence
  Synthetic Polypeptide
MLCLLLTLGVALVCGVPAMDIPQTKQDLELPKAPLRVHITSLLPTPEDNLEIVLHRWENNSCVEKKVLGEKTENPKKFKINYTVANEATLLDTDYDNFLFLCLQDTTTPIQSMMCQYLARVLVEDDEIMQGFIRAFRPLPRHLWYLLDLKQMEEPCRF

SEQ ID NO: 21 - protein, 247 aa, Artificial Sequence
  Synthetic Polypeptide
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 22 - protein, 901 aa, Artificial Sequence
  Synthetic Polypeptide
MQREAAFRLGHCHPLRIMGSVDQEEPNAHKVASPPSGPAYPDDVLDYGLKPYSPLASLSGEPPGRFGEPDRVGPQKFLSAAKPAGASGLSPRIEITPSHELIQAVGPLRMRDAGLLVEQPPLAGVAASPRFTLPVPGFEGYREPLCLSPASSGSSASFISDTFSPYTSPCVSPNNGGPDDLCPQFQNIPAHYSPRTSPIMSPRTSLAEDSCLGRHSPVPRPASRSSSPGAKRRHSCAEALVALPPGASPQRSRSPSPQPSSHVAPQDHGSPAGYPPVAGSAVIMDALNSLATDSPCGIPPKMWKTSPDPSPVSAAPSKAGLPRHIYPAVEFLGPCEQGERRNSAPESILLVPPTWPKPLVPAIPICSIPVTASLPPLEWPLSSQSGSYELRIEVQPKPHHRAHYETEGSRGAVKAPTGGHPVVQLHGYMENKPLGLQIFIGTADERILKPHAFYQVHRITGKTVTTTSYEKIVGNTKVLEIPLEPKNNMRATIDCAGILKLRNADIELRKGETDIGRKNTRVRLVFRVHIPESSGRIVSLQTASNPIECSQRSAHELPMVERQDTDSCLVYGGQQMILTGQNFTSESKVVFTEKTTDGQQIWEMEATVDKDKSQPNMLFVEIPEYRNKHIRTPVKVNFYVINGKRKRSQPQHFTYHPVPAIKTEPTDEYDPTLICSPTHGGLGSQPYYPQHPMVAESPSCLVATMAPCQQFRTGLSSPDARYQQQNPAAVLYQRSKSLSPSLLGYQQPALMAAPLSLADAHRSVLVHAGSQGQSSALLHPSPTNQQASPVIHYSPTNQQLRCGSHQEFQHIMYCENFAPGTTRPGPPPVSQGQRLSPGSYPTVIQQQNATSQRAAKNGPPVSDQKEVLPAGVTIKQEQNLDQTYLDDELIDTHLSWIQNIL

SEQ ID NO: 23 - protein, 396 aa, Artificial Sequence
  Synthetic Polypeptide
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 24 - protein, 54 aa, Artificial Sequence
  Synthetic Polypeptide
MPGKKARKNAQPSPARAPAELEVECATQLRRFGDKLNFRQKLLNLISKLFCSGT

SEQ ID NO: 25 - protein, 488 aa, Artificial Sequence
  Synthetic Polypeptide
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 26 - protein, 314 aa, Artificial Sequence
  Synthetic Polypeptide
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 27 - protein, 365 aa, Artificial Sequence
  Synthetic Polypeptide
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 28 - protein, 252 aa, Artificial Sequence
  Synthetic Polypeptide
MSSEVSARRDAKKLVRSPSGLRMVPEHRAFGSPFGLEEPQWVPDKECRRCMQCDAKFDFLTRKHHCRRCGKCFCDRCCSQKVPLRRMCFVDPVRQCAECALVSLKEAEFYDKQLKVLLSGATFLVTFGNSEKPETMTCRLSNNQRYLFLDGDSHYEIEIVHISTVQILTEGFPPGEKDIHAYTSLRGSQPASEGGNARATGMFLQYTVPGTEGVTQLKLTVVEDVTVGRRQAVAWLVAMHKAAKLLYESRDQ

SEQ ID NO: 29 - protein, 1184 aa, Artificial Sequence
  Synthetic Polypeptide
MVGTKAWVFSFLVLEVTSVLGRQTMLTQSVRRVQPGKKNPSIFAKPADTLESPGEWTTWFNIDYPGGKGDYERLDAIRFYYGDRVCARPLRLEARTTDWTPAGSTGQVVHGSPREGFWCLNREQRPGQNCSNYTVRFLCPPGSLRRDTERIWSPWSPWSKCSAACGQTGVQTRTRICLAEMVSLCSEASEEGQHCMGQDCTACDLTCPMGQVNADCDACMCQDFMLHGAVSLPGGAPASGAAIYLLTKTPKLLTQTDSDGRFRIPGLCPDGKSILKITKVKFAPIVLTMPKTSLKAATIKAEFVRAETPYMVMNPETKARRAGQSVSLCCKATGKPRPDKYFWYHNDTLLDPSLYKHESKLVLRKLQQHQAGEYFCKAQSDAGAVKSKVAQLIVIASDETPCNPVPESYLIRLPHDCFQNATNSFYYDVGRCPVKTCAGQQDNGIRCRDAVQNCCGISKTEEREIQCSGYTLPTKVAKECSCQRCTETRSIVRGRVSAADNGEPMRFGHVYMGNSRVSMTGYKGTFTLHVPQDTERLVLTFVDRLQKFVNTTKVLPFNKKGSAVFHEIKMLRRKEPITLEAMETNIIPLGEVVGEDPMAELEIPSRSFYRQNGEPYIGKVKASVTFLDPRNISTATAAQTDLNFINDEGDTFPLRTYGMFSVDFRDEVTSEPLNAGKVKVHLDSTQVKMPEHISTVKLWSLNPDTGLWEEEGDFKFENQRRNKREDRTFLVGNLEIRERRLFNLDVPESRRCFVKVRAYRSERFLPSEQIQGVVISVINLEPRTGFLSNPRAWGRFDSVITGPNGACVPAFCDDQSPDAYSAYVLASLAGEELQAVESSPKFNPNAIGVPQPYLNKLNYRRTDHEDPRVKKTAFQISMAKPRPNSAEESNGPIYAFENLRACEEAPPSAAHFRFYQIEGDRYDYNTVPFNEDDPMSWTEDYLAWWPKPMEFRACYIKVKIVGPLEVNVRSRNMGGTHRQTVGKLYGIRDVRSTRDRDQPNVSAACLEFKCSGMLYDQDRVDRTLVKVIPQGSCRRASVNPMLHEYLVNHLPLAVNNDTSEYTMLAPLDPLGHNYGIYTVTDQDPRTAKEIALGRCFDGTSDGSSRIMKSNVGVALTFNCVERQVGRQSAFQYLQSTPAQSPAAGTVQGRVPSRRQQRASRGGQRQGGVVASLRFPRVAQQPLIN

SEQ ID NO: 30 - protein, 1186 aa, Artificial Sequence
  Synthetic Polypeptide
MTRRCMPARPGFPSSPAPGSSPPRCHLRPGSTAHAAAGKRTESPGDRKQSIIDFFKPASKQDRHMLDSPQKSNIKYGGSRLSITGTEQFERKLSSPKESKPKRVPPEKSPIIEAFMKGVKEHHEDHGIHESRRPCLSLASKYLAKGTNIYVPSSYHLPKEMKSLKKKHRSPERRKSLFIHENNEKNDRDRGKTNADSKKQTTVAEADIFNNSSRSLSSRSSLSRHHPEESPLGAKFQLSLASYCRERELKRLRKEQMEQRINSENSFSEASSLSLKSSIERKYKPRQEQRKQNDIIPGKNNLSNVENGHLSRKRSSSDSWEPTSAGSKQNKFPEKRKRNSVDSDLKSTRESMIPKARESFLEKRPDGPHQKEKFIKHIALKTPGDVLRLEDISKEPSDETDGSSAGLAPSNSGNSGHHSTRNSDQIQVAGTKETKMQKPHLPLSQEKSAIKKASNLQKNKTASSTTKEKETKLPLLSRVPSAGSSLVPLNAKNCALPVSKKDKERSSSKECSGHSTESTKHKEHKAKTNKADSNVSSGKISGGPLRSEYGTPTKSPPAALEVVPCIPSPAAPSDKAPSEGESSGNSNAGSSALKRKLRGDFDSDEESLGYNLDSDEEEETLKSLEEIMALNFNQTPAATGKPPALSKGLRSQSSDYTGHVHPGTYTNTLERLVKEMEDTQRLDELQKQLQEDIRQGRGIKSPIRIGEEDSTDDEDGLLEEHKEFLKKFSVTIDAIPDHHPGEEIFNFLNSGKIFNQYTLDLRDSGFIGQSAVEKLILKSGKTDQIFLTTQGFLTSAYHYVQCPVPVLKWLFRMMSVHTDCIVSVQILSTLMEITIRNDTFSDSPVWPWIPSLSDVAAVFFNMGIDFRSLFPLENLQPDFNEDYLVSETQTTSRGKESEDSSYKPIFSTLPETNILNVVKFLGLCTSIHPEGYQDREIMLLILMLFKMSLEKQLKQIPLVDFQSLLINLMKNIRDWNTKVPELCLGINELSSHPHNLLWLVQLVPNWTSRGRQLRQCLSLVIISKLLDEKHEDVPNASNLQVSVLHRYLVQMKPSDLLKKMVLKKKAEQPDGIIDDSLHLELEKQAYYLTYILLHLVGEVSCSHSFSSGQRKHFVLLCGALEKHVKCDIREDARLFYRTKVKDLVARIHGKWQEIIQNCRPTQVSFCYTISCILNSFAEWHSSYCLK

SEQ ID NO: 31 - protein, 915 aa, Artificial Sequence
  Synthetic Polypeptide
MEKMLAGCFLLILGQIVLLPAEARERSRGRSISRGRHARTHPQTALLESSCENKRADLVFIIDSSRSVNTHDYAKVKEFIVDILQFLDIGPDVTRVGLLQYGSTVKNEFSLKTFKRKSEVERAVKRMRHLSTGTMTGLAIQYALNIAFSEAEGARPLRENVPRVIMIVTDGRPQDSVAEVAAKARDTGILIFAIGVGQVDFNTLKSIGSEPHEDHVFLVANFSQIETLTSVFQKKLCTAHMCSTLEHNCAHFCINIPGSYVCRCKQGYILNSDQTTCRIQDLCAMEDHNCEQLCVNVPGSFVCQCYSGYALAEDGKRCVAVDYCASENHGCEHECVNADGSYLCQCHEGFALNPDKKTCTRINYCALNKPGCEHECVNMEESYYCRCHRGYTLDPNGKTCSRVDHCAQQDHGCEQLCLNTEDSFVCQCSEGFLINEDLKTCSRVDYCLLSDHGCEYSCVNMDRSFACQCPEGHVLRSDGKTCAKLDSCALGDHGCEHSCVSSEDSFVCQCFEGYILREDGKTCRRKDVCQAIDHGCEHICVNSDDSYTCECLEGFRLAEDGKRCRRKDVCKSTHHGCEHICVNNGNSYICKCSEGFVLAEDGRRCKKCTEGPIDLVFVIDGSKSLGEENFEVVKQFVTGIIDSLTISPKAARVGLLQYSTQVHTEFTLRNFNSAKDMKKAVAHMKYMGKGSMTGLALKHMFERSFTQGEGARPLSTRVPRAAIVFTDGRAQDDVSEWASKAKANGITMYAVGVGKAIEEELQEIASEPTNKHLFYAEDFSTMDEISEKLKKGICEALEDSDGRQDSPAGELPKTVQQPTESEPVTINIQDLLSCSNFAVQHRYLFEEDNLLRSTQKLSHSTKPSGSPLEEKHDQCKCENLIMFQNLANEEVRKLTQRLEEMTQRMEALENRLRYR

SEQ ID NO: 32 - protein, 101 aa, Artificial Sequence
  Synthetic Polypeptide
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 33 - protein, 266 aa, Artificial Sequence
  Synthetic Polypeptide
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 34 - protein, 175 aa, Artificial Sequence
  Synthetic Polypeptide
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 35 - protein, 655 aa, Artificial Sequence
  Synthetic Polypeptide
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 36 - protein, 162 aa, Artificial Sequence
  Synthetic Polypeptide
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 194 aa, Artificial Sequence
  Synthetic Polypeptide
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 38 - protein, 292 aa, Artificial Sequence
  Synthetic Polypeptide
MDSKYSTLTARVKGGDGIRIYKRNRMIMTNPIATGKDPTFDTITYEWAPPGVTQKLGLQYMELIPKEKQPVTGTEGAFYRRRQLMHQLPIYDQDPSRCRGLLENELKLMEEFVKQYKSEALGVGEVALPGQGGLPKEEGKQQEKPEGAETTAATTNGSLSDPSKEVEYVCELCKGAAPPDSPVVYSDRAGYNKQWHPTCFVCAKCSEPLVDLIYFWKDGAPWCGRHYCESLRPRCSGCDEIIFAEDYQRVEDLAWHRKHFVCEGCEQLLSGRAYIVTKGQLLCPTCSKSKRS